(19) United States
(12) Patent Application Publication
(10) Pub. No.: US 2011/0256565 A1
(43) Pub. Date: Oct. 20, 2011

(54) METHODS FOR DIAGNOSIS AND TREATMENT OF NEURODEGENERATIVE DISEASES OR DISORDERS

(76) Inventors: Eric A. SCHON, Bronx, NY (US); Estela AREA-GOMEZ, New York, NY (US); Michael P. YAFFE, San Diego, CA (US)

(21) Appl. No.: 12/956,939

(22) Filed: Nov. 30, 2010

Related U.S. Application Data
(63) Continuation-in-part of application No. PCT/US09/45879, filed on Jun. 1, 2009.
(60) Provisional application No. 61/057,707, filed on May 30, 2008.
(60) Provisional application No. 61/386,350, filed on Sep. 24, 2010.

(51) Int. Cl. C12Q 1/60; C12Q 1/37; C12Q 1/54; C12Q 1/48; C12Q 1/26; C12Q 1/42; C12Q 1/02; C12Q 1/18

(52) U.S. Cl. 435/11; 435/29; 435/23; 435/32; 435/15; 435/25; 435/21; 435/14

(57) ABSTRACT

The present invention provides methods that are useful for the diagnosis of neurodegenerative disease or disorder and for the screening of compounds or therapeutic agents for treating a neurodegenerative disease or disorder. The methods pertain in part to the correlation of a neurodegenerative disease or disorder with abnormal or altered endoplasmic reticulum-mitochondrial-associated membranes (ER-MAM) integrity.

This application is a continuation-in-part application and claims the benefit of and priority to U.S. provisional patent application Ser. No. 61/057,707 filed May 30, 2008, International PCT application PCT/US09/45879, filed Jun. 1, 2009, and U.S. provisional patent application Ser. No. 61/386,350, filed Sep. 24, 2010, the disclosure of all of which is hereby incorporated by reference in its entirety for all purposes.

This invention was made with government support under NS39854 and HD32062 awarded by the National Institutes of Health. The government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### BACKGROUND OF THE INVENTION

Neurodegenerative diseases are a major public health concern. The increasing number of patients with neurodegenerative diseases imposes a major financial burden on health systems around the world.

Alzheimer disease (AD) is a neurodegenerative dementing disorder of late onset characterized by progressive neuronal loss, especially in the cortex and the hippocampus (Goedert M, Spillantini M G (2006) 314:777-781). The two main histopathological hallmarks of AD are the accumulation of extracellular neuritic plaques, consisting predominantly of β-amyloid (Aβ), and of neurofibrillary tangles, consisting mainly of hyperphosphorylated forms of the microtubule-associated protein tau (Goedert M, Spillantini M G (2006) Science 314:777-781). The majority of AD is sporadic (SAD), but variants in apolipoprotein E (ApoE) (Goedert M, Spillantini M G (2006) Science 314:777-781) and in SORL1, a neuronal sorting receptor (Rogaeva et al., (2007) Nature Genet. 39:168-177), can be predisposing genetic factors. At least three genes have been identified in the familial form (FAD): amyloid precursor protein (APP), presenilin-1 (PS1), and presenilin-2 (PS2).

More than half of the patients with dementia have Alzheimer's disease (AD). The prevalence for AD between the age 60-69 years is 0.3%, 3.2% between that age 70-79 years, and 10.8% between 80-89 years of age (Rocca, Hofman et al. 1991). Survival time after the onset of AD is in the range of 5 to 12 years (Friedland, 1993).

Although various diagnostic tests exist to detect AD (see U.S. Pat. Nos. 5,508,167, 6,451,547, 6,495,335 and 5,492,812), a major hurdle in developing anti-AD drugs has the lack of a defined causative event in the genesis of the disease. Thus there remains a need for improved methods diagnosis of AD and for methods to identify compounds suitable for the treatment, prevention or inhibition of AD.

### SUMMARY OF THE INVENTION

The present invention provides methods that are useful for the diagnosis of Alzheimer's disease (AD) and for the screening of compounds or therapeutic agents for treating AD. The methods pertain in part to the correlation of AD with abnormal or altered endoplasmic reticulum-mitochondrial-associated membranes (ER-MAM) integrity.

In one aspect, abnormal or altered ER-MAM integrity in AD cells is reflected by an increase in communication between the ER and mitochondria in the cell as compared to non-AD cells, or an increase in the “thickness” or cholesterol content in ER-MAM in the cell as compared to non-AD cells. Without being bound by theory, it is believed that abnormal or altered ER-MAM content or thickness causes a multitude of downstream effects, which downstream effects themselves can be correlated with AD. Additionally, abnormal or altered ER-MAM can be caused by upstream effects that are correlated with AD. Such upstream and downstream effects that correlate with abnormal or altered ER-MAM levels or thickness can be considered indicators of altered ER-MAM integrity.

Thus, in various aspects, an indicator of altered ER-MAM integrity can be any detectable parameter that directly or indirectly relates to a condition, process, or other activity involving ER-MAM and that permits detection of altered or abnormal ER-MAM function or state (as compared to ER-MAM from normal or non-AD cells) in a biological sample from a subject or biological source (detection can also be in the subject or animal model). Exemplary indicators of altered ER-MAM integrity can be, for example, a functional activity or expression level of an ER-MAM-associated protein, subcellular localization of an ER-MAM-associated protein, mitochondrial morphology in a cell, mitochondrial localization in a cell, communication between the ER and mitochondria in a cell, “thickness” of ER-MAM in a cell as reflected by cholesterol content in the ER-MAM, in situ morphology of ER-MAM in a cell, or other criteria as provided herein. In addition, the present methods can involve one or more of the above-mentioned indicators of altered ER-MAM integrity in combination with one or more general indicators of AD. General indicators of AD include, but are not limited to, altered APP processing, amyloid toxicity, tau hyperphosphorylation, altered lipid and cholesterol metabolism, altered glucose metabolism, aberrant calcium homeostasis, glutamate excitoxicity, inflammation and mitochondrial dysfunction.

In one aspect, the invention provides a method for diagnosing a neurodegenerative disease, the method comprising: (a) obtaining one or more cells from a subject suspected of having the neurodegenerative disease, and (b) testing the cells from (a) for one or more indicators of altered ER-MAM integrity. The neurodegenerative disease can be, for example, a dementia-related disease, such as Alzheimer's Disease. For example, the cells obtained in step (a) can be, but are not limited to, an AD model cell, a neuron, a fibroblast, a skin biopsy, a blood cell (e.g. a lymphocyte), an epithelial cell and cells found in urine sediment. The one or more indicators of altered ER-MAM integrity can comprise, for example, (1) the ratio of perinuclear mitonchondria to non-perinuclear mitochondria is greater in the cells from the subject as compared to cells from a normal control; (2) the communication between the ER and mitochondria or thickness of ER-MAM is increased in the cells from the subject as compared to cells from a normal control; (3) the ratio of punctate mitochondria to non-punctate mitochondria is greater in the cells from the subject as compared to cells from a normal control; and/or (4) the amount of mitochondria are in the extremities of the cells from the subject are reduced as compared to cells from a normal control.

In one aspect, the methods for diagnosing at least comprises a characteristic of ER-MAM itself, such as the communication between the ER and mitochondria, the lipid composition of ER-MAM, the cholesterol composition of ER-MAM, and the protein composition of ER-MAM.

In one aspect, the method further comprises testing the subject for one or more of: (1) elevated cholesterol levels; (2) altered brain glucose metabolism; (3) altered lipid metabolic profiles; (4) significant alterations in PC and PE; and/or (5) disturbed calcium homeostasis.

In one aspect of the method, the testing of the communication between the ER and mitochondria can comprise determining whether the level of protein-protein interactions between MAM-associated proteins is increased in the cells from the subject as compared to cells from a normal control. This can involve, for example, (1) transfecting the cells obtained from the subject and the control cells with one or more expression vectors that express a DGAT2-CFP fusion protein and an SCD1-YFP fusion protein (or other FRET proteins); (2) illuminating the transfected cells with an appropriate wavelength of light to excite the YFP; and (3) comparing the fluorescent signal levels emitted from CFP in the transfected cells from the subject and the control, wherein lower levels from the subject as compared to control indicates altered MAM-integrity.

In one aspect, the invention provides a method for diagnosing familial Alzheimer's Disease, the method comprising determining whether the communication between the ER and mitochondria is increased in cells from a subject as compared to cells from a normal control, wherein the subject has not been subjected to any genetic screen for PS1, PS2, or APP mutations.

In one aspect, the invention provides a method for selecting a test compound for treating Alzheimer's Disease, the method comprising: (a) contacting Alzheimer's Disease model cells with and without a test compound, and (b) selecting the test compound if it can cause an improvement in one or more indicators of ER-MAM integrity in the cells as compared to cells not contacted with the test compound. The Alzheimer's Disease model cells can comprise, but are not limited to, cells with a PS1 mutation, cells with a PS2 mutation, cells with an APP mutation, human skin fibroblasts derived from patients carrying FAD-causing presenilin mutations, mouse skin fibroblasts, cultured embryonic primary neurons, and any other cells derived from PS1-knock out transgenic mice (containing null mutation in the PS1 gene), cells having AD-linked familial mutations, cells having genetically associated AD allelic variants, cells having sporadic AD, cells having ApoE mutations or cells having mutations associated with sporadic AD. Exemplary AD mutations include, but are not limited to APP V717 I APP V717 F, APP V717G, APP A682G, APP K/M670/671N/L, APP A713V, APP A713T, APP E693G, APP T673A, APP N665D, APP I 716V, APP V715M, PS1 113Δ4, PS1 A79V, PS1 V82L, PS1 V96F, PS1 113Δ 4, PS1 Y115C, PS1 Y115H, PS1 T116N, PS1 P117L, PS1 E120D, PS1 E120K, PS1 E123K, PS1 N135D, PS1 M139, PS1 I M139T, PS1 M139V, I 143F, PS1 1143T, PS1 M461, PS1 I M146L, PS1 M146V, PS1 H163R, PS1 H163Y, PS1 S169P, PS1 S169L, PS1 L171P, PS1 E184D, PS1 G209V, PS1 I 213T, PS1 L219P, PS1 A231T, PS1 A231V, PS1 M233T, PS1 L235P, PS1 A246E, PS1 L250S, PS1 A260V, PS1 L262F, PS1 C263R, PS1 P264L, PS1 P267S, PS1 R269G, PS1 R269H, PS1 E273A, PS1 R278T, PS1 E280A, PS1 E280G, PS1 L282R, PS1 A285V, PS1 L286V, PS1 S290C (Δ9), PS1 E318G, PS1 G378E, PS1 G384A, PS1 L392V, PS1 C410Y, PS1 L424R, PS1 A426P, PS1 P436S, PS1 P436Q, PS2 R62H, PS2 N141I, PS2 V148I, or PS2 M293V.

The improvement in one or more indicators of ER-MAM integrity can comprise, for example, (1) the ratio of perinuclear mitonchondria to non-perinuclear mitochondria is decreased in the cells contacted with the test compound as compared to the cells not contacted with the test compound; (2) the communication between the ER and mitochondria is decreased in the cells contacted with the test compound as compared to the cells not contacted with the test compound; (3) the ratio of punctate mitochondria to non-punctate mitochondria is lower in the cells contacted with the test compound as compared to the cells not contacted with the test compound; (4) the amount of mitochondria in the extremities of the cells are increased in the cells contacted with the test compound as compared to the cells not contacted with the test compound; (5) the amount of phosphatidylserine converted to phosphatidylethanolamine is decreased in the cells contacted with the test compound as compared to the cells not contacted with the test compound; (6) the level of association between MAM-associated proteins is decreased in the cells contacted with the test compound as compared to the cells not contacted with the test compound; (7) the level of one or more MAM-associated proteins localized to the ER-MAM compartment is decreased as compared to the cells not contacted with the test compound; and/or (8) the activity level of one or more MAM-association proteins is altered as compared to the cells not contacted with the test compound.

In one aspect, the methods for selecting or screening for test compounds at least comprises testing a characteristic of ER-MAM itself, such as whether the test compound can affect the communication between the ER and mitochondria, the lipid composition of ER-MAM, the cholesterol composition of ER-MAM, or the protein composition of ER-MAM.

In one aspect of the screening methods, an increase in association between MAM-associated proteins can be between Diacylglycerol-O-acyltransferase 2 (DGAT2) and stearoyl-CoA desaturase 1 (SCD1).

In one aspect of the screening methods, measuring the association between MAM-associated proteins is conducted by (i) transfecting the Alzheimer's Disease model cells with vector(s) that express fusion proteins that comprise a MAM-associated protein or portion thereof and a FRET fluorescent donor or acceptor protein, (ii) exciting the FRET donor, and (iii) measuring the amount of fluorescence emitted from the FRET acceptor.

In one aspect of the screening methods, the method further comprises testing whether the test compound can cause a decrease in the amount of reactive oxygen species in the cells contacted with the test compound as compared to the cells not contacted with the test compound.

In one aspect, the invention provides a method for selecting test compounds for treating Alzheimer's Disease, the method comprising: (a) contacting Alzheimer's Disease model cells with cinnamycin in an amount sufficient to cause cell death of normal cells with and without a test compound, and (b) selecting the test compound if it causes the Alzheimer's Disease cells to be more susceptible (or have a different susceptibility) to cinnamycin-mediated death.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. PS1 fibroblasts are smaller than controls. Both photos at 40×. Red, mitochondria; green, microtubules.

FIG. 2. Control mitochondria are elongated; PS1 mitochondria are more punctate. 100× magnification.

FIG. 3 Western blot analysis of subcellular fractions of mouse liver and brain. Thirty mg of total protein were loaded in each lane and probed with the indicated antibodies.

FIG. 4. Immunohistochemistry to detect PS1 in cells. Cells were stained with MT Red (red) and with anti-PS1 (green); the merged photo is at bottom (yellow if MT Red and PS1 are co-localized). FIG. 4 A-B. Comparison of various fixation techniques. FIG. 4A. When cells were treated using “standard” techniques (fixation with PF and permeabilization with TX-100), there was poor co-localization of the two signals (the orange staining in the merge panel is the non-specific overlap of the MT Red stain with the diffuse anti-PS1 stain). 40×. FIG. 4B: However, if TX-100 was replaced with MeOH, whether in the absence or presence of PF, there was excellent colocalization with a subset of mitochondria that are predominantly perinuclear. Asterisks mark mitochondria that are cortical and do not co-localize with PS1. Note that PS1 does not stain mitochondria exclusively, as some non-mitochondrial staining is still observed. 40×. FIG. 4 C-E: Localization of PEMT and PS1 in human fibroblasts. MeOH fixation. As in FIG. 4B, both PS1 (C) and PEMT (D) co-localized with MT Red, mainly in regions proximal to the nucleus (yellow arrowheads), with a lower degree of co-localization in more distal mitochondria (red arrowheads). 63×. FIG. 4E. When stained simultaneously for PEMT (red) and PS1 (green), both proteins showed a high degree of co-localization, implying that PS1, like PEMT, is in the MAM. 100×.

FIG. 5. Proportion of ER, MAM, and mitochondria in control and FAD fibroblasts. Asterisks denote significance of avg±SD.

FIG. 6. Mitochondrial morphology in FAD^{PS1} fibroblasts. FIG. 6A: Example of staining of control and FAD^{PS1} (mutation indicated) fibroblasts with MTred (red) and anti-tubulin (green) (63×). FIG. 6B: Mitochondria in control cells have a reticulated network, whereas those in FAD^{PS1} (A246E) cells are more punctate (100×). FIG. 6C: Example of quantitation of the number of mitochondria located outside the circular region; n, # of cells examined; asterisks denote significance of avg±SEM (p<0.05).

FIG. 7. Mitochondrial morphology in COS7 cells expressing stably-transfected wild type (WT) or mutated (A246E) PS1 stained with MTred (red) and decorated with anti-tubulin (green). FIG. 7A: Transfection with empty vector. FIG. 7B: Transfection with wild-type PS1. FIG. 7C. Transfection with mutated (A246E) PS1.

FIG. 8. Mitochondrial morphology in FAD^{PS1} fibroblasts in PS1-knockdown mouse embryonic fibroblasts. FIG. 8A: Example of staining of control and FAD^{PS1} (A246E) fibroblasts with MTred (red) and anti-tubulin (green) (63×). FIG. 8B: Example of quantitation of the number of mitochondria located in the cell periphery (see Methods). Three replicate experiments were performed; n, number of cells examined; error bars denote standard error of the mean (SEM); asterisks denoted significant difference vs. control (p<0.05). FIG. 8C Mitochondria in control cells are a reticulated network, whereas those in FAD^{PS1} (A246E) cells are more punctate (100×). FIG. 8D: Relative proportion of protein in ER, ER-MAM, and mitochondrial fractions in control and FAD^{PS1} (A246E) human fibroblasts; error bars denote standard deviation; asterisks denote significant difference vs. control (p<0.05). FIG. 8E: Example of morphology in PS1-knockdown (shRNA) (>75% knockdown; right panel) and mismatch control (left panel) MEFs. Note “perinuclear” phenotype in PS1-knockdown cells. 63×. FIG. 8F: Quantitation as in (B).

FIG. 9. ApoE and APP are enriched in MAM. T, Total cellular protein; CM, crude mitochondria; PM, plasma membrane.

FIG. 10. Western blot analysis of subcellular fractions of mouse liver. Localization and molecular masses of the indicated polypeptides were determined using the antibodies listed at right. Thirty μg of protein were loaded into each lane.

FIG. 11. Immunolocalization of PEMT in human fibroblasts (FIG. 11A) Fixation with PF and permeabilization with TX100. Note poor co-localization of the two signals (the orange staining in the merge panel is the non-specific overlap of the MTred stain with the diffuse anti-PS1 stain). FIG. 11B: Fixation and permeabilization with MeOH. Note co-localization of PEMT and MTred in the perinuclear region (yellow arrowheads) but not in more distal regions (red arrowheads). Images captured by confocal microscopy (100×).

FIG. 12. Immunolocalization of PS1 (C-terminal antibody; Sigma P7854) in mouse 3T3 cells (upper and middle panels) and in human fibroblasts (lower panels). FIG. 12A: Fixation in PF and permeabilization in TX100. FIG. 12B: Fixation in PF and permeabilization in MeOH. FIG. 12C: Fixation and permeabilization in MeOH. Arrowheads as in FIG. 12A. Note similarity of the co-localization pattern to that with PEMT in FIG. 12A. Note also that the similarity of the results in (b) and (c) imply that it is the TX100, not the PF, that is responsible for the diffuse pattern of immunostain shown in (a). 63×

FIG. 13. Immunolocalization of PEMT and PS1 in human fibroblasts (FIG. 13A) Fixation with PF and permeabilization with TX100. FIG. 13B: Fixation and permeabilization with MeOH. Note the high degree of colocalization of the two signals in both sets of images. Images captured by confocal microscopy (100×).

FIG. 14. Immunohistochemistry to detect PS1 is various cells. Cells were stained with MTred (red) and with anti-PS1 (green); merged photos are at light (yellow if MTred and PS1 are co-localized). Cells were fixed and permeabilized with MeOH. FIG. 14A: Mouse 3T3 cells immunostained with Ab P4985 that detects the N-terminus of PS1. FIG. 14B: Rat neurons immunostained with Ab P7854 that detects the C-terminus of PS1. Note the co-localization PS1 with the MTred signal, mainly in mitochondria located proximal to the nucleus (yellow arrowheads); there is a lower degree of co-localization in more distal mitochondria (red arrowheads) Immunostaining of P7854 was suppressed in the presence of the peptide epitope used to generate the antibody, confirming its specificity. FIG. 14C: Human 293T cells immunostained with Ab P7854, photographed in a plane of focus to reveal the localization of PS1 to adherens junctions in confluent cells (arrowheads). Note absence of co-localization of PS1 with MTred in adherens junctions. 63×

FIG. 15. Western blot analysis of the subcellular fractions of interest (ER-MAM, mitochondria, and ER) from mouse liver and brain. FIG. 15A: Thirty μg of total liver protein were loaded in each lane, and were probed using the indicated marker antibodies (at right; approximate mass in parentheses) and various PS1 antibodies (at left). SSRI, signal sequence receptor α; CANX, calnexin; NDUFA9, subunit of mitochondrial respiratory complex I. FIG. 15B: Same as in (A), using brain. FIG. 15C: Relative abundance of each fraction, as determined by Bradford protein assay; the approximate averages are also indicated below each lane in (A) and (B).

FIG. 16. Co-localization of MTred, calnexin, and PS1 (antibody P7854) in human fibroblasts, viewed by confocal microscopy (63×). Regions a, b, and c within ovals are discussed in the text.

FIG. 17. Mitochondrial morphology in mouse embryonic fibroblasts deficient in PS1 due to sh-RNA treatment. Center. Western blot analysis of shRNA clones. Lanes 1-3, dilutions to quantitate PS1; lane 4, knockdown of PS1 compared to control in lane 5. Anti-tubulin loading controls at bottom. Side panel. MTred staining of test (left) vs. control (right) cells. Note “perinuclear” phenotype in PS1-knockdown cells. The specificity of the shRNA primer was confirmed by transducing a mismatch shRNA.

FIG. 18. Mitochondria are more perinuclear in PS1 fibroblasts than in controls. Red, mitochondria; green, microtubules.

FIG. 19. gamma-Secretase activity of mouse liver and brain fractions.

FIG. 20. Mitochondrial dynamics in PS1-knockdown neuroblastoma cells. Note the severely reduced accumulation of mitochondria in varicosities and at branch points (arrowheads) in cell processes in PS1-KD vs. control cells. In the enlargements in the center panels, mitochondria were enriched in “varicosities” (arrowheads) or uniformly distributed (brackets) in neuronal processes of control cells, but were markedly reduced in numbers, density, and intensity in PS1— KD cells. Quantification of MT Red staining (plots of intensity vs. length by Image J) in each process is at the right of the respective enlargements (note corresponding regions marked a-d). The plots are shown for illustrative purposes only, as they have different intensity scales and are not comparable quantitatively.

FIG. 21. Mitochondria in the hippocampal CA1 region of an FAD^{PS1} patient (A434C). Immunohistochemistry to detect the FeS subunit of complex III. Note perinuclear “rings” of mitochondria (arrowheads) and the dearth of mitochondria in the distal parts of the cell body (asterisks) in patient vs. control,

FIG. 22. Western blot of selected mitochondrial proteins. Rieske and Core B are subunits of complex III of the respiratory chain.

FIG. 23. PS1-mutant mouse MEFs have increased ROS. MitoSox staining is increased in both single- and double-KO cells.

FIG. 24. Bioenergetics. FIG. 24A: Oxygen consumption. FIG. 24B: ATP synthesis.

FIG. 25. Ca2+ homeostasis in control and PS1-knockdown cells. FIG. 25A: Cytosolic Ca2+ using fura-2 measured at 340/380 nm (example in inset). Note increase (vertical arrow) and delayed release (horizontal arrows) of Ca2+ upon ATP addition. FIG. 25B: Mitochondrial Ca2+ using pericams. Blue cells indicate elevated Ca2+ (example in inset). Note higher [Ca2+] in PS1-KD cells. F/F0, ratio of fluorescence at time x to that at time 0.

FIG. 26. Mitochondrial morphology in PACS2-KO mice. MT Red (red) and microtubule (green) staining of wt and KO MEFs. Note perinuclear distribution of mitochondria, and shape changes (“doughnuts” in enlargement of boxed region) in the KO cells.

FIG. 27. Analysis of PS1 and Ab in mouse brain cell fractions. FIG. 27A. Schematic of fractions associated with ER, MAM, and mitochondria. FIG. 27B. Western blots of the indicated fractions (15 mg loaded in each lane), using the indicated antibodies. Note concentration of PS1 in MAM, whereas Ab appears to be concentrated in those mitochondria that are associated with ER (“MER”); notably, neither PS1 nor Ab are associated with “free” mitochondria.

FIG. 28. Western blot analysis of subcellular fractions of mouse brain. Thirty μg of total protein were loaded in each lane. FIG. 28A: Localization and predicted molecular masses of the indicated polypeptides were determined using the antibodies listed at right (see text). PM, plasma membrane. FIG. 28B: Fractions were probed using the indicated antibodies against PS1 (Calbiochem PC267) and PS2 (Cell Signalling 2192) and to other components of the γ-secretase complex. In the blots shown here, the intensity of both the PS1 and the PS2 signals in MAM was enriched ˜8-fold over that in the ER.

FIG. 29. γ-Secretase activity assays. FIG. 29A: Activity using a FRET-based assay, in the absence and presence of Compound E, a γ-secretase inhibitor. Serial dilutions of the indicated subcellular fractions from mouse brain were assayed for APP cleavage activity (in arbitrary units/μg protein). Bars, SD; asterisk denotes significant difference in MAM compared to the other fractions (P<0.05); n=3 for all fractions. FIG. 29B: Activity using Western blotting to detect AICD, in the absence and presence of Compound E. The identity of the lower bands in the first and third lanes is unknown. The specificity of the AICD signal was confirmed in PS1/PS2 double-knockout mouse embryonic fibroblasts.

FIG. 30. Immunocytochemistry to detect FACL4 and presenilins in mammalian cells. FIG. 30A: Double-staining of human fibroblasts with MT Red and anti-FACL4. FACL4 co-localizes with MT Red in regions proximal to the nucleus (yellow arrowhead), with a lower degrees of co-localization in more distal mitochondria (red arrowhead). In an enlarged view of the perinuclear region from another merged field (rightmost panel), note discrete regions where the red and green signals (e.g. arrowheads) are in apposition and do not overlap. FIG. 30B: Double-staining of human fibroblasts with MT Red and anti-PS1. Note the similarity of the co-localization pattern to that seen with FACL4. FIG. 30C: Double-staining of human fibroblasts with anti-FACL4 (red) and anti-PS1 (green). There is significant overlap between the red and green signals, even in the enlarged merged view of the perinuclear region, implying that both proteins are in the same compartment (i.e. MAM). FIG. 30D: Double-staining of mouse 3T3 cells with MT Red and anti-P S2. Note the similarity of the co-localization pattern to that seen in panels A and B. FIG. 30E: Double-staining of confluent COS-7 cells human with MT Red and anti-PS1, photographed in a plane of focus to reveal the localization of PS1 to adherens junctions (AJ; arrowheads). The MT Red staining is fuzzy because almost all mitochondria are below the plane of focus. Note the absence of co-localization of PS1 with MT Red in AJ. Immunostaining of anti-PS1 (Ab P7854) was suppressed in the presence of the peptide epitope used to generate the antibody, confirming its specificity.

FIG. 31. Phospholipid biosynthetic pathways.

FIG. 32. Incorporation of 3H-Ser into phospholipids. FIG. 32A: Time course (0, 2, 4, 6 hours) of phospholipid synthesis in PS1+PS2 double knockout mouse embryonic fibroblasts (MEFs; courtesy of Bart de Strooper; Herreman et al. (1999) Proc. Natl. Acad. Sci. USA 96:11782), in medium lacking Etn and Ser. Note the increase in PtdSer and PtdEtn (and also PtdCho) in DKO MEFs vs. control MEFS. FIG. 32B: Same as in A, but using a different source of MEFs, from Alan Bernstein (Donoviel et al. (1999) Genes Dev. 13:2801). FIG. 32C: Time course (0, 1, 3 hours) of phospholipid synthesis in MEFs were null for PACS2, a gene required for the transport of proteins from the ER across the MAM to mitochondria (Simmen et al. (2005) EMBO J. 24:717; a gift of Gary Thomas). As such, PACS2 KO cells should be defective in MAM transport to mitochondria. Note the increase in PtdSer in PACS2-KO MEFS, but a decrease in PtdEtn and PtdCho, consistent with loss of MAM-mitochondrial communication. FIG. 32D: Fibroblasts from a FAD patient with a mutation in PS1 (A246E) and from PS1-KO MEFs were treated with 3H-Ser for 30 min at 37° C. and the ratio of PtdEtn/PtdSer was measured.

FIG. 33. Cholesterol content. FIG. 33A: Free and esterified cholesterol in mouse brain fractions. FIG. 33B: Free and esterified cholesterol in the crude mitochondrial fraction (essentially mitochondria+MAM) from WT and PS1-knock-in mice.

FIG. 34. Mitochondrial dynamics in PS1-knockdown (PS1-KD) neuroblastoma cells. Note the severely reduced accumulation of mitochondria in varicosities and at branch points (arrowheads) in cell processes in PS1-KD vs. control cells. In the enlargements at right (from other cells not shown here), mitochondria were enriched in “varicosities” (arrowheads) or uniformly distributed (brackets) in neuronal processes of control cells, but were markedly reduced in numbers, density, and intensity in PS1-KD cells.

FIG. 35. Mitochondria in the hippocampal CA1 region of an FAD^{PS1} patient (A434C). Immunohistochemistry (FeS subunit of complex III) to detect mitochondria. Note perinuclear “rings” of mitochondria (arrowheads) and the dearth of mitochondria in the distal parts of the cell body (asterisks) in patient vs. control, Left, low power; right, four neurons (a-d) at higher magnification.

FIG. 36. ApoE and APP are enriched in MAM. PM, plasma membrane.

FIG. 37. Western blot to detect the indicated proteins in standard subcellular fractionation of mouse tissues to isolate PM, MAM, mitochondria, and ER fractions (13). Na/K-ATPase and pancadherin are enriched in the PM; ACAT1 is enriched in the MAM. Note that the MAM fraction is essentially devoid of the PM marker.

FIG. 38. MAM displays the features of a lipid raft. FIG. 38A: Mouse liver Percoll-purified MAM treated with or without TX100 prior to centrifugation through a second Percoll gradient. The low density fraction (arrow) is detergent-resistant but solubilizable by methanol (MeOH), implying that it is a DRM. FIG. 38B: Western blot of fractions (as in FIG. 38A) from a 5%-30% sucrose gradient (triangle; lower density at left). The pellet (P) denotes TX100-soluble material. FIG. 38C: Western blot of gradient fractions of mouse liver PM and crude mitochondrial extract (CM) to detect Src (PM marker) and Pemt (MAM marker).

FIG. 39. Cholesterol metabolism in normal mouse brain (A, B) and in presenilin-mutant cells (CF). FIG. 39A: Total cholesterol (n=3, except PM rafts [n=2]). FIG. 39B: ACAT1 activity (n=4). Inset: Western blot to detect ACAT1; 20 mg protein loaded in each lane. FIGS. 39C-F: Cholesterol metabolism in presenilin-mutant MEFs. FIG. 39C: Content of cholesterol species. Numbers denote ˜ng/μg protein. FIG. 39D: EM of a DKO MEF. Note accumulation of lipid droplets (asterisks). M, mitochondrion. FIG. 39E: ACAT activity in MEFs after 6 h incubation with 3H-oleoyl-CoA (n=3). FIG. 39F: ACAT activity in isolated MAM after 20 min incubation (n=4). Error bars, SD; asterisks denote significant difference (p<0.05).

FIG. 40. Western blot to detect SSRα (signal sequence receptor a; a marker for bulk ER) and NDUFA9 (a subunit of complex I of the respiratory chain; a marker for mitochondria) in fractions from a 5%-30% sucrose gradient (triangle denotes increasing density from left to right) of purified bulk ER and mitochondria after treatment with 1% Triton X-100 at 4° C. for 1 hour. Note than neither bulk ER nor mitochondria contain low density DRMs; almost all of both fractions, as determined by the marker proteins, was in the detergent-soluble pellet (P).

FIG. 41. Phospholipid synthesis in MEFs. FIG. 41A: Synthesis of PtdSer and PtdEtn after 3H-Ser labeling for the indicated times (hours) (n=3). FIG. 41B: Pulse-chase. MEFs labeled for 1 h with 3H-Ser; chase with cold Ser (n=3). FIG. 41C: Phospholipid synthesis in crude mitochondria (n=3 or 4) Error bars, SE; p<0.05. FIG. 41D: Cinnamycin sensitivity in MEFs. Left: Live/dead assay (1 μM cinnamycin for 10 min at 37° C.). Right: Cinnamycin-sensitivity assay (10 min at 37° C.).

FIG. 42. Electron microscopy of WT (FIGS. 42 B and D) and DKO (FIG. 42A, C, E) MEFs. Note increased length of regions of contact between ER and mitochondria (M) (arrowheads) in DKO MEFs, and ER “sandwiched” between two mitochondria (FIG. 42E). FIG. 42F: Quantitation of ER-mitochondrial contact lengths.

FIG. 43. Cinnamycin sensitivity in fibroblasts from FAD patients with pathogenic mutations in PS1 (PS1) and from sporadic AD (SAD) patients. Cells were treated with 1 μM cinnamycin for 10 min at 37° C., and viabitlity was monitored by Live/Dead assay. Note especially the cinnamycin sensitivity in the SAD patients, who presumably have normal PS1 expression and function.

### DETAILED DESCRIPTION OF THE INVENTION

The issued patents, applications, and other publications that are cited herein are hereby incorporated by reference to the same extent as if each was specifically and individually indicated to be incorporated by reference.

### DEFINITIONS

The singular forms “a,” “an,” and “the” include plural reference unless the context clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the term “presenilin” refers to the family of related multi-pass transmembrane proteins that can function as a part of the γ-secretase protease complex. The term presenilin includes presenilin-1 (PS1) and presenilin-2 (PS2). There are at least 7 members of the presenilin family in humans including; PS1 (gene PSEN1; Chr 14q24.2), PS2 (gene PSEN2; Chr 1q42.13), PSL1 (gene SPPL2B; Chr 19p13.3), PSL2 (gene SPPL2A Chr 15q21.2; thought to be in endosomes), PSL3 (gene HM13; Chr 2001.21), PSL4 (gene SPPL3, Chr 12q24.31), PSL5 (gene IMP5; Chr 17q21.31; no introns)

The present invention provides methods that are useful the diagnosis of AD in a subject and methods useful for the identification of compounds or therapeutic agents for treating AD. The methods of the present invention pertain in part to the correlation of AD with abnormal or altered endoplasmic reticulum-mitochondrial-associated membrane (ER-MAM) integrity. As used herein, “altered ER-MAM integrity” or “abnormal ER-MAM integrity” are used interchangeably, and can refer to any condition or state, including those that accompany AD, where any structure or activity that is directly or indirectly related to a ER-MAM function has been changed relative to a control or standard.

In AD cells, abnormal or altered ER-MAM integrity can be, for example, an increase in communication between the ER and mitochondria in the cell as compared to non-AD cells, or an increase in the “thickness” or cholesterol content in ER-MAM in the cell as compared to non-AD cells. As used herein, an amount of communication between the ER and mitochondria can refer to an amount of ER-MAM function or activity. Thus, in certain embodiments described herein, an increase in communication between the ER and mitochondria refers to an increase in ER-MAM function or activity, whereas a decrease in communication between the ER and mitochondria refers to an decrease in ER-MAM function or activity Without being bound by theory, it is believed that abnormal or altered ER-MAM causes a multitude of downstream effects, which downstream effects themselves can be correlated with AD. Thus, in some embodiments, the present methods comprise the detection or assaying for an increased or decreased level of at least one indicator of altered ER-MAM integrity.

According to the present invention, an “indicator of altered ER-MAM integrity” can be any detectable parameter that directly or indirectly relates to a condition, process, or other activity involving ER-MAM and that permits detection of altered or abnormal ER-MAM function or state (as compared to ER-MAM from normal or non-AD cells) in a biological sample from a subject or biological source. Detection can also be in the subject or animal model. For example, indicators of altered ER-MAM integrity can be, but are not limited to, a functional activity or expression level of an ER-MAM-associated protein, subcellular localization of an ER-MAM-associated protein, mitochondrial morphology in a cell, mitochondrial localization in a cell, mitochondrial movement in a cell, communication between the ER and mitochondria “thickness” of ER-MAM in a cell as reflected by cholesterol content in the ER-MAM, in situ morphology of ER-MAM in a cell, or other criteria as provided herein. In addition, the present methods can involve one or more of the above-mentioned indicators of altered ER-MAM integrity in combination with one or more general indicators of AD. General indicators of AD include, but are not limited to, altered APP processing, amyloid toxicity, tau hyperphosphorylation, altered lipid and cholesterol metabolism, altered glucose metabolism, aberrant calcium homeostasis, glutamate excitoxicity, inflammation and mitochondrial dysfunction.

### Alzheimer's Disease

The present invention provides compositions and methods that are useful the diagnosis of Alzheimer's disease in a subject and in the identification of compounds or therapeutic agents for treating Alzheimer's disease.

Alzheimer disease (AD) is a neurodegenerative dementing disease of relatively long course and late onset. The neuronal loss is especially evident in the cortex and hippocampus. AD, a leading cause of dementia, is one of several disorders that cause the gradual loss of brain cells. Dementia is an umbrella term for several symptoms related to a decline in thinking skills. Symptoms include a gradual loss of memory, problems with reasoning or judgment, disorientation, difficulty in learning, loss of language skills and a decline in the ability to perform routine tasks. People with dementia also experience changes in their personalities and experience agitation, anxiety, delusions, and hallucinations.

Pathologies of AD include the atrophy of brain gray matter as a result of the massive loss of neurons and synapses, and protein deposition in the form of both intraneuronal neurofibrillary tangles and extracellular amyloid plaques within the brain parenchyma. In addition, affected areas of the AD brain exhibit a reactive gliosis that appears to be a response to brain injury. Surviving neurons from vulnerable populations in AD show signs of metabolic compromise as indicated by alterations in the cytoskeleton (Wang et al., Nature Med., 1996, 2, 871-875), Golgi complex (Salehi et al., J. Neuropath. Exp. Neurol., 1995, 54, 704-709) and the endosomal-lysosomal system (Cataldo et al., Neuron, 1995, 14, 671-680).

Biochemically, the disease is characterized by the appearance of neuritic senile plaques composed of β-amyloid, and neurofibrillary tangles composed of hyperphosphorylated and aggregated Tau proteins. The familial form (FAD) is associated with mutations in amyloid precursor protein (APP), in presenilin 1 (PS1), and in presenilin 2 (PS2). PS1 and PS2 are aspartyl proteases. They are components of the γ-secretase complex, that cleaves APP within the plasma membrane to ultimately produce amyloid β-peptide. The γ-secretase complex also contains APH1 (with at least 3 isoforms), PEN2, and NCT (nicastrin; also called APH2). Following cleavage of the amyloid precursor protein (APP) by α- and β-secretases, γ-secretase cleaves the remaining APP polypeptide to release small amyloidogenic fragments 40- and 42-aa in length (Aβ40 and Aβ42). These fragments have been implicated in the pathogenesis of AD. Presenilins cleave their target polypeptides within membranes (Wolfe and Kopan, 2004).

The vast majority of AD is sporadic (SAD), but at least five gene loci, and three genes, have been identified in the familial form (FAD). The three genes are amyloid β precursor protein (APP, on chromosome 21q21.3), presenilin 1 (PS1, on 14q24.2), and presenilin 2 (PS2, on 1q42.13).

### Presenilins

PS1 and PS2 share an overall 67% amino acid sequence homology. Primary structure analysis indicates they are integral membrane proteins with 6 to 8 trans-membrane domains (Slunt et al., Amyloid-Int. J Exp. Clin. Invest., 1995, 2, 188-190; Doan et al., Neuron, 1996, 17, 1023-1030). The presenilin proteins are processed proteolytically through two intracellular pathways. Under normal conditions, accumulation of 30 kDa N-terminal and 20 kDa C-terminal proteolytic fragments occurs in the absence of the full-length protein. This processing pathway is regulated and appears to be relatively slow, accounting for turnover of only a minor fraction of the full-length protein. The remaining fraction is degraded in a second pathway by the proteasome (Thinakaran et al., Neuron, 1996, 17, 181-190; Kim et al., J. Biol. Chem., 1997, 272, 11006-11010).

FAD linked to the presenilin mutations is highly penetrant. The aggressive nature of the disease indicates that the mutant protein participates in a seminal pathway of AD pathology. To date, over seventy FAD mutations have been identified in PS1, and three FAD mutations have been found in PS2. Most of the FAD mutations occur in conserved positions between the two presenilin proteins, indicating that they affect functionally or structurally important amino acid residues. All but two of the presenilin mutations are missense mutations. One exception results in an aberrant RNA splicing event that eliminates exon 9, creating an internally-deleted mutant protein (Perez-Tur et al., NeuroReport, 1995, 7, 297-301; Sato et al., Hum. Mutat. Suppl., 1998, 1, S91-94; and Prihar et al., Nature Med., 1999, 5, 1090). The other results in two deletion transcripts (Δ4 and Δ4cryptic) and one full-length transcript with the amino acid Thr inserted between 113 and 114 (DeJonghe et al., Hum. Molec. Genet., 1999, 8, 1529-1540). The latter transcript leads to the AD pathophysiology.

Presenilins form the catalytic subunit of the γ-secretase complex that produces the Aβ peptide. Most mutations in APP, PS1 and PS2 result in an increase in the ratio of a 42-residue form of Aβ (Aβ42) versus 40-residue Aβ (Aβ40). Aβ peptides ending at residue 42 or 43 (long tailed Aβ) are more fibrillogenic and more neurotoxic than Aβ ending at residue 40, which is the predominant isoform produced during normal metabolism of βAPP (St. George-Hyslop, P. H., & Petit, A., C. R. Biologies (2004) 328:119-130; Selkoe, D. J., J Clin Invest (2002) 110:1375-1381).

Elevated levels of Aβ1-42 are also found in cells transfected with mutant PS1 or PS2 and in mice expressing mutant PS1 (Borchelt et al., Neuron, 1996, 17, 1005-1013; Duff et al., Nature, 1996, 383, 710-713; Citron et al., Nature Med., 1997, 3, 67-72; Murayama et al., Prog. Neuro-Psychopharmacol. Biol. Psychiatr., 1999, 23, 905-913; Murayama et al., Neurosci. Lett., 1999, 265, 61-63; Nakano et al., Eur. J. Neurosci., 1999, 11, 2577-2581). The mechanism by which the mutant presenilins affect APP processing is not known. PS1-comprised γ-secretase and PS2-comprised γ-secretase, can also be involved in Notch signaling (Shen et al (1997)).

PS1 has been localized to numerous regions of the cell, including the plasma membrane (Georgakopoulos et al, 1999; Baki et al, 2001; Marambaud et al, 2002; Marambaud et al, 2003; Tarassishin, 2004), the Golgi (Siman et al, 2003; Kimura et al, 2001), and the endoplasmic reticulum (De Strooper et al, 1997; Wolfe et al, 2004), endosomes/lysosomes, the nuclear envelope (Wolfe et al, 2004), and adherens junctions (Marambaud et al, 2002). PS1 has not been found in mitochondria, except for reports from one group that used Western blotting and immunoelectron microscopy, not immunohistochemistry, to localize PS1 to the rat mitochondrial inner membrane (Ankarcrona et al, 2002; Hansson et al, 2005). Another group used immuno electron microscopy and found PS1 in the ER, in the perinuclear region, and at the plasma membrane (at areas of cell-to-cell contact), but not in mitochondria (Takashima et al, 1996). Using immunoelectron microscopy and Western blotting, APH1, NCT, and PEN2 were found to reside in rat mitochondria (Ankarcrona et al, 2002, Hansson et al, 2004).

### ER-MAM and ER-MAM-Associated Proteins

The present invention provides compositions and methods that are useful the diagnosis of neurodegenerative diseases, including Alzheimer's disease, and in the identification of compounds or therapeutic agents for treating neurodegenerative diseases, including dementia, and including Alzheimer's disease.

ER-MAM is a specific compartment involved in the synthesis and transfer of phospholipids between the ER and mitochondria (Vance (1990) JBC 265:7248). ER-MAM-localized proteins (ER-MAM-associated proteins) are involved in intermediate, sphingolipid, ganglioside, fatty acid, and cholesterol metabolism, as well as in apoptosis and calcium homeostasis (Table 1). ER-MAM can also contain enzymes involved in glycosylphosphatidylinositol synthesis (Rogaeva et al, 2007), the unfolded protein response (Zhou et al, 2007), proteasomal function (De Strooper, 2003; Siman and Velji, 2003), and mitochondrial import (Kaether et al, 2006) and fission (Tarassishin et al, 2004). The microsomal triglyceride transfer protein contains two subunits, a large subunit (MTTP), and a small subunit that has been identified as protein disulfide isomerase (PDI) (Cupers et al, 2001). MTTP is a ER-MAM-associated protein (Kimura et al, 2001), but it is unclear if PDI is also ER-MAM-associated (Vetrivel et al, 2004). Finally, ApoE, which is a secreted protein, is present intracellularly in high abundance in the ER-MAM fraction (Vance (1990) JBC 265:7248).

As used herein, an “ER-MAM-associated protein” includes, but is not limited to, proteins localized or concentrated in the ER-MAM such as those listed in Table 1.

TABLE 1
Proteins Localized or Concentrated in ER-MAM

| Gene | Protein | Function | Reference |
|---|---|---|---|
| ACSL1 | Fatty acid-CoA ligase, long-chain | Fatty Acid | T. M. Lewin, C. G. Van Horn, |
| | 1 (FACL1) (acyl-CoA synthetase | Metabolism | S. K. Krisans, R. A, Coleman, |
| | 1) | | Arch. Biochem. Biophys. 404, |
| | | | 263 (2002) |
| ACSL4 | Fatty acid-CoA ligase, long-chain | Fatty Acid | T. M. Lewin, C. G. Van Horn, |
| | 4 (FACL4) (acyl-CoA synthetase | Metabolism | S. K. Krisans, R. A, Coleman, |
| | 4) | | Arch. Bioohem. Biophys. 404, |
| | | | 263 (2002) |
| AKT1 | Protein kinase AKT1 (PKB) | Calcium | Giorgi (2010) Science |
| | | | 330: 1247 |
| AMFR | Autocrine motility factor receptor | Ubiquitination | J. G. Goetz, I. R Nabi, |
| | 2 (GP78) | | Biochem. Soc. Trans. 34, 370 |
| | | | (2006). |
| APOB | Apolipoprotein B | Cholesterol metab | Vance (1990) J Biol Chem |
| | | | 265: 7248 |
| APOC1 | Apolipoprotein C1 | Cholesterol metab | Vance (1990) J Biol Chem |
| | | | 265: 7248 |
| APOC2 | Apolipoprotein C2 | Cholesterol metab | Vance (1990) J Biol Chem |
| | | | 265: 7248 |
| APOC3 | Apolipoprotein C3 | Cholesterol metab | Vance (1990) J Biol Chem |
| | | | 265: 7248 |
| APOC4 | Apolipoprotein C4 | Cholesterol metab | Vance (1990) J Biol Chem |
| | | | 265: 7248 |
| APOE | Apolipoprotein E | Lipid/Cholesterol | J. E. Vance, J Biol. Chem. |
| | | Metabolism | 265, 7248 (1990). |
| APP | Amyloid beta precursor protein | Notch signaling | Described herein |
| ARV1 | Flippase | Glycolipid metab | Kajiwara (2008) MBC 19: 2069 |
| ATP2A1 | Sarco-endoplasmic reticulum | Calcium | de Meis (2010) PLoS One |
| | calcium-ATPase 1 | | 5: e9439 |
| B4GALNT1 | β-1,4 N- | Ganglioside Synthesis | D. Ardailefal., Biochem. J. |
| | acetylgalactosaminyltransferase | | 371, 1013 (2003). |
| | 1(SIAT2) | | |
| B4GALT6 | β-1,4-galactosyltransferase 6 | Glycophospholipid | D. Ardailefal., Biochem. J. |
| | (lactosyl-ceramide synthase) | Metabolism | 371, 1013 (2003). |
| BCL2 | BCL2 | Apoptosis | Meunier (2010) J Pharmacol Exp Ther |
| | | | 332: 388 |
| BSG | Basigin/CD147/EMMPRIN | Regulatory component | Hashimoto (2006) AJPEM |
| | | of g-secretase | 290: E1237 |
| C1RL | Complement component 1, r | Protein maturation | Wicher (2004) PNAS |
| | subcomponent | | 101: 14390 |
| CALR3 | Calreticulin 3 | Calcium | Wieckowski (2009) Nat |
| | | | Protocols 11: 1582 |
| CANX | Calnexin | Calcium Homeostasis | Myhill et al. (2008) Mol. Biol. |
| | | | Cell 19; 2777 |
| DGAT2 | Diacylglycerol O-acyltransferase | Triglyceride | W. C. Man, M. Miyazaki ,K. |
| | | Metabolism | Chu, J. Ntambi, J. Lipid Res. |
| | | | 47, 1928 (2006). |
| EROlL | ER oxidoreductin-1-L-α | ER stress | Gilady (2010) Cell Stress |
| | | | Chaperones 15: 619 |
| ERP44 | ER resident protein ERp44 | ER stress | Gilady (2010) Cell Stress |
| | | | Chaperones 15: 619 |
| G6PC | Glucose-6-phosphatase | Glucose Homeostasis | C. Bionda, J. Portoukalian, D. |
| | | | Schmitt, C. Rodriguez- |
| | | | Lafriasse, D. Ardail, Biochem. |
| | | | J 382, 527 (2004) |
| HCMVUL37 | Cytomegalus virus-encoded | Anti-apoptotic protein; | Bozidis (2008 J Virol 82: 2715 |
| | vMIA protein fr unspliced exon I | binds ANT | |
| | UL37 mRNA, N-term frag | | |
| HP | Prohaptoglobin | Protein maturation | Wassler (1993) J Cell Biol |
| | | | 123: 285 |
| HSPAS | Glucose-regulated protein 78-kDa | ER Stress: Chaperone | T. Hayashi, T. P. Su, Cell |
| | (BiP) | | 131,596 (2007). |
| HSPA9 | Glucose-regulated protein 75-kDa | Binds VDAC | Szabadkai (2006) JCB |
| | (GRP75; Mortalin-2) | | 175: 901 |
| ITPR1 | IP3 receptor, type 1 | Calcium | Szabadkai (2006) J Cell Biol |
| | | | 175: 901 |
| ITPR2 | IP3 receptor, type 2 | Calcium | Szabadkai (2006) J Cell Biol |
| | | | 175: 901 |
| ITPR3 | Inositol 1,4,5-triphosphate | Calcium Homeostasis | C. C. Mendes el al, J. Biol |
| | receptor, type 3 (IP3R3) | | Chem. 2 80, 40892 (2005). |
| LMAN1 | Lectin, mannose-binding 1 | Glycolipid metab | Lahtinen (1996) J Biol Chem |
| | | | 271: 4031 |
| MBOAT2 | Membrane bound O- | Phospholipid syn | Rieckhof (2007) JBC |
| | acyltransferase domain containing | | 282: 28344 |
| | 2 | | |
| MFN2 | Mitofusin-2 | Other | de Brito et al. (2008) Nature |
| | | | 456: 605 |
| MTTP | Microsomal trigyceride transfer | Lipoprotein Transport | A. E. Rusinol, Z. Cui, M. H. |
| | protein large subunit | | Chen, J. E. Vance, J. Biol. |
| | | | Chem. 269, 27494 (1994). |
| MTTP | Microsomal triglyceride transfer | Cholesterol | and Lipid Rusinol et al. (1994) J. Biol. |
| | protein large subunit | Metabolism | Chem. 269: 27494 |
| OPRS1 | Opioid receptor, sigmal | Calcium homeostasis | T. Hayashi, T. P. Su, Cell |
| | | | 131, 596 (2007). |
| p23 | Hepatitis C virus core protein | Lipid metab | Williamson (2009) Rev Med |
| | | | Virol 19: 147 |
| PACS2 | Phosphofurin acidic cluster | ER-MAM Integrity: | T. Simrnen et al., EMBOJ. 24, |
| | sorting protein 2* | Apoptosis | 717(2005). |
| PEMT | Phosphatidylethanolamine N- | Phospholipid | D. E. Vance, C. J. Walkey, Z. |
| | methyltransferase 2 (PEMT) | Metabolism | Cui, Biochim. Biophys Acta |
| | | | 1348, 142(1997). |
| PIGL | N-acetylglucosaminyl- | Glycophosphoinositol | A. Pottekat, A. K. Menon, J. |
| | phosphatidylinositol de-N- | Synthesis | Biol. Chem. 279, 15743 (2004). |
| | acetylase | | |
| PIGM | α1-4 mannosyltransferase | Glycolipid metab | Maeda (2001) EMBO J 20: 250 |
| PIGN | Ethanolaminephosphate | Glycolipid metab | Hong (1999) J Biol Chem |
| | transferase | | 274: 35099 |
| PML | Promyelocytic leukemia tumor | Calcium | Giorgi (2010) Science |
| | suppressor | | 330: 1247 |
| PPP2CA | Protein phosphatase 2A, subunit | Calcium | Giorgi (2010) Science |
| | C | | 330: 1247 |
| PPP2R1A | Protein phosphatase 2A, subunit | Calcium | Giorgi (2010) Science |
| | Aα | | 330: 1247 |
| PPP2R1B | Protein phosphatase 2A, subunit | Calcium | Giorgi (2010) Science |
| | Aβ | | 330: 1247 |
| PS1 | Presenilin 1 | ER-MAM Integrity | As described herein |
| PS2 | Presenilin 2 | ER-MAM Integrity | As described herein |
| PTDSS1 | Phosphatidylserine synthase 1 | Phospholipid | S. J. Stone, J. E. Vance, J. Biol |
| | (PSS1) | Metabolism | Chem. 275, 34534(2000). |
| PTDSS2 | Phosphatidylserine synthase 2 | Phospholipid | S. J. Stone, J. E. Vance, J. Biol |
| | (PSS2) | Metabolism | Chem. 275, 34534(2000). |
| RAB32 | A-kinase anchoring protein | Mito fission | Bui (2010) J Biol Chem |
| | | | 285: 31590 |
| RYR1 | Ryanodine Receptor type 1 | Calcium Homeostasis | O. Kopach, I. Kruglikov, T. |
| | | | Pivneva, N. Voitenko, N. |
| | | | Fedirko, Cell Calcium 43: 469 |
| | | | (2007). |
| RYR2 | Ryanodine Receptor type 2 | Calcium Homeostasis | O. Kopach, I. Kruglikov, T. |
| | | | Pivneva, N. Voitenko, N. |
| | | | Fedirko, Cell Calcium 43: 469 |
| | | | (2007). |
| RYR3 | Ryanodine Receptor type 3 | Calcium Homeostasis | O. Kopach, I. Kruglikov, T. |
| | | | Pivneva, N. Voitenko, N. |
| | | | Fedirko, Cell Calcium 43: 469 |
| | | | (2007). |
| SCD | Acyl-CoA desaturase (stearoyl- | Fatty Acid | W. C. Man, M. Miyazaki ,K. |
| | CoA desaturase 1) | Metabolism | Chu, J. Ntambi, J. Lipid. Res. |
| | | | 47, 1928 (2006). |
| SHC1 | Src homology and collagene | Redox | Lebiedzinska (2009) Arch |
| | | | Biochem Biophys 486: 73 |
| SIGMAR1 | Sigma-1 type opioid receptor | Calcium | Hayashi (2007) Cell 131: 596 |
| SLC27A4 | Fatty acid transport protein 4 | Fatty Acid Transport | W. Jia, C. L. |
| | (FATP4) | | Moulson, Z. Pei, J. H. Miner, P. |
| | | | A. Watkins, J. Biol Chem. |
| | | | 282, 20573 (2007). |
| SOAT1 | Acyl-CoA: cholesterol | Cholesterol | A. E. Rusinol, Z. Cui, M. H. |
| | acyltransferase (ACAT1) | Metabolism | Chen, J. E. Vance, J. Biol. |
| | | | Chem. 269, 27494 (1994). |
| ST3GAL1 | (β-galactoside α(2-3) | Ganglioside Synthesis | D. Ardailefal., Biochem. J. |
| | sialyltransferase (SIAT4) | | 371, 1013 (2003). |
| ST3GAL1 | Beta-galactoside alpha(2-3) | Phospholipid, | Ardail et al. (2003) Biochem. J. |
| | sialyltransferase (SIAT4) | glycolipid, and | 371: 1013 |
| | | triglyceride | |
| | | metabolism | |
| ST6GAL1 | (β-galactoside α(2-6) | Ganglioside Synthesis | D. Ardailefal., Biochem. J. |
| | sialyltransferase (SIAT1) | | 371, 1013 (2003). |
| ST6GAL1 | Beta-galactoside alpha(2-6) | Phospholipid, | Ardail et al. (2003) Biochem. J. |
| | sialyltransferase (SIAT1) | glycolipid, and | 371: 1013 |
| | | triglyceride | |
| | | metabolism | |
| UGCG | Ceramide glucosyltransferase | Glycophospholipid | D. Ardailefal., Biochem. J. |
| | | Metabolism | 371, 1013 (2003). |
| Unknown | Glucosaminylphosphatidylinositol | Glycolipid metab | Kinoshita (2000) Curr Opin |
| | acytransferase | | Chem Biol 4: 632 |
| VDAC1 | Voltage-dependent anion channel | Ion transp | Szabadkai et al. (2006) J. Cell |
| | 1 (Porin 1) | | Biol. 175: 901 |
| VDAC2 | Voltage-dependent anion channel | Ion transp | Szabadkai (2006) J Cell Biol |
| | 2 | | 175: 901 |
| VDAC3 | Voltage-dependent anion channel | Ion transp | Szabadkai (2006) J Cell Biol |
| | 3 | | 175: 901 |

Genetic linkage studies have revealed that FAD is heterogeneous and a majority of the cases have been linked to gene mutations on chromosomes 1, 14, 19, or 21 (reviewed in Siman and Scott, Curr. Opin. Biotech., 1996, 7, 601-607). Affected individuals develop the classical symptomatic and pathological profiles of the disease confirming that the mutations are associated with the development of the disease rather than a related syndrome.

Several proteins close to FAD-linked loci, as assessed by maximum LOD score, have been identified (Table 2).

TABLE 2
Comparison of AD loci (marker with highest LOD) (Top) to adjacent MAM protein
genes (Bottom)

| Symbol | Locus | mB | Reference |
|---|---|---|---|
| D1S218 | 1q25 | 172.7 | Liu et al. (2007) Am J Hum Genet 81: 17 |
| SOAT1/ACAT1 | 1q25.2 | 177.5 | Rusinol (1994) JBC 269: 27494 |
| AD4: PS2 | 1q42.13 | 225.1 | Levy-Lahad et al.(1995) Science 269: 973-977 |
| PS2 | 1q42.13 | 225.1 | Schon group |
| D3S2418 | 3q28 | 193.8 | Lee (et al. 2006) Arch Neurol 63: 1591 |
| ST6GAL1/SIAT1 | 3q27.3 | 188.1 | Ardail (2003) Biochem J 371: 1013 |
| D6S1051 | 6p21.31 | 36.7 | Lee (et al. 2006) Arch Neurol 63: 1591 |
| IPR3/ITPR3 | 6p21.31 | 33.7 | Mendes et al. (2005) J Biol Chem 280: 40892 |
| D7S2847 (DLD locus) | 7q31.31 | 118.5 | Brown et al.(2007) Neurochem Res 32: 857 |
| SOAT1/ACAT1 Exon | 7q31.31 | 120.5 | Li (1999) JBC 274 11060 |
| Xa | | | |
| D8S1119 | 8q21.2- | 87.2 | Gedraitis et al. (2006) JMG 43: 931 |
| | 21.3 | | |
| PTDSS1 | 8q22.1 | 97.3 | Stone et al. (2000) J Biol Chem 275: 34534 |
| D9S930 | 9q32 | 114.3 | Lee (et al. 2006) Arch Neurol 63: 1591 |
| UGCG | 9q31.3 | 113.7 | Ardail et al. (2003) Biochem J 371:1013 |
| DNMPB | 10q24.2 | 101.7 | Bettens et al. (2008) Neurobiol Aging 30: 2000 |
| SCD | 10q24.31 | 102.1 | Man et al. (2006) J Lipid Res 47: 1928 |
| D11S1320 | 11q25 | 131.4 | Liu et al. (2007) Am J Hum Genet 81: 17 |
| SIAT4c/ST3GAL4 | 11q24.2 | 125.8 | Ardail et al. (2003) Biochem J 371: 1013 |
| AD3: PS1 | 14q24.2 | 72.6 | Sherrington et al. (1995) Nature 375: 754-760 |
| PS1 | 14q24.2 | 72.6 | Schon group; |
| | | | Sherrington et al.(1995) Nature 375: 754 |
| D17S951 | 17q21 | 39.1 | Rademaker et al. (2002) Mol Psychiatry 7: 1064 |
| G6PC | 17q21.31 | 38.3 | Bionda (2004) Biochem J 382: 527 |
| AD2 | 19q13.2 | 43-48 | Pericak-Vance et al. (1991) Am J Hum Genet |
| | | | 48: 1034 |
| RYR1 | 19q13.2 | 43.7 | Beutner (2001) JBC 276: 21482; Hajnoczky02 |
| D195178 | 19q13.31 | 49.0 | Schon group; |
| APOE | 19q13.32 | 50.1 | Vance et al.(1990) J Biol Chem 265: 7248 |

In the certain embodiments of the invention, an ER-MAM-associated protein is a natural or recombinant protein, polypeptide, an enzyme, a holoenzyme, an enzyme complex, an enzyme subunit, an enzyme fragment, derivative or analog or the like, including a truncated, processed or cleaved enzyme (Enzymol. 260:14; Ernster et al., 1981 J. Cell Biol. 91:227s-255s, and references cited therein).

An ER-MAM-associated protein can optionally include one or more additional components. As a non-limiting example of an optional component, a ER-MAM-associated protein can further comprise a flexible region comprising a flexible spacer. Spacers can be useful to allow conformational flexibility when one or more peptides are joined in the context of a fusion protein (e.g. GFP fusion proteins or epitope tagged proteins). Non-limiting examples of a flexible spacer include, e.g., a polyglycine spacer or an polyalanine spacer. A flexible region comprising flexible spacers can be used to adjust the length of a polypeptide region in order to optimize a characteristic, attribute or property of a polypeptide. Such a flexible region is operably-linked in-frame to the ER-MAM-associated protein as a fusion protein. As one non-limiting example, a polypeptide region comprising one or more flexible spacers in tandem can be use to better present a donor fluorophore or acceptor, thereby facilitating the resonance transfer energy of the donor fluorophore and acceptor pair.

An ER-MAM-associated protein further can include, without limitation, one or more of the following: epitope-binding tags, such as. e.g., FLAG, Express™, human Influenza virus hemagglutinin (HA), human p62.sup.c-Myc protein (c-MYC), Vesicular Stomatitis Virus Glycoprotein (VSV-G), glycoprotein-D precursor of Herpes simplex virus (HSV), V5, and AU1; affinity-binding, such as. e.g., polyhistidine (HIS), streptavidin binding peptide (strep), and biotin or a biotinylation sequence; peptide-binding regions, such as. e.g., the glutathione binding domain of glutathione-S-transferase, the calmodulin binding domain of the calmodulin binding protein, and the maltose binding domain of the maltose binding protein; immunoglobulin hinge region; an N-hydroxysuccinimide linker; a peptide or peptidomimetic hairpin turn; or a hydrophilic sequence or another component or sequence that, for example, promotes the solubility or stability of the ER-MAM-associated protein. Non-limiting examples of specific protocols for selecting, making and using an appropriate binding peptide are described in, e.g., Epitope Tagging, pp. 17.90-17.93 (Sambrook and Russell, eds., Molecular Cloning A Laboratory Manual, Vol. 3, 3.sup.rd ed. 2001; Antibodies: A Laboratory Manual (Edward Harlow & David Lane, eds., Cold Spring Harbor Laboratory Press, 2.sup.nd ed. 1998; and Using Antibodies: A Laboratory Manual: Portable Protocol No. I Edward Harlow & David Lane, Cold Spring Harbor Laboratory Press, 1998), which are hereby incorporated by reference.

In addition, non-limiting examples of binding peptides as well as well-characterized reagents, conditions and protocols are readily available from commercial vendors that include, without limitation, BD Biosciences-Clontech, Palo Alto, Calif.; BD Biosciences Pharmingen, San Diego, Calif.; Invitrogen, Inc, Carlsbad, Calif.; QIAGEN, Inc., Valencia, Calif.; and Stratagene, La Jolla, Calif. These protocols are routine procedures well within the scope of one skilled in the art and from the teaching herein.

### Indicators of ER-MAM Integrity

The methods of the present invention pertain in part to the correlation of AD with an increased or decreased level of at least one indicator of altered ER-MAM integrity. For example, indicators of altered ER-MAM integrity include, but are not limited to: (1) whether the communication between the ER and mitochondria in FAD^{PS1} or FAD^{PS2} cells is increased as compared to controls, (2) whether the “thickness” of MAM or the amount of cholesterol in MAMs are increased in cells from subjects with AD, (3) whether mitochondrial distribution is different in fibroblasts between age-matched controls and patients with FAD harboring pathogenic mutations in presenilin, such as whether almost all the FAD^{PS1} or FAD^{PS2} mitochondria are in the perinuclear region and/or whether fewer FAD^{PS1} or FAD^{PS2} mitochondria are in the extremities of fibroblasts as compared to control, and (4) whether FAD^{PS1} or FAD^{PS2} mitochondria appear less elongated (e.g. less tubular) and more “punctate”. In some embodiments, present methods further comprise screening for: (1) elevated cholesterol levels, (2) altered brain glucose metabolism, (3) altered lipid metabolic profiles, (4) significant increases in PC and PE in sporadic AD patient brains, (5) disturbed calcium homeostasis as a feature of both SAD and FAD, and/or (6) cells with presenilin mutations and ApoE3/E4 or ApoE4/E4 genotype. In some embodiments, methods for screening for AD do not involve any genetic screen for P51, PS2, or APP mutations.

Thus, as previously stated, an “indicator of altered ER-MAM integrity” can be any detectable parameter that directly or indirectly relates to a condition, process, or other activity involving ER-MAM and that permits detection of altered or abnormal ER-MAM function or state (as compared to ER-MAM from normal or non-AD cells) in a biological sample from a subject or biological source (detection can also be in the subject or animal model). Exemplary indicators of altered ER-MAM integrity can be, for example, a functional activity or expression level of an ER-MAM-associated protein, subcellular localization of an ER-MAM-associated protein, mitochondrial morphology in a cell, mitochondrial localization in a cell, communication between the ER and mitochondria, “thickness” of ER-MAM in a cell as reflected by cholesterol content in the ER-, in situ morphology of ER-MAM in a cell, or other criteria as provided herein. In addition, the present methods can involve one or more of the above-mentioned indicators of altered ER-MAM integrity in combination with one or more general indicators of AD. General indicators of AD include, but are not limited to, altered APP processing, amyloid toxicity, tau hyperphosphorylation, altered lipid and cholesterol metabolism, altered glucose metabolism, aberrant calcium homeostasis, glutamate excitoxicity, inflammation, mitochondrial dysfunction, and genetic screens for PS1, PS2, and/or APP mutations associated with AD.

In another embodiment, the diagnosis can be performed by comparing the increase or a decrease an indicator of ER-MAM integrity in a test biological sample in comparison to an indicator of ER-MAM integrity in a control biological sample. Altered ER-MAM integrity can refer to any condition or state, including those that accompany AD, where any structure or activity that is directly or indirectly related to a ER-MAM function has been changed relative to a control or standard.

In certain embodiments of the present invention, AD can be correlated with an increased or decreased level of at least one “indicator of altered ER-MAM integrity”. An indicator of ER-MAM integrity refers to an indicator of altered ER-MAM function, as provided herein. In some embodiments, an alteration in ER-MAM function can be determined with at least one indicator of altered ER-MAM integrity. For example, and indicators of altered ER-MAM integrity can include, but are not limited to altered APP processing, amyloid toxicity, tau hyperphosphorylation, altered lipid and cholesterol metabolism, altered glucose metabolism, aberrant calcium homeostasis, glutamate excitoxicity, inflammation and mitochondrial dysfunction.

In one embodiment, the invention provides a method for diagnosing Alzheimer's disease in a subject, the method comprising: (a) obtaining a biological sample from an individual suspected of having Alzheimer's disease, (b) measuring an amount of an indicator of ER-MAM integrity in the biological sample of step (a), and (c) comparing the amount of the indicator of ER-MAM integrity measured in the biological sample of step (a) to the amount of an indicator of ER-MAM integrity measured in a control biological sample wherein, a reduced amount of the indicator of ER-MAM integrity measured in the biological sample of step (a) compared to the control biological sample indicates that the subject has Alzheimer's disease. In another embodiment, the invention provides a method for diagnosing Alzheimer's disease in a subject, the method comprising: (a) obtaining a biological sample from an individual suspected of having Alzheimer's disease, (b) measuring an amount of an indicator of ER-MAM integrity in the biological sample of step (a), and (c) comparing the amount of the indicator of ER-MAM integrity measured in the biological sample of step (a) to the amount of an indicator of ER-MAM integrity measured in a control biological sample wherein, a greater amount of the indicator of ER-MAM integrity measured in the biological sample of step (a) compared to the control biological sample indicates that the subject has Alzheimer's disease. In another aspect, the present methods for diagnosis can also be used with subjects as a method for predicting whether a subject has a higher probability of becoming afflicted with Alzheimer's disease.

Altered ER-MAM integrity can have its origin in extra ER-MAM structures or events as well as in ER-MAM structures or events, in direct interactions between ER-MAM-associated proteins and proteins outside of ER-MAM genes or in structural or functional changes that occur as the result of interactions between intermediates that can be formed as the result of such interactions, including metabolites, catabolites, substrates, precursors, cofactors and the like.

Additionally, altered ER-MAM integrity can include altered metabolic or other biochemical or biophysical activity in some or all cells of a biological source. As non-limiting examples, cholesterol metabolism can be related to altered ER-MAM integrity, as can be generation of phosphatidylethanolamine or defective ER-MAM-associated protein localization and/or function. As further examples, altered mitochondrial localization, altered mitochondrial morphology, induction of apoptotic pathways and formation of atypical chemical and biochemical protein complexes within a cell, whether by enzymatic or non-enzymatic mechanisms, can be regarded as indicative of altered ER-MAM integrity. These and other non-limiting examples of altered ER-MAM integrity are described in greater detail herein.

Without wishing to be bound by theory, pathogenic presenilin mutations altered can be related to altered ER-MAM integrity. Alterations in ER-MAM function play a role in the development of AD, for example by defects in mitochondrial distribution, and mitochondrial dysfunction. Altered ER-MAM integrity can result from direct or indirect effects of reduction, alteration or gain of function effects of mutations, in presenilin gene products or related downstream mediator molecules and/or ER-MAM genes, gene products or related downstream mediators, or from other known or unknown causes.

ER-MAM may contain gene products encoded by mitochondrial genes situated in mitochondrial DNA (mtDNA) and by extramitochondrial genes (e.g., nuclear genes) not situated in the circular mitochondrial genome. Accordingly, mitochondrial and extramitochondrial genes may interact directly, or indirectly via gene products and their downstream intermediates, including metabolites, catabolites, substrates, precursors, cofactors and the like. Alterations in ER-MAM integrity, for example altered APP processing, amyloid toxicity, tau hyperphosphorylation, altered lipid and cholesterol metabolism, altered glucose metabolism and mitochondrial dysfunction may therefore arise as the result of defective mtDNA, defective extramitochondrial DNA, defective mitochondrial or extramitochondrial gene products defective downstream intermediates or a combination of these and other factors.

### Indicators of Altered ER-MAM Integrity: ER-MAM Quantity

The communication between the ER and mitochondria in fibroblasts from patients with FAD harboring pathogenic mutations in or FAD^{PS1} or FAD^{PS2} is increased compared to controls. This increase in ER-MAM quantity also occurs in cells overexpressing presenilin and in cells where presenilin is reduced by shRNA technology. Accordingly, certain aspects of the invention are directed to methods for diagnosing Alzheimer's disease in a subject, the method comprising comparing the communication between the ER and mitochondria in a biological sample to ER-MAM content of a control sample, wherein a increase in communication between the ER and mitochondria in the biological sample compared to the control indicates that the biological sample is from a subject having AD. One skilled in the art can determine the communication between the ER and mitochondria in a biological sample using assays for total protein or and/or total lipids in ER-MAM or total amount of ER-MAM resident proteins or ER-MAM resident lipids.

Further, the mitochondrial distribution is different in fibroblasts between age-matched controls and patients with FAD harboring pathogenic mutations in PS1 (FAD^{PS1}): (1) Almost all the FAD^{PS1} mitochondria are in the perinuclear region; (2) Fewer FAD^{PS1} mitochondria are in the extremities of fibroblasts as compared to control; (3) FAD^{PS1} mitochondria appear less elongated (e.g. less tubular) and more “punctate”; and (4) The communication between the ER and mitochondria in FAD^{PS1} cells is significantly increased as compared to controls.

For Sporadic AD (SAD), there is also a difference in mitochondrial distribution. For SAD patients, there are three alleles of apolipoprotein E in humans: ApoE2, ApoE3, and ApoE4. People with at least one ApoE4 allele are at great risk for sporadic AD. ApoE4 is a MAM-localized protein. The mitochondrial distribution is: (1) Cells with E3/E3 have a normal MAM content; (2) Cells with E3/E4 have increased MAM, irrespective of whether or not the cells have a PS1 mutation; (3) Cells with PS1 mutation and E3/E3 genotype have normal amount of communication between the ER and mitochondria and normal mitochondrial distribution; (4) Cells with PS1 mutation and E3/E4 genotype have increased MAM and altered mitochondrial distribution; and (5) Similar results with brain tissue from PS1 patients: the communication between the ER and mitochondria in E3/E4 patients was increased compared to E3/E3.

Thus, without being bound by theory, the observation that there is a increased communication between the ER and mitochondria may help to explain the role of ApoE in the pathogenesis of AD, and may connect the familial and sporadic forms of the disease into one conceptual framework.

Biological samples can comprise any tissue or cell preparation in which at least one candidate indicator of altered ER-MAM integrity can be detected, and can vary in nature accordingly, depending on the indicator(s) of ER-MAM integrity to be compared. Biological samples can be provided by obtaining a blood sample, biopsy specimen, tissue explant, organ culture or any other tissue or cell preparation from a subject or a biological source. The subject or biological source can be a human or non-human animal, a primary cell culture or culture adapted cell line including but not limited to genetically engineered cell lines. For example, suitable biological samples for diagnosing Alzheimer's disease include cells obtained in a non-invasive manner. Examples include, but are not limited to an AD model cell, a neuron, a fibroblast, a skin biopsy, a blood cell (e.g. a lymphocyte), an epithelial cell and biological materials found in urine sediment. In some embodiments, for example embodiments of methods for screening for screening of compounds, yeast cells, fungi and other eukaryotic cells (e.g. plant cells) can also be used.

AD model disease cells suitable for use with the methods described herein include, but are not limited to, human skin fibroblasts derived from patients carrying FAD-causing presenilin mutations, mouse skin fibroblasts, cultured embryonic primary neurons, and any other cells derived from PS1-knock out transgenic mice (containing null mutation in the PS1 gene), cells having AD-linked familial mutations, cells having genetically associated AD allelic variants, cells having sporadic AD, or cells having mutations associated with sporadic AD. In some embodiments, for example embodiments of methods for screening for screening of compounds, yeast cells, fungi and other eukaryotic cells (e.g. plant cells) can also be used.

AD-linked familial mutations include AD-linked presenilin mutations (Cruts, M. and Van Broeckhoven, C., Hum. Mutat. 11:183-190 (1998); Dermaut, B. et al., Am. J. Hum. Genet. 64:290-292 (1999)), and amyloid β-protein precursor (APP) mutations (Suzuki, N. et al., Science 264:1336-1340 (1994); De Jonghe, C. et al., Neurobiol. Dis. 5:281-286 (1998)).

Genetically associated AD allelic variants include, but are not limited to, allelic variants of apolipoprotein E (e.g., APOE4) (Strittmatter, W. J. et al., Proc. Natl. Acad. Sci. USA 90:1977-1981 (1993)) and SORL1.

More specifically, AD model disease cells can include, but not limited to, one or more of the following mutations, for use in the invention: APP FAD mutations (e.g., E693Q (Levy E. et al., Science 248:1124-1126 (1990)), V717 I (Goate A. M. et al., Nature 349:704-706 (1991)), V717F (Murrell, J. et al., Science 254:97-99 (1991)), V717G Chartier-Harlin, M. C. et al., Nature 353:844-846 (1991)), A682G (Hendriks, L. et al., Nat. Genet. 1:218-221 (1992)), K/M670/671N/L (Mullan, M. et al Nat. Genet. 1:345-347 (1992)), A713V (Carter, D. A. et al., Nat. Genet. 2:255-256 (1992)), A713T (Jones, C. T. et al., Nat. Genet. 1:306-309 (1992)), E693G (Kamino, K. et al., Am. J. Hum. Genet. 51:998-1014 (1992)), T673A (Peacock, M. L. et al., Neurology 43:1254-1256 (1993)), N665D (Peacock, M. L. et al., Ann. Neurol. 35:432-438 (1994)), I 716V (Eckman, C. B. et al., Hum. Mol. Genet. 6:2087-2089 (1997)), and V715M (Ancolio, K. et al., Proc. Natl. Acad. Sci. USA 96:4119-4124 (1999))); presenilin FAD mutations (e.g., all point (missense) mutations except one - - - 113Δ4 (deletion mutation)); PS1 mutations (e.g., A79V, V82L, V96F, 113Δ4, Y115C, Y115H, T116N, P117L, E120D, E120K, E123K, N135D, M139, I M139T, M139V, I 143F, 1143T, M461, I M146L, M146V, H163R, H163Y, S169P, S169L, L171P, E184D, G209V, 1213T, L219P, A231T, A231V, M233T, L235P, A246E, L250S, A260V, L262F, C263R, P264L, P267S, R269G, R269H, E273A, R278T, E280A, E280G, L282R, A285V, L286V, S290C (Δ9), E318G, G378E, G384A, L392V, C410Y, L424R, A426P, P436S, P436Q); PS2 mutations (R62H, N141I, V148I, M293V). Other cell types are readily known to those of ordinary skill in the art.

A tissue can be treated to release one or more individual component cell or cells and the cells can then be treated to release the individual component organelles and so on. Partitioned samples (such as in cells, organelles, cellular fractions) can serve as a protein source for discrimination in 2-D gels and any further methodologies described herein as well as any methodologies known to one skilled in the art.

In the case of a tissue, a tissue sample can be obtained and prepared for separation of the proteins therein using a method that provides suitable levels of discrimination of the proteins of the cell. The proteins can be obtained by any of a variety known means, such as enzymatic and other chemical treatment, freeze drying the tissues, with or without a solubilizing solution, repeated freeze/thaw treatments, mechanical treatments, combining a mechanical and chemical treatment and using frozen tissue samples and so on.

To provide a more specific origin of protein, specific kinds of cells can be purified from a tissue using known materials and methods. To provide proteins specific for an organelle, the organelles can be partitioned, for example, by selective digestion of unwanted organelles, density gradient centrifugation or other forms of separation, and then the organelles can be treated to release the proteins therein and thereof.

Lipid rafts are lipid subdomains that are enriched in cholesterol, and are thicker than surrounding membrane lipids. Moreover, they are detergent insoluble and are resistant to the detergent Triton X-100 (TX-100). The results described herein show that ER-MAM is lipid TX-100-resistant, and is cholesterol-rich. Without being bound by theory, ER-MAM in subjects having, or at risk of having AD can be thicker or less fragile than normal ER-MAM (hence the increase in ER-MAM content in or FAD^{PS1} and or FAD^{PS2} patients). This difference can be exploited both in diagnosis and treatment by using a indicators of ER-MAM integrity to determine ER-MAM thickness/integrity. Thus, in one aspect, the invention provides methods for diagnosing AD in a subject or methods for determining whether a test compound is capable of treating Alzheimer's disease wherein the methods comprise characterization of subcellular membranes or subcellular fractionation.

A variety of methods have been developed aimed at the isolation of one or more subcellular fractions. For example, subcellular fractionation using density gradients and zonal centrifuges is known to one skilled in the art (Anderson, “The Development of Zonal Centrifuges and Ancillary Systems for Tissue Fractionation and Analysis” National Cancer Institute Monograph 21, 1966). Methods for isolating ER-MAM are also known to those skilled in the art (Vance, 1990; and see Example 3)

A crude protein preparation also can be exposed to a treatment that partitions the proteins based on a common property, such as size, subcellular location and so on. For example, the crude lysate can be partitioned prior to high-resolution separation of the proteins to reduce the number of proteins for ultimate separation and to enhance discrimination. Thus, the crude lysate can be fractionated by chromatography. Such a preliminary treatment can be useful when a sample is known to contain one or more abundant proteins. Removing abundant proteins can enhance the relative abundance of minor species of proteins that can be analyzed.

Multiple preliminary fractionation steps can be practiced, such as, using multiple chromatography steps, with the chromatography steps being the same or different, or multiple extraction or other partitioning steps. Suitable chromatography methods include those known in the art, such as immunoaffinity, size exclusion, lectin affinity and so on.

### Indicators of ER-MAM Integrity: Protein Quantity in ER-MAM

Methods for determining ER-MAM-associated protein quantity can depend on the physicochemical properties of an ER-MAM-associated protein. In some embodiments, determination of ER-MAM-associated protein quantity can involve quantitative determination of the level of a protein or polypeptide using routine methods in protein chemistry with which those having skill in the art. Depending on the nature and physicochemical properties of the ER-MAM-associated protein, determination of enzyme quantity can be by densitometric, mass spectrometric, spectrophotometric, fluorimetric, immunometric, chromatographic, electrochemical or any other means of quantitatively detecting a cellular component (See, e.g., Harlow and Lane, Antibodies: A Laboratory Manual, Cold Spring Harbor Laboratory, 1988; Weir, D. M., Handbook of Experimental Immunology, 1986, Blackwell Scientific, Boston). Methods for determining ER-MAM-associated protein quantity also include methods described that are useful for detecting products of enzyme catalytic activity, including those measuring enzyme quantity directly and those measuring a detectable label or reporter moiety.

The amount of an ER-MAM-associated protein, for example, can be determined in a gel pattern from a whole tissue, and in a gel pattern obtained using purified ER-MAM fraction. In the first pattern, the ER-MAM-associated protein can be a minor spot, in the latter, a major spot. The ratio of spot intensity for protein of a purified ER-MAM fraction can be referenced the ER-MAM-associated protein. The ratio between the ER-MAM-associated protein intensity on whole tissue gels and on the gels from isolated nuclei can be used as a multiplier to calculate the quantity of minor proteins in the whole tissue sample.

The proteins in a subcellular fraction can separated by a method that provides discrimination and resolution. For example, the proteins can be separated by known methods, such as chromatography, immunoelectrophoresis, mass spectrometry or electrophoresis. The proteins can be separated in a liquid phase in combination with a solid phase. For example, a suitable separation method is two-dimensional (2-D) gel electrophoresis.

In one embodiment, isolated ER-MAM can also be assayed for the ratio of Aβ42:Aβ40 by Western blot or ELISA, wherein a greater ratio of Aβ42 to Aβ40 in isolated ER-MAM in a biological sample compared to a the ratio of Aβ42 to Aβ40 in isolated ER-MAM in a control biological sample indicates that the subject has, or is at risk of having AD.

For example, assays can be performed in a Western blot format, wherein a preparation comprising proteins from a biological sample is submitted to gel electrophoresis, transferred to a suitable membrane and allowed to react with an antibody specific for an ER-MAM-associated protein. The presence of the antibody on the membrane can then be detected using a suitable detection reagent, as is well known in the art and described herein.

For these and other useful affinity techniques, see, for example, Scopes, R. K., Protein Purification: Principles and Practice, 1987, Springer-Verlag, NY; Weir, D. M., Handbook of Experimental Immunology, 1986, Blackwell Scientific, Boston; and Hermanson, G. T. et al., Immobilized Affinity Ligand Techniques, 1992, Academic Press, Inc., California; which are hereby incorporated by reference in their entireties, for details regarding techniques for isolating and characterizing complexes, including affinity techniques.

In certain embodiments of the invention, an indicator of altered ER-MAM integrity including, for example, an ER-MAM-associated protein as provided herein, can be present in isolated form. Affinity techniques can be used to isolate an ER-MAM-associated protein and can include any method that exploits a specific binding interaction involving an ER-MAM-associated protein to effect a separation.

### Indicators of Altered ER-MAM Function: Protein Activity

Certain aspects of the invention are directed to a method for diagnosing Alzheimer's disease in a subject comprising comparing measuring the activity of an ER-MAM-associated protein. In some embodiments of the invention, the activity of an ER-MAM-associated protein can be the indicator of altered ER-MAM integrity. In one embodiment, the indicator of altered ER-MAM integrity can refer to an indicator of altered ER-MAM integrity as provided herein, which is quantified in relation to activity of an ER-MAM-associated protein. For example, an indicator of altered ER-MAM integrity can be protein activity or enzymatic activity of an ER-MAM-associated protein determined on the basis of its level per unit ER-MAM-associated protein in a sample (e.g., ER-MAM-associated protein in the sample can be the non-enzyme indicator of altered ER-MAM integrity), but the invention need not be so limited.

In one aspect, the invention provides a method for diagnosing Alzheimer's disease in a subject, the method comprising: (a) obtaining a biological sample from an individual suspected of having Alzheimer's disease, (b) measuring the activity of an ER-MAM-associated protein in the biological sample of step (a), and (c) comparing the amount of ER-MAM-associated protein activity measured in the biological sample of step (a) to the amount of ER-MAM-associated protein activity measured in a control biological sample wherein, a reduced amount of ER-MAM-associated protein activity measured in the biological sample of step (a) compared to the control biological sample indicates that the subject has Alzheimer's disease.

In another aspect, the invention provides a method for diagnosing Alzheimer's disease in a subject, the method comprising: (a) obtaining a biological sample from an individual suspected of having Alzheimer's disease, (b) measuring the activity of an ER-MAM-associated protein in the biological sample of step (a), and (c) comparing the amount of ER-MAM-associated protein activity measured in the biological sample of step (a) to the amount of ER-MAM-associated protein activity measured in a control biological sample wherein, an increased amount of ER-MAM-associated protein activity measured in the biological sample of step (a) compared to the control biological sample indicates that the subject has Alzheimer's disease.

As provided herein, the activity of proteins suitable for use as indicators or ER-MAM integrity include, but is are not limited to: Acyl-CoA:cholesterol acyltransferase (ACAT1); Acyl-CoA desaturase (stearoyl-CoA desaturase 1); Apolipoprotein E; Autocrine motility factor receptor 2 (GP78); β-galactoside α(2-3) sialyltransferase (SIAT4); β-galactoside α(2-6) sialyltransferase (SIAT1); β-1,4 N-acetylgalactosaminyltransferase 1 (SIAT2); β-1,4-galactosyltransferase 6 (lactosyl-ceramide synthase); Ceramide glucosyltransferase; Diacylglycerol O-acyltransferase; Fatty acid-CoA ligase, long-chain 1 (FACL1) (acyl-CoA synthetase 1); Fatty acid-CoA ligase, long-chain 4 (FACL4) (acyl-CoA synthetase 4); Fatty acid transport protein 4 (FATP4); Glucose-6-phosphatase; Glucose-regulated protein 78-kDa (BiP); Inositol 1,4,5-triphosphate receptor, type 3 (IP3R3); Microsomal triglyceride transfer protein large subunit; N-acetylglucosaminyl-phosphatidylinositol de-N-acetylase; Opioid receptor, sigma1; Phosphatidylethanolamine N-methyltransferase 2 (PEMT); Phosphatidylserine synthase 1 (PSS1); Phosphatidylserine synthase 2 (PSS2); Phosphofurin acidic cluster sorting protein 2; Presenilin 1; Presenilin 2; Ryanodine Receptor type 1; Ryanodine Receptor type 2; Ryanodine Receptor type 3; Amyloid beta precursor protein; Basigin/CD147/EMMPRIN; Cytomegalus virus-encoded vMIA protein fr unspliced exon I UL37 mRNA, N-term frag; Glucose-regulated protein 75-kDa (GRP75; Mortalin-2); and Membrane bound O-acyltransferase domain containing 2.

The activity of a mitochondrial enzyme can also be an indicator of altered ER-MAM integrity as provided herein (see, e.g., Lehninger, Biochemistry, 1975 Worth Publishers, NY; Voet and Voet, Biochemistry, 1990 John Wiley & Sons, NY; Mathews and van Holde, Biochemistry, 1990 Benjamin Cummings, Menlo Park, Calif.; Lehninger, Biochemistry, 1975 Worth Publishers, NY; Voet and Voet, Biochemistry, 1990 John Wiley & Sons, NY; Mathews and van Holde, Biochemistry, 1990 Benjamin Cummings, Menlo Park, Calif.).

Products of enzyme catalytic activity can be detected by suitable methods that can depend on the quantity and physicochemical properties of the product. Thus, detection can be, for example by way of illustration and not limitation, by radiometric, calorimetric, spectrophotometric, fluorimetric, immunometric or mass spectrometric procedures, or by other suitable means that will be readily apparent to a person having ordinary skill in the art.

In certain embodiments of the invention, detection of a product of enzyme catalytic activity can be accomplished directly, and in certain other embodiments detection of a product can be accomplished by introduction of a detectable reporter moiety or label into a substrate or reactant such as a marker enzyme, dye, radionuclide, luminescent group, fluorescent group or biotin, or the like. The amount of such a label that is present as unreacted substrate and/or as reaction product, following a reaction to assay enzyme catalytic activity, can then be determined using a method appropriate for the specific detectable reporter moiety or label. For radioactive groups, radionuclide decay monitoring, scintillation counting, scintillation proximity assays (SPA) or autoradiographic methods are appropriate.

For many proteins having enzymatic activity, including ER-MAM-associated proteins, quantitative criteria for enzyme catalytic activity are well established.

Methods for measure the activity of lipid biosynthetic enzymes are also known to those skilled in the art. For example, the activity of 3-hydroxy-3-methylglutaryl-CoA reductase can be measured (George et al, 1990). Acyl-CoA:cholesterol acyltransferase and diacylglycerolacyltransferase can be assayed in the same reaction mixture by a modification of the procedure of Heider et al. (1983) using [14C]oleoyl-CoA as substrate. Two of the products of the reaction, triacylglycerol and cholesteryl esters, can be separated by thin-layer chromatography in the solvent system hexane:ethyl acetate 9:1 (v/v). Phosphatidylserine synthase (base-exchange enzyme) can be assayed by methods known to those skilled in the art (Vance and Vance, 1988). CDP-choline-1,2-diacylglycerol cholinephosphotransferase and CDP-ethanolamine-1,2-diacylglycerol ethanolaminephosphotransferase activities can be measured by established procedures (Vance and Vance, 1988). PtdEtn N-methyltransferase activity can be assayed using exogenously added phosphatidylmonomethylethanolamine as substrate (Vance and Vance, 1988). In some embodiments of any enzymatic assay described herein, Triton X-100 can be omitted from the protocol.

### Indicators of Altered ER-MAM Function: ATP Biosynthesis

In one embodiment of the invention, a mitochondrial protein activity can be the indicator of altered ER-MAM integrity. The enzyme may be a mitochondrial enzyme, which may further be an ETC enzyme or a Krebs cycle enzyme. In other embodiments, the indicator of ER-MAM integrity is any ATP biosynthesis factor. Accordingly, the indicator of ER-MAM integrity, may comprise a measure of the function of an electron transport chain (ETC) enzyme, which refers to any mitochondrial molecular component that is a mitochondrial enzyme component of the mitochondrial electron transport chain (ETC) complex associated with the inner mitochondrial membrane and mitochondrial matrix. An ETC enzyme may include any of the multiple ETC subunit polypeptides encoded by mitochondrial and nuclear genes. The ETC can comprise complex I (NADH:ubiquinone reductase), complex II (succinate dehydrogenase), complex III (ubiquinone: cytochrome c oxidoreductase), complex IV (cytochrome c oxidase) and complex V (mitochondrial ATP synthetase), where each complex includes multiple polypeptides and cofactors (for review see, e.g., Walker et al., 1995 Meths).

### Indicators of Altered ER-MAM Function: Phosphatidylethanolamine Synthesis

The ER-MAM is a locus of phospholipid synthesis. Phosphatidylserine (PS) is transported from the MAM to mitochondria, where it is decarboxylated to phosphatidylethanolamine (PE). The PE is then re-transported back to the ER-MAM, where it is methylated to phosphatidylcholine (PC). When ER-MAM is increased, the rate of transport of PS from the MAM to the mitochondria is increased, and the production of PE inside of mitochondria is also increased.

For example, by way of illustration and not limitation, an ER-MAM-associated protein that is an enzyme can refer to a trans-membrane transporter molecule that, through its enzyme catalytic activity, facilitates the movement of metabolites between cellular compartments. For example, such metabolites can include, but are not limited to phosphatidylserine, phosphatidylethanolamine or other cellular components involved in phosphatidylcholine synthesis, such as gene products and their downstream intermediates, including metabolites, catabolites, substrates, precursors, cofactors and the like.

In one aspect, the invention provides a method for diagnosing Alzheimer's disease in a subject, the method comprising: (a) obtaining a biological sample from an individual suspected of having Alzheimer's disease, (b) measuring the rate of conversion of phosphatidylserine to phosphatidylethanolamine in the biological sample of step (a), and (c) comparing the rate of conversion of phosphatidylserine to phosphatidylethanolamine measured in the biological sample of step (a) to the rate of conversion of phosphatidylserine to phosphatidylethanolamine measured in a control biological sample wherein, an increased or altered rate of conversion of phosphatidylserine to phosphatidylethanolamine measured in the biological sample of step (a) compared to the control biological sample indicates that the subject has Alzheimer's disease.

The rate of conversion of phosphatidylserine to phosphatidylethanolamine can be measured, for example, by adding ³H-Ser to cells and measuring the amount of [³H]PE (and [³H]PS) produced as a function of time (Achleitner et al. (1995) J. Biol. Chem. 270, 29836). In a diagnostic setting, ³H-Ser incorporation in any easily available cell from AD patients can be measured and compared to controls.

Cholesterol and phospholipids (e.g. PE, PS, and PC) are selectively reduced an AD “double-transgenic” (i.e. mutations in both APP and PS1) mouse model (Yao et al. (2008) Neurochem. Res. in press). When ER-MAM is increased, the steady-state levels of PE in cellular membranes, including the plasma membrane, will be increased.

In some embodiments of the invention, a increase in the amount of PE in an AD cell can be used as a diagnostic marker. Cinnamycin (also called Ro 09-0198) is a tetracyclic peptide antibiotic that can be used to monitor transbilayer movement of PE in biological membranes because it binds specifically to PE. When bound, cinnamycin forms a 1:1 complex with PE (Choung et al. (1988) Biochem. Biophys. Acta 940:171). Cinnamycin has been used to identify mutants defective in PS transport through the MAM (Emoto et al. (1999) PNAS 96:12400). Pore formation and hemolysis occurs upon binding of cinnamycin to PE containing membranes and thus control cells (as a result of greater amount of PE in cell membranes) will be more susceptible to cytolysis and cinnamycin-induced killing at lower concentrations of cinnamycin as compared to AD cells.

In one aspect, the invention provides a method for diagnosing Alzheimer's disease in a subject, the method comprising: (a) contacting control cells with an amount of cinnamycin sufficient to kill control cells and measuring the amount of cell death, (b) obtaining a biological sample from an individual suspected of having Alzheimer's disease, and (c) contacting cells from the biological sample with the same amount of cinnamycin used in step and measuring cell death, and (d) comparing the amount of cell death measured in step (a) to the amount of cell death measured in control sample of step (d) wherein, a greater or different amount of cell death measured in step (d) indicates that the subject has Alzheimer's disease. In one embodiment, cell death can be measured with a “live-dead” assay (e.g. living cells are green whereas dead cells are red). In another assay, cell death can be measured with a turbidity assay in erythrocytes (i.e. release of hemoglobin).

### Indicators of Altered ER-MAM Integrity: Presenilin Localization

As described herein, PS1 and PS2 are enriched in a specific subcompartment of the endoplasmic reticulum (ER) that is associated intimately with mitochondria, called ER mitochondria-associated membrane (ER-MAM). ER-MAM forms a physical bridge between the two organelles. When ER-MAM integrity is compromised (e.g. by treating cells with methanol or with the pro apoptotic agent staurosporin), ER-MAM-localized PS1 and PS2, as well as other known ER-MAM localized proteins, such as phosphatidylserine-N-methyltransferase 2 (PEMT; involved in phospholipid metabolism) and acyl-CoA:cholesterol-transferase (ACAT 1; involved in cholesterol metabolism) redistribute to mitochondria located in the perinuclear region (where the ER-MAM is concentrated). In certain embodiments, the localization of PS1 or PS2 to perinuclear regions is an indicator of altered ER-MAM integrity

Thus in one aspect, the invention described herein provides a method for diagnosing Alzheimer's disease in a subject, the method comprising: obtaining a biological sample from an individual suspected of having Alzheimer's disease, measuring the amount of presenilin in ER-MAM in the biological sample and comparing the amount of presenilin in ER-MAM measured in the biological sample to the amount of presenilin in ER-MAM measured in a control cell wherein, an altered amount of presenilin in ER-MAM measured in the control cell indicates that the subject has Alzheimer's disease.

In one aspect, the invention provides a method for diagnosing Alzheimer's disease in a subject, the method comprising: (a) obtaining a biological sample from an individual suspected of having Alzheimer's disease, (b) measuring an amount of ER-MAM localized presenilin in the biological sample of step (a), and (c) comparing the amount of ER-MAM localized presenilin measured in the biological sample of step (a) to the amount of ER-MAM localized presenilin measured in a control biological sample wherein, an increased amount of ER-MAM localized presenilin measured in the biological sample of step (a) compared to the control biological sample indicates that the subject has Alzheimer's disease.

Methods for measuring the amount of in ER-MAM are known to those skilled in the art. For example, total presenilin protein in a ER-MAM can be determined by subcellular fractionation and Western blotting. Total presenilin protein in a ER-MAM can also be determined by immunohistochemistry by comparing the amount of co-localization between presenilin and a known ER-MAM resident protein, for example PEMT.

### Indicators of Altered ER-MAM Integrity: Mitochondrial Localization or Morphology

Mitochondria are organelles found in most mammalian cells. They are the location of many “housekeeping” functions, foremost among them the production of energy in the form of ATP via the respiratory chain/oxidative phosphorylation system. This aspect of mitochondrial function is unique, because the production of oxidative energy is a joint venture between the mitochondrion and the nucleus: genes from both organelles are required. Mitochondria are plastic, with shapes that vary from small spheres (˜1 μm in diameter) to highly elongated tubular structures. In normal cells, they can exist as linear “strings” or as highly branched, reticular structures.

All but 13 of the ˜1,000 proteins present in mitochondria are encoded by nuclear DNA (nDNA). They are synthesized in the cytoplasm and are targeted to mitochondria via mitochondrial targeting signals (MTS's) that direct the polypeptides not only to mitochondria, but also to the proper compartment within the organelle (the outer membrane (MOM), the intermembrane space (IMS), the inner membrane (MIM), and the matrix). The MTS's that target polypeptides to the inner membrane and matrix can have N-terminal presequences that are cleaved following importation. However, much less is known regarding the MTS's of polypeptides that are targeted to the MOM or to the IMS: some are C-terminal and some are “internal,” located within the “business end” of the protein. These MTS's are not cleaved off following importation.

The results described herein show that the distribution of mitochondria in fibroblasts from patients with FAD harboring pathogenic mutations in presenilin is different from the distribution of mitochondria in age-matched normal control fibroblasts. Most mitochondria in FAD^{PS1} or FAD^{PS2} cells are in the perinuclear region, with fewer mitochondria in the “extremities” of the fibroblasts as compared to control cells. In addition, the mitochondria appear less elongated (e.g. less tubular) and more “punctate.” In certain embodiments, the localization of PS1 or PS2 is a indicator of altered ER-MAM integrity.

Thus, in one aspect, the invention described herein provides a method for diagnosing Alzheimer's disease in a subject, the method comprising obtaining one or more cells from an individual suspected of having Alzheimer's disease, measuring the ratio of perinuclear mitochondria to non-perinuclear mitochondria in the cell, and comparing the ratio of perinuclear mitochondria to non-perinuclear mitochondria measured in the cell to the ratio of perinuclear mitochondria to non-perinuclear mitochondria measured in a control cell wherein, a greater ratio of perinuclear mitochondria to non-perinuclear mitochondria measured in the cell compared to the control cell indicates that the subject has Alzheimer's disease.

In another embodiment, the diagnosis can be performed by comparing the ratio of punctate to non-punctate mitochondria in a test cell to a control cell. In another aspect, the invention provides a method for diagnosing Alzheimer's disease in a subject, the method comprising: (a) obtaining one or more cells from an individual suspected of having Alzheimer's disease, (b) measuring the ratio of punctate mitochondria to non-punctate mitochondria in the cell of step (a), and (c) comparing the ratio of punctate mitochondria to non-punctate mitochondria measured in the cell of step (a) to the ratio of punctate mitochondria to non-punctate mitochondria measured in a control cell wherein, a greater ratio of punctate mitochondria to non-punctate mitochondria measured in the cell of step (a) compared to the control cell indicates that the subject has Alzheimer's disease.

Methods for determining the ratio perinuclear mitochondria to non-perinuclear mitochondria and the ratio of punctate mitochondria to non-punctate mitochondria in a cell are known to those skilled in the art.

### Immunometric Measurements

Several methods for performing morphometric analysis of mitochondria are known in the art. For example the amount of perinuclear mitochondria is a cell can be determined by confocal microscopy. Confocal imaging z sections can be projected into a single image. An area between the nucleus and the cell periphery, as determined by microtubule staining, can be outlined, and the midpoint between the nucleus and the farthest point at the cell periphery can be determined. Using the midpoint, the outlined area is then divided into two parts: regions proximal (A) and distal (B) to the nucleus. Mean grayness values of the MitoTracker stain are recorded for the proximal and distal parts. For quantification of mitochondria in the outer edges of a cell, the grayness value for the distal part can be divided by the grayness value for the total area (proximal+distal). Grayness value for the total area=([GraynessA×AreaA]+[GraynessB×AreaB])/(AreaA+AreaB).

For immunometric measurements, suitably labeled antibodies can be prepared including, for example, those labeled with radionuclides, with fluorophores, with affinity tags, with biotin or biotin mimetic sequences or those prepared as antibody-enzyme conjugates (see, e.g., Weir, D. M., Handbook of Experimental Immunology, 1986, Blackwell 45 Scientific, Boston; Scouten, W. H., Methods in Enzymology 135:30-65, 1987; Harlow and Lane, Antibodies: A Laboratory Manual, Cold Spring Harbor Laboratory, 1988; Haugland, 1996 Handbook of Fluorescent Probes and Research Chemicals—Sixth Ed., Molecular Probes, Eugene, Oreg.; Scopes, R. K., Protein Purification: Principles and Practice, 1987, Springer-Verlag, NY; Hermanson, G. T. et al., Immobilized Affinity Ligand Techniques, 1992, Academic Press, Inc., NY; Luo et al., 1998 J. Biotechnol. 65:225 and references cited therein). Various methods can be used to detect dyes (including, for example, colorimetric methods of measuring enzyme catalytic activity are known products of enzyme reactions), luminescent groups and to those having ordinary skill in the art and depend on the activity to be determined

According to certain embodiments, the invention is directed to a method for determining whether a subject has, or is at risk of having Alzheimer's disease, the method comprising comparing mitochondrial localization (e.g. perinuclear or non-perinuclear) or mitochondrial morphology (e.g. punctate or non-punctate) or ER-MAM-associated protein localization in a biological sample with a control sample. Methods for quantifying mitochondrial localization or mitochondrial morphology are known in the art, and can include, for example, quantitative staining of a representative biological sample. By way of example, quantitative staining of mitochondrial can be performed using organelle-selective probes or dyes, including but not limited to mitochondrial selective reagents such as fluorescent dyes that bind to mitochondrial components (e.g., nonylacridine orange, MitoTrackers™) or potentiometric dyes that accumulate in mitochondria as a function of mitochondrial inner membrane electrochemical potential (see, e.g., Haugland, 1996 Handbook of Fluorescent Probes and Research Chemicals—Sixth Ed., Molecular Probes, Eugene, Oreg.)

Mitochondrial mass, volume and/or number can be quantified by morphometric analysis (e.g., Cruz-Orive et al., 1990 Am. J. Physiol. 258:L148; Schwerzmann et al., 1986 J. Cell Biol. 102:97). These or any other means known in the art for quantifying mitochondrial localization or mitochondrial morphology in a sample are within the scope of the invention. Calculations of mitochondrial density can be performed, can include, but are not limited to the use of such quantitative determinations. In some embodiments, mitochondrial localization or mitochondrial morphology can be determined using well known procedures. For example, a person having ordinary skill in the art can readily prepare one or more cells from a biological sample using established techniques, and therefrom determine mitochondrial localization or mitochondrial morphology protein content using any of a number of visualization methodologies well known in the art.

### Indicators of Altered ER-MAM Integrity: Mitochondrial Movement

Mitochondria can fuse and divide, and are also mobile. In mammalian cells they move predominantly along microtubules. This movement, which requires a membrane potential, can be important in neurons, where mitochondria travel from the cell body to the cell's extremities at the ends of axons and dendrites, in order to provide energy for pre-synaptic transmission and for post-synaptic uptake of critical small molecules (e.g. neurotransmitters). Mitochondria attach to microtubules via kinesins and dyneins (Zhang et al, 2004). At least three mitochondrial-binding kinesins have been identified: KIF1B, KIF5B, and KLC3. The binding of kinesins is regulated by phosphorylation by glycogen synthase kinase 3β (GSK3β). The kinetics of mitochondrial attachment to kinesin is also mediated by the microtubule associated protein tau (Trinczek et al, 1999), which is also a target of GSK3β (Tatebayashi et al, 2004). Tau affects the frequency of attachment and detachment of mitochondria to the microtubule tracks (Trinczek et al, 1999). In S. cerevisiae mitochondria move along actin cables, but in S. pombe and mammalian cells they move mainly along microtubules. This movement is important in neurons, where mitochondria travel from the cell body to the cell's extremities at the ends of axons and dendrites, in order to provide energy for pre synaptic transmission and for post-synaptic uptake of critical small molecules (e.g. neurotransmitters). Without mitochondrial movement, metazoan life would not exist.

The dynamics of mitochondrial fusion and fission can be examined using mitochondrially-targeted photo-activatable fluorescent probes (“mitoDendra”) and live-cell imaging of neuronal cells to examine the effects of presenilin mutations in mitochondrial distribution. Mitochondrial maldistribution AD can occur as a result of defects in anterograde and retrograde axonal transport of mitochondria. Mitochondrial maldistribution AD can also occur as a consequence of retention and/or accumulation of mitochondria the extremities of cells. For example, defects in anterograde and retrograde axonal transport of mitochondria, on retention and accumulation of mitochondria in nerve terminals, and on the dynamics of mitochondrial fusion and fission in AD can be performed in primary neuronal cells derived from normal, FAD^{PS1} and FAD^{PS2} mice.

Thus, in one aspect, the invention described herein provides a method for diagnosing Alzheimer's disease in a subject by comparing mitochondrial movement in a test cell to mitochondrial movement in a control cell, wherein a reduced amount of mitochondrial movement in a control cell to the test cell indicated that the subject has Alzheimer's disease, the method comprising: (a) obtaining a cell from an individual suspected of having Alzheimer's disease, (b) measuring an amount of mitochondrial movement in the cell step (a), and (c) comparing the amount of mitochondrial movement measured in the cell of step (a) to the amount of mitochondrial movement measured in a control cell wherein, a reduced an amount of mitochondrial movement measured in the cell of step (a) compared to the control cell indicates that the subject has Alzheimer's disease.

In one embodiment, mitochondrial movement is measured using a mitochondrially targeted Mitotracker dye and live-cell imaging. In another embodiment, mitochondrial movement is measured using a mitochondrially targeted photo-activatable GFP (“mitoDendra”) and live-cell imaging. Dendra is a monomeric variant of GFP (“dendGFP”) that changes from green to red fluorescent states when photoactivated by 488-nm light. Dendra is stable at 37° C. and photoconversion of the photoactivatable GFP from green to red is irreversible and photostable (Gurskaya et al., (2006) Engineering of a monomeric green-to-red photo-activatable fluorescent protein induced by blue light. Nat. Biotechnol. 24:461-465). For example, individual mitochondria can be converted to red fluorescence to track movement in the cell body. to determine whether they appear in a specified distance downstream in an axon, and how long it took to get there.

Because the mitochondrial mislocalization phenotype can be due to (1) a reduced ability of mitochondria to move efficiently along microtubules, or (2) a reduced ability of mitochondria to attach to microtubules (or some combination of the two), mitochondria can be visualized in living cells by colocalizing red mito-Dendra with TubulinTracker Green (a bi-acetylated version of Oregon Green 488 paclitaxel; Molecular Probes T34075) to determine if they are attached to microtubules.

Mitochondrial movement can be examined along with interaction with microtubules and microtubule-based motors in presenilin-ablated neurons focusing on the relationship between presenilin, GSK3β, tau, and kinesins. Presenilin-associated defects in mitochondrial distribution can also be examined to determine if they affect energy mobilization, and the extent to which mitochondrial distribution defects contribute to neuronal dysfunction in presenilin-ablated neurons.

Alterations in mitochondrial function, for example impaired electron transport activity, defective oxidative phosphorylation or increased free radical production, can also arise as the result of defective mitochondria movement or localization. In one embodiment of the invention, a mitochondrial protein activity can be the indicator of altered ER-MAM integrity. The enzyme can be a mitochondrial enzyme, which can further be an electron transport chain enzyme or a Krebs cycle enzyme, or other enzymes or cellular components related to ATP production.

### Indicators of Altered ER-MAM Integrity: Free Radical Production

In certain embodiments of the invention, free radical production in a biological sample can be detected as an indicator of altered ER-MAM integrity. Without wishing to be bound by theory, increased communication between ER and mitochondria can result in elevated reactive oxygen species (ROS).

Accordingly, an indicator of altered ER-MAM integrity can be a free radical species present in a biological sample (e.g. reactive oxygen species). Methods of detecting free radicals are known in the art, and such methods include, but are not limited to fluorescent and/or chemiluminescent indicators (see Handbook of Methods for Oxygen Radical Research, 1985 CRC Press, Boca Raton, Fla.; Molecular Probes On-line Handbook of Fluorescent Probes and Research Chemicals, at http://www probes.com/handbook/toc.html). Free radical mediated damage to mitochondria can also result in collapse of the electrochemical potential maintained by the inner mitochondrial membrane. Methods for detecting changes in the inner mitochondrial membrane potential are described herein and in U.S. patent application Ser. No. 09/161,172.

Although mitochondria are a primary source of free radicals in biological systems (see, e.g., Murphy et al., 1998 in Mitochondria and Free Radicals in Neurodegenerative Diseases, Beal, Howell and Bodis-Wollner, Eds., Wiley-Liss, New York, pp. 159-186 and references cited therein), the invention should not be so limited and free radical production can be an indicator of altered ER-MAM integrity regardless of the subcellular source site.

In one aspect, the invention provides a method for diagnosing Alzheimer's disease in a subject, the method comprising: (a) obtaining a cell from an individual suspected of having Alzheimer's disease, (b) measuring an amount reactive oxygen species in the cell step (a), and (c) comparing the amount reactive oxygen species measured in the cell of step (a) to the amount reactive oxygen species measured in a control cell wherein, a greater amount reactive oxygen species measured in the cell of step (a) compared to the control cell indicates that the subject has Alzheimer's disease.

In one embodiment, reactive oxygen species (e.g. superoxide, hydrogen peroxide, singlet oxygen, and peroxynitrite) can be measured by using Mitosox Red (Molecular Probes). Mitosox Red is live-cell permeant and is selectively targeted to mitochondria. Once inside the mitochondria, the reagent is oxidized by superoxide and binds to nucleic acids, resulting in a red fluorescence. Increased MitoSox staining occurs in presenilin mutant cells compared to control cells (see Example 1).

In another embodiment, reactive oxygen species can be measured with 5-(and -6)-carboxy-2′,7′-dichlorodihydrofluorescein diacetate (carboxy-H₂DCFDA) in a “Image-iT Live” assay (Molecular Probes). Carboxy-H₂DCFDA is a fluorogenic marker for reactive oxygen species and is deacetylated by nonspecific intracellular esterases. In the presence of reactive oxygen species, the reduced fluorescein compound is oxidized and emits bright green fluorescence. Methods for detecting a free radical that may be useful as an indicator of altered ER-MAM integrity are known in the art and will depend on the particular radical.

### Indicators of ER-MAM Integrity: Alterations of Calcium Levels

Certain aspects of the present invention, as it relates to the correlation of Alzheimer's disease with an indicator of altered ER-MAM integrity, involve monitoring intracellular calcium homeostasis and/or cellular responses to perturbations of this homeostasis, including physiological and pathophysiological calcium regulation.

As described herein, PS1 is a regulator of Ca2+ storage in the ER and PS1 exerts an effect on ER-mitochondrial Ca2+ transfer, sensitizing mitochondria to permeabilization in FAD^{PS1} cells, leading to cell injury. Thus, according to certain embodiments of the present invention, release of ER-stored Ca2+ can potentiate influx of cytosolic free calcium into the mitochondria, as can occur under certain physiological conditions including those encountered by cells of a subject having increased communication between ER and mitochondria. Detection of such changes in calcium concentrations can be accomplished by a variety of means (see, e.g., Ernster et al., Cell Biol. 91:227 (1981); Haugland, 1996 Handbook of Fluorescent Probes and Research Chemicals—Sixth Ed., Molecular Probes, Eugene, Oreg.; Murphy et al., 1998 in Mitochondria & Free Radicals in Neurodegenerative Diseases, Beal, Howell and Bodis-Wollner, Eds., Wiley-Liss, New York; and Molecular Probes On-line Handbook of Fluorescent Probes and Research Chemicals, at http://www probes.com/handbook/toc.html).

In one aspect, the method of the present invention is directed to identifying a whether a compound is suitable for treating Alzheimer's disease by comparing a cellular response to elevated intracellular calcium in a biological sample from the subject with that of a control subject. The range of cellular responses to elevated intracellular calcium is broad, as is the range of methods and reagents for the detection of such responses. Many specific cellular responses are known to those having ordinary skill in the art. These responses can depend on the cell types present in a selected biological sample (see, e.g., Clapham, 1995 Cell 80:259; go Cooper, The Cell—A Molecular Approach, 1997 ASM Press, Washington, D.C.; Alberts, B., Bray, D., et al., Molecular Biology of the Cell, 1995 Garland Publishing, NY). 40 45 50 35 Acta 1016:87; Gunter and Gunter, 1994 /. Bioenerg. Biomembr. 26:471; Gunter et al., 1998 Biochim. Biophys. Acta 1366:5; McCormack et al., 1989 Biochim. Biophys. Acta 973:420; Orrenius and Nicotera, 1994 J. Neural. Transm. Suppl. 43:1; Leist and Nicotera, 1998 Rev. Physiol. Biochem. Pharmacol. 132:79; and Haugland, 1996 Handbook of Fluorescent Probes and Research Chemicals—Sixth Ed., Molecular Probes, Eugene, Oreg.)

As described herein, mutations in presenilins (or loss of presenilin function) can cause variation of ER, mitochondrial or cytosolic calcium levels from standard physiological ranges. In Alzheimer disease cells, mitochondrial calcium levels can be increased about 50% above the values in normal cells, and cytosolic Ca²⁺ can be increased by about 25% (i.e. from around 175 nM in normal cells to around 220 nM in AD cells after stimulation by exogenously-added ATP).

In one aspect, the invention provides a method for diagnosing Alzheimer's disease in a subject, the method comprising: (a) obtaining a biological sample from an individual suspected of having Alzheimer's disease, (b) measuring an amount of intracellular calcium in the biological sample of step (a), and (c) comparing the amount of intracellular calcium measured in the biological sample of step (a) to the amount of intracellular calcium measured in a control biological sample wherein, a increased amount of intracellular calcium measured in the biological sample of step (a) compared to the control biological sample indicates that the subject has Alzheimer's disease.

### Indicators of ER-MAM Integrity: Protein Interactions

Methods for determining ER-MAM-associated protein interactions can depend on the physicochemical properties of an ER-MAM-associated protein. In some embodiments, determination of ER-MAM-associated protein interactions can involve quantitative determination of the level of a protein or polypeptide interaction using routine methods known in the art (See, e.g., Harlow and Lane, Antibodies: A Laboratory Manual, Cold Spring Harbor Laboratory, 1988; Weir, D. M., Handbook of Experimental Immunology, 1986, Blackwell Scientific, Boston).

In some embodiments of the invention, the association between one of more ER-MAM-associated proteins can be the indicator of altered ER-MAM integrity.

In one aspect, the invention provides a method for diagnosing Alzheimer's disease in a subject, the method comprising: (a) obtaining a biological sample from an individual suspected of having Alzheimer's disease, (b) measuring an amount of an association between one or more ER-MAM-associated proteins in the biological sample of step (a), and (c) comparing the amount of an association between one or more ER-MAM-associated proteins measured in the biological sample of step (a) to the amount of an association between one or more ER-MAM-associated proteins measured in a control biological sample wherein, a increased amount of an association between one or more ER-MAM-associated proteins measured in the biological sample of step (a) compared to the control biological sample indicates that the subject has Alzheimer's disease.

As provided herein, associating ER-MAM-associated proteins can include, but are not limited to Acyl-CoA:cholesterol acyltransferase (ACAT1); Acyl-CoA desaturase (stearoyl-CoA desaturase 1); Apolipoprotein E; Autocrine motility factor receptor 2 (GP78); β-galactoside α(2-3) sialyltransferase (SIAT4); β-galactoside α(2-6) sialyltransferase (SIAT1); β-1,4 N-acetylgalactosaminyltransferase 1(SIAT2); β-1,4-galactosyltransferase 6 (lactosyl-ceramide synthase); Ceramide glucosyltransferase; Diacylglycerol O-acyltransferase; Fatty acid-CoA ligase, long-chain 1 (FACL1) (acyl-CoA synthetase 1); Fatty acid-CoA ligase, long-chain 4 (FACL4) (acyl-CoA synthetase 4); Fatty acid transport protein 4 (FATP4); Glucose-6-phosphatase; Glucose-regulated protein 78-kDa (BiP); Inositol 1,4,5-triphosphate receptor, type 3 (IP3R3); Microsomal triglyceride transfer protein large subunit; N-acetylglucosaminyl-phosphatidylinositol de-N-acetylase; Opioid receptor, sigma1; Phosphatidylethanolamine N-methyltransferase 2 (PEMT); Phosphatidylserine synthase 1 (PSS1); Phosphatidylserine synthase 2 (PSS2); Phosphofurin acidic cluster sorting protein 2; Presenilin 1; Presenilin 2; Ryanodine Receptor type 1; Ryanodine Receptor type 2; Ryanodine Receptor type 3; Amyloid beta precursor protein; Basigin/CD147/EMMPRIN; Cytomegalus virus-encoded vMIA protein fr unspliced exon I UL37 mRNA, N-term frag; Glucose-regulated protein 75-kDa (GRP75; Mortalin-2); and Membrane bound O-acyltransferase domain containing 2.

In one embodiment, an indicator of ER-MAM integrity is a modulation of the amount or character of a presenilin containing complex. The protein complexes and component proteins can be obtained by methods well known in the art for protein purification and recombinant protein expression. For example, the presenilin interaction partners can be isolated by immunoprecipitation from whole cell lysates or from purified cell fractions (e.g. ER-MAM cell fractions). In another embodiment, an indicator of ER-MAM integrity is a increase in the association of ER-MAM-associated proteins in a test biological sample (e.g. Diacylglycerol-O-acyltransferase 2 (DGAT2) and stearoyl-CoA desaturase 1 (SCD1)) to the association of ER-MAM-associated proteins in a control biological sample.

For recombinant expression of one or more of the proteins, the nucleic acid containing all or a portion of the nucleotide sequence encoding the protein can be inserted into an appropriate expression vector, i.e., a vector that contains the necessary elements for the transcription and translation of the inserted protein coding sequence. The necessary transcriptional and translational signals can also be supplied by the native promoter of the component protein gene, and/or flanking regions.

Assays for detecting, isolating and characterizing protein complexes are well known in the art (e.g., immunoassays, activity assays, mass-spectrometry . . . etc.) and can be used to determine whether one or more presenilin interaction partners are present at either increased or decreased levels, or are absent, in samples from patients suffering from AD, or having a predisposition to develop AD, as compared to the levels in samples from subjects not having AD, or having a predisposition to develop AD. Additionally, these assays can be used to determine whether the ratio of the complex to the un-complexed components in a presenilin containing protein complex, is increased or decreased in samples from patients suffering from AD, or having a predisposition to develop AD, as compared to the ratio in samples from subjects not having AD, or not having a predisposition to develop AD.

In the event that levels of one or more protein complexes (i.e., presenilin containing protein complexes) are determined to be increased in patients suffering from AD, or having a predisposition to develop AD, then the AD, or predisposition for AD, can be diagnosed, have prognosis defined for, be screened for, or be monitored by detecting increased levels of the one or more protein complexes, increased levels of the mRNA that encodes one or more members of the one or more protein complexes, or by detecting increased complex functional activity.

In the event that levels of one or more protein complexes (i.e., presenilin containing protein complexes) are determined to be altered in patients suffering from AD, or having a predisposition to develop AD, then the AD, or predisposition for AD, can be diagnosed, have prognosis defined for, be screened for, or be monitored by detecting altered levels of the one or more protein complexes, increased levels of the mRNA that encodes one or more members of the one or more protein complexes, or by detecting increased complex functional activity.

Accordingly, in one embodiment of the invention, AD involving aberrant compositions of presenilin containing protein complexes can be diagnosed, or their suspected presence can be screened for, or a predisposition to develop such disorders can be detected, by detecting the component proteins of one or more complexes from a whole cell lysate or from a subcellular fraction of a cellular lysate (e.g. an ER-MAM fraction).

Methods for screening for a molecule that binds a presenilin protein complex can be performed using cell-free and cell-based methods known in the art (e.g. in vitro methods, in vivo methods or ex vivo methods). For example, an isolated PS1 protein complex can be employed, or a cell can be contacted with the candidate molecule and the complex can be isolated from such contacted cells and the isolated complex can be assayed for activity or component composition.

Methods for screening can involve labeling the component proteins of the complex with, for example, radioligands, fluorescent ligands or enzyme ligands. Presenilin protein complexes can be isolated by any technique known in the art, including but not restricted to, co-immunoprecipitation, immunoaffinity chromatography, size exclusion chromatography, and gradient density centrifugation.

Suitable binding conditions are, for example, but not by way of limitation, in an aqueous salt solution of 10-250 mM NaCl, 5-50 mM Tris-HCl, pH 5-8, and a detergent. Suitable detergents can include, but are not limited to non-ionic detergents (for example, NP-40) or other detergents that improves specificity of interaction. One skilled in the art will readily be able to determine a suitable detergent and a suitable concentration for the detergent. Metal chelators and/or divalent cations can be added to improve binding and/or reduce proteolysis. Complexes can be assayed using routine protein binding assays to determine optimal binding conditions for reproducible binding.

Binding species can also be covalently or non-covalently immobilized on a substrate using any method well known in the art, for example, but not limited to the method of Kadonaga and Tjian, 1986, Proc. Natl. Acad. Sci. USA 83:5889-5893, i.e., linkage to a cyanogen-bromide derivatized substrate such as CNBr-Sepharose 4B (Pharmacia). Non-covalent attachment of proteins to a substrate include, but are not limited to, attachment of a protein to a charged surface, binding with specific antibodies and binding to a third unrelated interacting protein.

Proteins of the complex can be cross-linked to enhance the stability of the complex. Different methods to cross-link proteins are well known in the art. As will be apparent to a person skilled in the art, the optimal rate of cross-linking need to be determined on a case by case basis. This can be achieved by methods well known in the art, some of which are exemplary described herein.

### Indicators of ER-MAM Integrity as Measured by Fluorescence Resonance Energy Transfer

One indicator of ER-MAM integrity suitable for the purposes described herein is based on the known interaction between two ER-MAM-associated proteins—diacylglycerol-O-acyltransferase 2 (DGAT2) and stearoyl-CoA desaturase 1 (SCD1). These proteins form a dimeric complex in ER-MAM (Man et al. (2006) J. Lipid Res. 47:1928). When yellow fluorescent protein (CFP) is fused to DGAT2 (DGAT2-YFP) and cyan fluorescent protein (YFP) is fused to SCD1 (SCD1-YFP), illumination with light of the appropriate wavelength results in energy transfer from the YFP to the CFP (i.e. fluorescence resonant energy transfer) to yielding a visible signal. Fluorescence resonant energy transfer (FRET) occurs when the two proteins are within a few nanometers of one another. If the two polypeptides are separated from each other by even a few tens of nanometers, FRET does not occur.

Without wishing to be bound be theory, normal cells will have a weak FRET signal because in “thin” ER-MAM membranes. Conversely, when ER-MAM is increased in AD, ER-MAM becomes “thick” and the two traverse the membrane laterally in an altered manner. This will result in a increased FRET signal. This increase in FRET can be exploited as tool for diagnosis of AD and as a tool for identifying compounds useful for the treatment or prevention of AD. For example, fibroblasts (or other cells) from AD patients can be transfected with DGAT2-CFP and SCD1-YFP and the FRET can be assayed. Increased signal will be indicative of compromised ER-MAM, due to AD (or to any other pathology that affects ER-MAM integrity). Similarly, compounds can be screened for an ability to decrease a FRET signal in AD cells. For example, FAD^{PS1} or FAD^{PS2} cells can be transfected stably with a bicistronic vector containing DGAT2-CFP and SCD1-YFP, but owing to the ER-MAM defect they will have low FRET. These cells can be treated with a library of compounds to identify compounds that reduce FRET signals.

In one embodiment, ER-MAM integrity can be determined by measuring FRET between yellow fluorescent protein (YFP) fused to DGAT2 (DGAT2-CFP) and cyan fluorescent protein (CFP) fused to SCD1 (SCD1-YFP) upon illumination with light of the appropriate wavelength and energy transferred from the YFP to the CFP (i.e. fluorescence resonant energy transfer (FRET) to yield a signal). In another embodiment, the donor fluorophore and acceptor are selected so that the donor fluorophore and acceptor exhibit resonance energy transfer when the donor fluorophore is excited. A fluorescence resonance energy transfer (FRET) pair comprises a donor fluorophore and an acceptor where the overlap between the emissions spectrum of the donor fluorophore and the absorbance spectrum of the acceptor is sufficient to enable FRET.

In another embodiment, ER-MAM integrity can be determined by using “dark FRET” by measuring energy transfer between an ER-MAM-associated protein (e.g. DGAT2) fused to a fluorescent donor and an ER-MAM (e.g. SCD1) protein fused to a non-fluorescent chromoprotein (Ganesan et al, Proc Natl Acad Sci USA. 2006 Mar. 14; 103(11): 4089-4094). Suitable combinations of donor fluorophores and acceptor non-fluorescent chromoproteins, include, but are not limited to EYFP and REACh (Resonance Energy Accepting Chromoprotein) (Ganesan et al, Proc Natl Acad Sci USA. 2006 Mar. 14; 103(11): 4089-4094). A non-fluorescent chromoprotein can be any fluorescent protein (or variant thereof) that retains its absorption properties and can act as a quencher for the donor fluorescence. FRET with a non-fluorescent chromoprotein can be visualized by changes in the donor emission: its reduced lifetime by fluorescence lifetime imaging, quenched emission in relation to a reference fluorophore, and delayed photobleaching kinetics.

ER-MAM-associated proteins fused to a fluorescent proteins (or non-fluorescent chromoproteins) can be readily generated by methods known in the art. Such fluorescent fusion proteins (or non-fluorescent chromoproteins) can be used to detect protein interaction by several methods, including but not limited to immunoprecipitation and fluorescence resonance energy transfer (FRET). A fluorescent protein (or non-fluorescent chromoprotein) can be specifically linked to the amino- or carboxyl-terminus of an ER-MAM-associated protein sequence using well known chemical methods, see, e.g., Chemical Approaches to Protein Engineering, in Protein Engineering: A Practical Approach (Eds. Rees et al., Oxford University Press, 1992). A fluorescent protein (or non-fluorescent chromoprotein) can also be specifically inserted in-frame within an ER-MAM-associated protein using well known chemical methods.

The ER-MAM fluorescent-fusion proteins (or non-fluorescent chromoproteins) disclosed in the present specification include, in part, donor fluorophore. As used herein, the term “fluorophore” is synonymous with the term “fluorochrome” or “fluorescent molecule.” As used herein, the term “donor fluorophore” means a molecule that, when irradiated with light of a certain wavelength, emits light of a different wavelength, also denoted as fluorescence. Thus, a donor fluorophore can be a fluorescent molecule.

The ER-MAM fluorescent fusion proteins disclosed in the present specification include, in part, acceptor. As used herein, the term “acceptor” means a molecule that can absorb energy from a donor fluorophore and is a term that encompasses fluorescent molecules as well as non-fluorescent molecules. As used herein, the term “acceptor fluorophore” means an acceptor comprising a fluorescent molecule or any non-fluorescent chromoprotein. Any fluorescent molecules can serve as a donor fluorophore or an acceptor fluorophore, including, without limitation, a fluorescent protein, a fluorophore binding protein and a fluorescent dye.

A donor fluorophore or an acceptor fluorophore disclosed in the present specification can be, in part, a fluorescent protein. As used herein, the term “fluorescent protein” means a peptide which absorbs light energy of a certain wavelength and emits light energy of a different wavelength and encompasses those which emit in a variety of spectra, including violet, blue, cyan, green, yellow, orange and red. Fluorescent proteins derived from any of a variety of species can be useful in aspects of the present invention including, but not limited to, Aequorea fluorescent proteins, Anemonia fluorescent proteins, Anthozoa fluorescent proteins, Discosoma fluorescent proteins, Entacmeae fluorescent proteins, Heteractis fluorescent proteins, Montastrea fluorescent proteins, Renilla fluorescent proteins, Zoanthus fluorescent proteins, and fluorescent proteins from other organisms. Fluorescent proteins useful in the invention encompass, without limitation, wild type fluorescent proteins, naturally occurring variants, and genetically engineered variants, produced, e.g., by random mutagenesis or rational designed, and active peptide fragments derived from an organism.

Fluorescent proteins (or non-fluorescent chromoproteins) useful in aspects of the invention include, e.g., those which have been genetically engineered for superior performance such as, without limitation, altered excitation or emission wavelengths; enhanced brightness, pH resistance, stability or speed of fluorescent protein formation; photoactivation; or reduced oligomerization or photobleaching, see, e.g., Brendan P. Cormack et al., FACS-optimized Mutants of the Green Fluorescent Protein (GFP), U.S. Pat. No. 5,804,387 (Sep. 8, 1998); Roger Y. Tsien & Roger Heim, Modified Green Fluorescent Proteins, U.S. Pat. No. 6,800,733 (Oct. 5, 2004); Roger Y. Tsien et al., Long Wavelength Engineered Fluorescent Proteins, U.S. Pat. No. 6,780,975 (Aug. 24, 2004); and Roger Y. Tsien et al., Fluorescent Protein Sensors For Measuring the pH of a Biological Sample, U.S. Pat. No. 6,627,449 (Sep. 30, 2003).

A fluorescent protein (or non-fluorescent chromoprotein) can be engineered for improved protein expression by converting wild type codons to other codons more efficiently utilized in the cells which serve to express the ER-MAM-associated protein, see, e.g., Brian Seed and Jurgen Haas, High Level Expression of Proteins, U.S. Pat. No. 5,795,737 (Aug. 18, 1998). A fluorescent protein (or non-fluorescent chromoprotein) can be operably-linked to an ER-MAM-associated protein to create a fusion protein using standard molecular genetic techniques. In one aspect, the ER-MAM-associated protein can be any of Acyl-CoA:cholesterol acyltransferase (ACAT1); Acyl-CoA desaturase (stearoyl-CoA desaturase 1); Apolipoprotein E; Autocrine motility factor receptor 2 (GP78); β-galactoside α(2-3) sialyltransferase (SIAT4); β-galactoside α(2-6) sialyltransferase (SIAT1); β-1,4 N-acetylgalactosaminyltransferase 1(SIAT2); β-1,4-galactosyltransferase 6 (lactosyl-ceramide synthase); Ceramide glucosyltransferase; Diacylglycerol O-acyltransferase; Fatty acid-CoA ligase, long-chain 1 (FACL1) (acyl-CoA synthetase 1); Fatty acid-CoA ligase, long-chain 4 (FACL4) (acyl-CoA synthetase 4); Fatty acid transport protein 4 (FATP4); Glucose-6-phosphatase; Glucose-regulated protein 78-kDa (BiP); Inositol 1,4,5-triphosphate receptor, type 3 (IP3R3); Microsomal triglyceride transfer protein large subunit; N-acetylglucosaminyl-phosphatidylinositol de-N-acetylase; Opioid receptor, sigma1; Phosphatidylethanolamine N-methyltransferase 2 (PEMT); Phosphatidylserine synthase 1 (PSS1); Phosphatidylserine synthase 2 (PSS2); Phosphofurin acidic cluster sorting protein 2; Presenilin 1; Presenilin 2; Ryanodine Receptor type 1; Ryanodine Receptor type 2; Ryanodine Receptor type 3; Amyloid beta precursor protein; Basigin/CD147/EMMPRIN; Cytomegalus virus-encoded vMIA protein fr unspliced exon I UL37 mRNA, N-term frag; Glucose-regulated protein 75-kDa (GRP75; Mortalin-2); and Membrane bound O-acyltransferase domain containing 2.

Any of a variety of fluorescently active protein fragments can be useful in aspects of the present invention with the proviso that these active fragments retain the ability to emit light energy in a range suitable for the proper operation of aspects of the present invention, such as, e.g. about 420-460 nm for blue emitting fluorescent proteins, about 460-500 nm for cyan emitting fluorescent proteins, about 500-520 nm for green emitting fluorescent proteins, about 520-550 nm for yellow emitting fluorescent proteins and about 550-740 nm for red emitting fluorescent proteins (Table 3).

TABLE 3
Excitation and Emission Maxima of Exemplary
Fluorescent Proteins Fluorescent proteins

| | Excitation | Emission |
|---|---|---|
| | Maxima (nm) | Maxima (nm) |
| EBFP | 380 | 440 |
| ACFP | 439 | 476 |
| AmCyan | 458 | 489 |
| AcGFP | 475 | 505 |
| ZsGreen | 493 | 505 |
| Vitality .RTM. | 500 | 506 |
| hrGFP | | |
| Monster Green | 505 | 515 |
| EYFP | 512 | 529 |
| ZsYellow | 529 | 539 |
| DsRed-Express | 557 | 579 |
| DsRed2 | 563 | 582 |
| DsRed | 558 | 583 |
| AsRed2 | 576 | 592 |
| HcRed1 | 588 | 618 |

Non-limiting examples of fluorescent proteins that can be operably-linked to an ER-MAM-associated protein include, e.g., photoproteins, such as, e.g., aequorin; obelin; Aequorea fluorescent proteins, such, e.g., green fluorescent proteins (GFP, EGFP, AcGFP.sub.1), cyan fluorescent proteins (CFP, ECFP), blue fluorescent proteins (BFP, EBFP), red fluorescent proteins (RFP), yellow fluorescent proteins (YFP, EYFP), ultraviolet fluorescent protein (GFPuv), their fluorescence-enhancement variants, their peptide destabilization variants, and the like; coral reef fluorescent proteins, such, e.g., Discosoma red fluorescent proteins (DsRed, DsRed1, DsRed2, and DsRed-Express), Anemonia red fluorescent proteins (AsRed and AsRed2), Heteractis far-red fluorescent proteins (HcRed, HcRed1), Anemonia cyan fluorescent proteins (AmCyan, AmCyan1), Zoanthus green fluorescent proteins (ZsGreen, ZsGreen1), Zoanthus yellow fluorescent proteins (ZsYellow, ZsYellow1), their fluorescence-enhancement variants, their peptide destabilization variants, and the like; Renilla reniformis green fluorescent protein (Vitality hrGFP), its fluorescence-enhancement variants, its peptide destabilization variants, and the like; and Great Star Coral fluorescent proteins, such, e.g., Montastrea cavernosa fluorescent protein (Monster Green® Fluorescent Protein), its fluorescence-enhancement variants, its peptide destabilization variants, and the like. It is apparent to one skilled in the art that these and a variety of other fluorescent proteins can be useful as a fluorescent protein in aspects of the invention, see, e.g., Jennifer Lippincott-Schwartz & George H. Patterson, Development and Use of Fluorescent Protein Markers in Living Cells, 300(5616) Science 87-91 (2003); and Jin Zhang et al., 3(12) Nat. Rev. Mol. Cell. Biol. 906-918 (2002).

It is apparent to one skilled in the art that these and many other fluorescent proteins, including species orthologs and paralogs of the herein described naturally occurring fluorescent proteins as well as engineered fluorescent proteins can be useful as a fluorescent protein disclosed in aspects of the present specification. ER-MAM-associated proteins disclosed in the present specification containing, in part, such fluorescent proteins can be prepared and expressed using standard methods see, e.g., Living Colors® User Manual PT2040-1 (PRI1Y691), BD Biosciences-Clontech, (Nov. 26 2001); BD Living Colors™ User Manual Volume II: Reef Coral Fluorescent Proteins, PT3404-1 (PR37085), BD Biosciences-Clontech, (Jul. 17, 2003); Monster Green Florescent Protein pHMCFP Vector, TB320, Promega Corp., (May, 2004); and Vitality hrGFP Mammalian Expression Vectors, Instruction Manual (rev. 064007g), Stratagene, Inc. Expression vectors suitable for bacterial, mammalian and other expression of fluorescent proteins are available from a variety of commercial sources including BD Biosciences Clontech (Palo Alto, Calif.); Promega Corp. (Madison, Wis.) and Stratagene, Inc. (La Jolla, Calif.).

### Indicators of ER-MAM Integrity as Measured by Lanthanide Donor Complex Luminescence

A luminescence resonance energy transfer (LRET) pair comprises a lanthanide donor complex and an acceptor where the overlap between the emissions spectrum of the lanthanide donor complex and the absorbance spectrum of the acceptor is sufficient to enable LRET.

Aspects of the present invention can rely on a recombinant ER-MAM-associated protein which contains a donor fluorophore comprising a lanthanide donor complex. In other aspects, a donor fluorophore is a lanthanide donor complex. An ER-MAM-associated protein comprising a lanthanide donor complex exploits the luminescent properties of lanthanides, which are their long, millisecond to submillisecond lifetimes, narrow and multiple emission bands in the visible spectrum, and unpolarized emission.

A lanthanide donor complex includes a lanthanide ion such as, without limitation, a terbium ion, europium ion, samarium ion or dysprosium ion. Lanthanide ions, or “rare earth” elements, are a group of elements whose trivalent cations emit light at well-defined wavelengths and with long decay times. Lanthanides include, without limitation, elements with atomic numbers 57 through 71: lanthanide (La); cerium (Ce); praseodymium (Pr); neodymium (Nd); promethium (Pm); samarium (Sm); europium (Eu); gadolinium (Gd); terbium (Tb); dysprosium (Dy); holmium (Ho); erbium (Er); thulium (Tm); ytterbium (Yb); and lutetium (Lu). Lanthanides can further include, without limitation, yttrium (Y; atomic number 39) and scandium (Sc; atomic number 21).

A lanthanide-binding site useful in a lanthanide donor complex can be a peptide or peptidomimetic, such as, e.g., an EF-hand motif. As used herein, the term “EF-hand motif” means two α-helices flanking the coordination site of an EF-hand motif A variety of naturally occurring EF-hands are known in the art, as described, e.g., Hiroshi Kawasaki and Robert H. Kretsinger, Calcium-Binding Proteins 1: EF-Hands, 1(4) Protein Profile 343-517 (1994); and Susumu Nakayama and Robert H. Kretsinger, Evolution of the EF-Hand Family of Proteins, Annu. Rev. Biophys. Biomol. Struct. 473-507 473-507 (1994); Hiroshi Kawasaki et al., Classification and Evolution of EF-Hand Proteins, 11(4) Biometals 277-295 (1998); and Yubin Zhou et al., Prediction of EF-Hand Calcium-Binding Proteins and Analysis of Bacterial Proteins 65(3) Proteins 643-655 (2006).

Indicators of Altered ER-MAM Integrity: Cell death

In another aspect, the invention relates to the correlation of Alzheimer's disease with an indicator of altered ER-MAM integrity involving cell death. In one aspect, the invention provides a method for determining whether a test compound is capable of treating Alzheimer's disease by comparing a cellular response to an apoptogenic stimulus, where such response is an indicator of altered ER-MAM integrity as provided herein. Altered mitochondrial physiology can be involved in programmed cell death (Zamzami et al., Exp. Med. 182:367-77, 1995; Zamzami et al., Exp. Med. 181:1661-72, 1995; Marchetti et al., Cancer Res. 56:2033-38, 1996; Monaghan et al., J. Histochem. Cytochem. 40:1819-25, 1992; Korsmeyer et al, Biochim. Biophys. Act. 1271:63, 1995; Nguyen et al., Biol. Chem. 269:16521-24, 1994). Thus, changes in mitochondrial physiology can be important mediators of cell death. Altered mitochondrial function, as can be used for determining whether a test compound is capable of treating Alzheimer's disease according to the present disclosure, can therefore increase the threshold for induction of cell death by an apoptogen. A variety of apoptogens are known to those familiar with the art (see, e.g., Green et al., 1998 Science 281:1309 and references cited therein).

In one embodiment of the subject invention method wherein the indicator of altered ER-MAM integrity is a cellular response to an apoptogen, cells in a biological sample that are suspected of undergoing apoptosis can be examined for morphological, permeability or other changes that are indicative of an apoptotic state. For example by way of illustration and not limitation, apoptosis in many cell types can cause altered morphological appearance such as plasma membrane blebbing, cell shape change, caspase activation, translocation of cell membrane phosphatidylserine from the inner to the outer leaflet of the plasma membrane, loss of substrate adhesion properties or other morphological changes that can be readily detected by a person having ordinary skill in the art, for example by using light microscopy.

A person having ordinary skill in the art will readily appreciate that there can be other suitable techniques for quantifying apoptosis, and such techniques for purposes of determining an indicator of ER-MAM integrity that is a cellular response to an apoptogenic stimulus are within the scope of the methods provided by the present invention.

### Indicators of Altered ER-MAM Integrity: APP Cleavage or β-Secretase Activity

Any known marker or correlate to AD can be used as a marker of altered ER-MAM integrity. While not wishing to be bound to theory, inhibition of β-secretase activity is thought to inhibit production of β amyloid β peptide (Aβ). Reduction of APP cleavage at the β-secretase cleavage site compared with an untreated or inactive control can be used to determine inhibitory activity. Methods for determining β-secretase activity are known in the art. Exemplary systems include, but are not limited to assay systems are described in U.S. Pat. No. 5,942,400. Thus, in one embodiment, the extent rate or amount cleavage of APP at the β-secretase cleavage site can be used as a marker of ER-MAM integrity. Assays that demonstrate inhibition of β-secretase-mediated cleavage of APP can utilize any of the known forms of APP (see, for example, U.S. Pat. No. 5,766,846 and also Hardy, 1992, Nature Genet. 1:233-234).

### Indicators of Altered ER-MAM Integrity: Reduced Glucose Metabolism

Reduced glucose utilization and deficient energy metabolism occur in the pathogenesis of AD (Castellani, R. et al. Role of mitochondrial dysfunction in Alzheimer's disease. J. Neurosci. Res. 70, 357-360 (2002)).

In one aspect, the invention provides a method for diagnosing Alzheimer's disease in a subject, the method comprising: (a) obtaining a biological sample from an individual suspected of having Alzheimer's disease, (b) measuring the amount of glucose metabolism in the biological sample of step (a), and (c) comparing the amount of glucose metabolism measured in the biological sample of step (a) to the amount of glucose metabolism measured in a control biological sample wherein, a reduced amount of glucose metabolism measured in the biological sample of step (a) compared to the control biological sample indicates that the subject has Alzheimer's disease. Methods for measuring glucose metabolism in a biological sample are well known in the art (e.g. glucose-6-phosphate phosphatase can be assayed by established procedures (Vance and Vance, 1988).

### Indicators of Altered ER-MAM Integrity: Cholesterol Content

Cholesterol is selectively reduced an AD “double-transgenic” (i.e. mutations in both APP and PS1) mouse model (Yao et al. (2008) Neurochem. Res. in press).

Thus, in one aspect, the invention provides a method for diagnosing Alzheimer's disease in a subject, the method comprising: (a) obtaining a biological sample from an individual suspected of having Alzheimer's disease, (b) measuring the amount of cholesterol in the biological sample of step (a), and (c) comparing the amount of cholesterol measured in the biological sample of step (a) to the amount of cholesterol measured in a control biological sample wherein, a reduced amount of cholesterol measured in the biological sample of step (a) compared to the control biological sample indicates that the subject has Alzheimer's disease. Methods for measuring cholesterol content of a biological sample are well known in the art (e.g. fillipin staining).

### Correlation of Apolipoprotein Genotype

In humans, there are three alleles of apolipoprotein E: ApoE2, ApoE3, and ApoE4. Individuals harboring at least one ApoE4 allele are at risk for developing sporadic AD (SAD). Like PS1 and PS2, ApoE4 is a ER-MAM-localized protein. The results described herein show that the mitochondrial maldistribution phenotype, as well as the increase in communication between the ER and mitochondria (both indicators of altered ER-MAM integrity) are correlated to the ApoE4 genotype. Specifically, cells with E3/E3 have normal ER-MAM content, whereas those with E3/E4 have increased communication between the ER and mitochondria, irrespective of whether or not the cells harbor a presenilin mutation (e.g. cells with a PS1 mutation and an E3/E3 genotype have normal communication between the ER and mitochondria and normal mitochondrial distribution, whereas PS1 cells with E3/E4 have increased communication between the ER and mitochondria and altered mitochondria). Similarly, the amount of communication between the ER and mitochondria in E3/E4 brain tissue from FAD^{PS1} or FAD^{PS2} patients is increased compared to that in E3/E3 brain tissue from FAD^{PS1} or FAD^{PS2} patients. This result explains the role of ApoE in the pathogenesis of AD, and connects the familial and sporadic forms of the disease into one conceptual framework.

The finding that presenilin is a ER-MAM-associated protein, that the amount of communication between the ER and mitochondria is increased in FAD^{PS1} or FAD^{PS2} cells, and that ApoE4 allele status can affect ER-MAM integrity show that the fundamental problem in both FAD and SAD is altered ER-MAM integrity. Thus in one aspect, the invention described herein provides a method for determining whether a subject has an ApoE3/E4 genotype, the method comprising, obtaining a biological sample from an individual suspected of having Alzheimer's disease, measuring an indicator of ER-MAM integrity in the biological sample and comparing the indicator of ER-MAM integrity in the biological sample of step to the indicator of ER-MAM integrity in a control sample wherein, a change in the indicator of ER-MAM integrity measured in the biological sample compared to the control sample indicates that the subject has an ApoE3/E4 genotype. In another aspect, the invention described herein provides a method for determining whether a subject has an ApoE4/E4 genotype, the method comprising, obtaining a biological sample from an individual suspected of having Alzheimer's disease, measuring an indicator of ER-MAM integrity in the biological sample and comparing the indicator of ER-MAM integrity in the biological sample of step to the indicator of ER-MAM integrity in a control sample wherein, a change in the indicator of ER-MAM integrity measured in the biological sample compared to the control sample indicates that the subject has an ApoE4/E4 genotype.

### Screening Methods and Compound Libraries

The invention also provides methods useful for identifying compounds or agents which are capable of treating Alzheimer's disease (or more generally, neurodegenerative diseases that have altered ER-MAM) in a subject. Generally, test compounds are selected if they can reverse an indicator of ER-MAM in a biological sample, model AD cell or animal-model to a state or condition or level comparable to a wild-type or normal cell or animal. In one embodiment, a test compound can be examined for an ability to increase or a decrease an indicator of ER-MAM integrity in a cell. In another embodiment, a test compound can be examined for an ability to cause an increase or a decrease in the ratio of perinuclear mitochondria to non-perinuclear mitochondria in a cell. For example, a suitable test compound may be (but is not limited to) a compound which can reduce the ratio of perinuclear mitochondria to non-perinuclear mitochondria in an AD cell. In another embodiment, a test compound can be examined for an ability to cause an increase or a decrease in the amount of communication between the ER and mitochondria in a biological sample. For example, a suitable test compound may be (but is not limited to) a compound which can decrease the communication between the ER and mitochondria in an AD cell. In another embodiment, a test compound can be examined for an ability to increase or a decrease the ratio punctate to non-punctate mitochondria in a cell. For example, a suitable test compound may be (but is not limited to) a compound which can reduce ratio of punctate to non-punctate mitochondria in an AD cell. In another embodiment, a test compound can be examined for an ability to increase or a decrease the conversion of phosphatidylserine to phosphatidylethanolamine in a biological sample. For example, a suitable test compound may be (but is not limited to) a compound which can decrease the conversion of phosphatidylserine to phosphatidylethanolamine in an AD cell. In another embodiment, a test compound can be examined for an ability to increase or a increase survival of a cell contacted with cinnamycin. For example, a suitable test compound may be (but is not limited to) a compound which increase survival of an AD cell contacted with cinnamycin. In another embodiment a test compound can be examined for an ability to increase or a decrease the association of ER-MAM-associated proteins (e.g. Diacylglycerol-O-acyltransferase 2 (DGAT2) and stearoyl-CoA desaturase 1 (SCD1)). For example, a suitable test compound may be (but is not limited to) a compound which can decrease the association of ER-MAM-associated proteins (e.g. Diacylglycerol-O-acyltransferase 2 (DGAT2) and stearoyl-CoA desaturase 1 (SCD1)) in an AD cell. In another embodiment, a test compound can be examined for an ability to increase or a decrease the amount of one or more reactive oxygen species in a cell. For example, a suitable test compound may be (but is not limited to) a compound which can decrease the amount of one or more reactive oxygen species in an AD cell.

Suitable biological samples for identifying compounds or agents which are capable of treating Alzheimer's disease can comprise any tissue or cell preparation in which at least one candidate indicator of altered ER-MAM integrity can be detected, and can vary in nature accordingly, depending on the indicator(s) of ER-MAM integrity to be compared. Biological samples can be provided by obtaining a blood sample, biopsy specimen, tissue explant, organ culture or any other tissue or cell preparation from a subject or a biological source. The subject or biological source can be a human or non-human animal, a primary cell culture or culture adapted cell line including but not limited to genetically engineered cell lines. For example, suitable biological samples for diagnosing Alzheimer's disease include cells obtained in a non-invasive manner. Examples include, but are not limited to a neuron, a fibroblast, a skin biopsy, a blood cell (e.g. a lymphocyte), an epithelial cell and biological materials found in urine sediment.

AD model disease cells suitable for use with the methods described herein include, but are not limited to, human skin fibroblasts derived from patients carrying FAD-causing presenilin mutations, mouse skin fibroblasts, cultured embryonic primary neurons, and any other cells derived from PS1-knock out transgenic mice (containing null mutation in the PS1 gene), cells having AD-linked familial mutations, cells having genetically associated AD allelic variants, cells having sporadic AD, or cells having mutations associated with sporadic AD.

AD-linked familial mutations include AD-linked presenilin mutations (Cruts, M. and Van Broeckhoven, C., Hum. Mutat. 11:183-190 (1998); Dermaut, B. et al., Am. J. Hum. Genet. 64:290-292 (1999)), and amyloid β-protein precursor (APP) mutations (Suzuki, N. et al., Science 264:1336-1340 (1994); De Jonghe, C. et al., Neurobiol. Dis. 5:281-286 (1998)).

Genetically associated AD allelic variants include, but are not limited to, allelic variants of apolipoprotein E (e.g., APOE4) (Strittmatter, W. J. et al., Proc. Natl. Acad. Sci. USA 90:1977-1981 (1993)).

More specifically, AD model disease cells can include, but not limited to, one or more of the following mutations, for use in the invention: APP FAD mutations (e.g., E693Q (Levy E. et al., Science 248:1124-1126 (1990)), V717 I (Goate A. M. et al., Nature 349:704-706 (1991)), V717F (Murrell, J. et al., Science 254:97-99 (1991)), V717G Chartier-Harlin, M. C. et al., Nature 353:844-846 (1991)), A682G (Hendriks, L. et al., Nat. Genet. 1:218-221 (1992)), K/M670/671N/L (Mullan, M. et al Nat. Genet. 1:345-347 (1992)), A713V (Carter, D. A. et al., Nat. Genet. 2:255-256 (1992)), A713T (Jones, C. T. et al., Nat. Genet. 1:306-309 (1992)), E693G (Kamino, K. et al., Am. J. Hum. Genet. 51:998-1014 (1992)), T673A (Peacock, M. L. et al., Neurology 43:1254-1256 (1993)), N665D (Peacock, M. L. et al., Ann. Neurol. 35:432-438 (1994)), 1716V (Eckman, C. B. et al., Hum. Mol. Genet. 6:2087-2089 (1997)), and V715M (Ancolio, K. et al., Proc. Natl. Acad. Sci. USA 96:4119-4124 (1999))); presenilin FAD mutations (e.g., all point (missense) mutations except one - - - 113Δ4 (deletion mutation)); PS1 mutations (e.g., A79V, V82L, V96F, 113Δ4, Y115C, Y115H, T116N, P117L, E120D, E120K, E123K, N135D, M139, I M139T, M139V, I 143F, 1143T, M461, I M146L, M146V, H163R, H163Y, S169P, S169L, L171P, E184D, G209V, 1213T, L219P, A231T, A231V, M233T, L235P, A246E, L250S, A260V, L262F, C263R, P264L, P267S, R269G, R269H, E273A, R278T, E280A, E280G, L282R, A285V, L286V, S290C (Δ9), E318G, G378E, G384A, L392V, C410Y, L424R, A426P, P436S, P436Q); PS2 mutations (R62H, N141I, V148I, M293V). Other cell types are readily known to those of ordinary skill in the art.

Animal models useful in testing the such compounds include those expressing elevated levels of Aβ, demonstrating an enhanced amount of Aβ deposits, and/or increased number or size of β amyloid plaques as compared with control animals. Suitable animal models include, but are not limited to transgenic mammals. Transgenic mice expressing native and mutant forms of the presenilin proteins have been described (Borchelt et al., Neuron, 1996, 17, 1005-1013; Duff et al., Nature, 1996, 383, 710-713; Borchelt et al., Neuron, 1997, 19, 939-945; Citron et al., Nature Med., 1997, 3, 67-72; Chui et al., Nature Med., 1999, 5, 560-564; Nakano et al., Eur. J. Neurosci., 1999, 11, 2577-2581; Chui et al., Nature Med., 1999, 5, 560-564; Borchelt et al., Neuron, 1997, 19, 939-945; Holcomb et al., Nature Med., 1998, 4, 97-100; Lamb et al., Nature Neurosci., 1999, 2, 695-697; Wong et al., Nature, 1997, 387, 288-292; Shen et al., Cell, 1997, 89, 629-639; DeStrooper et al., Nature, 1998, 391, 387-390; Ganes et. al., 1995, Nature 373:523). Other examples of suitable transgenic animal models include those described in, for example, U.S. Pat. Nos. 5,877,399, 5,612,486, 5,850,003, 5,877,015, 5,877,399, 5,612,486, 5,387,742, 5,720,936, and 5,811,633.

Examples of such compounds include, but are not limited to, small organic molecules including pharmaceutically acceptable molecules. Examples of small molecules include, but are not limited to, polypeptides, peptidomimetics, amino acids, amino acid analogs, nucleic acids, nucleic acid analogs, nucleotides, nucleotide analogs, organic or inorganic compounds (i.e., including heteroorganic and organometallic compounds) having a molecular weight of less than about 10,000 grams per mole, salts, esters, and other pharmaceutically acceptable forms of such compounds. Examples of other compounds that can be tested in the methods of this invention include polypeptides, antibodies, nucleic acids, and nucleic acid analogs, natural products and carbohydrates.

A compound can have a known chemical structure but not necessarily have a known function or biological activity. Compounds can also have unidentified structures or be mixtures of unknown compounds, for example from crude biological samples such as plant extracts. Large numbers of compounds can be randomly screened from chemical libraries, or collections of purified chemical compounds. or collections of crude extracts from various sources. The chemical libraries can contain compounds that were chemically synthesized or purified from natural products. Methods of introducing test compounds to cells are well known in the art.

Those having ordinary skill in the art will appreciate that a diverse assortment of compound libraries can be prepared according to established procedures, and tested for their influence on an indicator of altered ER-MAM integrity. The test compounds can be obtained using any of the numerous approaches in combinatorial library methods known in the art (see Lam K S, Anticancer Drug Des. 12:145-67 (1997)). Such compound libraries are also available from commercial sources such as ComGenex (U.S. Headquarters, South San Francisco, Calif.), Maybridge (Cornwall, UK), and SPECS (Rijswijk, Netherlands), ArQule, Tripos/PanLabs, ChemDesign and Pharmacopoeia.

Therapeutic agents or combinations of agents suitable for the treatment or prevention of AD can be identified by screening of candidate agents on normal, AD or cybrid cells constructed with patient mitochondria. The invention also provides methods of identifying an agent suitable for treating a subject suspected of being at risk for having AD by comparing the level of at least one indicator of altered ER-MAM integrity, in the presence and absence of a candidate compound, to determine the suitability of the agent for treating AD. The compounds identified in the screening methods of this invention can be novel or can be novel analogs or derivatives of known therapeutic agents.

In some embodiments, a compound can be tested for the ability to modulate an indicator of ER-MAM integrity, modulate the ratio of perinuclear mitochondria to non-perinuclear mitochondria is a cell, modulate the amount of communication between the ER and mitochondria in a biological sample, modulate the ratio punctate to non-punctate mitochondria in a cell, modulate the conversion of phosphatidylserine to phosphatidylethanolamine in a biological sample, modulate the amount of cell survival in a cell contacted with cinnamycin, modulate the association of ER-MAM-associated proteins (e.g. Diacylglycerol-O-acyltransferase 2 (DGAT2) and stearoyl-CoA desaturase 1 (SCD1)), modulate the amount of one or more reactive oxygen species, or modulate an indicator of mitochondria-associated integrity in a cell.

### Expression of Presenilin

In one aspect, the invention described herein provides methods for determining whether a test compound is capable of treating Alzheimer's disease. In one embodiment, the method comprises overexpressing presenilin or reducing presenilin expression with shRNA technology, contacting a cell or biological sample with a test compound, measuring an measuring an indicator of ER-MAM integrity in the cell, and comparing the indicator of mitochondria-associated membrane integrity measured in the cell or biological sample with an indicator of ER-MAM integrity measured in a control cell or biological sample that has not been contacted with a test compound, wherein an increase or decrease in the indicator of mitochondria-associated membrane integrity measured in the cell or biological sample relative to the indicator of mitochondria-associated membrane integrity measured in the control cell or biological sample indicates that the test compound is capable of treating Alzheimer's disease.

### Classification of AD

The present invention provides compositions and methods that are useful in pharmacogenomics, for the classification of a subject or patient population without the use of a genetic test. In one embodiment, for example, such classification can be achieved by identification in a subject or patient population of one or more distinct profiles of at least one indicator ER-MAM integrity that correlate with AD. Such profiles can define parameters indicative of a subject's predisposition to develop AD, and can further be useful in the identification of new subtypes of AD. In another embodiment, correlation of one or more traits in a subject with at least one indicator of altered ER-MAM integrity can be used to gauge the subject's responsiveness to, or the efficacy of, a therapeutic treatment.

As described herein, determination of levels of at least one indicator of altered ER-MAM integrity can also be used to classify a AD patient population (i.e., a population classified as having AD by independent criteria). In another embodiment of the invention, determination of levels of at least one indicator of altered ER-MAM integrity in a biological sample from a AD subject can provide a useful correlative indicator for that subject. An AD subject so classified on the basis of levels of at least one indicator of altered ER-MAM integrity can be monitored using AD clinical parameters, such that correlation between levels of at least one indicator of altered ER-MAM integrity and any clinical score used to evaluate AD can be monitored as a useful marker with which to correlate the efficacy of any candidate therapeutic agent being used in AD subjects.

### Recombinant Expression Vectors and Host Cells

The recombinant expression vectors for expression of polypeptides of this invention in prokaryotic or eukaryotic cells can be designed. For example, polypeptide of this invention can be expressed in bacterial cells such as insect cells (e.g., using baculovirus expression vectors), yeast cells, amphibian cells, or mammalian cells. Suitable host cells are well known to one skilled in the art. For other suitable expression systems for both prokaryotic and eukaryotic cells, see chapters 16 and 17 of Sambrook, J., Fritsh, E. F., and Maniatis, T. Molecular Cloning: A Laboratory Manual, 2nd ed., Cold Spring Harbor Laboratory, Cold Spring Harbor Laboratory Press, Cold Spring Harbor, N.Y., 1989.

A number of these methodologies can also be applied in vivo, systemically or locally, in a complex biological system such as a human. For example, increased copy number of nucleic acids encoding ER-MAM-associated proteins in expressible from (by DNA transfection), can be employed.

### Animal Models

Once an test compound is identified to be able, modulate an indicator of ER-MAM integrity, modulate the ratio of perinuclear mitochondria to non-perinuclear mitochondria is a cell, modulate the amount of communication between the ER and mitochondria in a biological sample, modulate the ratio punctate to non-punctate mitochondria in a cell, modulate the conversion of phosphatidylserine to phosphatidylethanolamine in a biological sample, modulate the amount of cell survival in a cell contacted with cinnamycin, modulate the association of ER-MAM-associated proteins (e.g. Diacylglycerol-O-acyltransferase 2 (DGAT2) and stearoyl-CoA desaturase 1 (SCD1)), modulate the amount of one or more reactive oxygen species in a cell, or modulate an indicator of mitochondria-associated integrity in a cell, the agent can be tested for its ability treat Alzheimer's disease in animal models. Animal models useful in testing the such compounds include those expressing elevated levels of Aβ, demonstrating an enhanced amount of Aβ deposits, and/or increased number or size of β amyloid plaques as compared with control animals. Suitable animal models include, but are not limited to transgenic mammals, including but not limited to ApoE4 mice (e.g. mice having human a ApoE4 transgene or a knock-in to “humanize” the mouse ApoE gene).

Transgenic mice expressing native and mutant forms of the presenilin proteins have been described (Borchelt et al., Neuron, 1996, 17, 1005-1013; Duff et al., Nature, 1996, 383, 710-713; Borchelt et al., Neuron, 1997, 19, 939-945; Citron et al., Nature Med., 1997, 3, 67-72; Chui et al., Nature Med., 1999, 5, 560-564; Nakano et al., Eur. J. Neurosci., 1999, 11, 2577-2581; Chui et al., Nature Med., 1999, 5, 560-564; Borchelt et al., Neuron, 1997, 19, 939-945; Holcomb et al., Nature Med., 1998, 4, 97-100; Lamb et al., Nature Neurosci., 1999, 2, 695-697; Wong et al., Nature, 1997, 387, 288-292; Shen et al., Cell, 1997, 89, 629-639; DeStrooper et al., Nature, 1998, 391, 387-390; Ganes et. al., 1995, Nature 373:523). Other examples of suitable transgenic animal models include those described in, for example, U.S. Pat. Nos. 5,877,399, 5,612,486, 5,850,003, 5,877,015, 5,877,399, 5,612,486, 5,387,742, 5,720,936, and 5,811,633.

### Compromised ER-MAM Integrity in Neurodegenerative Diseases and Disorders

In a further aspect, the diagnostic methods disclosed herein can be used for determining whether a subject has, or is at risk of having a neurodegenerative disease or disorder. In another aspect, the screening methods disclosed herein can be used to identify a compound useful in the treatment, prevention or reduction of a neurodegenerative disease or disorder.

Exemplary neurodegenerative diseases or disorders include, but are not limited to, Alexander disease, Alper's disease, Alzheimer's disease (Sporadic and Familial), Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease (Spinocerebellar ataxia type 3), Multiple sclerosis, Multiple System Atrophy, Parkinson disease, Pelizaeus-Merzbacher Disease, Pick's disease, Primary lateral sclerosis, Refsum's disease, Sandhoff disease, Schilder's disease, Schizophrenia, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Tabes dorsalis, Angelman syndrome, Autism, Fetal Alcohol syndrome, Fragile X syndrome, Tourette's syndrome, Prader-Willi syndrome, Sex Chromosome Aneuploidy in Males and in Females, William's syndrome, Smith-Magenis syndrome, 22q Deletion, or any combination thereof.

The following examples illustrate the present invention, and are set forth to aid in the understanding of the invention, and should not be construed to limit in any way the scope of the invention as defined in the claims which follow thereafter.

### EXAMPLES

### Example 1

### ER-Mitochondrial Interaction in Familial Alzheimer Disease

Clinically, FAD is similar to SAD but has an earlier age of onset. PS1 and PS2 are ubiquitously-expressed aspartyl proteases that are about 50-kDa in size. The active forms of PS1 and PS2 are N- and C-terminal fragments (NTF and CTF, respectively), which are produced by cleavage of full-length presenilin in its “loop” domain (Zhou S, Zhou H, Walian P J, Jap B K (2007) Regulation of γ-secretase activity in Alzheimer's disease. Biochemistry 46:2553-2563). PS1 and PS2 are components of the γ-secretase complex that processes a number of plasma-membrane proteins, including Nothc, Jagged and APP. The γ-secretase complex also contains three other structural subunits: APH1, nicastrin, and PEN2 (De Strooper B (2003) Aph-1, Pen-2, and nicastrin with presenilin generate an active γ-secretase complex. Neuron 38:9-12).

Following cleavage of APP by β-secretase, γ-secretase cleaves the C-terminal “β-stub” to release small amyloidogenic fragments, 40- and 42-aa in length (Aβ40 and Aβ42), that have been implicated in the pathogenesis of AD (Goedert M, Spillantini M G (2006) A century of Alzheimer's disease. Science 314:777-781).

Whereas the components of the γ-secretase complex are localized predominantly intracellularly (Siman R, Velji J (2003) Localization of presenilin-nicastrin complexes and γ-secretase activity to the trans-Golgi network. J. Neurochem. 84:1143-1153), its substrates, including APP, are located mainly in the plasma membrane (PM) (Kaether C, Schmitt S, Willem M, Haass C (2006) Amyloid precursor protein and Notch intracellular domains are generated after transport of their precursors to the cell surface. Traffic 7:408-415). Moreover, while active γ-secretase is present in the PM, APP is apparently processed by an intracellular γ-secretase (Tarassishin L, Yin Y I, Bassit B, Li Y M (2004) Processing of Notch and amyloid precursor protein by γ-secretase is spatially distinct. Proc. Natl. Acad. Sci. USA 101:17050-17055).

The diverse sites of γ-secretase activity, APP processing, and Aβ production are the basis of what has been called the “spatial paradox” (Cupers P et al, (2001)

The discrepancy between presenilin subcellular localization and γ secretase processing of amyloid precursor protein. J. Cell Biol. 154:731-740). Besides the ER and Golgi, PS1 has been localized to the nuclear envelope (Kimura et al, (2001) Age-related changes in the localization of presenilin-1 in cynomolgus monkey brain. Brain Res. 922:30-41), endosomes (Vetrivel et al, (2004) Association of γ-secretase with lipid rafts in post-Golgi and endosome membranes. J. Biol. Chem. 279:44945-44954), lysosomes (Pasternak et al, (2003) Presenilin-1, nicastrin, amyloid precursor protein, and γ-secretase activity are co-localized in the lysosomal membrane. J. Biol. Chem. 278:26687-26694), mitochondria (Hansson et al., (2004) Nicastrin, presenilin, APH-1, and PEN-2 form active γ-secretase complexes in mitochondria. J. Biol. Chem. 279:51654-51660), and the plasma membrane (Tarassishin L, Yin Y I, Bassit B, Li Y M (2004) Processing of Notch and amyloid precursor protein by γ-secretase is spatially distinct. Proc. Natl. Acad. Sci. USA 101:17050-17055), where it is especially enriched at intercellular contacts known as adherens junctions (Marambaud et al., (2002) A presenilin-1/γ-secretase cleavage releases the E-cadherin intracellular domain and regulates disassembly of adherens junctions. EMBO J. 21:1948-1956). Furthermore, PS1 and other γ-secretase components are present in cholesterol- and sphingolipid-rich membrane microdomains (“lipid rafts”) (Vetrivel et al., (2005) Spatial segregation of γ-secretase and substrates in distinct membrane domains. J. Biol. Chem. 280:25892-25900) involved in signaling and trafficking (Hancock J F (2006) Lipid rafts: contentious only from simplistic standpoints. Nature Rev. Mol. Cell. Biol. 7:456-462).

The ER is the cell's main store of calcium, which is released upon stimulation by input signals such as inositol 1,4,5-triphosphate (IP3) and sphingosine-1 phosphate (Berridge M J (2002) The endoplasmic reticulum: a multifunctional signaling organelle. Cell Calcium 32:235-249), while the main site of calcium uptake is the mitochondrion. The ER and mitochondria are linked not only biochemically but also physically (Csordas et al., (2006) Structural and functional features and significance of the physical linkage between ER and mitochondria. J. Cell Biol. 174:915-921; Jousset et al., (2007) STIM1 knockdown reveals that store-operated Ca2+ channels located close to sarco/endoplasmic Ca2+ ATPases (SERCA) pumps silently refill the endoplasmic reticulum. J. Biol. Chem. 282:11456-11464; Rizzuto et al., (1998) Close contacts with the endoplasmic reticulum as determinants of mitochondrial Ca2+ responses. Science 280:1763-1766). Endoplasmic reticulum-mitochondrial-associated membranes (ER-MAM, or ER-MAM) are ER-contiguous membranes associated with mitochondria (Rusinol et al., (1994) A unique mitochondria-associated membrane fraction from rat liver has a high capacity for lipid synthesis and contains pre Golgi secretory proteins including nascent lipoproteins. J. Biol. Chem. 269:27494-27502) that constitute a physical bridge that connects the ER to mitochondria (Csordas et al., (2006) Structural and functional features and significance of the physical linkage between ER and mitochondria. J. Cell Biol. 174:915-921; Rusinol et al., (1994) A unique mitochondria-associated membrane fraction from rat liver has a high capacity for lipid synthesis and contains pre Golgi secretory proteins including nascent lipoproteins. J. Biol. Chem. 269:27494-27502).

More than a dozen proteins are concentrated in ER-MAM, involved mainly in lipid and intermediate metabolism (e.g. phosphatidylethanolamine N-methyltransferase [PEMT] Vance et al, (1997) Phosphatidylethanolamine N-methyltransferase from liver. Biochim. Biophys. Acta 1348:142-150); acyl-coenzyme A:cholesterol acyltransferase 1 (ACAT1) (Csordas et al., (2006) Structural and functional features and significance of the physical linkage between ER and mitochondria. J. Cell Biol. 174:915-921), and in the transfer of lipids between the ER and mitochondria (Csordas et al., (2006) Structural and functional features and significance of the physical linkage between ER and mitochondria. J. Cell Biol. 174:915-921; Man et al., (2006) Colocalization of SCD1 and DGAT2: implying preference for endogenous monounsaturated fatty acids in triglyceride synthesis. J. Lipid Res. 47:1928-1939).

A few non enzymatic proteins are also concentrated in ER-MAM, including the IP3 receptor (IP3R) (Hajnoczky et al., (2002) Old players in a new role: mitochondria-associated membranes, VDAC, and ryanodine receptors as contributors to calcium signal propagation from endoplasmic reticulum to the mitochondria. Cell Calcium 32:363-377), autocrine motility factor receptor (AMFR; an E3 ubiquitin ligase that targets ER proteins for proteasomal degradation (Registre et al., (2004) The gene product of the gp78/AMFR ubiquitin E3 ligase cDNA is selectively recognized by the 3F3A antibody within a subdomain of the endoplasmic reticulum. Biochem. Biophys. Res. Commun. 320:1316-1322), and apolipoprotein E (ApoE) (Csordas et al., (2006) Structural and functional features and significance of the physical linkage between ER and mitochondria. J. Cell Biol. 174:915-921; Vance J E (1990) Phospholipid synthesis in a membrane fraction associated with mitochondria. J. Biol. Chem. 265:7248-7256). Phosphofurin acidic cluster sorting protein 2 (PACS2) controls the apposition of mitochondria with the ER and stabilizes and regulate the interaction of ER and mitochondria (Simmen et al., (2005) PACS-2 controls endoplasmic reticulum mitochondria communication and Bid-mediated apoptosis. EMBO J. 24:717-729).

Mammalian mitochondria move predominantly along microtubules (Rube D A, van der Bliek A M (2004) Mitochondrial morphology is dynamic and varied. Mol. Cell. Biochem. 256:331-339). Such movement is important in neurons, where mitochondria travel from the cell body to the extremities at the ends of axons and dendrites (Li et al., (2004) The importance of dendritic mitochondria in the morphogenesis and plasticity of spines and synapses. Cell 119:873-887) to provide energy for pre-synaptic transmission and for postsynaptic uptake of critical small molecules (e.g. neurotransmitters; Ca²⁺). Anterograde and retrograde mitochondrial movements are driven by kinesins and dyneins, respectively. The binding of kinesins to mitochondria is dynamic, and depends on the degree of phosphorylation of kinesin (De Vos et al., (2000) Tumor necrosis factor induces hyperphosphorylation of kinesin light chain and inhibits kinesin-mediated transport of mitochondria. J. Cell Biol. 149:1207-1214) by glycogen synthase kinase 3β (GSK3β) (Morfini et al., (2002) Glycogen synthase kinase 3 phosphorylates kinesin light chains and negatively regulates kinesin-based motility. EMBO J. 21:281-293).

PS1 and PS2. The results and analysis described herein that relate to the effect of PS1 on ER-MAM integrity (for example, results relating to PS1 mutations, overexpression of PS1 and reduced expression) also apply to PS2. For example, the effects on ER-MAM integrity that occur as a result of loss or reduction of PS1 function, also occur where PS2 function is lost or reduced.

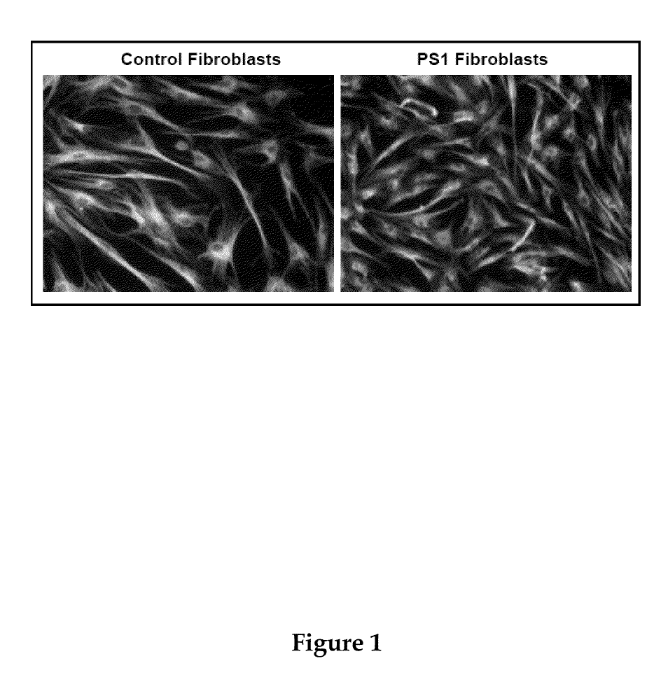

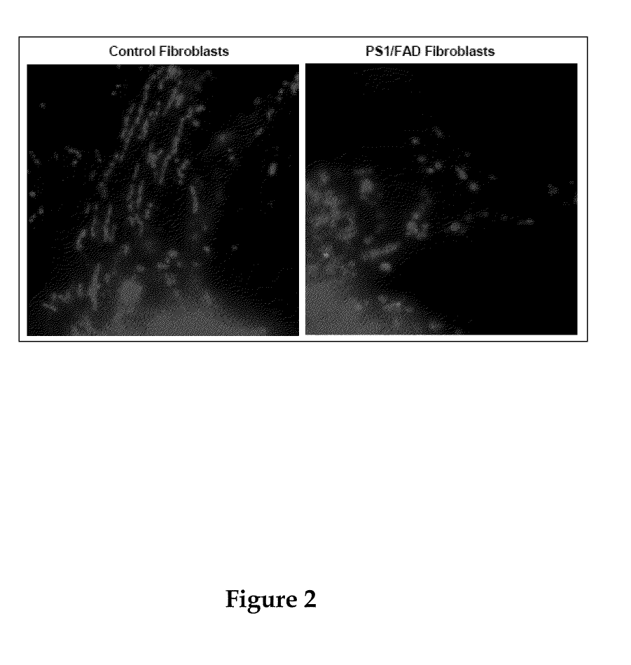

Morphology of AD Fibroblasts. Skin fibroblasts from patients with FAD due to a mutation in PS1 are significantly smaller, more “spherical” and less elongated, and have an altered perinuclear distribution of mitochondria as compared to control fibroblasts. Notably, these three properties are consistent with the mislocalization of mitochondrial as described herein. PS1-mutant fibroblasts are smaller than age- and sex-matched control fibroblasts (FIG. 1). This was confirmed in a more objective way by trypsinizing PS1 and control fibroblasts to de-attach them from the plates, and then analyzing them by fluorescent-activated cell sorting (FACS). This analysis confirmed that PS1 fibroblasts are significantly smaller than controls, and that unattached PS1 fibroblasts are significantly less elongated than controls (i.e. they have a smaller aspect ratio) (FIG. 2). This sphericity may occur if organelles are no longer attached to microtubules.

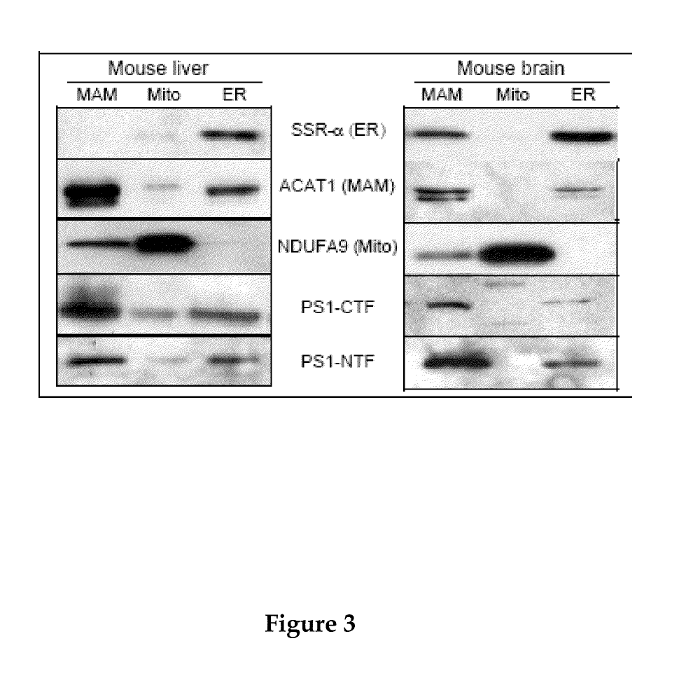

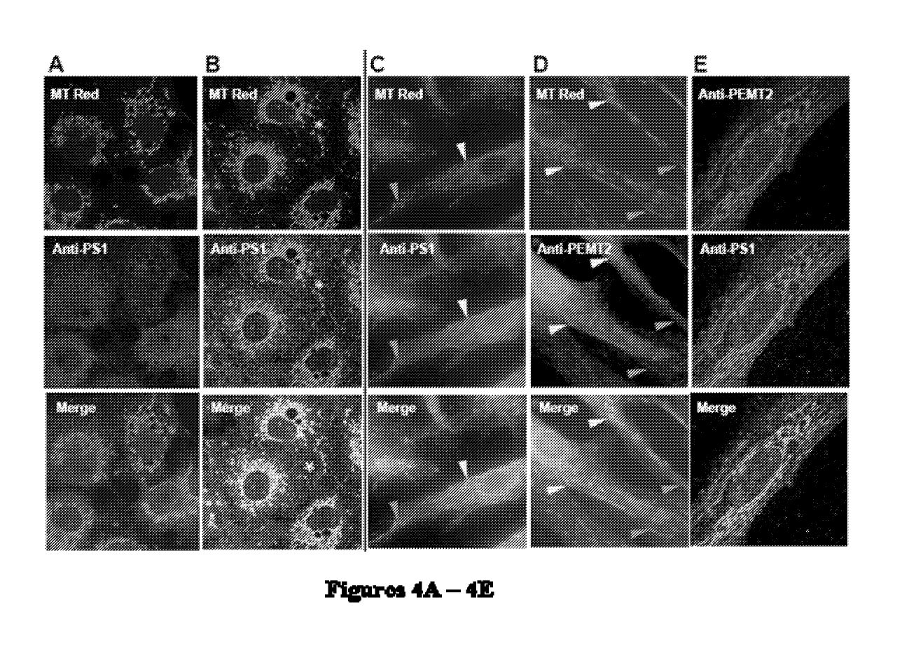

Presenilins are ER-MAM-associated protein. Plasma membrane (PM), ER, and crude mitochondria (CM) was isolated from mouse liver, and then fractionated CM into a ER-MAM fraction and a purified mitochondrial fraction (Vance et al., (1997) Phosphatidylethanolamine N-methyltransferase from liver. Biochim. Biophys. Acta 1348:142-150); the purity of the fractions was confirmed by Western blotting. Western blot analysis was performed on ER, ER-MAM, and mitochondria isolated from mouse liver and brain using relevant antibodies for the 3 compartments (SSR-α, ACAT1, and NDUFA9, respectively) as well as antibodies that recognize both the N- and C-terminal fragments of PS1 (FIG. 3). The majority of PS1 (both NTF and CTF) in both tissues was present in the ER-MAM fraction, similar to the pattern seen for ACAT1, a known ER-MAM-associated protein. The localization of PS1 to that of PEMT, another known ER-MAM-associated protein, was compared using immunocytochemistry and staining with MitoTracker Red (MTred) to visualize mitochondria. Using cold methanol (MeOH) to dehydrate and fix the cells, it was found that PEMT colocalized with MTred staining (FIG. 4D). This result is consistent with the fact that PEMT is enriched in a compartment bridging mitochondria and ER. The colocalization of PEMT with MTred was most pronounced in the region around the nucleus (red arrowheads in FIG. 4D). This results shows that that ER-MAM is located predominantly in the perinuclear region under these fixation conditions. PS1 also co-localized with MTred, also predominantly in the perinuclear region (FIG. 4C). Double-staining of cells for both PS1 and PEMT showed that the two proteins colocalized almost exactly (FIG. 4E). These results show that PS1, like PEMT and ACAT1, is a ER-MAM-associated protein.

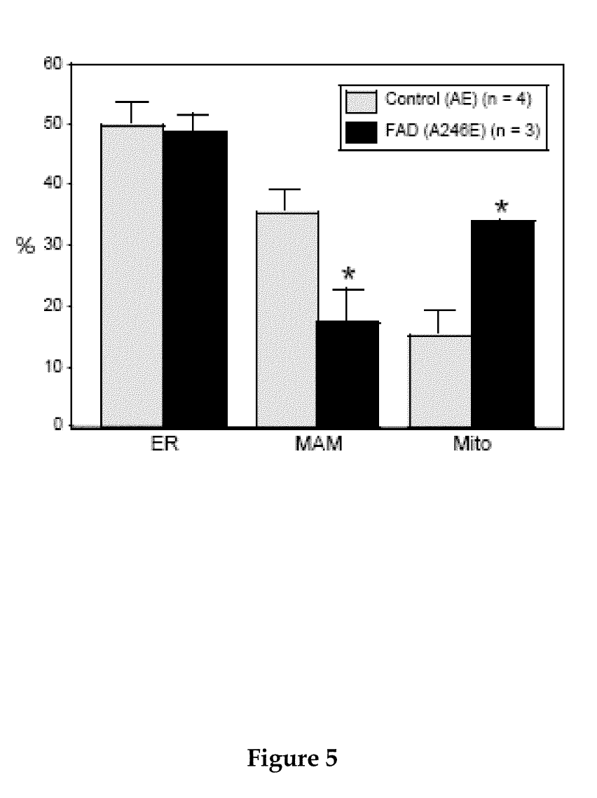

MAM and mitochondria in fibroblasts FAD^{PS1} or FAD^{PS2} patients. Because presenilin is located in a domain connecting ER with mitochondria, subcellular fractionation of control and FAD^{PS1} (A246E mutation) fibroblasts was performed and total protein recovered in ER, ER-MAM, and mitochondrial fractions was measured to determine if pathogenic mutations in PS1 affect these compartments, qualitatively or quantitatively. A significant decrease in the amount of ER-MAM protein, increase in ER-MAM function and a significant increase in the amount of mitochondria in FAD^{PS1} cells vs. controls was observed (FIG. 5).

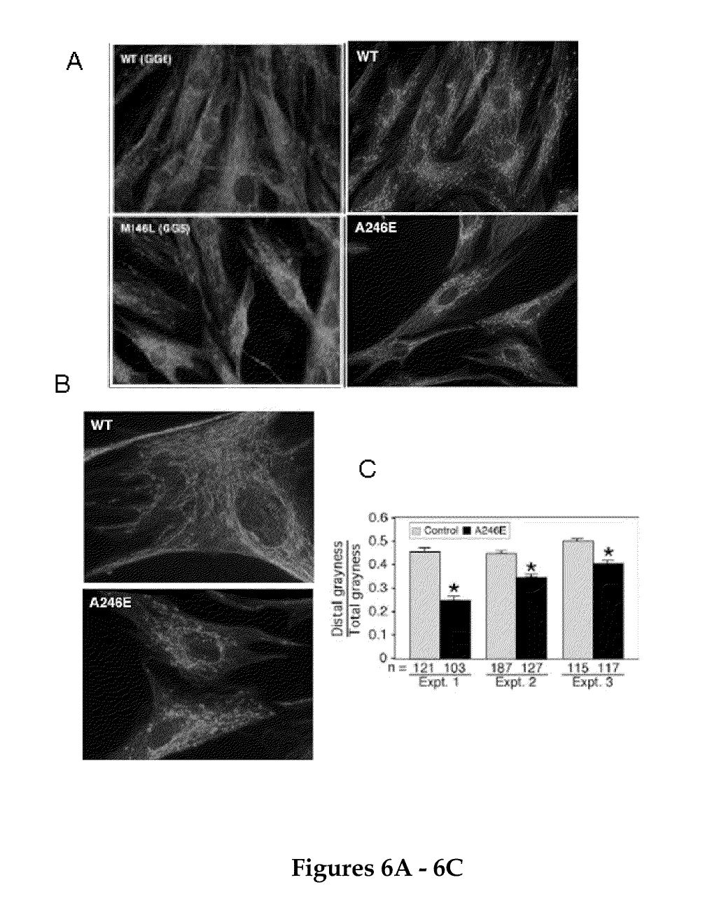

The morphology and distribution of MTred-labeled mitochondria in control and FAD^{PS1} fibroblasts (mutations A246E and M146L) was examined. To define cell boundaries, the microtubule cytoskeleton was visualized by indirect immunofluorescence with anti-tubulin antibodies in the same cells. Mitochondria in PS1-mutant fibroblast lines were more concentrated around the nucleus than were mitochondria in controls and fewer mitochondria were observed at the extremities of FAD^{PS1} cells (representative result in FIG. 6A). This effect was quantitated by measuring the intensity of the MTred signal in the periphery of the cell. A circle of uniform size that occupied about ⅔ of the cell's area and also encompassed the nucleus was drawn and the amount of MTred signal outside the circle was measured. Using this approach, it was confirmed that there indeed were significantly fewer mitochondria in the cells' extremities in FAD^{PS1} cells as compared to controls (FIG. 6C). The proportion of MAM in the cells was increased significantly (FIG. 6). These findings support the concept that PS1 contributes to the stabilization of MAM. This “perinuclear” result is consistent with a defect in microtubular transport of mitochondria to the edges of the cells.

Besides an altered subcellular distribution, the mitochondria in FAD^{PS1} cells also had an altered morphology. Whereas mitochondria in control fibroblasts had an elongated, tubular morphology, mitochondria in patient fibroblasts were more punctate (FIG. 6B). The FAD^{PS1} cells showed no obvious deficit in respiratory chain function. To reproduce the mitochondrial distribution defect observed in FAD^{PS1} patient cells, COS-7 cells were transfected with a construct expressing wild-type PS1 or the A246E mutation (7). Visualization of mitochondria and the microtubule cytoskeleton in transfected cells showed that mitochondria in the cells over-expressing mutant PS1, but not control cells, accumulated in the perinuclear region of the cell. This is a phenotype similar to that observed in FAD^{PS1} cells.

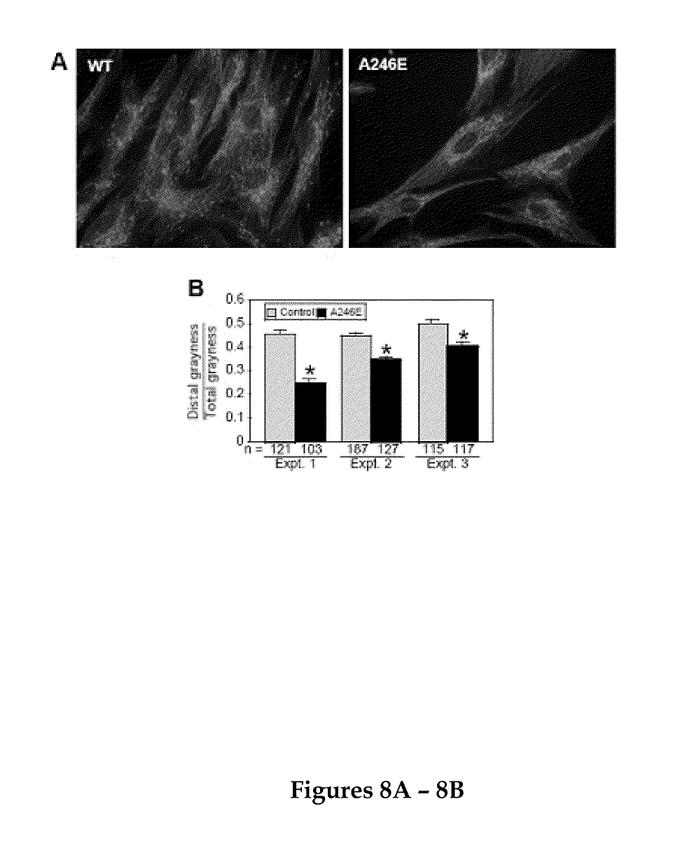

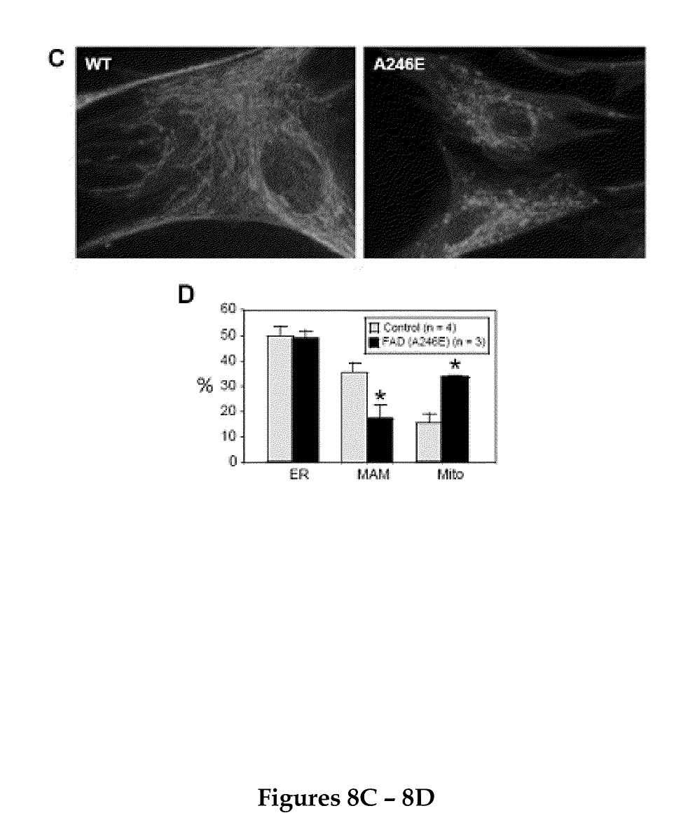

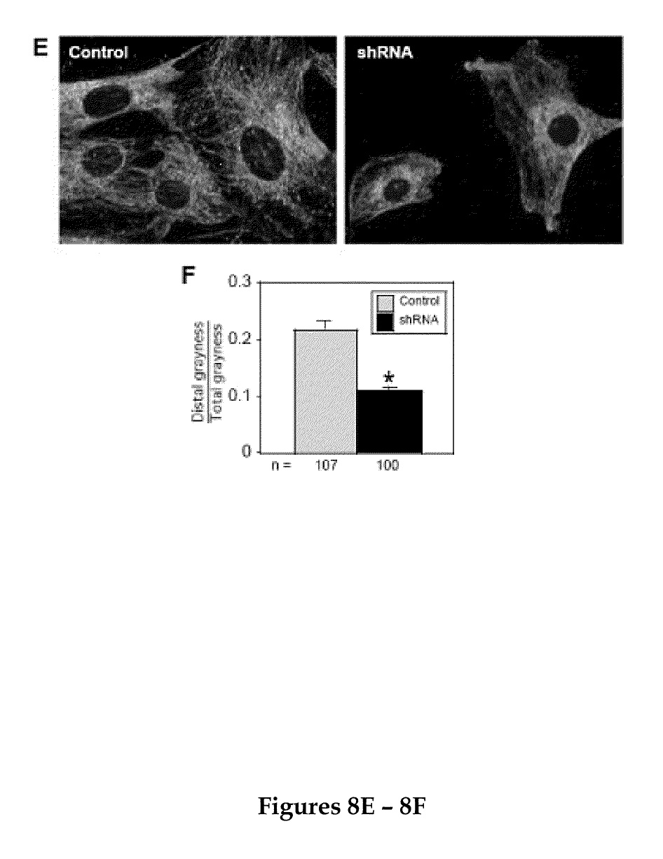

Small hairpin RNA (sh-RNA) technology was used to knock down PS1 expression in mouse embryonic fibroblasts to reproduce the mitochondrial mislocalization phenotype. The “perinuclear” phenotype observed in cells that overexpress mutant PS1 or in cells from FAD^{PS1} patients was recapitulated using cells in which PS1 expression was reduced by about 75% (FIG. 8E-F). The specificity of the shRNA primer was confirmed by transducing a mismatch shRNA, which did not alter the mitochondrial distribution or morphology.

These results show that a pathogenic problem in FAD due to mutations in presenilin is the mislocalization of mitochondria in affected cells. Presenilins are part of the machinery required for the reversible, kinesin-mediated, binding of mitochondria to microtubules, and that mutations in presenilins cause mitochondria to dissociate from microtubules. Mitochondria are key players in FAD, and dysfunction of mitochondria in FAD may include mislocalization and ATP synthesis defects, increased ROS, and elevated Ca²⁺ homeostasis. Since mitochondria are the main source of energy in the cell, failure to bind to microtubules efficiently can result in energy deficits in those parts of the cell that are relatively devoid of organelles. This mislocalization of mitochondria need not be deleterious in most cells, which are essentially “spherical” (i.e. they are not diffusion-limited for ATP), but can be more problematic in elongated neurons, which require that mitochondria travel vast distances along microtubules in order to provide ATP for energy-intensive processes at, for example, synaptic junctions.

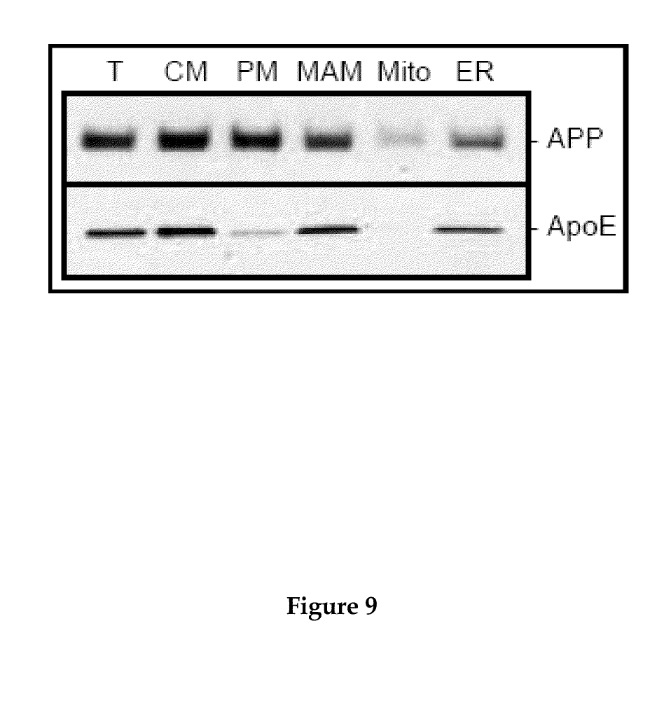

ApoE and APP Are Present in ER-MAM. The enrichment of ApoE in ER-MAM was noted more than 15 years ago (Rusinol et al., (1994) A unique mitochondria-associated membrane fraction from rat liver has a high capacity for lipid synthesis and contains pre Golgi secretory proteins including nascent lipoproteins. J. Biol. Chem. 269:27494-27502; Vance J E (1990) Phospholipid synthesis in a membrane fraction associated with mitochondria. J. Biol. Chem. 265:7248-7256). In addition to confirming that ApoE is enriched in ER-MAM (FIG. 9) the results described herein show that APP is also present in abundant amounts in ER-MAM (FIG. 9). These findings show that ER-MAM is involved not only in familial AD, but in sporadic AD as well.

The findings that presenilins are enriched in ER-MAM, that presenilins affect the stability of this compartment, and that presenilins affect the distribution of mitochondria which can be associated with ER-MAM, point to an additional role for presenilins in the development of AD. Numerous hypotheses have been put forward to explain the etiology and progression of the disease. Foremost among these are hypotheses invoking β-amyloid and tau, but alterations in cholesterol, glucose, and lipid metabolism, and in calcium homeostasis, play roles in AD pathogenesis. ER-MAM harbors proteins involved in lipid metabolism (e.g. fatty acid-CoA ligase (Lewin et al., (2002) Rat liver acyl-CoA synthetase 4 is a peripheral-membrane protein located in two distinct subcellular organelles, peroxisomes, and mitochondrial-associated membrane. Arch. Biochem. Biophys. 404:263-270), phosphatidylserine synthase (Stone S J, Vance J E (2000) Phosphatidylserine synthase-1 and -2 are localized to mitochondria-associated membranes. J. Biol. Chem. 275:34534-34540), ceramide glucosyltransferase (Ardail et al., (2003) The mitochondria-associated endoplasmic-reticulum subcompartment (MAM fraction) of rat liver contains highly active sphingolipid-specific glycosyltransferases. Biochem. J. 371:1013-1019), diacylglycerol O-acyltransferase 2 (Man et al., (2006) Colocalization of SCD1 and DGAT2: implying preference for endogenous monounsaturated fatty acids in triglyceride synthesis. J. Lipid Res. 47:1928-1939), in cholesterol metabolism (e.g. acyl-CoA:cholesterol acyltransferase (Rusinol et al., (1994) A unique mitochondria-associated membrane fraction from rat liver has a high capacity for lipid synthesis and contains pre Golgi secretory proteins including nascent lipoproteins. J. Biol. Chem. 269:27494-27502)), and in glucose metabolism (e.g. glucose-6-phosphatase (Bionda et al., (2004) Subcellular compartmentalization of ceramide metabolism: ER-MAM (mitochondria-associated membrane) and/or mitochondria? Biochem. J. 382:527-533)). Besides enzymatic functions, ER-MAM is also enriched in proteins involved in lipoprotein transport (e.g. microsomal triglyceride transfer protein large subunit (Rusinol et al., (1994) A unique mitochondria-associated membrane fraction from rat liver has a high capacity for lipid synthesis and contains pre Golgi secretory proteins including nascent lipoproteins. J. Biol. Chem. 269:27494-27502)), ubiquitination (e.g. autocrine motility factor receptor 2 (Goetz J G, Nabi I R (2006) Interaction of the smooth endoplasmic reticulum and mitochondria. Biochem. Soc. Trans. 34:370-373), calcium homeostasis (e.g. IP3 receptor (Csordas et al., (2006) Structural and functional features and significance of the physical linkage between ER and mitochondria. J. Cell Biol. 174:915-921), and apoptosis (phosphofurin acidic cluster sorting protein 2 (Simmen et al., (2005) PACS-2 controls endoplasmic reticulum mitochondria communication and Bid-mediated apoptosis. EMBO J. 24:717-729)), and can also contain enzymes involved in the unfolded protein response (Sun et al., (2006) Localization of GRP78 to mitochondria under the unfolded protein response. Biochem. J. 396:31-39) and mitochondrial fission (Breckenridge et al., (2003) Caspase cleavage product of BAP31 induces mitochondrial fission through endoplasmic reticulum calcium signals, enhancing cytochrome c release to the cytosol. J. Cell Biol. 160:1115-11127).

Given these functions, alterations in ER-MAM structure, function, and integrity can explain many of the biochemical changes found in cells and tissues from AD patients. Moreover, because PS1, ApoE and APP are present in ER-MAM, the familial and sporadic forms of AD can be related in a fundamental way, in which altered ER-MAM integrity is the common denominator. The results described herein take AD research in a new direction, as it predicts a cause-and-effect relationship between altered ER-MAM integrity, mitochondrial dynamics, and neurodegeneration. This relationship is not unreasonable, since mitochondrial mislocalization plays a role in the pathogenesis of other neurodegenerative diseases. These include (1) hereditary spastic paraplegia type 7, due to mutations in paraplegin (SPG7), a mitochondrial AAA protease, which is associated with abnormal mitochondria and impaired axonal transport (Ferreirinha et al., (2004) Axonal degeneration in paraplegin-deficient mice is associated with abnormal mitochondria and impairment of axonal transport. J. Clin. Invest. 113:231-242); (2) Charcot-Marie-Tooth disease type 2A, a peripheral neuropathy caused by mutations in the kinesin motor KIF1B and in mitofusin 2 (MFN2; a mitochondrial outer membrane protein required for organellar fusion); both cause altered axonal transport (Baloh et al., (2007) Altered axonal mitochondrial transport in the pathogenesis of Charcot-Marie-Tooth disease from mitofusin 2 mutations. J. Neurosci. 27:422-430; Zhao et al., (2001) Charcot-Marie-Tooth disease type 2A caused by mutation in a microtubule motor KIF1Bβ. Cell 105:587-597); (3) Charcot-Marie-Tooth disease type 4A, due to mutations in ganglioside-induced differentiation associated protein 1 (GDAP1), a mitochondrial outer membrane protein that regulates organellar morphology (Niemann et al. (2005) Ganglioside-induced differentiation associated protein 1 is a regulator of the mitochondrial network: new implications for Charcot-Marie-Tooth disease. J. Cell Biol. 170:1067-1078); and (4) autosomal-dominant optic atrophy, due to mutations in OPA1 (a mitochondrial dynamin-related protein that interacts with mitofusin-1 (Cipolat et al., (2004) OPA1 requires mitofusin 1 to promote mitochondrial fusion. Proc. Natl. Acad. Sci. USA 101:15927-15932)) and which is characterized by a maldistribution of mitochondria in affected cells (Delettre et al., (2000) Nuclear gene OPA1, encoding a mitochondrial dynamin-related protein, is mutated in dominant optic atrophy. Nat. Genet. 26:207-210). The results described herein are supported by (1) the observation that a PS1 mutation (M146V) in a mouse PS1 knock-in model impairs axonal transport and also increases tau phosphorylation (Pigino et al., (2003) Alzheimer's presenilin 1 mutations impair kinesin-based axonal transport. J. Neurosci. 23:4499-4508), (2) the finding of axonal defects, consisting of the abnormal accumulation of molecular motor proteins, organelles, and vesicles, in SAD patients and in mouse models of AD (Stokin et al., (2005) Axonopathy and transport deficits early in the pathogenesis of Alzheimer's disease. Science 307:1282-1288), and (3) the identification of a few rare patients with inherited frontotemporal dementia (Pick disease) Dermaut et al., (2004) A novel presenilin 1 mutation associated with Pick's disease but not β-amyloid plaques. Ann. Neurol. 55:617-626; Halliday (et al, 2005) Pick bodies in a family with presenilin-1 Alzheimer's disease. Ann. Neurol. 57:139-143) and inherited dilated cardiomyopathy (Li et al., (2006) Mutations of Presenilin genes in dilated cardiomyopathy and heart failure. Am. J. Hum. Genet. 79:1030-1039) who had mutations in PS1 but who did not accumulate Aβ deposits in affected tissues; these outlier patients indicate that a clinical presentation due to mutations in presenilins can be “uncoupled” from the morphological hallmarks of AD. The finding that presenilins are physically and functionally associated with ER-MAM, and that mutations in presenilins which affect ER-MAM can contribute to the FAD warrants further investigation.

### Example 2

### Mitochondrial Maldistribution

The result that mitochondria are mislocalized in AD indicates a cause-and effect relationship between mitochondrial mislocalization and neurodegeneration, as opposed to a model in which APP and amyloid are primary determinants in the pathogenesis of FAD due to presenilin mutations. The accumulation of β amyloid, tau, neurofibrillary tangles, and other sequellae of APP processing are downstream events.

The results described herein show that (1) PS1 is targeted to a specific compartment of the ER that is intimately associated with mitochondria, called ER-mitochondria-associated membranes (ER-MAM, or ER-MAM), (2) there is a significant change in the amount of ER-MAM protein in cells from FAD^{PS1} patients, and (3) there are defects in mitochondrial distribution and morphology in fibroblasts from FAD^{PS1} patients and in shRNA-mediated PS1-knockdown cells: mitochondria in these cells fail to reach the cell periphery and exhibit abnormal fragmentation. ER-MAM has known functions in lipid metabolism (including ApoE) and glucose metabolism, and in calcium homeostasis, all of which are functions known to be compromised in Alzheimer's disease (AD). These results show that mutations in presenilins inhibit mitochondrial distribution and neuronal transmission through effects on mitochondrial ER interactions. Because Ca²⁺ regulates the attachment of mitochondria to microtubules, the defects in mitochondrial distribution observed FAD^{PS1} cells can be due to defects in ER-MAM-mediated calcium homeostasis that alter axonal mitochondrial transport. Alternatively, because ER-MAM has been shown to contribute to the anchorage of mitochondria at sites of polarized cell surface growth, accumulation of mitochondria in the nerve terminal can be compromised in presenilin mutants. These two models are not mutually exclusive. This analysis has been performed to determine the mechanism underlying defects in mitochondrial distribution in presenilin mutants, and to address the role of ER-MAM-localized presenilin.

To determine if the mitochondrial maldistribution phenotype is clinically relevant cultured fibroblasts, mitochondrial distribution in neurons and in other cells and tissues from humans and from transgenic mice harboring pathogenic mutations in presenilin can be examined Brain tissue from autopsies of FAD patients with presenilin mutations can be examined for mitochondrial distribution defects. A correlation between ApoE allele status, the mitochondrial distribution phenotype and the amount of communication between the ER and mitochondria in patient cells and tissues can be determined

To determine the role of presenilin in ER-MAM blue-native gels, immunoprecipitation, and protein identification techniques can be used to determine if ER-MAM-localized presenilin interacts with other partners in the ER-MAM subcompartment and to determine the effects of mutations in presenilin binding partners on ER-MAM localization.

To determine how mutant presenilin causes mitochondrial maldistribution the effect of presenilin mutations on anterograde and retrograde axonal transport of mitochondria, on retention and accumulation of mitochondria in nerve terminals, and on the dynamics of mitochondrial fusion and fission can be examined using mitochondrially-targeted photo-activatable fluorescent probes (“mitoDendra”) and live-cell imaging of neuronal cells. In order to determine the relevance of these observations to AD, these studies can be conducted in primary neuronal cells derived from normal, FAD^{PS1} and FAD^{PS2} mice of different ages.

Role of Presenilin in Mitochondrial Mislocalization. Mutated presenilins can be transfected into normal fibroblasts in order to recapitulate the morphological abnormalities observed in FAD^{PS1} or FAD^{PS2} fibroblasts (obtained from the Coriell Cell Repository). Since FAD is a dominant disorder, both the wild-type and mutant presenilin alleles are present in these cells. The normal and mutated presenilin (for example, E280A mutation in PS1) alleles from this cell line can be amplified and subcloned it into a mammalian expression vector, such as pCDNA3.1 (Stratagene). In order to be sure that the presenilin expressed from the transfected constructs is targeted to mitochondria, a His6 epitope tag can be attached to the C-terminus of the polypeptide, and anti-His-tag immunohistochemistry can be used to confirm the subcellular localization to mitochondria and to adherens junctions. Western blots and in-vitro importation assays can be performed to determine submitochondrial localization. Normal fibroblasts can be transiently co-transfected with a 10:1 ratio of the presenilin constructs and a construct encoding mitochondrially-targeted GFP, so that the cells containing “green” mitochondria can also be expressing the presenilin construct to allow investigation of mitochondrial morphology (i.e. on the green mitochondria) without having to distinguish between the morphology of transfected vs. untransfected cells.

Mitochondrial mislocalization in FAD brain. Brain tissue from autopsies of FAD patients with presenilin mutations can be examined to see if morphological abnormalities can be observed in neurons similar to those observed in fibroblasts.

Reversal of the mitochondrial mislocalization phenotype. The mitochondrial mislocalization phenotype can be reversed using pharmacological approaches designed to inhibit GSK3B a PS1-binding protein that controls the attachment of mitochondria to microtubules via phosphorylation/dephosphorylation of kinesin light chain. Control and presenilin cells can be treated with lithium, TDZD-8, and SB415286 (Bijur G N, Jope R S. (2003) Glycogen synthase kinase-3β is highly activated in nuclei and mitochondria. Neuroreport., 14, 2415-2419; King et al., (2001) Caspase-3 activation induced by inhibition of mitochondrial complex I is facilitated by glycogen synthase kinase-3β and attenuated by lithium. Brain Res., 919, 106-114; Barry et al., (2003) Regulation of glycogen synthase kinase 3 in human platelets: a possible role in platelet function? FEBS Lett., 553, 173-178), all of which inhibit GSK3B activity. Lithium and SB415286 inhibit neurite outgrowth (Orme et al., (2003) Glycogen synthase kinase-3 and Axin function in a β-catenin-independent pathway that regulates neurite outgrowth in neuroblastoma cells. Mol. Cell. Neurosci., 24, 673-86).

Characterization of the Mitochondrial Maldistribution Phenotype. Further characterization of ER-MAM in neurons. The immunohistochemical and Western blot data show that presenilins are ER-MAM-associated proteins. The association of presenilin with ER-MAM and the disposition of this compartment in neurons can be further characterized using antibodies to the ER-MAM markers PEMT, PACS2, and FACL4 (Abgent A P2536b). ER-MAM has not been studied in neurons. Such analysis can contribute to the general understanding of neurons, and the effect of disrupting ER-MAM on neuronal function.

### Example 3

Analysis of other Mutations. The preliminary studies were carried out on fibroblasts isolated from FAD^{PS1} patients with the A246E and M146L mutations. Fibroblasts from FAD patients with other PS1 mutations (lines EB [G209V], GF [I143T], WA [L418F]), and WL [H163R]), a fibroblast line carrying a PS2 mutation (line DD [N141I]) and a line carrying a pathogenic (“Swedish”) mutation in APP can be studied as described herein.

### Example 4

### Presenilins are Enriched in Mitochondria-Associated Membranes

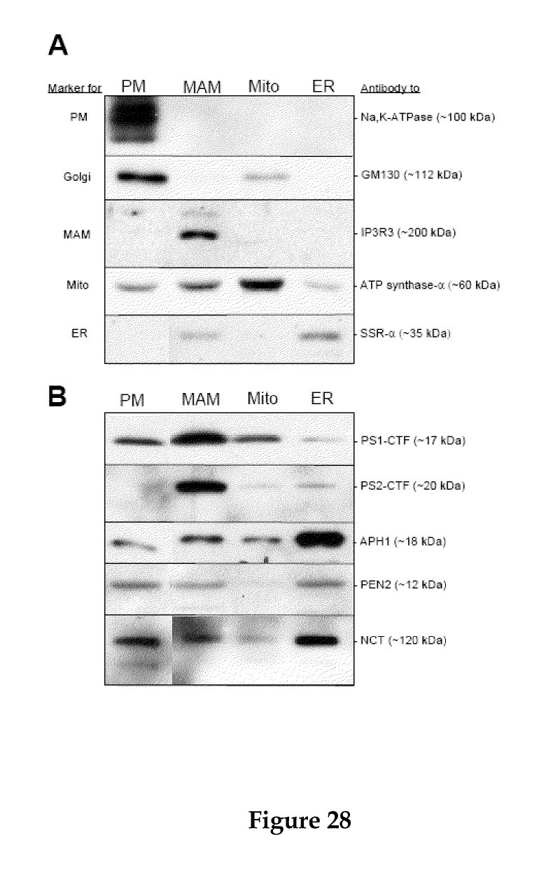

Plasma membrane (PM), crude mitochondria, and ER were isolated from mouse brain, and fractionated crude mitochondria further by isopycnic centrifugation (Vance et al, Biochim. Biophys. Acta 1997, 1348:142-150) into a MAM fraction and a purified mitochondrial fraction. Each of these fractions were evaluated by Western blot analysis, using antibodies to Na,K-ATPase as a marker for PM, to SSR as a marker for ER, to Golgi matrix protein GM130 (GOLGA2) as a marker for Golgi, to IP3R3 as a marker for MAM, and to the αsubunit of ATP synthase (ATP synthase-α) as a marker for mitochondria (FIG. 28A). All five markers were enriched in their respective compartments, but low levels of mitochondrial ATP synthase-α were also present in the plasma membrane. ATP synthase-α has been found in this compartment by others (Bae et al, Proteomics 2004, 4:3536-3548). The MAM fraction was enriched for IP3R3, a known MAM marker, (Mendes et al, Biol. Chem. 2005, 280:40892-40900) confirming separation of MAM from bulk ER and mitochondria to a degree sufficient for further analysis. The amount of protein recovered in each of the subcellular fractions analyzed from whole mouse brain was quantitated. Of the total amount of protein recovered in the ER fraction, ˜13%±0.3% (n=6) was in the MAM subfraction. This value reflects the analysis of total mouse brain, and can vary in different brain regions and in different tissues.

Western blot analysis was then performed on these same fractions from mouse brain, using antibodies against PS1 and PS2 (FIG. 28B). PS1 was found in the plasma membrane/Golgi fractions, as reported previously, (De Strooper et al, J. Biol. Chem. 1997, 272:3590-3598) however, as described herein, PS1 is essentially an ER-resident protein (FIG. 28B). However, within the ER, PS1 was not distributed homogeneously, but rather was enriched in ER membranes that are in close contact with mitochondria (i.e. MAM) (FIG. 28B). Like PS1, PS2 was also enriched in the MAM (FIG. 28B). Analysis of the blots revealed that the amount of PS1 was enriched by 5- to 10-fold in MAM over that in “bulk” ER (n=12).

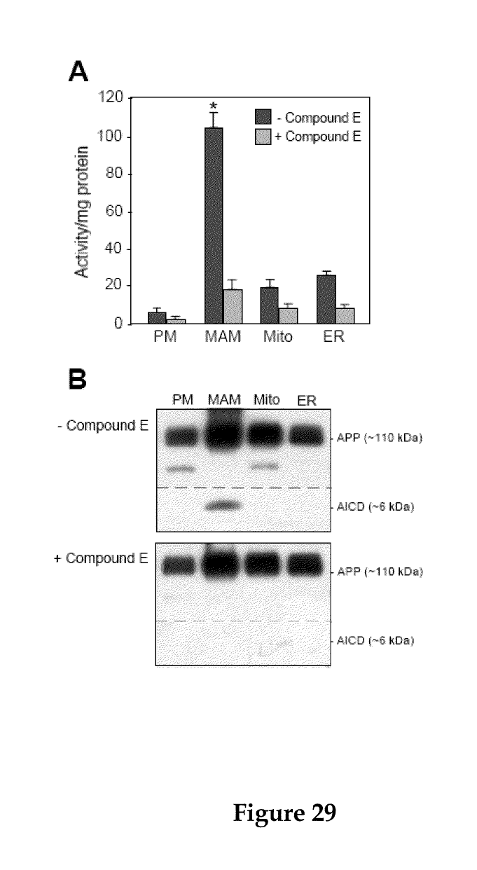

The various subcellular fractions of mouse brain where then assayed for the presence and amount of γ-secretase activity, using two different assays (FIGS. 29A and 29B). Most of the γ-secretase activity was detected in MAM compared to the other fractions assayed, showing not only that PS1 and PS2 are enriched in this fraction, but that the other components of the γ-secretase complex—APH1, NCT, and PEN2—are present there as well (Sato et al, J. Biol. Chem. 2007, 282:33985-33993). Using Western blotting, it was observed that those three polypeptides were enriched in the bulk ER, but were present in significant amounts in the MAM as well. Why the amount of the various γ-secretase components are not distributed proportionally in the two compartments can be a reflection of the different steps in the assembly pathway for the holoprotein (Spasic et al, J. Cell Sci. 2008, 121:413-420). Moreover, APP itself was also present in high amounts in the MAM (FIG. 2B). Thus, MAM contains both the enzymatic activity to cleave APP (i.e. γ-secretase) and the APP substrate itself. The localization of γ-secretase activity in MAM could help explain the unexpected presence of Aβ in mitochondria (Du et al, Nat. Med. 2008, 14:1097-1105)

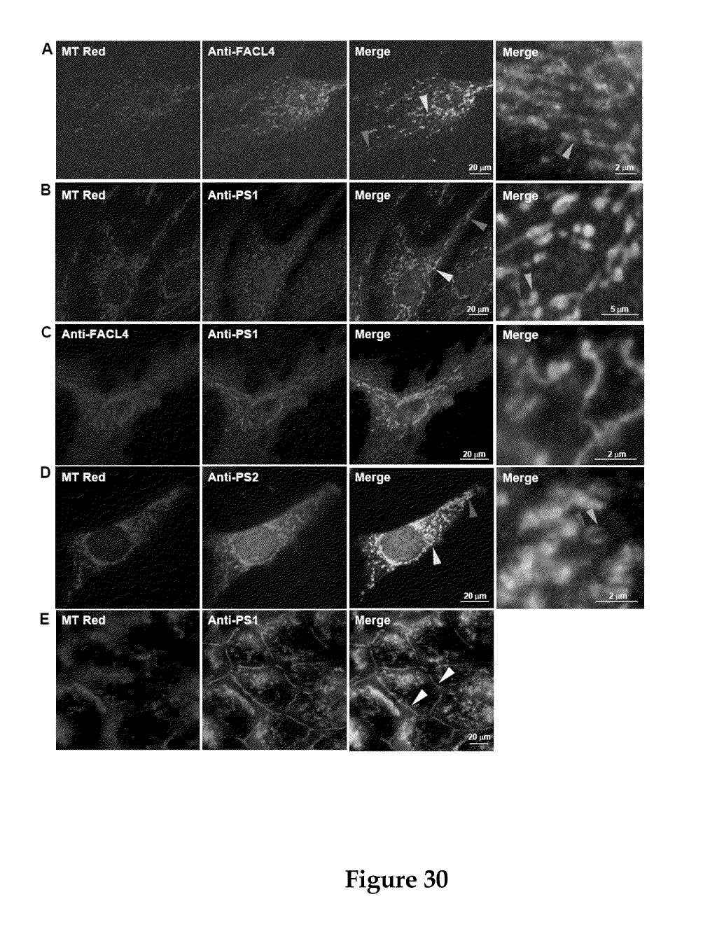

To further confirm that PS1 is a MAM-enriched protein, the immunocytochemical localization of PS1 in human fibroblasts was compared with that of FACL4, a known MAM-localized protein (Lewin et al, Arch. Biochem. Biophys. 2002, 404:263-270). Cells were stained with MT Red and then detected FACL4 by immunocytochemistry (FIG. 30A). FACL4 immunostain (green) was found to be “co-localized” with MT Red (red), but only partially: the “co-localization” was most predominant in the region around the nucleus (yellow arrowhead in FIG. 30A), but not in the more distal regions of the cell (red arrowhead in FIG. 30A). This result shows that the much of the yellow signal reflected the juxtaposition of MAM with mitochondria (see enlarged merge panel at right in FIG. 30A). Like FACL4, PS1 partially co-localized with MT Red, and also predominantly in the perinuclear region (FIG. 30B). The co-localization of PS1 with MT Red in the perinuclear region was revealed to actually consist of small discrete regions of PS1 immunostain apposed to discrete MT Red-positive regions (enlarged merge panel at right in FIG. 30B), a pattern highly similar to that observed with FACL4 (FIG. 30A) and with the sigma-1 receptor, another MAM-resident protein (Hayashi et al, Cell 2007, 131:596-610). This result is also consistent with the finding that PS1 was not imported into mitochondria in an in vitro import assay. Finally, when cells were double-stained for both PS1 and FACL4, the two proteins co-localized almost exactly, even at enlarged magnification (FIG. 30C). These results show that both PS1 and FACL4 reside in the same compartment, namely MAM. Quantitative analysis of the degree of co-localization confirmed these conclusions. In particular, the co-localization of PS1 with MT Red (as a decimal fraction) was 0.51±0.08, which was not statistically different than the value of 0.47±0.05 for the co-localization of FACL4, an authentic MAM protein, with MT Red. The quantitative data support the immunocytochemical results, namely, that PS1 is not a mitochondrial protein, but resides in a compartment adjacent to mitochondria, in a manner essentially identical to that of FACL4 (i.e. MAM).

The immunocytochemical results were confirmed in other cell types, including primary rat cortical neurons and mouse 3T3 cells. Importantly, a similar result was obtained using immunocytochemistry to detect human PS2 in mouse cells (FIG. 30D). Finally, besides the immunocytochemical localization to MAM, PS1 staining at adherens junctions in the plasma membrane was also observed in confluent COS-7 (FIG. 3E) and in human 293T and mouse 3T3 cells.

Taken together, the Western blotting, γ-secretase activity, and immunocytochemistry results show that PS1 and PS2 are indeed MAM-enriched proteins, in both neuronal and non-neuronal cells. The difference between the results described herein and reports in which presenilins were found in fractions enriched in markers characteristic of other subcellular compartments, such as ER (Annaert et al, J. Cell Biol. 1999, 147:277-294), Golgi (Annaert et al, J. Cell Biol. 1999, 147:277-294), the trans-Golgi network (Siman et al, J. Neurochem. 2003, 84:1143-1153), the ER-Golgi intermediate compartment (ERGIC) (Annaert et al, J. Cell Biol. 1999, 147:277-294), the nuclear envelope (Kimura et al, Brain Res. 2001, 922:30-41), endosomes (Vetrivel et al, J. Biol. Chem. 2004, 279:44945-44954), lysosomes (Pasternak et al, J. Biol. Chem. 2003, 278:26687-26694), and mitochondria (Ankarcrona et al, Biochem. Biophys. Res. Commun. 2002, 295:766-770), is due mainly to technical issues. In some analyses of subcellular fractions, other organelles, including MAM, co-purified with ER (Annaert et al, J. Cell Biol. 1999, 147:277-294), Golgi (Annaert et al, J. Cell Biol. 1999, 147:277-294), or mitochondria (Ankarcrona et al, Biochem. Biophys. Res. Commun 2002, 295:766-770). For example, after careful fractionation, sphingolipid-specific glycosyltransferase activity, which previously had been ascribed to the Golgi, was actually found to be in MAM (Ardail et al, Biochem. J. 2003, 371:1013-1019); in fact, MAM has been described as a pre-Golgi compartment for the secretory pathway (Rusino et al, J. Biol. Chem. 1994, 269:27494-27502). In other cases, the subcellular fractionation separated PS1 into a compartment that was almost certainly MAM, but in the absence of specific MAM markers was either not identified clearly or was identified in non-specific terms as an ER-related subcompartment (Kim et al, Neurobiol. Dis. 2000, 7:99-117).

As described herein, presenilins residing in the MAM are functionally active, acting as the catalytic core of the γ-secretase complex however PS1 and/or PS2 can also be involved in other functions in the MAM compartment. The finding that most of the γ-secretase activity is located in ER-mitochondria connections explains the observation of mitochondrial oxidative damage associated with abnormal APP processing (Atamna et al, Mitochondrion 2007, 7:297-310). Moreover, it explains how Aβ accumulates in mitochondria (Du et al, Nat. Med. 2008, 14:1097-1105), as well as provide the basis for the interaction between PS1 and a number of known mitochondrial proteins.

Numerous hypotheses have been proposed to explain the pathogenesis of AD, including altered APP processing and amyloid toxicity (Hardy et al, Science 2002, 297:353-356; Small et al, Nature Rev. Neurosci. 2001, 2:595-598), tau hyperphosphorylation (Takashima et al, Proc. Natl. Acad. Sci. USA 1998, 95:9637-9641), altered lipid (Jin et al, Neurosci. Lett. 2006, 407:263-267), cholesterol (Neurochem. Res. 2007, 32:739-750), and glucose metabolism (Gong et al, J. Alzheimer's Dis. 2006, 9:1-12), aberrant calcium homeostasis (Smith et al, Cell Calcium 2005, 38:427-437), glutamate excitotoxicity (Ringheim et al, Curr. Pharm. Des. 2006, 12:719-738), inflammation (Ringheim et al, Curr. Pharm. Des. 2006, 12:719-738), and mitochondrial dysfunction and oxidative stress (Atamna et al, Mitochondrion 2007, 7:297-310). A localization of presenilin in MAM, a compartment intimately involved in lipid, glucose, cholesterol, and calcium homeostasis, may help reconcile these disparate hypotheses, and could explain many seemingly unrelated features of this devastating neurodegenerative disorder.

### Example 5

### Alzheimer Disease and Presenilins

Alzheimer disease (AD) is a neurodegenerative dementing disorder of late onset, with a relatively long course (Mattson M P (2004) Pathways towards and away from Alzheimer's disease. Nature 430:631-639) There is progressive neuronal loss, especially in the cortex and the hippocampus. The two main histopathological hallmarks of AD are the accumulation of extracellular neuritic plaques, consisting mainly of β-amyloid (Aβ), and of neurofibrillary tangles, consisting mainly of hyperphosphorylated forms of the microtubule-associated protein tau (Goedert M, Spillantini M G (2006) A century of Alzheimer's disease. Science 314:777-781; Roberson et al. (2007) Reducing endogenous tau ameliorates amyloid beta-induced deficits in an Alzheimer's disease mouse model. Science 316:750-754). The majority of AD is sporadic (SAD), but at least three genes—amyloid precursor protein (APP), presenilin-1 (PS1), and presenilin-2 (PS2)— have been identified in the familial form (FAD). Clinically, FAD due to mutations in PS1/2 is similar to SAD (including elevated Aβ42 levels) but has an earlier age of onset. Variants in two genes predispose people to SAD: apolipoprotein E isoform 4 (ApoE4) (Corder et al. (1993) Science 261:921-923) and polymorphisms in SORL1, a neuronal sorting receptor (Rogaeva et al. (2007) Nature Genet. in press) PS1 and PS2 are aspartyl proteases (Wolfe M S, Kopan R (2004) Science 305:1119-1123) that are “signal peptide peptidases” (SPPs) (Weihofen et al. (2002) Science 296:2215-2218; Brunkan A L, Goate A M (2005) J. Neurochem. 93:769-792); they are members of a gene family that includes at least five PS-like proteins (Weihofen et al. (2002) Science 296:2215-2218; Ponting et al. (2002) Hum. Mol. Genet. 11:1037-1044). The ˜50-kDa full-length protein is cleaved in the “loop” domain to produce N- and C-terminal fragments (NTF and CTF) that comprise the active form of the enzyme (Wolfe M S, Kopan R (2004) Science 305:1119-1123).

Most relevant to AD, they are components of the V complex. Following cleavages of APP by β-secretase, γ-secretase cleaves the remaining APP polypeptide to release small amyloidogenic fragments, 40- and 42-aa in length (Aβ 40 and Aβ 42) that have been implicated in the pathogenesis of AD (Brunkan A L, Goate A M (2005) J. Neurochem. 93:769-792; Chen Q, Schubert D (2002) Expert Rev. Mol. Med. 4:1-18; Gandy S (2005) J. Clin. Invest. 115:1121-1129). PS1 and PS2 are unusual in that they cleave their target polypeptides within membranes (Wolfe M S, Kopan R (2004) Science 305:1119-1123). As such, they belong to one of three classes of intramembrane proteases: site 2 protease (S2P) metalloproteases; rhomboid serine proteases; and the γ-secretase and SPP aspartyl proteases (Wolfe M S, Kopan R (2004) Science 305:1119-1123). Of these, γ-secretase has the broadest substrate specificity. While the exact sequence of physiological events leading to impairment of memory and ultimately to dementia in AD is unclear, mounting evidence points to a decline in hippocampal synaptic function prior to neuronal degeneration as a key factor in this process (Selkoe D J (2002) Science 298:789-791). Patients with both mild and early-onset AD had fewer synapses in the outer molecular layer of the dentate gyms compared to controls, indicating that loss of afferents from the entorhinal cortex underlie the synapse loss seen in AD (Scheff (2006) Neurobiol. Aging 27:1372-1384). Synaptic density in these brain regions was also reduced in transgenic mice expressing the “Swedish” mutation in APP (Dong et al. (2007) J. Comp. Neurol. 500:311-321), and conditional double-knockout (KO) mice lacking both PS1 and PS2 in forebrain exhibited impairments in hippocampal memory and synaptic plasticity (Saura et al. (2004) Neuron 42:23-36).

Finally, hippocampal cultures from transgenic mice expressing the PS1 A246E mutation had depressed evoked synaptic currents, due to reduced synaptic density (Priller et al. (2007) J. Biol. Chem. 282:1119-1127). These results indicate that AD is ultimately a disease of synaptic transmission (Selkoe D J (2002) Science 298:789-791; Walsh D M, Selkoe D J (2004) Neuron 44:181-193) wherein the pathogenesis of AD involves a relationship between two or more of amyloid, presenilins, predisposing factors, and other cellular processes.

PS1 has been localized to numerous membranous compartments in cells. These include the endoplasmic reticulum (ER) (Walter et al. (1996) Mol. Med. 2:673-691; Kimura et al. (2001) Brain Res. 922:30-41), the Golgi apparatus (Walter et al. (1996) Mol. Med. 2:673-691; De Strooper et al. (1997) J. Biol. Chem. 272:3590-3598; Annaert (1999) J. Cell Biol. 147:277-294; Siman R, Velji J (2003) J. Neurochem. 84:1143-1153), endosomes/lysosomes (Runz et al. (2002) J. Neurosci. 22:1679-1689; Vetrivel (2004) J. Biol. Chem. 279:44945-44954), the nuclear envelope (Kimura et al. (2001) Brain Res. 922:30-41), the perinuclear region (Takashima (1996) Biophys. Res. Commun 227:423-426), mitochondria (Ankarcrona M, Hultenby K (2002) Biochem. Biophys. Res. Commun. 295:766-770; Hansson (2005) J. Neurochem. 92:1010-1020), and the plasma membrane (Schwarzman et al. (1999) Proc. Natl. Acad. Sci. USA 96:7932-7937; Singh et al. (2001) Exp. Cell Res. 263:1-13; Baki et al. (2001) Proc. Natl. Acad. Sci. USA 98:2381-6; Marambaud et al. (2003) Cell 114:635-645; Tarassishin et al. (2004) Proc. Natl. Acad. Sci. USA 101:17050-17055), where they are especially enriched at adherens junctions (Takashima (1996) Biochem. Biophys. Res. Commun. 227:423-426; Georgakopoulos et al. (1999) Mol. Cell. 4:893-902; Marambaud et al. (2002) EMBO J. 21:1948-1956). Besides PS1/2, the γ-secretase complex contains five other proteins: APH1, PEN2, nicastrin (NCT, also called APH2) (De Strooper B (2003) Neuron 38:9-12), and two regulatory subunits, CD147 (Zhou et al. (2005) Proc. Natl. Acad. Sci. USA 102:7499-7504) and TMP21 (Chen et al. (2006) Nature 440:1208-1212). Since γ-secretase complexes with different molecular masses and subunit compositions have been found (Gu et al. (2004) J. Biol. Chem. 279:31329-31336), different subunits may affect the localization and/or function of the complex. Using biochemical approaches, PS1, APH1, NCT, and PEN2 have been found in the plasma membrane (Hansson et al. (2005) J. Neurochem. 92:1010-1020). Using immunoelectron microscopy and Western blotting, APH1, NCT, and PEN2 have been localized to mitochondria (Ankarcrona M, Hultenby K (2002) Biochem. Biophys. Res. Commun 295:766-770; Chada S R, Hollenbeck P J (2003) J. Exp. Biol. 206:1985-1992).

Mitochondria and mitochondrial movement. Mitochondria are not free floating in the cytoplasm, as mitochondria are enriched at sites of high ATP utilization (Kaasik et al. (2001) Circ. Res. 89:153-159); in mammals mitochondria move mainly along microtubules (MTs) (Friede R L, Ho K C (1977) J. Physiol. 265:507-519; Nangaku et al. (1994) Cell 79:1209-1220; Pereira et al. (1997) J. Cell Biol. 136:1081-1090; Rube D A, van der Bliek A M (2004) Mol. Cell. Biochem. 256:331-339). Their movement is “saltatory”: they stop and go in response to physiologic events (Chang et al. (2006) Neurobiol. Dis. 22:388-400) and intracellular signals (Chada S R, Hollenbeck P J (2003) J. Exp. Biol. 206:1985-1992), regulated in part in response to changes in the local Ca2+ gradient (Yi et al. (2004) J. Cell Biol. 167:661-672) and the bioenergetic state of mitochondria (Miller K E, Sheetz M P (2004) J. Cell Sci. 117:2791-2804).

Mitochondria with normal membrane potential tend to move towards the periphery (anterograde movement); loss of membrane potential and of ATP synthesis result in increased retrograde transport to the cell body (Miller K E, Sheetz M P (2004) J. Cell Sci. 117:2791-2804). Mitochondria are positioned strategically at neuronal sites where the metabolic demand is high, such as active growth cones, nodes of Ranvier, and synapses in axons and dendrites (Chang et al. (2006) Neurobiol. Dis. 22:388-400; Li et al. (2004) Cell 119:873-887). Presynaptic terminals require mitochondria for Ca2+ homeostasis and to operate plasma membrane Ca2+ ATPases (Zenisek D, Matthews G (2000) Neuron 25:229-237), as well as to power the actin motors necessary for vesicle cycling and synaptic plasticity (Dillon C, Goda Y (2005) Annu. Rev. Neurosci. 28:25-55). Mitochondria are also abundant in post synaptic dendritic terminals, supporting energy-dependent processes in these areas (Chang et al. (2006) Neurobiol. Dis. 22:388-400). Transport on microtubules requires kinesins for anterograde transport and dyneins for retrograde transport (Hollenbeck P J (1996) Front. Biosci. 1:d91-d102).

Mitochondria are associated with kinesins via KIF1B (Nangaku et al. (1994) Cell 79:1209-1220), KIF5B (Pereira et al. (1997) J. Cell Biol. 136:1081-1090; Tanaka et al. (1998) Cell 93:1147-1158), KLC3 (Zhang et al. (2004) Dev. Biol. 275:23-33), kinectin (Santama et al. (2004) J. Cell Sci. 117:4537-4549), and syntabulin (Cai et al. (2005) J. Cell Biol. 170:959-969). Dynein also binds mitochondria (Pilling et al. (2006) Mol. Biol. Cell 17:2057-2068). Two cargo adaptor proteins discovered recently in Drosophila—Miro and Milton—are implicated in the specific linkage of mitochondrial to kinesin-1 in neurons. Miro (mitochondrial rho-like GTPase) (Guo et al. (2005) Neuron 47:379-393) is a calcium-binding protein that binds to the mitochondrial-specific adaptor protein Milton, which in turn is linked to the kinesin-1 heavy chain (KHC) (Glater et al. (2006) J. Cell Biol. 173:545-557). Alterations in any of these molecules affect mitochondrial movement and distribution (Tanaka et al. (1998) Cell 93:1147-1158; Guo et al. (2005) Neuron 47:379-393; Fransson et al. (2003) J. Biol. Chem. 278:6495-6502; Fransson et al. (2006) Biochem. Biophys. Res. Commun 344:500-510; Stowers et al. (2002) Neuron 36:1063-1077). Mutations in Miro result in aggregation of mitochondria in the perinuclear region (Fransson et al. (2003) J. Biol. Chem. 278:6495-6502; Fransson et al. (2006) Biochem. Biophys. Res. Commun 344:500-510). Miro may be an important regulator of mitochondrial motility in neurons, in essence operating as a sensor of local concentrations of Ca2+ and ATP.

The “calcium hypothesis” in FAD. The predominant “amyloid hypothesis” invokes the toxic effects of APP and amyloid in the pathogenesis of AD (Hardy J, Selkoe D J (2002) Science 297:353-356). The role of calcium in the pathogenesis of AD is more controversial, but there is a growing body of evidence to implicate calcium, at least in FAD due to mutations in PS1 (FAD^{PS1}). The overall thrust of the “calcium hypothesis” is that presenilin mutations affect ER Ca2+ signaling (Mattson et al. (2000) Trends Neurosci. 23:222-229; Smith et al. (2005) Cell Calcium 38:427-437), resulting, in some as-yet undefined way, in neuronal degeneration.

For example, inositol 1,4,5-triphosphate (IP3) mediated release of Ca2+ was enhanced in both FAD and SAD fibroblasts (Ito et al. (1994) Proc. Natl. Scad. Sci. USA 91:534-538), and expression of mutated PS1 (Leissring et al. (1999) J. Neurochem. 72:1061-1068) and PS2 (Leissring et al. (1999) J. Biol. Chem. 274:32535-32538) in Xenopus oocytes potentiated IP3 mediated Ca2+ signaling. Fibroblasts from knock-in mice (Leissring et al. (2000) J. Cell Biol. 149:793-798) and cortical neurons (Yoo et al. (2000) Neuron 27:561-572) harboring PS1 mutations had increased Ca2+ in the ER (Leissring et al. (2000) J. Cell Biol. 149:793-798), whereas ablation of PS1 had the opposite effect (Yoo et al. (2000) Neuron 27:561-572). Transgenic mice expressing mutant PS1 and PS2 also had altered Ca2+ homeostasis (Begley et al. (1999) J. Neurochem. 72:1030-1039; Barrow et al. (2000) Neurobiol. Dis. 7:119-126), including increased ER Ca2+, a lower threshold for kainic acid-induced glutamate release, and increased glutamate-induced Ca2+ signals (Schneider et al. (2001) J. Biol. Chem. 276:11539-11544). PS2 was found to associate with sorcin, a Ca2+-binding modulator of the muscle calcium channel/ryanodine receptor (RyR) (Pack-Chung et al. (2000) J. Biol. Chem. 275:14440-14445) that is in close apposition to both ER and mitochondria (Pickel et al. (1997) J. Comp. Neurol. 386:625-634). Finally, presenilins are required for Ca2+ influx into cells from “store operated Ca2+” (SOC) channels located in the plasma membrane (“capacitative calcium entry” [CCE]).

In cells lacking PS1, ER [Ca2+] was decreased (Leissring et al. (2002) Proc. Natl. Acad. Sci. USA 99:4697-4702) and CCE was activated (Yoo et al. (2000) Neuron 27:561-572; Ris et al. (2003) J. Biol. Chem. 278:44393-44399), whereas in cells with FAD-linked mutations ER [Ca2+] increased (Leissring et al. (2000) J. Cell Biol. 149:793-798; Nelson et al. (2007) J. Clin. Invest. 117:1230-1239) and CCE was inhibited (Leissring et al. (2000) J. Cell Biol. 149:793-798; Yoo et al. (2000) Neuron 27:561-572; Leissring et al. (2002) Proc. Natl. Acad. Sci. USA 99:4697-4702). In transgenic mice lacking neuronal PS1, CCE activation triggered long term potentiation of synapses in hippocampal slices (R is et al. (2003) J. Biol. Chem. 278:44393-44399). Taken together, these results indicate that PS1 acts to refill ER Ca2+ stores from SOC channels, an event that is triggered by depletion of ER [Ca2+]. During CC E, two elements are required to reduce diffusion of Ca2+ into the cytosol in the vicinity of SOC channels: (1) an active sarcoplasmic/endoplasmic reticulum calcium ATPase (SERCA) and (2) neighboring mitochondria (Jousset et al. (2007) J. Biol. Chem. 282:11456-11464). In CC E, the mitochondria play two roles: they scavenge remaining Ca2+ that cannot be handled by the SERCA, and they provide local ATP to buffer [Ca2+] (Jousset et al. (2007) J. Biol. Chem. 282:11456-11464). Both functions require that mitochondria be located in the vicinity of the SOC channels near the narrow and extended ER-PM junctions in “microdomains” linking the SOC channels to the SERCA (Jousset et al. (2007) J. Biol. Chem. 282:11456-11464). Thus, the subcellular distribution of mitochondria in microdomains is critical not only for providing ATP as a source of oxidative energy for various cellular processes, such as neurotransmission (Hollenbeck P J, Saxton W M (2005) J. Cell Sci. 118:5411-5419; Hollenbeck P J (2005) Neuron 47:331-333), but is also critical for “non-oxidative” functions, such as Ca2+ homeostasis (Hollenbeck P J (2005) Neuron 47:331-333; Alonso et al. (2006) Cell Calcium 40:513-525). Thus, mutations in PS1 can have devastating effects on neuronal function.

The “amyloid hypothesis” and the “calcium hypothesis” need not be mutually exclusive explanations for the pathogenesis of AD, as connections among PS1, APP, and Ca2+ signaling may actually exist. First, there is evidence that PS1 functions as a passive ER Ca2+ leak channel (Nelson et al. (2007) J. Clin. Invest. 117:1230-1239; Tu et al. (2006) Cell 126:981-993) and that expression of FAD-linked PS1 mutations disrupt this functionality (Nelson et al. (2007) J. Clin. Invest. 117:1230-1239). γ-secretase-mediated cleavage of APP yields an intracellular fragment, called the APP intracellular domain (AICD), which then translocates to the nucleus (Cupers et al. (2001) J. Neurochem. 78:1168-1178).

However, AICD also regulates phosphoinositide-mediated Ca2+ signaling in a mechanism involving modulation of ER Ca2+ stores (Leissring et al. (2002) Proc. Natl. Acad. Sci. USA 99:4697-4702); notably, only the AICD fragment of APP has this property (Leissring et al. (2002) Proc. Natl. Acad. Sci. USA 99:4697-4702). Thus, the proteolysis of APP may be required for intracellular Ca2+ signaling, and a defect in such processing in PS1-mutated cells can explain alterations in the pleiotropic effects on Ca2+ handling described herein.

Endoplasmic reticulum—mitochondria-associated membranes (ER-MAM). The ER is the cell's main store of calcium, which is released upon stimulation by input signals such as IP3 and sphingosine-1-phosphate (Berridge M J (2002) Cell Calcium 32:235-249). The main site of calcium uptake is the mitochondrion, but mitochondria are not passive “sinks”—they use calcium actively, for example, to activate dehydrogenases for intermediate metabolism (Robb-Gaspers et al. (1998) EMBO J. 17:4987-5000). Thus, the ER and the mitochondria can be linked not only biochemically but also physically (Jousset et al. (2007) J. Biol. Chem. 282:11456-11464; Rizzuto et al. (1998) Science 280:1763-1766; Csordas et al. (2006) J. Cell Biol. 174:915-921). In 1993, Cui et al. (Cui et al. (1993) J. Biol. Chem. 268:16655-16663) described the localization of phosphatidylethanolamine N-methyltransferase 2 (PEMT), an enzyme of phospholipid metabolism, in a unique membrane subfraction of the ER that was subsequently termed ER-MAM (Rusinol et al. (1994) J. Biol. Chem. 269:27494-27502). The same compartment was also found in yeast (Gaigg et al. (1995) Biochim. Biophys. Acta 1234:214-220; Prinz et al. (2000) J. Cell Biol. 150:461-474).

Since then, about two dozen proteins have been found to be concentrated in the ER-MAM, most of which are enzymes involved in the metabolism of glucose (e.g. glucose-6-phosphatase [G6PC) (Bionda et al. (2004) Biochem. J. 382:527-533), phospholipids (PEMT; diacylglycerol acyltransferase 2 [DGAT2] (Man et al. (2006) J. Lipid Res. 47:1928-1939), ceramide (ceramide glucosyltransferase [UCGC] (Ardail et al. (2003) Biochem. J. 371:1013-1019), gangliosides (β-galactoside α(2-6) sialyltransferase (SIAT1/ST6GAL1] (Ardail et al. (2003) Biochem. J. 371:1013-1019), cholesterol (sterol Oacyltransferase 1 [SOAT1], also called acyl-coenzyme A:cholesterol acyltransferase [ACAT1] (Rusinol et al. (1994) J. Biol. Chem. 269:27494-27502), and fatty acids (stearoyl-CoA desaturase [SCD] (Man et al. (2006) J. Lipid Res. 47:1928-1939); fatty acid-CoA ligase 4 [FACL4] (Lewin et al. (2002) Arch. Biochem. Biophys. 404:263-270), and in lipoprotein transport (microsomal triglyceride transfer protein large subunit [MTTP] (Rusinol et al. (1994) J. Biol. Chem. 269:27494-27502). ER-MAM is a physical bridge that connects the ER to mitochondria (Csordas et al. (2006) J. Cell Biol. 174:915-921). This result explains why it has been almost impossible to subfractionate pure mitochondria uncontaminated by ER (on the other hand, the reverse—the isolation of ER uncontaminated by mitochondria—is relatively easy). Moreover, the IP3 receptor (IP3R), which binds IP3 to stimulate Ca2+ transfer to mitochondria, is also a ER-MAM protein (Csordas et al. (2006) J. Cell Biol. 174:915-921), as is the ryanodine receptor (Hajnoczky et al. (2002) Cell Calcium 32:363-377) and, most recently, the sigma-1 type opioid receptor (SIG1R/OPRS1) (Hayashi T, Su T P (2007) Cell 131:596-610), emphasizing the intimate relationship between ER and mitochondria in regulating calcium.

RyRs interact with, and are regulated by, both PS1 (Rybalchenko et al. (2008) Int. J. Biochem. Cell Biol. 40:84-97) and PS2 (Hayrapetyan et al. (2008) Cell Calcium in press), and IP3R interacts with PS2 (Cai et al. (2006) J. Biol. Chem. 281:16649-16655). Moreover, there is a functional coupling between RyRs and mitochondria that allows for “tunneling” of Ca2+ from the ER to mitochondria (Kopach et al. (2008) Cell Calcium 43:469-481). Only one protein has been implicated in the regulation of ER mitochondrial communication via the ER-MAM: phosphofurin acidic cluster sorting protein 2 (PACS2), which controls the apposition of mitochondria with the ER (Simmen et al. (2005) EMBO J. 24:717-729). PACS2 interacts with transient receptor potential protein 2 (TRPP2/PKD2), a Ca2+-permeable cation channel (Köttgen et al. (2005) EMBO J. 24:705-716). PACS2 is found predominantly in the perinuclear region of cells (Simmen et al. (2005) EMBO J. 24:717-729), as is TRPP2 (Köttgen et al. (2005) EMBO J. 24:705-716) and PS1 itself (De Strooper et al. (1997) J. Biol. Chem. 272:3590-3598; Levitan D, Greenwald I (1998) Development 125:3599-3606). PACS2 translocates to mitochondria upon stimulation with pro-apoptotic agents such as staurosporin (Simmen et al. (2005) EMBO J. 24:717-729).

PS1 is enriched in the ER-MAM. Various cells were stained for mitochondria (using the mitochondrion-specific dye MitoTracker Red[MT Red; Molecular Probes]) and immunohistochemistry was performed to detect PS1 (Abcam ab10281). Initial investigations using “standard” immunohistochemistry (i.e. paraformaldehyde (PF) fixation followed by digitonin and/or Triton X-100 [TX-100] permeabilization of the cells prior to application of antibodies) revealed nonspecific staining of numerous membranous compartments (e.g. ER, Golgi, plasma and nuclear membranes), similar to the results reported by others; a representative result for monkey COS-7 cells is shown in FIG. 4A. When the permeabilization technique was modified by omitting the treatment with TX-100 and by fixing the cells with either cold methanol (MeOH) alone (FIG. 4B) or with PF followed by MeOH, a different result was obtained where PS1 co-localized with the MT Red stain, predominantly in the perinuclear region. PS1 was also present diffusely in areas that were devoid of mitochondria (fainter green regions in FIG. 4B, asterisks); presumably these are plasma membrane, ER, and/or Golgi.

To confirm that PS1 is a ER-MAM-enriched protein, immunocytochemical localization of PS1 in human fibroblasts was compared with that of PEMT, an authentic ER-MAM protein (Cui et al. (1993) J. Biol. Chem. 268:16655-16663; Rusinol et al. (1994) J. Biol. Chem. 269:27494-27502). PEMT co-localized with a subset of mitochondria, as visualized by staining with MT Red, as expected for a protein that is localized in a compartment that serves as a bridge between mitochondria and ER (i.e. ER-MAM) (FIG. 4D). The colocalization with MT Red was most pronounced in the region around the nucleus, indicating that the ER-MAM subcompartment is located predominantly in the perinuclear region of the cell. Like PEMT, PS1 also colocalized with MT Red, and also predominantly in the perinuclear region (FIG. 4C). Finally, double staining of cells for both PS1 and PEMT shows that they co-localized almost exactly (FIG. 4E).

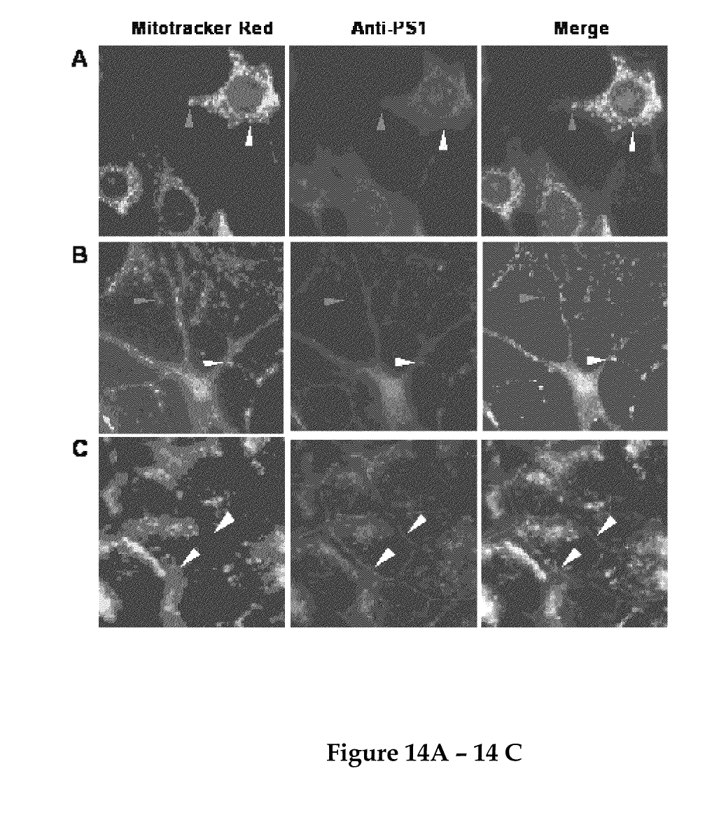

PS1 staining was also observed at adherens junctions in the plasma membrane in confluent COS-7 (FIG. 14A) and in human 293T and mouse 3T3 cells, also as seen by others (Georgakopoulos et al. (1999) Mol. Cell. 4:893-902; Marambaud et al. (2002) EMBO J. 21:1948-1956), confirming a known location for PS1 even when cells were fixed in MeOH. Since PS1 is associated with neurodegeneration, PS1 localization was studied in primary rat neurons. PS1 co-localized more with MT Red signal that is perinuclear and within the cell body compared to processes away from the cell body (FIG. 14B).

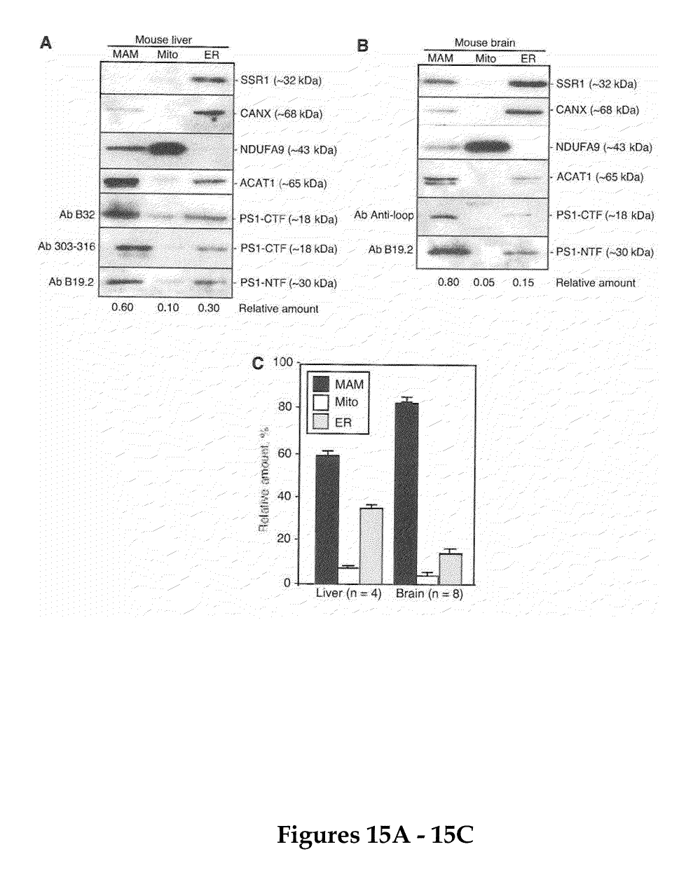

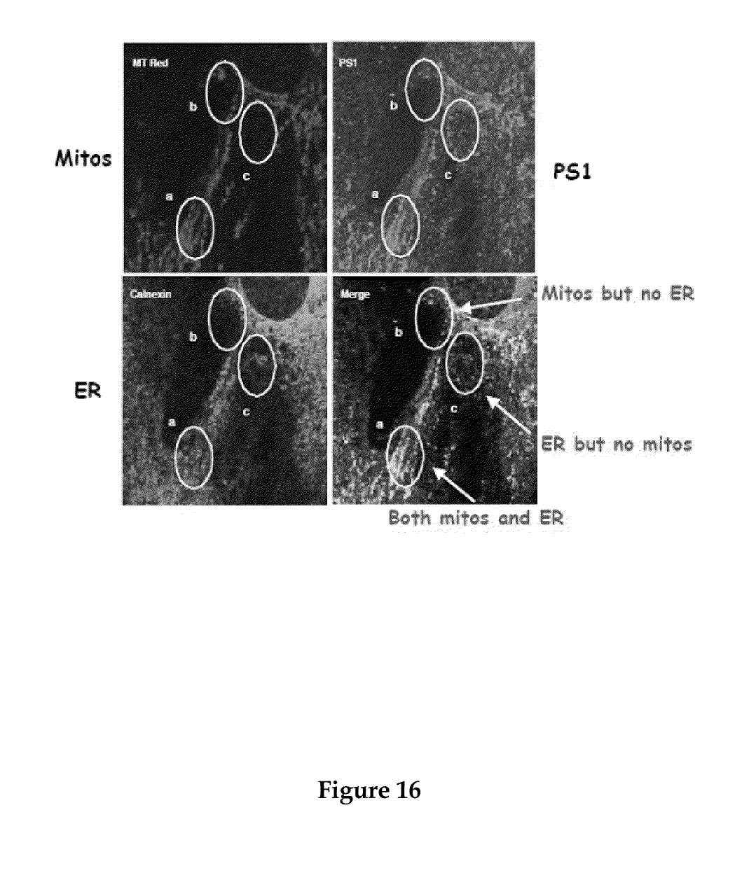

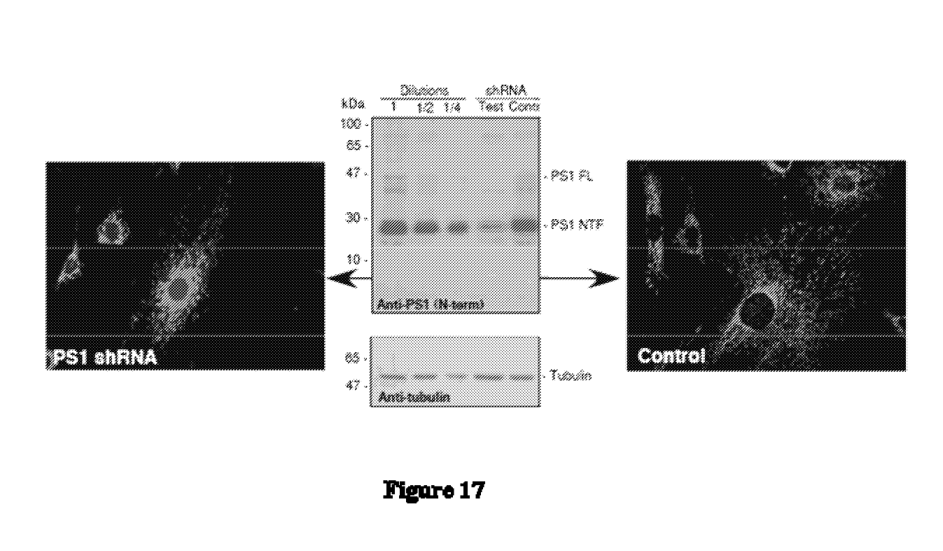

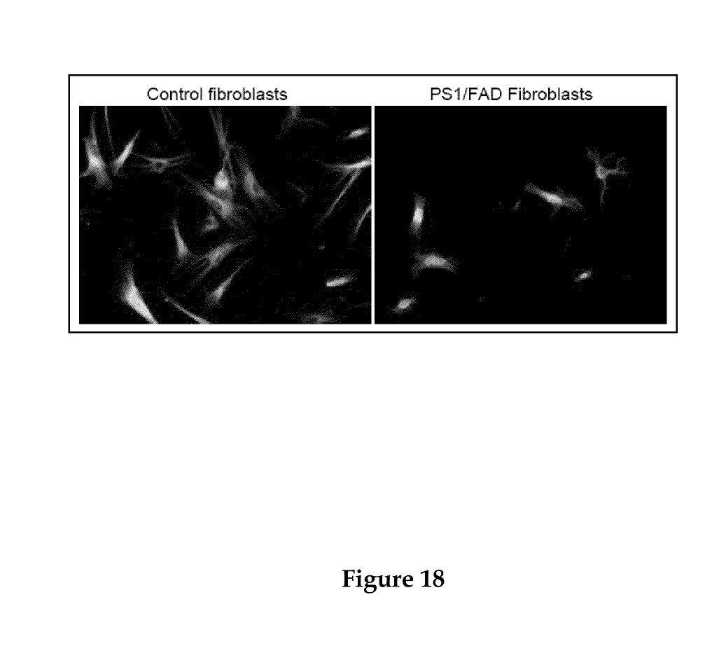

The use of TX-100 to permeabilize the cells prior to immunohistochemical detection has a profound effect on PS1 localization. This finding is consistent with the observation that TX-100 permeabilization alters immunolocalization of mitochondrial proteins (Melan M A, Sluder G (1992) J. Cell Sci. 101:731-743; Brock et al. (1999) Cytometry 35:353-362). Equally important, the results described herein indicate that PS1 localizes to a subset of perinuclear mitochondria in neurons and non-neuronal cells. Since PS1 is not targeted to all mitochondria and since import of PS1 into mitochondria in an in-vitro import assay was not detected, and since it has a subcellular distribution essentially identical to that of PEMT, the results described herein show that PS1 is not a mitochondrial-targeted polypeptide, but is rather an ER-MAM polypeptide that is “mitochondria-associated” under some circumstances. The immunocytochemical data support a localization of PS1 to ER-MAM. However, since there is no a priori reason to believe that MeOH fixation without TX-100 gives a more accurate result than methods using TX-100, subcellular fractionation was used to evaluate the association of PS1 with ER-MAM. Plasma membrane (PM), crude mitochondria (CM), and ER was isolated as described (Stone S J, Vance J E (2000) J. Biol. Chem. 275:34534-34540; Vance J E (1990) J. Biol. Chem. 265:7248-7256), and crude mitochondria was further fractionated by isopycnic centrifugation (Vance et al. (1997) Biochim. Biophys. Acta 1348:142-150) into a ER-MAM and a purified mitochondrial fraction. The fractions were evaluated by Western blot analysis using antibodies to cadherin (CDH2; marker for PM), calnexin (CANX; for ER), signal sequence receptor αSSRI; for ER), Golgi matrix protein GM130 (GOLGA2; for Golgi), ACAT1, G6PC, and PEMT (for ER-MAM [and to a lesser extent, ER]), and the NDUFA9 subunit of complex I of the respiratory chain (for mitochondria) (FIG. 15).

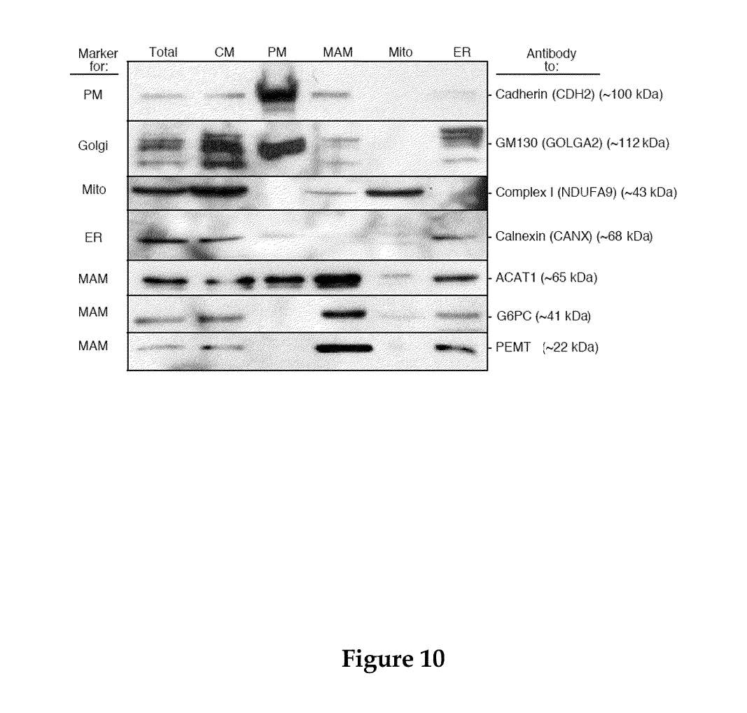

The analysis indicated that the ER-MAM fraction is distinct from ER or purified mitochondrial fractions. Specifically, the ER-MAM fraction was enriched for PEMT, G6PC, and ACAT1, known ER-MAM markers. Conversely, marker proteins for the PM, Golgi, ER and mitochondria were selectively depleted from the ER-MAM fraction (FIG. 10).

Analysis by Western blot of the ER, ER-MAM, and mitochondria fractions from mouse liver and brain showed that the majority of PS1 was present in the ER-MAM fraction, similar to the pattern seen for ACAT1 (FIGS. 15A-15B). This finding, together with the immunohistochemistry studies, indicate that PS1 is localized to a subcompartment of mitochondria associated with ER, i.e., ER-MAM.

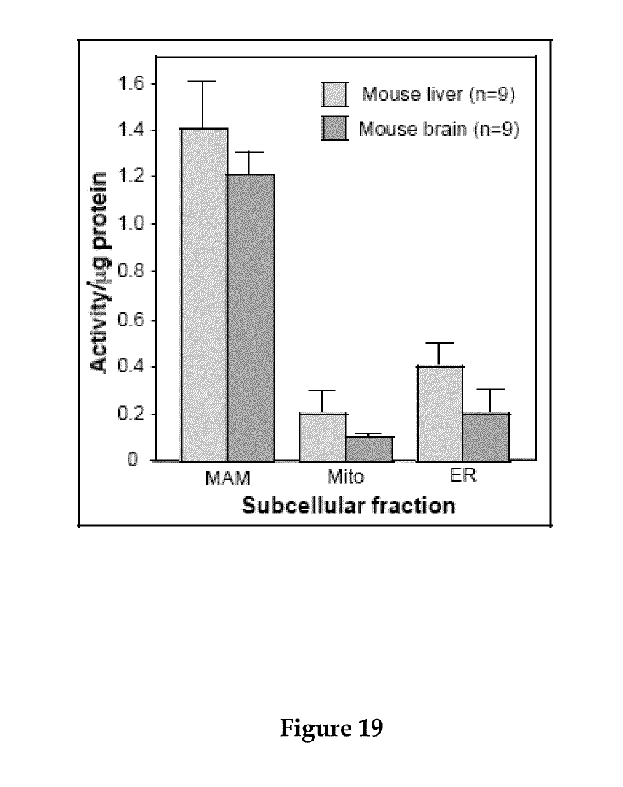

Using a FRET-based assay (R&D Systems #FP003) on subcellular fractions from mouse, the γ-secretase specific activity was observed to be about 5 times higher in the ER-MAM than in the ER, in both liver and brain (FIG. 19). This result shows that that PS1 is enriched in ER-MAM.

Mitochondrial dynamics in cells expressing mutated PS1. To determine if PS1 has functionally significant interactions with this compartment, the morphology and distribution of MT Red-labeled mitochondria in fibroblasts from a control and an FAD^{PS1} patient (mutation A246E [Coriell AG06840]) was studied. To define cell boundaries, the microtubule cytoskeleton (with anti-tubulin) in the same cells was also visualized.

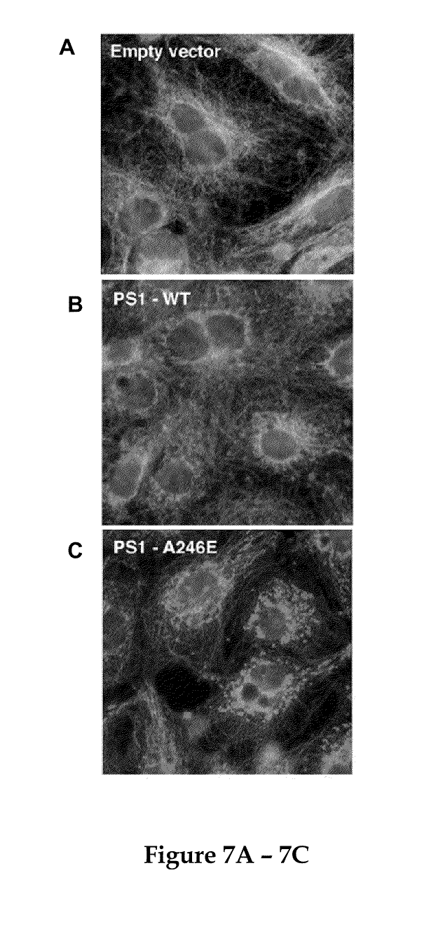

Overexpression of mutant PS1 in stably-transfected transfected COS-7 cells showed that mitochondria in the cells over-expressing mutant PS1, but not control cells, accumulated in the perinuclear region of the cells (FIG. 7), similar to the results observed in FAD^{PS1} patient and PS1-KD cells (as described herein).

Mitochondria in PS1-mutant fibroblasts were more concentrated around the nucleus than were mitochondria in controls, with fewer mitochondria at the extremities of FAD cells (FIG. 8A), and had an altered, more punctate, morphology (FIG. 8C). This effect was quantitated by measuring the intensity of the MT Red signal in the extremities of the cells to confirm that there were significantly fewer mitochondria in the cells' extremities in FAD cells vs. controls (FIG. 8B). ER-MAM protein in the cells was reduced significantly when PS1 is mutated but the amount of communication was increased with mutated PS1 (FIG. 8D). This result shows that when PS1 is mutated, the ER-MAM connections are tighter relative to the wild-type PS1 condition. (FIG. 8D). Given that protein is used a surrogate marker, the lower amount of protein in the mutated SP1 condition is correlated with an increase in the level of non-proteinaceous material in ER-MAM, thereby resulting in a reduction in the density of ER-MAM. Thus, these results show that PS1 contributes to the destabilization of ER-MAM.

Small hairpin RNA (sh-RNA) technology was used to reproduce the mitochondrial maldistribution phenotype by knocking down PS1 expression in mouse embryonic fibroblasts (MEFs). The perinuclear phenotype was recapitulated using cells in which PS1 expression was reduced by >75% (FIG. 8E,F).

FAD^{PS1} is a dominant disorder, but the exact nature of the dominant effect is unclear. Reproduction of the mitochondrial distribution defect in cells in which PS1 had been knocked down by shRNA shows that the mitochondrial maldistribution phenotype can be due to haploinsufficiency rather than a gain-of-function effect of the PS1 mutation (see also Giannakopoulos et al. (1999) Acta Neuropathol (Berl) 98:488-492; Shen J, Kelleher R J, III (2007) Proc. Natl. Acad. Sci. USA 104:403-409).

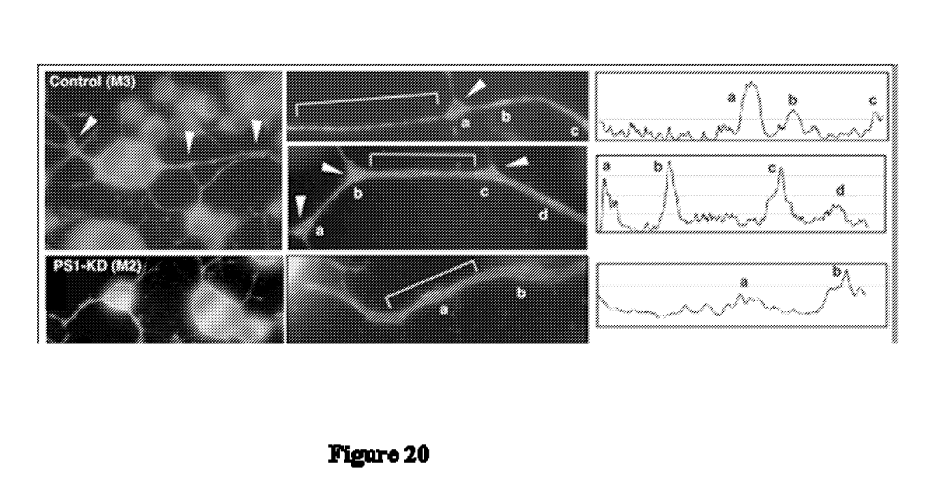

A finding that mutations in PS1 cause haploinsufficiency rather than a gain of function is highly relevant to treatment strategies for FAD^{PS1}. PS1 expression was knocked down by >75% in CCL131 mouse neuroblastoma cells (FIG. 20). The cells were transfected stably with control or PS1 knockdown constructs, differentiated with retinoic acid for 3 days, stained with MT Red and anti-tubulin, and were analyzed in the Imaging Core. In mismatched control (M3) cells, mitochondria were distributed relatively uniformly and densely along the processes (FIG. 20, brackets) and were enriched in varicosities, especially at branch points (FIG. 20, arrowheads). In shRNA-treated cells, however, there was a severely reduced number of mitochondria in cell processes, which was confirmed by scanning of the MT Red intensity along the length of the processes (FIG. 20, right panels) This result indicates that the alterations in mitochondrial dynamics observed in fibroblasts isolated from FAD^{PS1} patients and in PS1-KD MEFs can operate in neuronal tissue as well. This finding will be confirmed in neurons isolated from PS1-mutated mice, as well as in PACS2-knockout mice.

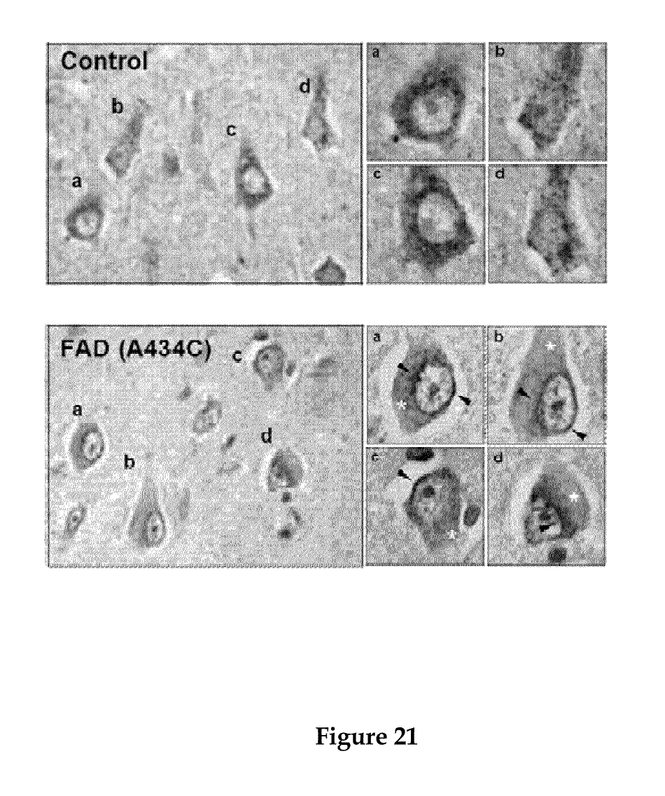

While mutations in PS1 cause a mitochondrial mislocalization phenotype in fibroblasts and neuroblastoma cells, it was not clear if a similar or related phenotype is present in brain, the clinically relevant tissue in FAD. Autoptic brain tissue from patients was obtained and analyzed. The brain tissue was obtained from a sample of hippocampus from the autopsy of a patient with FAD^{PS1} (A434C mutation) (Devi et al. (2000) Arch. Neurol. 57:1454-1457) Immunohistochemistry was performed to detect the FeS subunit of complex III of the mitochondrial respiratory chain in the CA1 region of the hippocampal formation (FIG. 21). There were two observations compared to control: (1) The mitochondria were concentrated in the perinuclear region of the neurons, often forming a “ring” of immunostain around the nucleus, and (2) there was an apparently corresponding absence of immunostain in the distal regions of the cell body.

These findings are in accord with data on cells in tissue culture and are consistent with the finding of axonal transport defects in PS1 transgenic mice (Pigino et al. (2003) J. Neurosci. 23:4499-4508). Analysis of more brain samples will be carried out using method described herein and methods know to those skilled in the art. Taken together, these data indicate that mutations in PS1 have a profound effect on mitochondrial morphology and distribution in somatic and neuronal cells.

Biochemical function in PS1-mutant cells. In order to establish feasibility, initial investigations focused on analysis of respiratory chain function in mitochondria isolated from Percoll-purified mouse brain mitochondria (MBM) from one PS1-transgenic (Tg) mouse and a wild type littermate (Duff et al. (1996) Nature 383:710-713). Isolated mitochondria were well-coupled (respiratory control index [RCI]>9) and exhibited normal rates of the phosphorylating (State 3) and resting (State 4) respiration, and high phosphorylation efficiency (ADP:O≧3) (Table 5)

TABLE 5
Respiration rates of MBM at 37° C. The
phosphorylating (State 3) respiration was
initiated by the addition of 200 nmol ADP
to the mitochondrial suspension. RCI and the
ADP:O ratio were calculated by conventional procedures.

| Genotype | State 3 | State 4 | RCI | ADP:O |
|---|---|---|---|---|
| Wild-Type | 298 | 36 | 9.3 | 3.5 |
| PS1 Tg | 292 | 31 | 9.7 | 3.9 |

Note that values above 3 are due to the contribution of the substrate level phosphorylation, as 2-oxoglutarate was included in the respiratory substrate mixture. However, the RCI and ADP:O ratio in mitochondria from the PS1 Tg animal was higher than in WT mitochondria. Analysis of the activity of the respiratory chain and tricarboxylic acid cycle enzymes (Table 6) did not reveal significant differences between PS1-Tg and WT mitochondria, except that the activity of Complex I was higher in the PS1 transgenic mitochondria. As the difference is beyond the normal range of variability for these measurements, which is ˜10-15%, this requires further investigation in more mice. In spite of the higher Complex I activity in PS1-Tg mitochondria, there was no difference in the respiration rates supported by the oxidation of NAD-linked substrates (Table 5). Activity of Complexes III-V remain to be assessed.

TABLE 6
Enzyme activities of MBM. PDHC, pyruvate
dehydrogenase complex activity by
following the reduction of NAD+ by pyruvate;
MDH, malate dehydrogenase activity by
following the oxaloacetate-induced NADH oxidation,
CS, citrate synthase. All activities in
nmol (NADH, NAD+, DCIP, or DTNB, respectively)
per min per mg mitochondrial protein.

| | | | | MDH | |
|---|---|---|---|---|---|
| Genotype | Complex I | Complex II | PDHC | (× 100) | CS |
| Wild-Type | 1314 | 440 | 52 | 35 | 750 |
| PS1 Tg | 1751 | 451 | 54 | 33 | 740 |

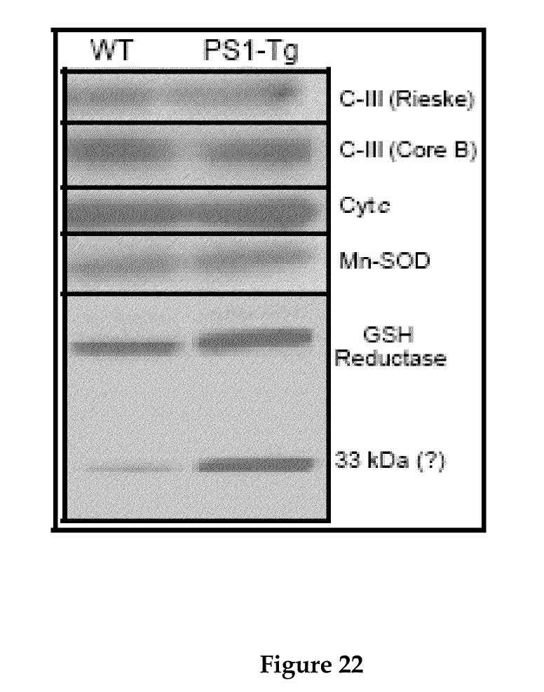

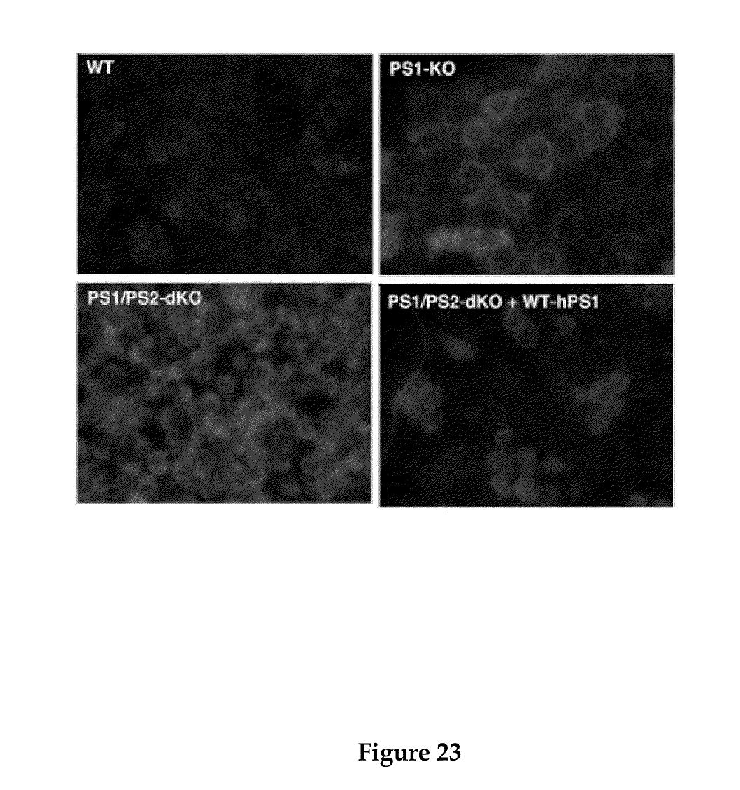

Western blot analyses (FIG. 22) demonstrated similar contents of Complex III in the PS1-Tg and WT mitochondria. The levels of cytochrome c and MnSOD were also similar, indicating an equal level of structural integrity of the isolated WT and PS1-Tg mitochondria, as cytochrome c is a marker of the intermembrane space and MnSOD is a marker of the matrix space. The content of a matrix antioxidant enzyme, GSH reductase, appeared somewhat elevated in the PS1 mitochondria. The monoclonal antibody to GSH reductase cross-reacted with an unknown protein of ˜33 kDa, with a much more intense signal in the PS1-Tg animal that appeared to be specific to PS1-Tg mitochondria (FIG. 22). This can indicate an enhanced detoxifying capacity of these mitochondria toward H₂O₂ and lipid radicals, and is consistent with the results described herein of elevated reactive oxygen species (ROS) in mouse PS1 KO and PS1/PS2-dKO blastocysts and MEFs stained with MitoSox (Molecular Probes) (FIG. 23).

Data were generated on a single pair of matched WT and Tg mice, as an initial pilot study. The same analyses will be carried out on a statistically relevant group of animals. This Tg mouse expresses PS1 from three alleles: two WT mouse PS1 alleles and the mutant human PS1 transgene. Given that FAD^{PS1} may be due to a haploinsufficiency, the bioenergetic “profile” of this Tg line may represent the smallest effect due to mutations in PS1. Analysis of mitochondria isolated from brain and cells from PS1/PS2 dKO mice, which have no WT PS1 alleles, and from PACS2-KO mice in which ER-MAM function is compromised, will be even more informative.

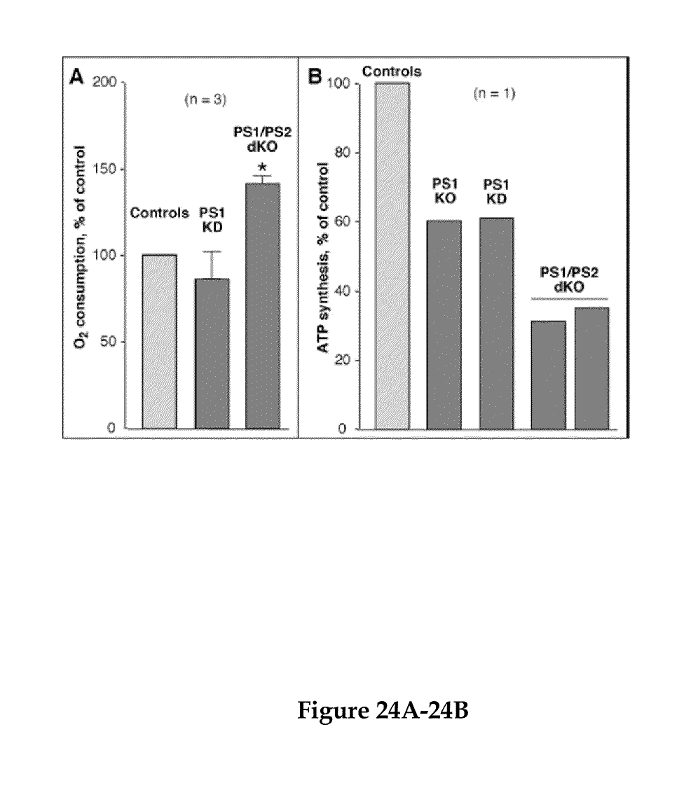

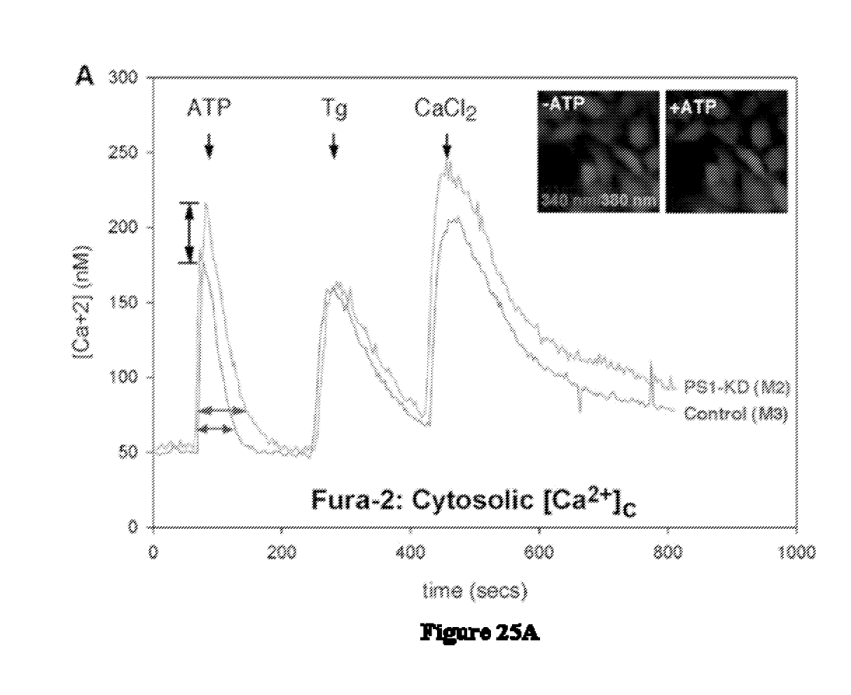

Oxygen consumption was measured polarographically in PS1-knockdown (PS1-KD) 3T3 cells and in PS1-KO and PS1/PS2— dKO MEFs. No difference in O₂ consumption was observed in the KD cells, but a statistically significant 40% increase was observed in the dKO cells (FIG. 24A). Using HPLC, a reduction of about 40% in ATP synthesis in PS1-KD and PS1-KO cells was observed, and about 60% reduction was observed in the dKO MEFs (FIG. 24B). The finding of reduced ATP synthesis but normal respiratory chain activity can be connected to the increase in ROS in these cells and the increase in complex I activity that was observed in the Tg mice.

### Example 6

### Calcium Homeostasis

The close association between ER and mitochondria at the ER-mitochondrial interface is important for calcium signal propagation from IP3 receptors (IP3R) to the mitochondria (Csordas et al. (2006) J. Cell Biol. 174:915-921; Rizzuto et al. (1998) Trends Cell Biol. 8:288-292). Because the results described herein show preferential localization of PS1 at the ER mitochondrial interface, the effect of PS1 and PS1 depletion on mitochondrial calcium signaling was evaluated.

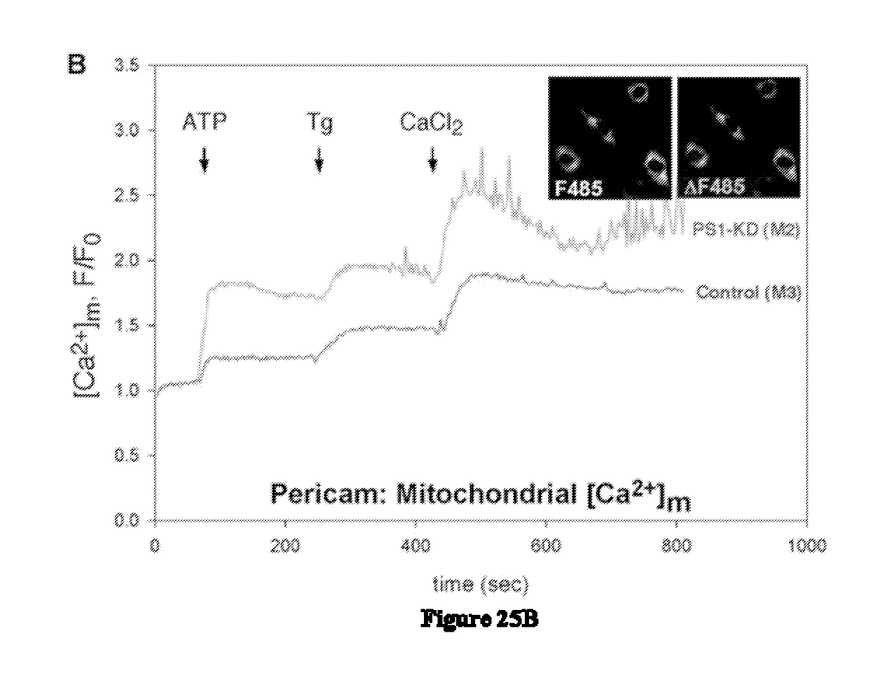

Previous studies have shown PS depletion to cause changes in ER Ca2+ storage and in IP3R function (Smith et al. (2005) Cell Calcium 38:427-437; Ito et al. (1994) Proc. Natl. Scad. Sci. USA 91:534-538; Tu et al. (2006) Cell 126:981-993). Cytoplasmic Ca2+ ([Ca2+]c) was monitored simultaneously with mitochondrial matrix Ca2+ ([Ca2+]m) in 3T3 cells transfected with a PS1-scrambled (control) or PS1-specific knockdown (PS1-KD) shRNA constructs (>75% reduction in PS1). The cells were transfected with a non-ratiometric mitochondrial matrix-targeted Ca2+-sensitive fluorescent protein (inverse pericam (Zhang et al. (2008) BMC Neurosci. in press)) to record [Ca2+]m and were loaded with fura2/AM for ratiometric imaging of [Ca2+]c at 340/380 nm to record [Ca2+]c in single cells (FIG. 25). The cells were stimulated sequentially with ATP (to induce IP3R-mediated Ca2+ mobilization), with thapsigargin (Tg; an inhibitor of the SERCA to complete depletion of Ca2+ from the ER into the cytosol), and finally with extracellular CaCl₂ (to allow for store-depletion-induced Ca2+ entry into the cytosol). Addition of ATP evoked a cytosolic [Ca2+]c spike in both control and PS1-KD cells, but the [Ca2+]c spike was relatively large in the PS1-KD cells (n=7 experiments), a result consistent with a recent report on the effect of mutant PS1 and PS2 on Ca2+ mobilization (Tu et al. (2006) Cell 126:981-993). Release of the residual ER Ca2+ by Tg and the store-depletion operated Ca2+ influx caused similar elevations in [Ca2+]c in both WT and PS1 KD cells (FIG. 25A). Thus, the ER Ca2+ storage was greater, and allowed for larger IP3 induced Ca2+ mobilization in the PS1-KD cells. Simultaneous measurements of mitochondrial [Ca2+]m (FIG. 25B) showed a rapid transfer of the IP3-induced [Ca2+]c signal to the mitochondria. However, the [Ca2+]m signal was >2-fold higher in the PS1-KD cells (n=7). As expected, Tg and CaCl₂ induced similar [Ca2+]m increases in both WT and PS1 KD cells (FIG. 25B). Thus, IP3-dependent Ca2+ transfer to mitochondria was massively increased in the PS1-KD cells.

For measurements of mitochondrial matrix [Ca2+] ([Ca2+]m), the cells were transfected with a mitochondrial matrix targeted inverse pericam construct (Nagai et al. (2001) Proc. Natl. Acad. Sci. USA 98:3197-3202) by electro oration 24-48 h prior to the imaging experiment. Cells were preincubated in an extracellular medium as described (Yi et al. (2004) J. Cell Biol. 167:661-672; Duff et al. (1996) Nature 383:710-713). To monitor [Ca2+]c cells were loaded with 5 μM Fura2/AM for 20-30 min in the presence of 200 μM sulfinpyrazone and 0.003% (w/v) pluronic acid at room temperature. Before start of the measurement the buffer was replaced by a Ca2+-free 0.25% BSA/ECM ([Ca2+]<μM). Coverslips were mounted on the thermo stated stage (35° C.) of a Leica IRE2 inverted microscope fitted with a 40×(Olympus UApo, NA 1.35) oil immersion objective. Fluorescence images were collected using a cooled CCD camera (PXL, Photometrics).

Excitation was rapidly switched among 340 and 380 nm for fura2 and 495 nm for pericam, using a 510 nm longpass dichroic mirror and a 520 nm longpass emission filter. For evaluation of [Ca2+]c, Fura2 fluorescence was calculated for the total area of individual cells. [Ca2+]c was calibrated in terms of nM using in vitro dye calibration. For evaluation of [Ca2+]m, the pericam-mt signal was masked. Recordings obtained from all transfected cells on the field (8-15 cells) were averaged for comparison in each experiment. Significance of differences from the relevant controls was calculated by Student's t test. Cells will be challenged with compounds that affect intracellular Ca2+ concentration, such as 300 nM bradykinin (which stimulates IP3-mediated Ca2+ release) and 5 μM ionomycin (a Ca2+ ionophore that induces formation of Ca2+-permeable pores, leading to emptying of ER Ca2+ stores independent of IP3-mediated receptor activation (Nelson et al. (2007) J. Clin. Invest. 117:1230-1239)).

In summary, silencing of PS1 caused an increase in the IP3-dependent Ca2+ mobilization and massive potentiation of the ensuing mitochondrial Ca2+ accumulation, confirming that PS1 is an important regulator of Ca2+ storage in the ER. This result indicates that PS1 exerts a major effect on ER-mitochondrial Ca2+ transfer, sensitizing mitochondria to permeabilization in FAD^{PS1} cells, leading to cell injury.

### Example 7

### Functional Assays of MAM

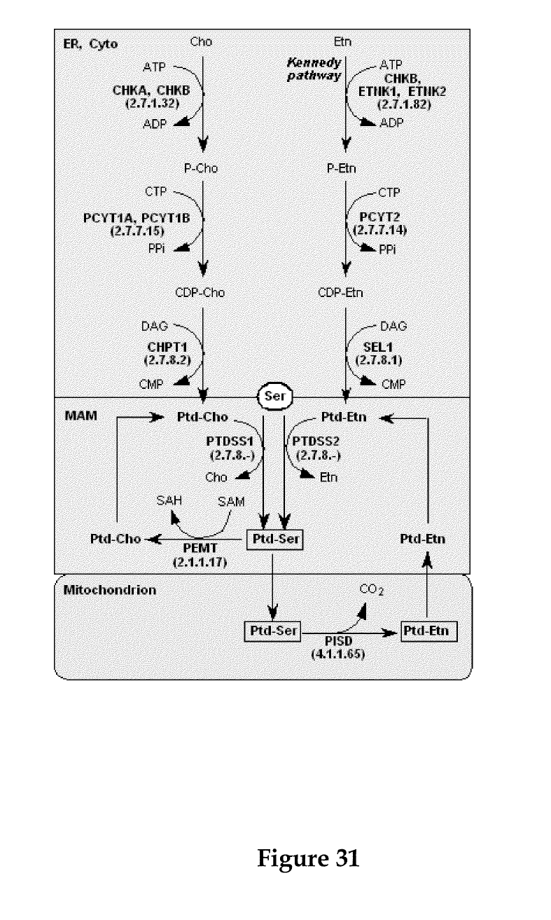

One of the described functions of MAM is to regulate the transport of selected lipids from the ER into the mitochondria. For example, phosphatidylserine (PtdSer) moves from the MAM to mitochondria, where it is decarboxylated to phosphatidylethanolamine (PtdEtn); PtdEtn then moves back to the MAM, where it is methylated to phosphatidylcholine (PtdCho) (FIG. 31). Thus, the kinetics of trafficking of PtdSer from the MAM to mitochondria is a recognized measurement of MAM function (Schumacher et al. (2002) J. Biol. Chem. 277:51033). In one embodiment, a MAM function assay is based on the measurement of the incorporation of 3H-Ser into phospholipids, as described by Voelker (Schumacher et al. (2002) J. Biol. Chem. 277:51033). As shown in the schematic in FIG. 31, exogenously added serine (Ser) is incorporated into PtdSer in the MAM, via an exchange reaction in which serine replaces ethanolamine (Etn) in PtdEtn or choline (Cho) in PtdCho via the action of phosphatidylserine synthase 1 and 2 (PTDSS1 and PTDSS2 in humans), respectively. The resulting PtdSer is then transported from the MAM to mitochondria, where it is decarboxylated to PtdEtn by mitochondrial phosphatidylserine decarboxylase (PISD). The resulting PtdEtn is transported back to the MAM, where it can be methylated to PtdCho by phosphatidylethanolamine methyltransferase (PEMT). In the MAM activity assay, 3H-Ser is added to cells in medium lacking Etn but containing Cho, so that PtdSer is made from PtdCho via PTDSS1, but not from PtdEtn via PTDSS2, at least not initially, because there is no exogenous source of Etn to form PtdEtn via the Kennedy pathway. Thus, the only way PtdEtn can be made is via the MAM pathway, and the amount of 3H incorporated into 3H-PtdSer and 3H-PtdEtn is a measurement of MAM function.

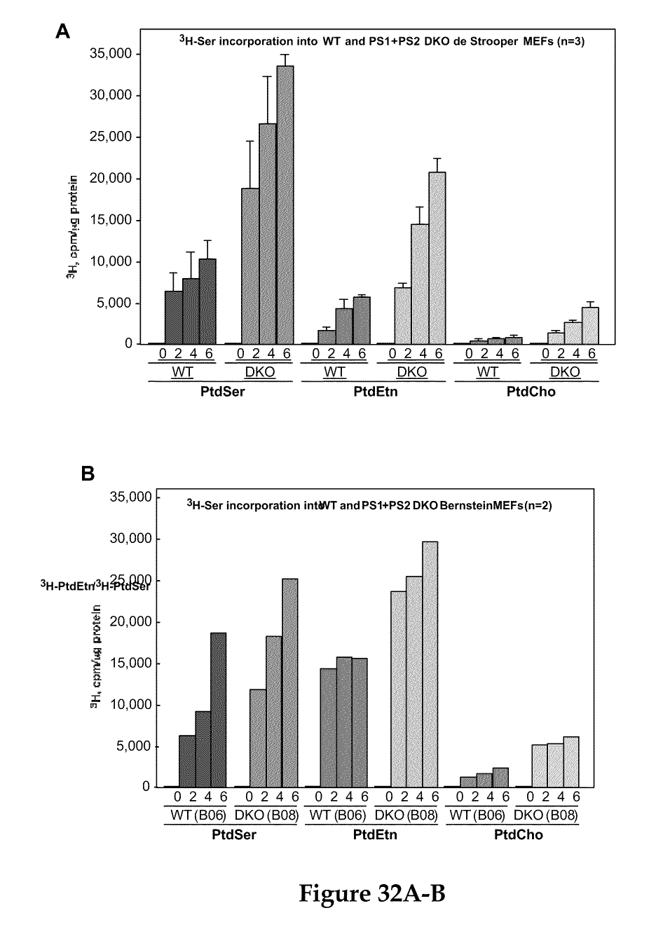

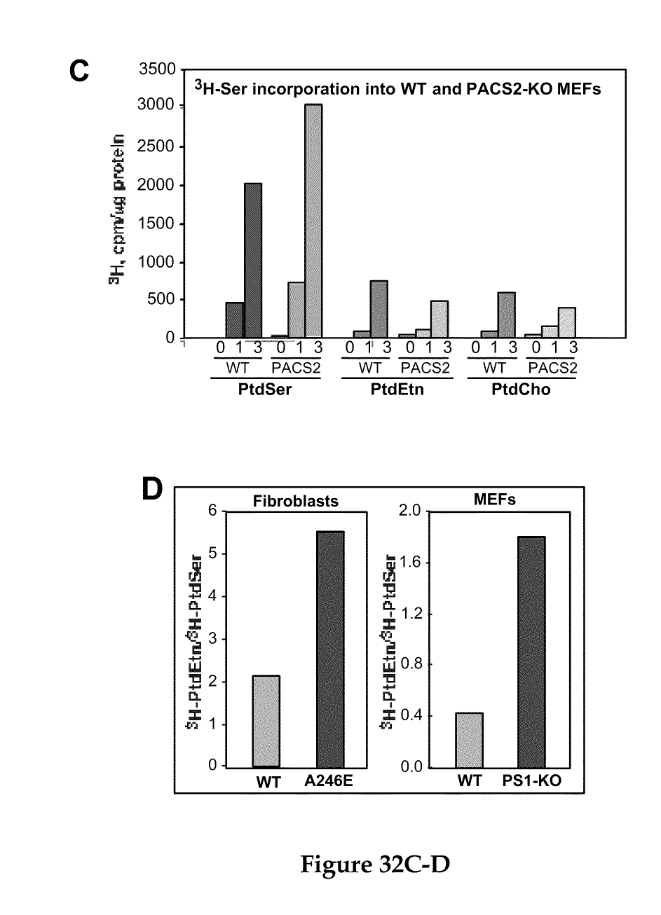

Applying this technique to PS1 mutant fibroblasts and to PS1 knock-out (PS1-KO) mouse embryonic fibroblasts (MEFs) vs. controls, a significant increase in PtdEtn synthesis was detected in PS1-mutant cells (FIG. 32D), reflecting an upregulated transport of PtdSer into mitochondria, and implying that defects in PS1 indeed affect MAM function. As a control, MEFs in which MAM-mitochondrial communication had been abrogated by knocking out PACS2 (Simmen et al. (2005) EMBO J. 24:717) were shown to retain their ability to synthesize 3H-PtdSer but had decreased formation of PtdEtn (and PtdCho).

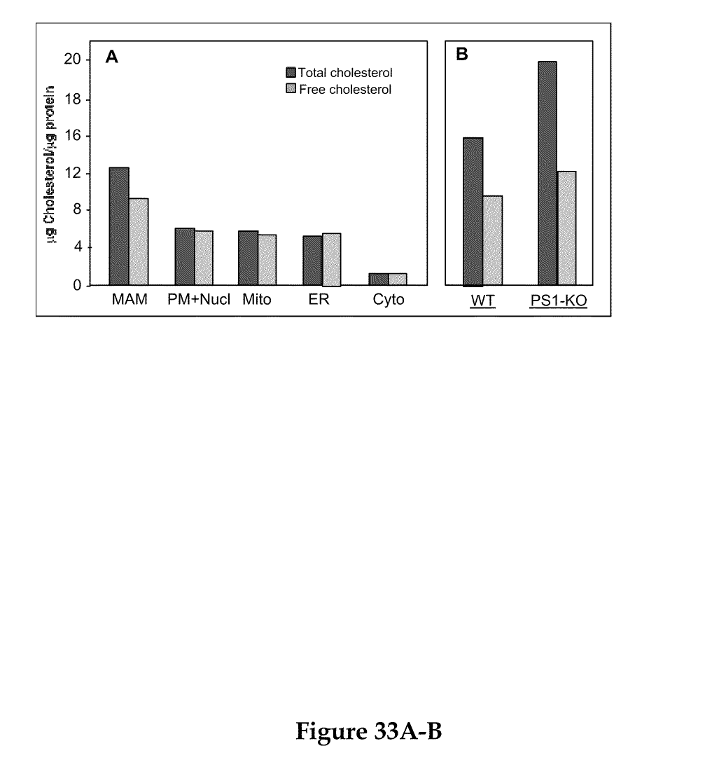

There is elevated cholesterol in patients with AD. Mutations in PS1 causing altered MAM function should also show altered cholesterol content. Moreover, if MAM function is reduced in PS1-mutant cells and tissues, cholesterol content can be increased concomitantly. MAM indeed contains high levels of cholesterol, both as free cholesterol and as cholesterol esters (FIG. 33A). Moreover, when the crude mitochondrial fraction from the brains of WT and PS1-knock-in mice (M146L mutation; courtesy of Mark Mattson; Guo et al. (1999) Nature Med 5:101) are examined, the amount of both total and free cholesterol was increased in the KI vs. the WT mice (FIG. 33).

This result can be explained by the role of a key MAM protein, acyl-coA:cholesterol acyltransferase (ACAT1 [gene SOAT1]), which not only synthesizes cholesterol esters in the MAM, but also is important for the generation of Aβ by modulating the equilibrium between of free and esterified cholesterol (Puglielli et al. (2005) Nature Cell Biol. 3:905; Puglielli et al. (2004) J. Mol. Neurosci. 24:93). In addition, that steroid biosynthesis requires ER-mitochondrial communication, across the MAM, as cholesterol must be imported from the ER into mitochondria, where it is converted into pregnenolone, which is then exported back to the ER for further steroid synthesis (e.g. testosterone and estradiol). Thus, tighter communication between the MAM and mitochondria could increase cholesterol biosynthesis and hence, Aβ production.

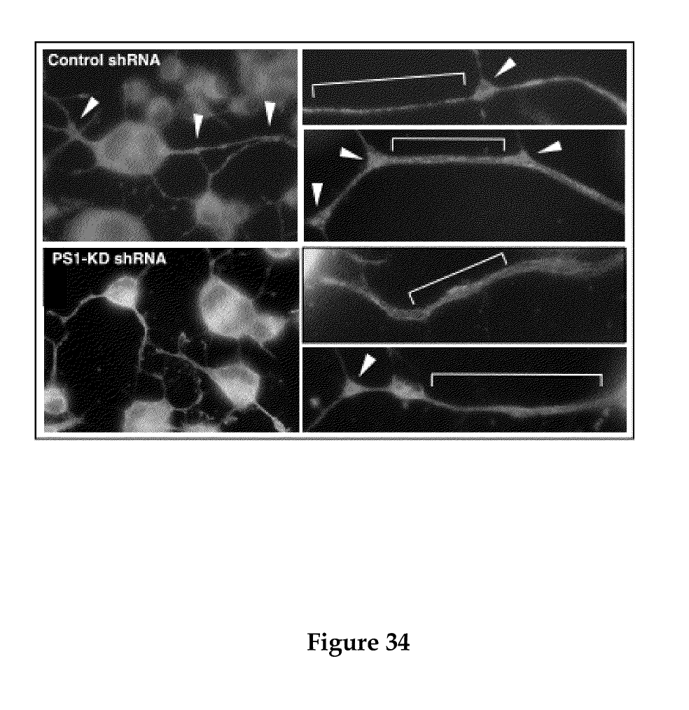

Mitochondrial dynamics in PS1-mutant neuronal-like cells. Since AD is a brain disorder, PS1 expression was knocked down by >75% in CCL131 mouse neuroblastoma cells and stained the cells with MitoTracker Red and anti-tubulin (FIG. 34). In control cells, mitochondria were distributed relatively uniformly and densely along the processes (FIG. 34, brackets) and were enriched in varicosities, especially at branch points (FIG. 34, arrowheads). In PS1-knockdown (KD) shRNA-treated cells, however, there was a severely reduced number of mitochondria in cell processes, which was confirmed by scanning of the MT Red intensity along the length of the processes. This result shows that the alterations in mitochondrial dynamics observed in fibroblasts isolated from FAD^{PS1} patients and in PS1-KD MEFs operate in neuronal tissue as well.

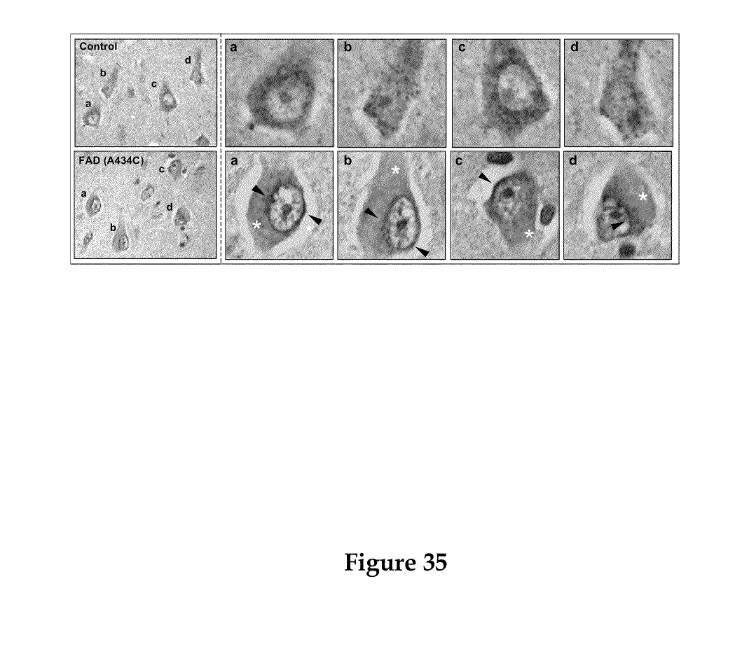

Mitochondrial maldistribution in AD brain. A similar or related mitochondrial maldistribution phenotype present in brain, the clinically relevant tissue in FAD. A sample of hippocampus was obtained from the autopsy of a patient with FAD^{PS1} (A434C mutation) Immunohistochemistry was performed to detect the FeS subunit of complex III of the mitochondrial respiratory chain in the CA1 region of the hippocampal formation (FIG. 35). This analysis resulted in at least two observations (1) The mitochondria were concentrated in the perinuclear region of the neurons, often forming a “ring” of immunostain around the nucleus, and (2) there was a corresponding absence of immunostain in the distal regions of the cell body. Both results are consistent with a perinuclear localization of mitochondria in FAD^{PS1} brain. These findings are similar to results on cells in tissue culture and are consistent with the finding of axonal transport defects in PS1 transgenic mice (Stokin et al. (2005) Science 307:1282) and in human SAD patients (Stokin et al. (2005) Science 307:1282; Wang X, et al. (2008) Am. J. Pathol. 173:470).

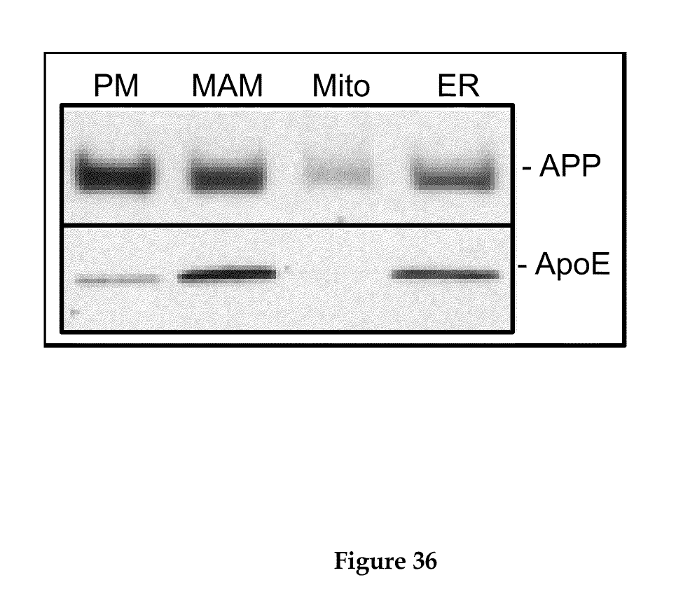

ApoE and APP are also present in MAM. ApoE activity is enriched in MAM (Vance (1990) J. Biol. Chem. 265:7248). ApoE protein is enriched in MAM (˜3-fold over that in ER) (FIG. 36). In addition, APP is also present in abundant amounts in MAM (FIG. 36). These findings show that MAM is implicated not only in familial AD, but in sporadic AD as well. Also, the localization of both PS1 (a component of γ-secretase) and APP (a γ-secretase substrate) in the same compartment explain how Aβ is transported to adjacent mitochondria (Lustbader et al. (2004) Science 304:448), reportedly via a so-called “unique” pathway (Hansson Petersen et al. (2008) Proc. Natl. Acad. Sci. USA 105:13145.), thus providing a solution to the so-called “spatial paradox.”

A number of proteins associated either directly with AD—PS1, PS2, APP, ApoE, CD147—or indirectly via the other functions are known to be altered in AD—calcium, lipid, ceramide, and glucose metabolism—are enriched in the MAM.

Mutations in PS1 and PS2, rather than reducing MAM-mitochondrial communication, increase it. This tighter link between mitochondria and ER via the MAM explains the altered phospholipid profiles and elevated cholesterol seen in AD, and explains not only the elevated Aβ synthesis, but also the inability of mitochondria to get “off” the ER and get “on” to microtubules for subsequent movement away from the cell body. This difficulty can be especially catastrophic in neurons that require mitochondria to move vast distance from the cell body to axons and dendrites in order to maintain normal brain function. Thus, altered MAM function is a cause of the pathogenesis of both familial and sporadic AD.

### Example 8

### Presenilins are Negative Regulators of ER-Mitochondrial Communication

Presenilin-1 (PS1) and -2 (PS2), and γ-secretase activity, are enriched in a subcompartment of the endoplasmic reticulum (ER) which physically and functionally interacts with mitochondria, called ER membranes associated with mitochondria (MAM). As described herein, MAM displays the features of an intracellular lipid raft, and that the absence of presenilins upregulates the communication between ER and mitochondria, as measured by two key biochemical assays of MAM behavior, phospholipid transport and cholesteryl ester synthesis. Cells lacking presenilins also displayed a significant increase in the physical association of these two compartments. The results described herein demonstrate that presenilins are negative regulators of ER-mitochondrial communication, and that this upregulation plays a key role in the pathogenesis of Alzheimer disease.

Alzheimer disease (AD) is a late onset neurodegenerative disorder characterized by progressive neuronal loss, especially in the cortex and the hippocampus (Goedert and Spillantini (2006) Science 314, 777-781). The two main histopathological hallmarks of AD are the accumulation of neurofibrillary tangles, consisting mainly of hyperphosphorylated forms of the microtubule-associated protein tau, and of extracellular neuritic plaques, consisting mainly of β-amyloid (Aβ) species (predominantly Aβ40 and Aβ42), a 4-kDa peptide derived from the cleavage of the amyloid precursor protein (APP) by β- and γ-secretases (Goedert and Spillantini (2006) Science 314, 777-781). The vast majority of AD is sporadic, but mutations in PS1, and PS2, as well as in APP, have been identified in the familial form. PS1 and PS2 are aspartyl proteases that are components of the γ-secretase complex that processes a number of membrane-bound proteins, including APP.

Lipid rafts (LR) are specialized domains enriched in cholesterol and sphingolipids that form spontaneous nonionic detergent-insoluble aggregates, or DRMs, in cell membranes (Simons and Vaz (2004) Annu. Rev. Biophys. Biomol. Struct. 33, 269-295). These regions have a lower density liquid-ordered structure that differs from the rest of the cell's liquid disordered membranes, due to the interaction of cholesterol with phospholipid acyl chains that allow for a very densely-packed structure with unique biophysical characteristics compared to those of non-raft membranes (Simons and Vaz (2004) Annu. Rev. Biophys. Biomol. Struct. 33, 269-295). While lipid rafts have been described to be present exclusively in the plasma membrane (PM), recent evidence has pointed to the existence of intracellular lipid rafts different in protein composition from those in the PM (Browman et al., (2006) J. Cell Sci. 119, 3149-3160; Mellgren (2008) J. Biochem. Biophys. Methods 70, 1029-1036.). Presenilins, APP, Aβ, and γ-secretase activity itself are particularly enriched in LR/DRMs domains that are highly concentrated in cholesterol, and which do not comigrate with bulk ER or Golgi markers in sucrose gradients (Lee et al., 1998) Nat. Med. 4, 730-734; Kim et al., (2000) Neurobiol. Dis. 7, 99-117; Urano et al., (2005) J. Lipid. Res 46, 904-912). ER membranes associated with mitochondria, or MAM, comprise a subcompartment of the ER that is physically and biochemically linked with mitochondria (Hayashi et al., (2009) Trends Cell Biol. 19, 81-88). It is involved in a number of key metabolic functions (Hayashi et al., (2009) Trends Cell Biol. 19, 81-88), including the synthesis and transfer of phospholipids between the ER and mitochondria (Vance (2003) Prog. Nucl. Acid Res. Mol. Biol. 75, 69-111), cholesterol metabolism (Rusinol et al., (1994) J. Biol. Chem. 269, 27494-27502), and calcium homeostasis (Rizzuto et al., (1998) Science 280, 1763-1766; Csordas et al., (2006) J. Cell Biol. 174, 915-921).

As described herein, both PS1 and PS2, and γ-secretase activity itself, are enriched in the MAM. We now show that MAM is a DRM displaying the characteristics of an intracellular lipid raft. Moreover, the results described herein show that the loss of presenilins affects MAM structure profoundly and increases functions associated with MAM, suggesting that presenilins act as negative regulators of ER-mitochondrial communication.

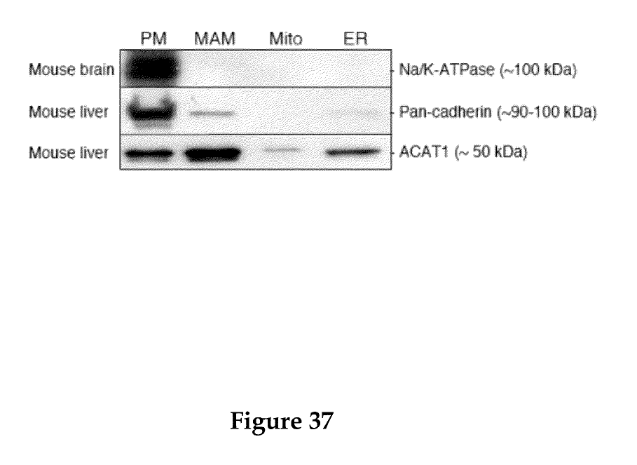

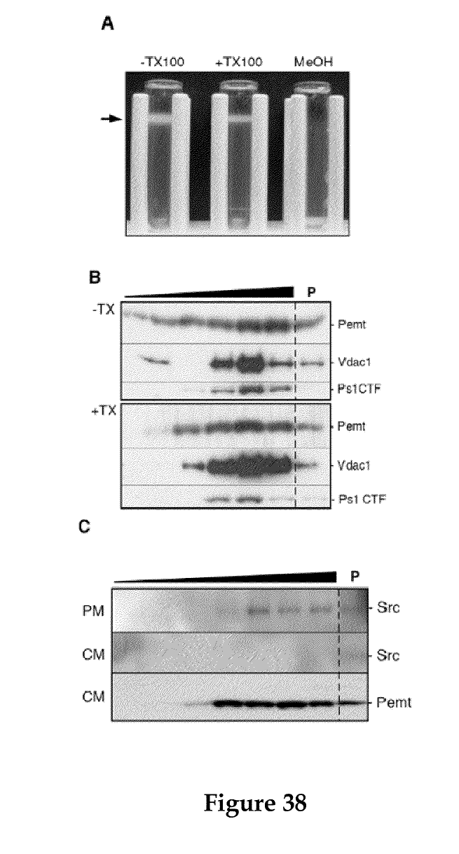

The differentially lower density of MAM in a gradient as compared to that of bulk ER or mitochondria, as described herein, led us to speculate that MAM has a composition similar to that of lipid rafts (Simons and Vaz (2004) Annu. Rev. Biophys. Biomol. Struct. 33, 269-295). Purified MAM from mouse tissues was therefore incubated with and without Triton-X 100 (TX100), and loaded both samples onto a Percoll gradient under the same conditions used for its initial isolation. FIG. 37 shows the fractionation of mouse tissues to isolate MAM. The TX100-treated MAM sample was fundamentally intact and migrated to the identical position in the gradient as did the untreated sample, consistent with the behavior of a DRM (FIG. 38A).

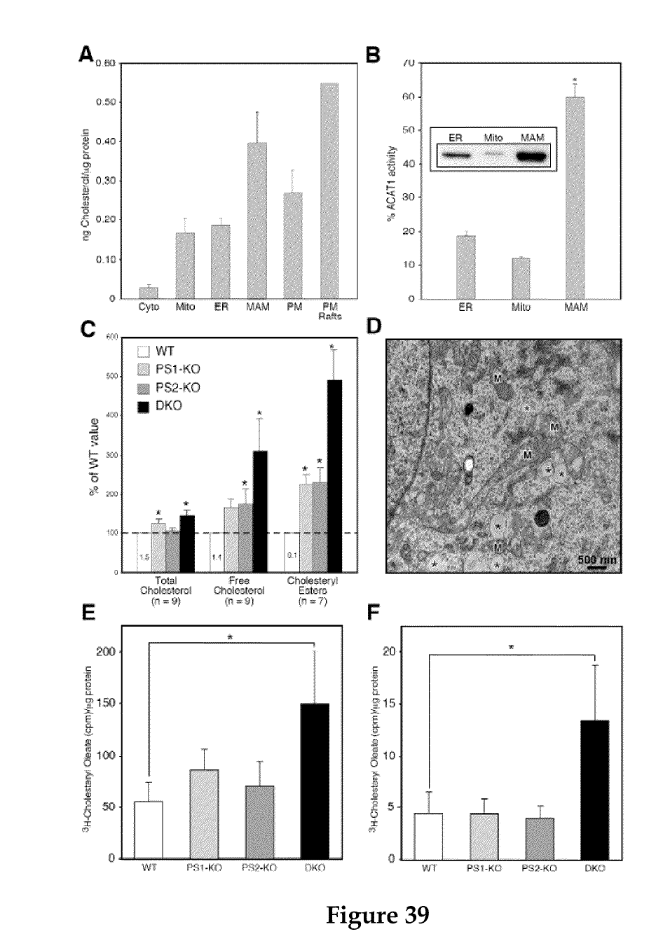

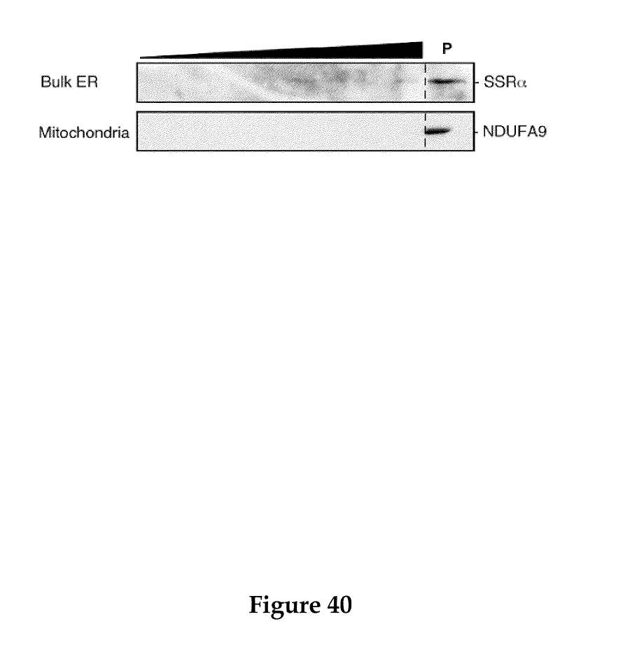

To separate LR from other cell contents, TX100-treated and control MAM fractions were loaded onto a sucrose gradient (Ostrom and Liu (2007) Meth. Mol. Biol. 400, 459-468), and analyzed fractions by Western blotting to detect known MAM markers: Pemt (phosphatidylethanolamine N-methyltransferase) (Vance (1990) J. Biol. Chem. 265, 7248-7256), Vdac1 (voltage-dependent anion channel 1) (Hayashi et al., (2009) Trends Cell Biol. 19, 81-88), and PS1 (FIG. 38B). The proteins migrated at similar positions in the lower density fractions, and the migration pattern was unaffected by detergent treatment (FIG. 38B). Importantly, MAM was not contaminated with LR/DRMs from plasma membrane (PM), as Src, a marker for PM LR/DRMs (Morrow and Parton (2005) Traffic 6, 725-740), was observable in sucrose gradient fractions from purified PM, but not from the crude mitochondrial fraction (CM) from which the MAM fraction was derived (FIG. 38C). Moreover, the cholesterol content of mouse brain MAM was higher than that found in the cytoplasm, mitochondria, bulk ER, and total PM, and was comparable to that of LR from PM (Simons and Vaz (2004) Annu. Rev. Biophys. Biomol. Struct. 33, 269-295) (FIG. 39A). By contrast, purified mitochondria and bulk ER from the bottom of the gradient behaved like detergent-soluble fractions (FIG. 40), indicating the absence of DRMs, as expected (Zheng et al., (2009) J. Lipid Res. 50, 988-998). FIG. 40 shows that neither bulk ER nor mitochondria are detergent-resistant membranes.

MAM/mitochondria markers, such as VDAC (FIG. 38B) or calnexin (Hayashi et al., (2009) Trends Cell Biol. 19, 81-88; Foster and Chan (2007) Subcell. Biochem. 43, 35-47) have been found in PM because, apart from the lack of appropriate markers to detect MAM, most LR isolation methods do not separate PM from intracellular membranes (Macdonald and Pike (2005) J. Lipid Res. 46, 1061-1067); such PM raft preparations will therefore be cross-contaminated with MAM. Moreover, several authors have described or suggested the existence of intracellular rafts in the ER or mitochondria (Browman et al., (2006) J. Cell Sci. 119, 3149-3160; Mellgren (2008) J. Biochem. Biophys. Methods 70, 1029-1036; Martinez-Abundis et al., (2009) FEBS J. 276, 5579-5588). These intracellular LR/DRMs are in fact MAM (Hayashi and Fujimoto (2010) Mol. Pharmacol. 77, 517-528). Conversely, crude mitochondrial preparations contain MAM, and can be misinterpreted to suggest that mitochondria contain LR (Martinez-Abundis et al., (2009) FEBS J. 276, 5579-5588), when in fact they do not (Zheng et al., (2009) J. Lipid Res. 50, 988-998) (FIG. 40).

With respect to Alzheimer disease, it has long been known that presenilins, APP, Aβ, and γ-secretase activity, are enriched in LR-like DRMs (Lee et al., 1998) Nat. Med. 4, 730-734; Kim et al., (2000) Neurobiol. Dis. 7, 99-117; Urano et al., (2005) J. Lipid. Res 46, 904-912). Thus, the localization of presenilins in an intracellular raft could help resolve the discrepancy between the putative site of Aβ generation at the PM and the predominantly intracellular location of PS1 and γ-secretase activity (the “spatial paradox” (Cupers et al., (2001) J. Cell Biol. 154, 731-740)).

As MAM is a LR/DRM, the regulation of cholesterol metabolism may be an important determinant of its structure and function. Acyl-CoA:cholesterol acyltransferase (ACAT), which catalyzes the conversion of free cholesterol to cholesteryl esters, is enriched in MAM (Rusinol et al., (1994) J. Biol. Chem. 269, 27494-27502). ACAT controls the equilibrium between membrane-bound free cholesterol and cholesteryl esters stored in cytoplasmic lipid droplets (23). ACAT1, the predominant ACAT isoform in brain, was confirmed to not only be more abundant in MAM compared to bulk ER and mitochondria (FIG. 39B, inset), but also that is has a correspondingly higher enzymatic activity (FIG. 39B).

In order to determine if presenilins play a role in regulating MAM function, mouse embryonic fibroblasts (MEFs) lacking PS1 (PS1-KO), PS2 (PS2-KO), or both proteins (DKO) were analyzed (Herreman et al., (2000) Nat. Cell Biol. 2, 461-462), focusing first on cholesterol synthesis and ACAT activity. Compared to WT MEFs, the mutant lines showed increased levels of total cholesterol (FIG. 39C), in agreement with others (Grimm et al., (2005) Nat. Cell Biol. 7, 1118-1123). Further analysis showed higher free cholesterol contents in mutant vs. WT MEFs, but more importantly, the relative differences in the content of cholesteryl esters (CE) were even greater (FIG. 39C). Notably, significantly higher ACAT activity in DKO MEFs was observed, measured both in cultured cells in vivo (FIG. 39E) and in isolated MAM in vitro (FIG. 39F). Numerous CE-containing lipid droplets in the DKO cells were detected that were absent in control MEFs (FIG. 39D).

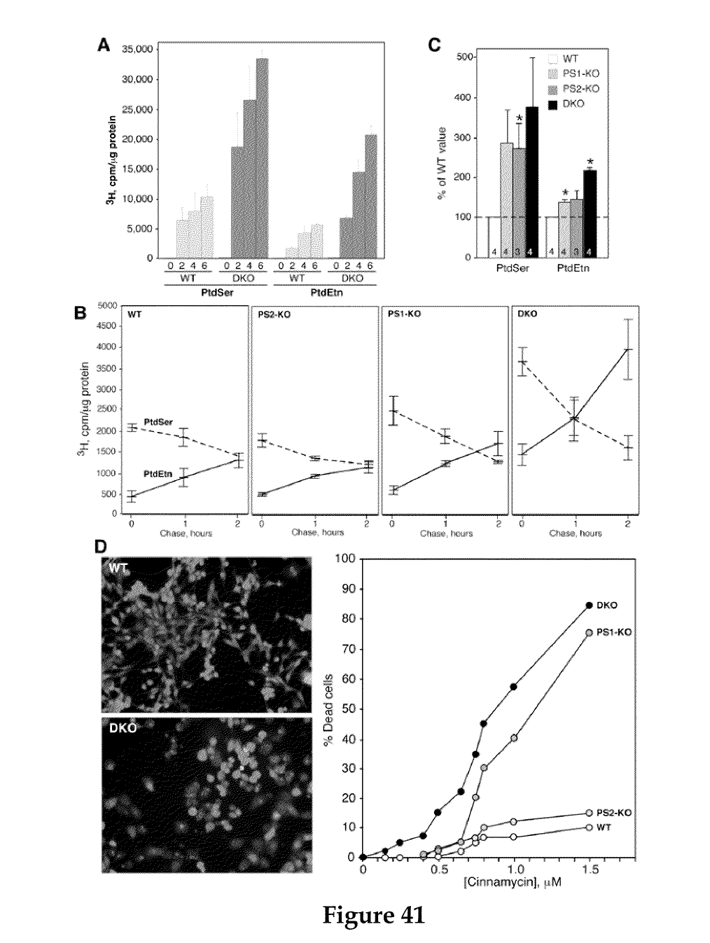

These results are relevant to the pathogenesis of AD. AD patients have elevated cholesterol (Stefani and Liguri (2009) Curr. Alz. Res. 6, 15-29), elevated ACAT1 levels (Pani et al., (2009) J. Alzheimers Dis. 18, 829-841), and neuronal deposition of lipid droplets (Gómez-Ramos and Asunción Morán (2007) J. Alzheimers Dis. 11, 53-59). In addition, there is evidence that ACAT activity affects Aβ production (Puglielli et al., (2001) Nat. Cell Biol. 3, 905-912.) and that MAM plays a role in lipid droplet formation (Walther and Farese (2009) Biochim. Biophys. Acta 1791, 459-466). Moreover, altered cholesterol and lipid composition may change the topology of the MAM membrane, thereby influencing the orientation of APP and its cleavage by γ-secretase, and hence, the production of total Aβ and/or the ratio of Aβ42:Aβ40 (Grimm et al., (2005) Nat. Cell Biol. 7, 1118-1123; Wang et al., (2007) Biophys. J. 92, 2819-2830; Grziwa et al., (2003) J. Biol. Chem. 278, 6803-6808). Taken together, the results described herein show that upregulated communication between ER and mitochondria in presenilin-mutant cells results in increased ACAT1 activity, and can account for the increased cholesterol levels, lipid droplet formation, and altered content of Aβ species found in AD. MAM is also required for the synthesis of most of the cell's phosphatidylethanolamine (PtdEtn) (Voelker (2000) Biochim. Biophys. Acta 1486, 97-107). Phosphatidylserine (PtdSer) is synthesized in the MAM via phosphatidylserine synthase 2 (Hayashi et al., (2009) Trends Cell Biol. 19, 81-88); PtdSer translocates to mitochondria, where it is converted to PtdEtn by phosphatidylserine decarboxylase; finally, PtdEtn translocates back to the MAM, where it is methylated by phosphatidylethanolamine methyltransferase (PEMT) (Vance (2008) J. Lipid Res. 49, 1377-1387) to generate phosphatidylcholine. The trafficking of PtdSer from MAM to mitochondria is a recognized measure of MAM function {Voelker, 2005 #113}. Presenilin-mutant MEFs were incubated in medium containing 3H-serine and analyzed the incorporation of the label into newly synthesized PtdSer and PtdEtn. The levels of both labeled species were highly elevated in the DKO MEFs compared to WT (FIG. 41A), suggesting upregulation of MAM-mitochondrial crosstalk.

Pulse-chase analysis was performed by incubating the MEFs with 3H-Ser for 1 hour, followed by a chase with cold serine (FIG. 41B). As expected, the incorporation of label into PtdSer during the pulse was higher in the mutant MEFs (time 0 in FIG. 41B). During the chase, the amount of 3H-PtdSer decreased and 3H-PtdEtn increased, consistent with the conversion of the former into the latter, with increased conversion rates in mutant MEFs, again indicating increased MAM-mitochondrial communication. To rule out the possibility that other cellular factors might be contributing to this effect, 3H-PtdSer and 3H-PtdEtn synthesis was measured in vitro on isolated MEF crude mitochondrial fractions (containing essentially only ER, MAM, and mitochondria [not shown]) (FIG. 41C). As before, the synthesis of both phospholipid species was higher in the CM from mutant MEFs vs. control, confirming that the loss of presenilins resulted in upregulation of the interaction between ER and mitochondria. While the increase in lipid synthesis was least pronounced in the PS2-DKO MEFS, it is nevertheless clear that PS2, like PS1, contributes to ER-mitochondrial cross-talk, as the synthesis in the PS1+PS2 double knockout was much more pronounced than in the PS1 knockout alone.

AD patients have aberrant phospholipid profiles, both in fibroblasts and in brain (Pettegrew et al., (2001) Neurochem. Res. 26, 771-782; Murphy et al., (2006) Brain Res. Bull. 69, 79-85). PtdSer and PtdEtn are exported to the inner leaflet of the plasma membrane (Vance (2008) J. Lipid Res. 49, 1377-1387). To determine if they are also elevated in the PM of mutant MEFs, MEFs were treated with the antibiotic cinnamycin (also called Ro 09-0198), a 19-aa cyclic peptide “lantibiotic” that forms a 1:1 complex specifically with PtdEtn and induces transbilayer phospholipid movement that leads to the “flipping” of inner leaflet PtdEtn to the outer leaflet; this results in pore formation in the PM and subsequent cell death, in a PtdEtn concentration dependent manner (Makino et al., (2003) J. Biol. Chem. 278, 3204-3209.). In agreement with the 3H-Ser labeling experiments, PS-mutant MEFs were more sensitive to cinnamycin than were controls (FIG. 41D). Both the 3H-Ser and cinnamycin results are relevant to the pathogenesis of AD.

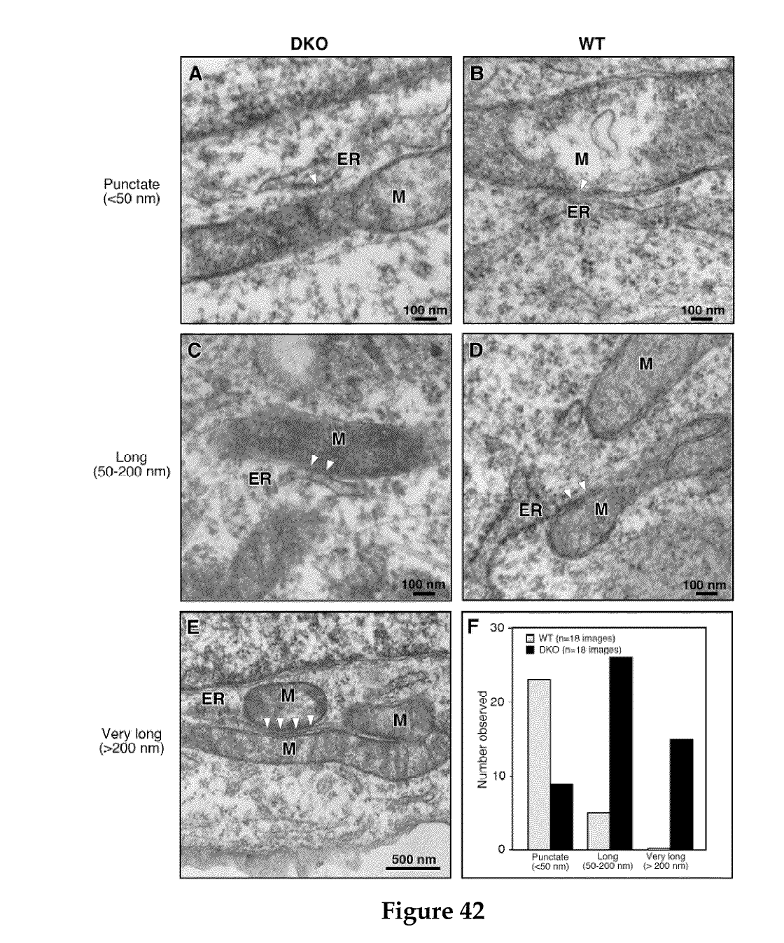

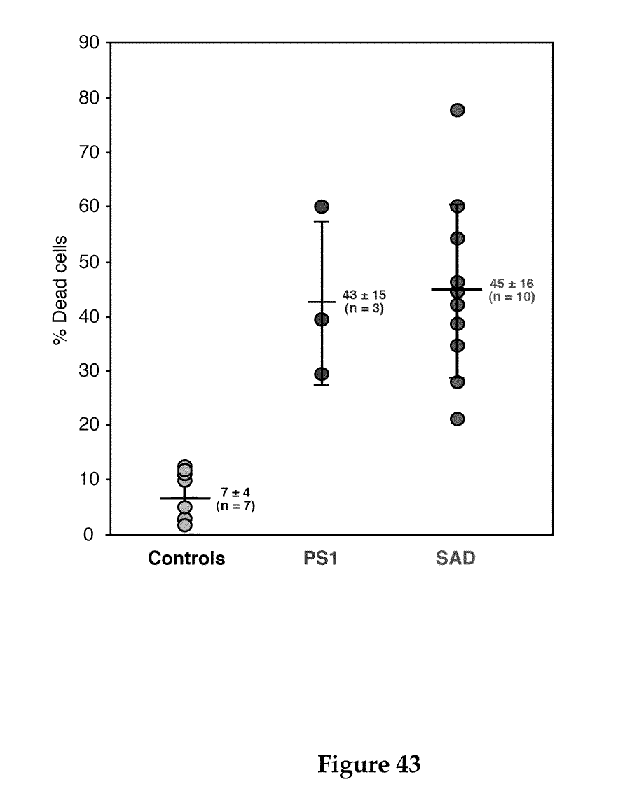

Electron microscopy of WT and DKO MEFs was performed to examine the association between ER and mitochondria (FIG. 42). An increase in the length of mitochondrial-ER contacts (i.e. MAM) was observed. There were significantly more numerous long (50-200 nm) and very long (>200 nm) contacts in DKO MEFs than in WT, whereas connections in WT MEFs were predominantly punctate (<50 nm) (FIG. 42). This result shows that the increased biochemical activity of MAM in PS-mutant cells is due, at least in part, to an increased physical association between the two organelles.

Besides aberrant cholesterol and phospholipid metabolism, calcium homeostasis is clearly perturbed in AD (Bezprozvanny and Mattson (2008) Trends Neurosci. 31, 454-463). MAM facilitates the efficient transmission of Ca2+ from the ER to mitochondria and is highly enriched in proteins that regulate calcium levels (Hayashi et al., (2009) Trends Cell Biol. 19, 81-88).

Thus, a presenilin-mediated increase in ER-mitochondrial communication could lead to calcium overload of the latter, leading to mitochondrial dysfunction and apoptosis (Csordas et al., (2006) J. Cell Biol. 174, 915-921), as well as causing the altered mitochondrial dynamics (e.g. shape, distribution, and movement) and function (e.g. oxidative energy metabolism, calcium buffering capacity, and free radical production) found in AD (Pratico and Delanty (2000) Am. J. Med. 109, 577-585; Su et al., (2010) Mol. Neurobiol. 41, 87-96; Simmen et al., (2010) Biochim. Biophys. Acta, in press).

In view of the enrichment of presenilins in the MAM and the alterations in MAM function and morphology in PS-deficient cells described here, the results described herein show that MAM is an intracellular LR/DRM in which presenilins negatively regulate the connection of ER with mitochondria, and that upregulated MAM function plays a hitherto unrecognized role in the pathogenesis of AD (Schon and Area-Gomez (2010) J. Alzheimers Dis., in press).

### Example 9

### Materials and Methods

The following methods can be used in connection with the embodiments of the invention.

Subcellular fractionation and Western blotting. Purification of ER, MAM, and mitochondria was performed and analyzed as described herein.

Isolation of lipid rafts. To identify detergent-resistant domains, samples were resuspended in 400 μl of isolation buffer (IB: 250 mM mannitol, 5 mM HEPES pH 7.4, and 0.5 mM EGTA) containing 1% Triton X-100 (TX100) and incubated at 4° C. with rotation for 1 h. Samples were adjusted to 80% sucrose, placed at the bottom of a 5-30% sucrose gradient, and centrifuged at 250,000×g for 18 h. After fractionation, equal volumes of each fraction were loaded on an SDS-PAGE gel and analyzed by Western blot.

Measurement of cholesterol and cholesteryl esters. Quantification of total cholesterol and cholesteryl esters was performed using the Cholesterol/Cholesteryl Ester Quantitation kit).

Analysis of phospholipid synthesis in cultured cells. Cells were incubated for 2 h with serum free medium to ensure removal of exogenous lipids. The medium was then replaced with MEM containing 2.5 μCi/ml of 3H-serine for the indicated periods of time. The cells were washed and collected in DPBS, pelleted at 2500×g for 5 min at 4° C., and resuspended in 0.5 ml water, removing a small aliquot for protein quantification. Lipid extraction was done following the Folch method. Briefly, 3 volumes of chloroform:methanol 2:1 were added to the samples and vortexed. After centrifugation at 8000×g for 5 min, the organic phase was washed twice with 2 volumes of methanol/water 1:1, and the organic phase was blown to dryness under nitrogen. Dried lipids were resuspended in 60 μl of chloroform:methanol 2:1 and applied to a TLC plate. Phospholipids were separated using two solvents composed of petroleum ether/diethyl ether/acetic acid 84:15:1 v/v, and chloroform/methanol/acetic acid/water 60:50:1:4 v/v. Development was performed by exposure of the plate to iodine vapor. The spots corresponding to the relevant phospholipids were scraped and counted in a scintillation counter (Packard Tri-Carb 2900TR).

Analysis of phospholipid synthesis in subcellular fractions. Crude mitochondrial (CM) fractions were isolated from WT, PS1-KO, PS2-KO and DKO MEFs as described herein. Two hundred μg were incubated in a final volume of 200 μl of phospholipid synthesis buffer (10 mM CaCl₂, 25 mM HEPES pH 7.4 and 3 μCi/ml 3H-Ser) for 30 min at 37° C. The reaction was stopped by addition of 3 volumes of chloroform/methanol 2:1. Lipid extraction and TLC analysis was performed as described above.

Assay of ACAT activity. To measure ACAT activity in vivo, whole cells were incubated in serum-free medium for 2 h to remove all exogenous lipids. After that, 2.5 μCi/ml of ³Hcholesterol was added to FBS-free DMEM containing 2% FFA-BSA, allowed to equilibrate for at least 30 min at 37° C., and the radiolabeled medium was added to the cells for the indicated periods of time. Cells were then washed and collected in DPBS, removing a small aliquot for protein quantification. Lipids were extracted as described above and samples were analyzed by TLC along with an unlabeled cholesteryl ester standard. A mixture of chloroform/methanol/acetic acid 190:9:1 was used as solvent. Iodine stains corresponding to cholesteryl ester bands were scraped and counted.

To measure ACAT activity in vitro, subcellular fractions were isolated from different tissues, as described herein. Immediately after fractionation, 100 μg of each sample were assayed by mixing it with Buffer A (20 mM Tris-HCl, 1 mM EDTA; pH 7.7) containing 10 mg/ml FAFBSA and 50 μg/ml cholesterol. After 5 min incubation at 37° C., the reaction was started by adding 50 μl Buffer A containing 2 mg/ml FAF-BSA and 3H-oleoyl-CoA, and incubating at 37° C. for 20 min. The reaction was stopped by adding chloroform:methanol (1:1) containing 15 μg cholesteryl oleate as a carrier. Known amounts (2-5 μCi) of 3H-cholesterol were added as an internal standard. Lipids extraction and TLC analysis were as above.

Transmission electron microscopy. Cells were fixed with 2.5% glutaraldehyde in 0.1 M Sorenson's phosphate buffer (pH 7.2) for at least 1 h. Cells were then postfixed for 1 h with 1% OsO4 in Sorenson's buffer. Staining was performed using 1% tannic acid. After dehydration, cells were embedded in a mixture of Lx-112 (Ladd Research Industries) and Embed-812 (EMS, Fort Washington, Pa.). Thin sections, cut on an MT-7000 ultramicrotome, were stained with uranyl acetate and lead citrate, and examined in a JEOL JEM-1200 EXII electron microscope. Pictures were taken on an ORCA-HR digital camera (Hamamatsu) and recorded with an AMT Image Capture Engine.

Cinnamycin sensitivity assays. To measure cinnamycin binding (Emoto et al. (1999) Proc. Natl. Acad. Sci. USA 96:12400-12405), cells are incubated with 125I-labeled streptavidin complexed with cinnamycin (Ro 09-0198) peptide complex (1251-SA-Cin; 50,000 cpm/ml; Sigma) for 1 h at 39.5° C. The radioactivities of 125I-SA-Cin bound to the cells is analyzed by bioimage analyzer. To measure cell viability (Choung et al. (1988) Biochim. Biophys. Acta 940:171-179), cells are incubated with varying concentrations of cinnamycin (0.01-100 mM) from 1-30 min at 37° C. in order to determine the MIC and/or time to kill 50% of the cells (LC50; ˜1 mM at ˜2 min for human erythrocytes). Viability will be measured by “live/dead” assay (Molecular Probes).

Knockdown of PS1 expression. Small hairpin (sh) RNA oligonucleotides M2@nt 179-197 in NM_{—}008943: (gacaggtggtggaacaaga) and mismatch control shRNAs (Medema R H (2004) Biochem. J. 380:593-603) M3 (gacaggaggaggaacaaga, mismatches underlined) were inserted into pSUPER-Retro vector pSR (OligoEngine). In some experiments the puromycin-resistance cassette was replaced with a blasticidine-resistance cassette, generating pSR-Blast to allow for “double transduction” using two different selection markers to increase shRNA expression. Viral supernatants (3 ml) from plasmid-transfected Amphotrophic Phoenix phi-X-A packaging cells (Kinsella T M, Nolan G P (1996) Hum. Gene Ther. 7:1405-1413) supplemented with polybrene were added to MEFs, seeded 1 day prior to infection at 100,000/well in 6-well culture plates, and infection was allowed for 24 hours. Cells were selected in medium containing puromycin, blasticidin, or both antibiotics, for 14 days.

Analysis of the role of PS1 in mitochondrial bioenergetics. The results described herein indicate that PS1-mutant cells have altered mitochondrial function (e.g. O₂ consumption; ATP synthesis; free radical production), consistent with data already in the literature (e.g. Hirai et al. (2001) J. Neurosci. 21:3017-3023)), but the degree and extent of such dysfunction requires further exploration.

The bioenergetics—respiratory chain activity, oxygen consumption, ATP synthesis, membrane potential, and ROS production under different metabolic conditions—will be examined in a wider range of PS1-mutant cells (e.g. patient fibroblasts, mouse KD neuroblastoma cells, KO, and dKO cells), and where available, in mitochondria isolated from brains of WT and PS1-mutant mice. The mitochondrial ROS production will be examined as H2O2 emission fluorimetrically (see Andreyev et al. (2005) Biochemistry (Moscow) 70:200-214) for details). Using Amplex Red, H2O2 emission rates can be measured with NAD+- and FAD-linked respiratory substrates such as pyruvate, malate, and succinate, and compared with rates of O₂ consumption and the membrane potential of isolated mitochondria. To measure H2O2 scavenging capacity, two protocols can be used (as described herein) that employ physiologically realistic concentrations of H2O2 (up to 4 μM) and which measure two characteristics of the ROS-scavenging system: tolerance to acute H2O2 insult and ability to withstand a continuous H2O2 challenge. The H2O2 data will be correlated with a visual readout of ROS, using MitoSox.

The rates of ROS production obtained by these protocols in the absence of respiratory chain inhibitors also depend upon the magnitude of the membrane potential in mitochondria. Therefore, upon detecting any differences in mitochondrial ROS production between genetically modified mice and their littermates, the amplitude of their membrane potential will be measured under the identical conditions.

Many procedures are standard and are not described here. These include tissue homogenization, isolation of mitochondria by isopycnic centrifugation (Sims NR (1990) J. Neurochem. 55:698-707; Starkov et al. (2002) J. Neurochem. 83:220-228), disruption of mitochondria by digitonin (Rosenthal et al. (1987) J. Cereb. Blood Flow Metab. 7:752-758) or nitrogen cavitation (Kristian et al. (2006) J. Neurosci. Meth. 152:136-143), cell permeabilization with digitonin (Hardy J, Selkoe D J (2002) Science 297:353-356), and measurements of relevant mitochondrial enzyme activities (Lai J C, Cooper A J (1986) et al. J. Neurochem. 47:1376-1386; Klivenyi et al. (2004) J. Neurochem. 88:1352-1360; Starkov et al. (2004) J. Neurosci. 24:7779-7788; Rose I A, O'Connell E L (1967) J. Biol. Chem. 242:1870-1879; Shepherd D, Garland P B (1969) Biochemical J. 114:597-610; Endo et al. (1999) Biochim. Biophys. Acta 1450:385-396; Leong et al. (1984) J. Neurochem. 42:1306-1312). Oxygen consumption. Isolated mitochondria will be resuspended in high ionic strength buffer which reasonably approximates the known ionic composition of cell cytosol. This buffer will be supplemented with physiological oxidative substrates, pyruvate and malate, and the rates of oxygen consumption by mitochondrial suspension under various metabolic conditions will be recorded on a Hansatech Oxygraph (Villani G, Attardi G (2007) Methods Cell Biol. 80:121-133). ATP synthesis. Measures of AMP, ADP, and ATP synthesis (in nmol/min/mg protein) by HPLC of pelleted cell supernatants following addition of malate/pyruvate to digitonin-permeabilized cells will be determined using cells treated in parallel with oligomycin as a baseline control (Manfredi et al. (2001) Methods Cell Biol. 65:133-145).

H2O2 production. H2O2 production is measured with a horseradish peroxidase/Amplex Red detection system. Mitochondria are resuspended in standard incubation buffer (SIB) supplemented with either pyruvate and malate or with succinate and with 40 U/ml superoxide dismutase (Starkov et al. (2002) J. Neurochem. 83:220-228; Starkov et al. (2004) J. Neurosci. 24:7779-7788; Smaili et al. (2003) Brazil. J. Med. Biol. Res. 36:183-190). Calibration is performed by infusion of known amounts of H2O2 with a microdialysis pump. H2O2 scavenging capacity of mitochondria. A robust microtiter plate protocol that is quick, reproducible, and requires no more than 2-5 μg of mitochondria per assay can be used. The incubation buffer (IB) is composed of SIB and desired oxidative substrates. Two sets of microtiter plate wells are loaded with IB supplemented with variable H2O2 (0-800 pmol H2O2) per well. The reaction is triggered by adding mitochondria suspended in IB free of H2O2 to one set of wells; the second set is loaded with an equivalent volume of IB free of H2O2. After 5 min incubation at 370 C, both sets are loaded with H2O2 detection mixture composed of 20 U/ml horseradish peroxidase and 10 μM Amplex Red in IB, and the fluorescence intensity of formed resorufin is measured with multifunction plate reader (SpectraMax M5, Molecular Devices, USA). Residual H2O2 is calculated from a calibration curve obtained by measuring the fluorescence of a standard solution of resorufin, which is the reaction product of Amplex Red with H2O2/horseradish peroxidase. Scavenging capacity=difference in H2O2 between wells±mitochondria. Membrane potential. Besides using TMRM/TMRE to visualize mitochondrial membrane potential in cells, membrane potential will be quantitated using the membrane potential-sensitive dye safranin 0, added at 20:1 (mM dye:mg protein) Feldkamp et al. (2005) Am. J. Physiol. Renal Physiol. 288:F 1092-F1102, either spectrophotometrically or with a TPP+ selective electrode (Capell et al. (1997) J. Neurochem. 69:2432-2440).

Isolation and purification of subcellular fractions. Purification of ER, ER-MAM, and mitochondria was performed essentially as described (Stone S J, Vance J E (2000) J. Biol. Chem. 275:34534-34540; Vance J E (1990) J. Biol. Chem. 265:7248-7256). Cells and tissues were washed and immersed in isolation buffer (250 mM mannitol, 5 mM HEPES pH 7.4, 0.5 mM EGTA, and 0.1% BSA). Tissues were homogenized gently by four strokes in a loose Potter-Elvehjem grinder (Kontes). The homogenate was centrifuged for 5 min at 600 g to remove cells debris and nuclei. The supernatant was subjected to centrifugation for 15 min at 10,500 g, yielding two fractions: the supernatant, containing the ER/microsomal fraction, and the pellet, containing the crude mitochondrial fraction. The supernatant was subjected to centrifugation for 1 h at 100,000 g to pellet the ER/microsomal fraction. The crude mitochondrial fraction was layered on top of a 30% Percoll gradient and centrifuged for 30 min at 95,000 g in a Beckman Coulter Ultracentrifuge (Vance et al. (1997) Biochim. Biophys. Acta 1348:142-150). Two clear bands were visible in the gradient, an upper (lower-density) band containing the ER-MAM fraction and a lower (higher density) band containing mitochondria free of ER. Both fractions were recovered and washed with isolation buffer and pelleted at 10,500 g for 15 min, twice, to eliminate the Percoll.

To obtain the plasma membrane (PM) fraction, tissues were homogenized in STM 0.25 buffer (0.25 M sucrose, 10 mM Tris.Cl pH 7.4, 1.0 mM MgCl2), using a loose-fitting Potter-Elvehjem grinder (10 strokes). Homogenates were centrifuged for 5 min at 260 g and the supernatant was kept on ice. The pellet, containing nuclei and cell debris, was resuspended in half the volume of the same buffer and homogenized with three strokes on the same loose grinder and pelleted again for 5 min at 260 g. Both supernatants were combined and centrifuged for 10 min at 1,500 g. The pellet, containing the PM, was resuspended in twice the volume of STM 0.25 used initially and was further homogenized by three strokes, but using a tight-fitting grinder. The homogenate was diluted by adding an equal volume of STM 2 buffer (2 M sucrose, 10 mM Tris.Cl pH 7.4, 1.0 mM MgCl2), and centrifuged for 1 h at 113,000 g. The resulting low-density thin layer located near the top of the gradient, enriched in PM, was resuspended in 0.5-1 volume of STM 0.25 buffer.

Cinnamycin Binding Assay. Binding assay (modified from Emoto et al. (1999) Proc. Natl. Acad. Sci. USA 96:12400). Wild-type or PS1-mutant cells are seeded into 100-mm diameter dishes at 5×10³ cells per dish and cultivated at 33° C. for 20 days. The cell colonies are replicated onto polyester disks. The polyester discs are incubated for 24 h in growth medium at 39.5° C., washed twice with F-12 medium, and then incubated with ¹²⁵I -labeled streptavidin complexed with cinnamycin (Ro 09-0198) peptide complex (¹²⁵I-SA-Cin; 50,000 cpm/ml) for 1 h at 39.5° C. The radioactivities of ¹²⁵I-SA-Cin bound to the colonies is analyzed by bioimage analyzer. Mutant cells will exhibit a lower binding activity than control cells.

Cinnamycin Viability Assay. Viability assay (modified from Choung et al. (1988) Biochem. Biophys. Acta 940:171). Normal fibroblasts are incubated with varying concentrations of cinnamycin (0.01-100 mM in log dilutions for times ranges from 1-30 min at 37° C. in order to determine the normal concentration and/or time to kill 50% and 100% of the cells (LC₅₀ and LC₁₀₀; the LC₅₀ for normal human erythrocytes is ˜1 mM with an incubation time of ˜2 min) Viability can be measured many ways. In one embodiment, cell viability can be measured with a “live/dead” assay (Molecular Probes) that stains living cells as green and dead cells red. PS1-mutant cells are treated under the same conditions to determine if the are resistant to cinnamycin. In another embodiment, the viability of cells in the presence of cinnamycin can be determined by measuring the LC₅₀ and LC₁₀₀ for PS1-mutant cells compared to control cells.

Culturing of explanted primary mouse neurons. Mice are sacrificed in CO2 and soaked in 80% ethanol for 10 min. Fetuses are removed (E15 mouse embryos) and kept in PBS on ice. After removal of the meninges, the cortex is dissected, and washed with Hank's balanced salt solution (HBSS). Cortical neurons are released from tissue by trypsin treatment, followed by trituration, and plated on polylysine coated culture dishes at a density of ˜106 cells/35-mm dish (Friedman et al., (1993) Differential actions of neurotrophins in the locus coeruleus and basal forebrain. Exp. Neurol. 119:72-78). Prior to experiments, cells are maintained for 4-5 days in serum-free medium and 0.5 mM 1-glutamine (Rideout H J, Stefanis L (2002) Proteasomal inhibition-induced inclusion formation and death in cortical neurons require transcription and ubiquitination. Mol. Cell. Neurosci. 21:223-238) to yield a relatively pure culture of neurons. To ensure that this is the case, immunostaining for α-internexin, an intermediate filament protein expressed by differentiated postmitotic neurons of the developing CNS, but not by neuroblasts or cells of the glial lineage, can be performed (Fliegner et al., (1994) Expression of the gene for the neuronal intermediate filament protein α-internexin coincides with the onset of neuronal differentiation in the developing rat nervous system. J. Comp. Neurol. 342:161-173).

Subcellular fractionation. Purification of ER, ER-MAM, and mitochondria was performed essentially as described (Stone and Vance, J. Biol. Chem. 275, 34534 (2000); Vance, Biol. Chem. 265, 7248 (1990)). Cells and tissues were washed and immersed in isolation buffer (250 mM mannitol, 5 mM HEPES pH 7.4, 0.5×EGTA, and 0.1% BSA). Tissues were homogenized gently by four strokes in a loose Potter-Elvehjern grinder (Kontes). The homogenate was centrifuged for 5 min at 600 g to remove cells debris and nuclei. The supernatant was subjected to centrifugation for 15 min at 10,500 g, yielding two fractions: the supernatant, containing the ER/microsomal fraction, and the pellet, containing the crude mitochondrial (CM) fraction. The supernatant was subjected to centrifugation for 1 h at 100,000 g to pellet the microsomal fraction. The crude mitochondrial fraction was layered on top of a 30% Percoll gradient and centrifuged for 30 min at 95,000 g in a Beckman Coulter Ultracentrifuge: two clear bands were visible in the gradient, an upper (lower-density) band containing the ER-MAM fraction and a lower (higher density) band containing mitochondria free of ER; both fractions were recovered and washed with isolation buffer and pelleted at 10,500 g for 15 min, twice, to eliminate the Percoll. All fractions were quantitated for total protein content using the Bradford system (BioRad).

To obtain the plasma membrane (PM) fraction, tissues were homogenized in STM 0.25 buffer (0.25 M sucrose, 10 mM Tris-Cl pH 7.4, 1.0 mM MgCl; 4.5 ml/g tissue), using a loose-fitting Potter-Elvehjem grinder (Kontes) (10 strokes). Homogenates were centrifuged for 5 min at 260 g and the supernatant was kept on ice. The pellet, containing nuclei and cell debris, was resuspended in half the volume of the same buffer and homogenized with three strokes on the same loose grinder and pelleted again for 5 min at 260 g. Both supernatants were combined and centrifuged fox 10 min at 1,500 g. The pellet, containing the PM, was resuspended in twice the volume of STM 0.25 used initially and was further homogenized by three strokes, but using a tight-fitting grinder (Kontes). The homogenate was diluted by adding an equal volume of STM 2 buffer (2 M sucrose, 10 mM Tris-C1 pH 7.4, 1.0 mM MgCl₂), and centrifuged for 1 h at 113,000 g. The resultant low-density thin layer located near the top of the gradient, enriched in PM, was resuspended in 0.5-1 volume of STM 0.25 buffer (D. E. Vance, C. J. Wakey, Z. Cui, Biochim. Biophys. Acta 1348, 142 (1997).

Purification and analysis of subcellular fractions from mouse liver. Plasma membrane (PM), crude mitochondria (CM), and ER was isolated as described herein (Stone and Vance, J. Biol. Chem. 275, 34534 (2000); Vance, Biol. Chem. 265, 7248 (1990)), and fractionated crude mitochondria further by isopycnic centrifugation (Vance, et al., Biochim. Biophys. Acta 1348, 142 (1997)) into a ER-MAM fraction and a purified mitochondrial fraction. Each of these fractions was evaluated by Western blot analysis using antibodies to cadherin (CDH2) as a marker for PM, to calnexin (CANX) as a marker for ER, to Golgi matrix protein GM130 (GOLGA2) as a marker for Golgi, to ACAT1, G6PC, and PEMT as markers for ER-MAM (and to a lesser extent, ER), and to the NDUFA9 subunit of complex I of the respiratory chain as a marker for mitochondria. The ER-MAM fraction is distinct from ER or purified mitochondrial fractions. specifically the, ER-MAM fraction was enriched for the three ER-MAM markers. These three proteins were significantly less enriched in the ER and mitochondrial fractions compared to ER-MAM. Similarly, marker proteins for the PM, Golgi, ER and mitochondria were selectively depleted from the ER-MAM fraction (FIG. 10).

Purification of ER, ER-MAM, and mitochondria was performed as described (S. J. Stone, J. E. Vance, J Biol. Chem. 275, 34534 (2000); J. E. Vance, 3: Biol. Chem. 265, 7248 (1990)). Cells and tissues were washed and immersed in isolation buffer (250 mM mannitol, 5 mM HEPES pH 7.4, 0.5 mM EGTA, and 0.1% BSA). Tissues were homogenized gently by four strokes in a loose Potter-Elvehjem grinder (Kontes). The homogenate was centrifuged for 5 min at 600 g to remove cells debris and nuclei. The supernatant was subjected to centrifugation for 15 min at 10,500 g, yielding two fractions: the supernatant, containing the ER/microsomal fraction, and the pellet, containing the crude mitochondrial fraction. The supernatant was subjected to centrifugation for 1 h at 100,000 g to pellet the ER/microsomal fraction. The crude mitochondrial fraction was layered on top of a 30% Percoll gradient and centrifuged for 30 min at 95,000 g in a Beckman Coulter ultracentrifuge. Two clear bands were visible in the gradient, an upper (lower-density) band containing the ER-MAM fraction and a lower (higher density) band containing mitochondria free of ER. Both fractions were recovered and washed with isolation buffer and pelleted at 10,500 g for 15 min, twice, to eliminate the Percoll. All fractions were quantitated for total protein content using the Bradford system (BioRad).

To obtain the plasma membrane (PM) fraction, tissues were homogenized in STM 0.25 buffer (0.25 M sucrose, 10 mM TrisC1 pH 7.4, 1.0 mM MgCl₂; 4.5 ml/g tissue), using a loose-fitting Potter-Elvehjem grinder (Kontes) (10 strokes). Homogenates were centrifuged for 5 min at 260 g and the supernatant was kept on ice. The pellet, containing nuclei and cell debris, was resuspended in half the volume of the same buffer and homogenized with three strokes on the same loose grinder and pelleted again for 5 min at 260 g. Both supernatants were combined and centrifuged for 10 min at 1,500 g. The pellet, containing the PM, was resuspended in twice the volume of STM 0.25 used initially and was further homogenized by three strokes, but using a tight-fitting grinder (Kontes). The homogenate was diluted by adding an equal volume of STM 2 buffer (2 M sucrose, 10 mM Tris-Cl pH 7.4, 1.0 mM MgCl₂), and centrifuged for 1 h at 113,000 g. The resultant low-density thin layer located near the top of the gradient, enriched in PM, was resuspended in 0.5-1 volume of STM 0.25 buffer.

Other methods for isolating ER-MAM are also known to those skilled in the art. As one non-limiting example, ER-MAM fractions can be obtained by immersing a biological sample (e.g. tissues or cells) in an ice-cold isolation medium (250 mM mannitol, 5 mM HEPES, pH 7.4, 0.5 mM EGTA, and 0.1% bovine serum albumin) If the sample is a tissue, it can be minced with scissors and homogenized gently by four strokes in a Potter-Elvehjem motor driven homogenizer. The homogenate can then centrifuged twice at 600×g for 5 min to remove large debris and nuclei. The supernatant is centrifuged for 10 min at 10,300×g to pellet the crude mitochondria. Microsomes can be obtained by centrifugation of the resultant supernatant at 100,000×gₘₐₓ for 1 hour in a Beckman Ti-70 rotor. For further purification of mitochondria, the crude mitochondrial pellet can be suspended by hand homogenization in approximately 4 ml of isolation medium, and the suspension can be layered on top of 20 ml of medium containing 225 mM mannitol, 25 mM HEPES, pH 7.4, 1 mM EGTA, 0.1% bovine serum albumin, and 30% (v/v) Percoll, in each of four 30-ml polycarbonate ultracentrifuge tubes. The tubes can then be centrifuged for 30 min at 95,000×gₘₐₓ, after which a dense band, containing purified mitochondria, can be recovered from approximately ⅔ down the tube. The mitochondria are removed with a Pasteur pipette, diluted with isolation medium, and washed twice by centrifugation at 6,300×gm for 10 min to remove the Percoll. The final pellet is resuspended in isolation medium and can be stored at −70° C. ER-MAM can be isolated from the Percoll gradient from the band immediately above the mitochondria, by centrifugation first at 6,300 gₘₐₓ for 10 min then further centrifugation of the supernatant at 100,000×gₘₐₓ, for 1 h in a Beckman Ti-70 rotor. The pellet of ER-MAM, can be resuspended in approximately 0.5 ml of buffer containing 0.25 M sucrose, 10 mM Tris-HCl, pH 7.4, and 0.1 mM phenylmethylsulfonyl fluoride, and stored at −70° C.

For subfractionation of mitochondria into inner and outer membranes, the pure mitochondrial pellet can be suspended in buffer (20 mg/ml) containing 70 mM sucrose, 200 mM mannitol, and 2 mM HEPES, pH 7.4. The mitochondria (2.5 mg) can be mixed gently with 125 μl of 0.6% digitonin solution made in the above buffer and incubated on ice for 15 min. The mixture can be diluted with the above buffer containing 50 mg of bovine serum albumin/100 ml, then centrifuged for 10 min at 12000×gₘₐₓ. The supernatant is enriched in mitochondrial outer membranes, and the pellet is enriched in inner membranes. Methods for isolating Golgi, plasma membrane, and rough and smooth endoplasmic reticulum fractions are known to one skilled in the art (for example see Croze, E. M., and Morre, D. J. (1984) J. Cell. Physiol. 119, 46-52. Dennis, E. A., and Kennedy, E. P. (1970) J. Lipid Res. 11, 394-403).

Methods for isolating crude mitochondria are known to those skilled in the art (for example see, Vance, 1990 or Croze and Morre, 1984). ER-MAM and purified mitochondria can be separated on a self-forming 30% Percoll gradient (Vance, 1990; Hovius et al., 1990). Golgi membranes and two ER fractions (ERI and ERII) can be isolated (Croze and Morre, 1984). ERI can be obtained from the final sucrose gradient at the interface between sucrose solutions of 1.5 and 2.0M, whereas ERII can be isolated from the interface between sucrose solutions of 1.5 and 1.3 M. ERI is enriched in rough ER membranes, and ERII in smooth ER membranes (Croze and Morre, 1984).

Various methods to examine the activity of biomarkers of subcellular fractions have been described in the art. The ER marker enzymes NADPH:cytochrome c reductase and glucose-6-phosphate phosphatase can be assayed by established procedures (Vance and Vance, 1988). Enzymatic activity for UDP:N-acetylglucosamine-1-phosphotransferase (Rusiol et al., 1993), UDP:N-galactose-acetylglucosaminegalactosyltransferase (Rusiol et al., 1993b), and cytochrome C oxidase (Vance and Vance, 1988) can also be measured by methods known in the art.

Mitochondrial Superoxide Stress Fluorescence Assay (“MitoSox”). Mitosox Red (Molecular Probes) is live-cell permeant and that is selectively targeted to mitochondria. Once inside the mitochondria, the reagent is oxidized by superoxide and binds to nucleic acids, resulting in a red fluorescence. Normal fibroblasts do not stain with MitoSox, whereas PS1-mutant cells. Staining of mitochondria indicates superoxide radical production. A more general assay that detects many forms of reactive oxygen species (ROS) (e.g. superoxide, hydrogen peroxide, singlet oxygen, and peroxynitrite) can also be used. One technique is to use “Image-iT Live” assay (Molecular Probes), which is based on 5-(and -6)-carboxy-2′,7′-dichlorodihydrofluorescein diacetate (carboxy-H2DCFDA). Carboxy-H2DCFDA is a fluorogenic marker for ROS. Non-fluorescent carboxy-H2DCFDA permeates live cells and is deacetylated by nonspecific intracellular esterases. In the presence of ROS, which are produced throughout the cell (particularly during oxidative stress), the reduced fluorescein compound is oxidized and emits green fluorescence.

Immunofluorescence. To detect mitochondria, living cells were labeled with 1 nM MitoTracker Red CMXRos (MTred; Invitrogen) in DMEM for 20 min at 37° C. The cells were fixed after washing the cells in DMEM twice for 10 min, as described herein.

For immunolocalization, cells were fixed and permeabilized using three different methods: (1) fixation in 4% paraformaldehyde (PF) for 30 min at RT and permeabilization in either 0.1% or 0.4% Triton X-100 (TX100) for 15 min at RT; (2) fixation in 4% PF for 30 min at RT and permeabilization in chilled methanol for 20 min at −20° C.; and (3) fixation and permeabilization in chilled methanol for 20 min at −20° C. The fixed cells were then washed twice for 5 min in phosphate-buffered saline (PBS), and incubated in blocking solution (2.5% normal goat serum [NGS], 1% bovine serum albumin [BSA], and 0.1% TWEEN-20 in 1×PBS) in a humid chamber. Incubation with primary antibodies was performed at room temperature (RT) as recommended. Secondary antibodies were used according to the manufacturers' instructions. For simultaneous detection of PEMT and PS1, PEMT was detected by treating the cells first with rabbit anti-PEMT, then with mouse anti-rabbit IgG (“bridge” antibody), and finally with goat anti-mouse IgG conjugated to Alexa Fluor 594 (red) (Invitrogen), while PS1 was detected by treating cells with rabbit anti-PS1 and then with goat anti-rabbit IgG conjugated to Alexa Fluor 488 (green). Detection was also performed reversely (i.e. PEMT using goat anti-rabbit IgG and PS1 using mouse anti-rabbit followed by goat anti-mouse). For detection of cdnexin, secondary antibodies conjugated to Alexa Fluor 350 (blue) were used.

Cells were imaged on an Olympus 1×70 inverted microscope. Red, green, and blue images were captured sequentially using a SPOT RT digital camera and merged using SPOT RT software (New York/New Jersey Scientific, Inc.). Confocal microscopy was performed with a Zeiss LSMSIO microscope using a 63× Plan-Neofluor, 1-25 NA objective lens. The pinhole was set to give an optical section of 1.1 μm. Excitation was at 488 nm (for green), 543 nm (for red), and 350 nm (for blue). To quantitate the localization of mitochondria, a region that extended from portions of the nuclear envelope to points midway to the plasma membrane was defined, and the amount of MitoTracker Red signal outside this region was measured. Since organelles are more sparse in the extremities, the dynamic range of signal was higher, and presumably more linear, than signal from the perinuclear region. Specifically, a z-series. of images covering the total cell thickness was collected with a Zeiss LSM510 microscope using a Plan-Neofluar, 0.9 NA objective lens. The pinhole was set to give an optical section of 1.4 pm—The interval between z slices was set to 1.4 μm to give non-overlapping sections. Excitation was at 488 nm (for green) and 543 nm (for red). Z sections were projected onto a single image, and an area between the nucleus and the cell periphery, as determined by microtubule staining, was outlined. In that area, the midpoint between the nucleus and the farthest point at the cell periphery was determined at various positions around the nucleus. Using the midpoints, the outlined area was divided into two parts, one proximal (A) and one distal (B) to the nucleus. Mean grayness values were recorded for the proximal and distal parts. For quantification of mitochondria in the outer edges of cell, the grayness value for the distal part was divided by the grayness value for the total area (proximal+distal). Calculation of grayness value for the total area was ((grayness_{A}×area_{A}+(grayness_{B}×area_{B}))/(area_{A}+area_{B}).

Mitochondrial Distribution Assay. Mitochondria in many PS1-mutant fibroblasts are more concentrated around the nucleus than are mitochondria in controls, with fewer mitochondria at the extremities of PS1. This effect can be quantitated by measuring the intensity of the orange signal in the extremities of Mitotracker-stained cells. Measurements are performed by projecting confocal imaging z sections into a single image. An area between the nucleus and the cell periphery, as determined by microtubule staining. The area is outlined, and the midpoint between the nucleus and the farthest point at the cell periphery is determined. Using the midpoint, the outlined area is then divided into two parts: regions proximal (A) and distal (B) to the nucleus. Mean grayness values of the MitoTracker stain are recorded for the proximal and distal parts. For quantification of mitochondria in the outer edges of a cell, the grayness value for the distal part is divided by the grayness value for the total area (proximal+distal). Calculation of grayness value for the total area=([Grayness_{A}×Area_{A}]+[Grayness_{B}×Area_{B}])/(Area_{A}+Area_{B}). Significantly fewer mitochondria are observed in the extremities of PS1-mutant cells as compared to control cells.

Immunohistochemistry in brain. Immunohistochemistry in brain can be performed on 10-μ-thick paraffin-embedded sections using the ABC method or by double-labeling methods with different fluorochromes to human mitochondrial proteins (e.g. COX II, ND1, the iron-sulfur [FeS] protein subunit of Complex III, and TOM20) (Tanji K, Bonilla E (2001) Optical imaging techniques (histochemical, immunohistochemical, and in situ hybridization staining methods) to visualize mitochondria. Methods Cell Biol. 65:311-332). Monoclonal anti-MAP2, a perikaryon and dendritic marker, and monoclonal anti-MAPS, a marker for neuronal axons can be used for neuronal probes. Additional sections can be stained with H-E for conventional microscopic study, with thioflavine S for localization of amyloid deposits, and with a modified Bielschowsky silver stain for evaluation of plaques and neurofibrillary tangles.

Cells and reagents. Mutant FAD^{PS1}-A246E (AG06840 and AG06848) human fibroblasts were obtained from the Coriell Institute for Medical Research (Camden, N.J.). FAD^{PS1} M146L (GG1, GG3, and GG5) and control (GG2, GG4, and GG6) fibroblasts have been described elsewhere (R. Sherrington et al., Nature 375, 754 (1995)). Normal human fibroblasts (line AE) were also used. Other human fibroblast samples were obtained from the University of Washington Alzheimer Disease Research Center. Cultured primary rat neurons were obtained from Columbia University. Human fibroblasts (line 97) and 3T3 and COS-7 cells were available in the laboratory. Mouse embryonic fibroblasts (MEFs) were derived from pups with a heterogeneous C57BL161129Sv background at E 12.5-14.5. Spontaneously-immortalized MEFs were obtained by passage through the replication bottleneck using a 3T3 subculture schedule. Cells went into a metabolic crisis between passages 9-12, and recovered by passage 16. Retroviral transduction was carried out between passages 20-25. Cells were cultured in DMEM medium supplemented with 10% FBS (Invitrogen) and penicillin/streptomycin.

Transfection of mitoDendra. MitoDendra can be transfected into neurons as described (Ackerley et al., (2000) Glutamate slows axonal transport of neurofilaments in transfected neurons. J. Cell Biol. 150:165-176; Nikolic et al., (1996) The cdk5/p35 kinase is essential for neurite outgrowth during neuronal differentiation. Genes Dev. 10:816-825). Typically, 10% of the cells are transfected. This provides a sufficient number of cells to allow for multiple measurements. To improve gene expression efficiency and to minimize non-specific toxicity derived from transfection approaches, the mitoDendra construct can be transferred into an adenoviral vector. Neurons can be imaged 36 hr after transfection.

Antibodies. The following polyclonal antibodies recognizing different regions of PS1 were used: aa 31-46 (Sigma P4985), aa 450-467 (Sigma W854), aa 303-316 (Calbiochem PC267), and aa 263-407 (“loop” domain; Calbiochem 529592); polyclonal antibodies recognizing aa 32-46 (B 19.2) and aa 310-330 (B32.1) of mouse PS1 were used (W. G. Annaert et al., J. Cell Biol. 147, 277 (1999)). Antibodies recognizing cadherin (monoclonal; Sigma C 1821), calnexin (monoclonal; Chemicon MAB3 1261, fatty acid-CoA ligase 4 (polyclonal; Abgent A P 2536b), glucose-6-phosphatase (polyclonal (A. Eautier-Stein et al., Nucl. Acids Res. 31, 5238 (2003)), Golgi matrix protein GM130/GOLGA2 (polyclonal; Calbiochem CB 1008), NDUFA9 (monoclonal; Molecular Probes A2 13441, ACAT1 (polyclonal; Abcam ab39327), PEMT (polyclonal (Z. Cui, J. E. Vance, M. H, Chen, D. R. Voelker, D, E. Vance, J Biol. Chem. 268 16655 (1993)), protein disulfide isomerase (PDI) (monoclonal; Stressgen SPA-8911, PACS2 (polyclonal M. Köttgen et al., EMBO J. 24, 705 (2005)), SSRI (polyclonal (G. Migliaccio, C. V. Nicchitta, G. Blohel, J. Cell Biol. 117, 15 (1992)), and tubulin (monoclonal; Sigma T4026) were also used. Goat secondary antibodies (A-11008, A-11012, and A-11046) were from Molecular Probes. Mouse monoclonal anti-rabbit “bridge” antibodies (R1008; used at 1:2000) were from Sigma. Secondary HRP-linked mouse (NXA931) and rabbit (NA934V) antibodies were from GE Healthcare Life Sciences.

Antibodies to APH-1 (ABR PA1-2010), APP (Landman et al, Proc. Natl. Acad. Sci. USA 2006, 103:19524-19529), ATP synthase subunit a (Molecular Probes A21350), FACL4 (Abgent A P 2536b), Golgi matrix protein GM130/GOLGA2 (Monoclonal BD transduction #610822), IP3R3 (Millipore AB9076), Na,K-ATPase (Abcam ab7671), nicastrin (Covance PRB-364P), PEN2 (Abcam ab62514), and SSRα (Migliaccio et al, J. Cell Biol. 1992, 117:15-25). Mouse monoclonal anti-rabbit “bridge” antibodies were from Sigma (R1008; used at 1:2000).

Western blotting. Samples were resuspended in Laemmli buffer, heated for 10 min at 60° C., subjected to polyacrylamide gel electrophoresis, transferred to PVDF membranes (BioRad), and probed with antibodies. Immunostaining bands were revealed by chemiluminescence (West Rco ECL Kit, Pierce).

Small hairpin (sh) RNA oligonucleotides. Small hairpin (sh) RNA (Medema, Biochem. J. 380, 593 (2004) oligonucleotides M2@nt 179-197 in NM_{—}008943 (gacaggtggtggaacaaga) (SEQ ID NO: 1) and mismatch control shRNAs M3 (gacaggaggaggaacaaga; mismatches underlined) (SEQ ID NO: 2) were inserted into pSUPER-Retro vector pSR (OligoEngine). In some experiments, the puromycin-resistance cassette was replaced with a blasticidine resistance cassette (NheI-DraIII), generating pSR-Blast to allow for “double transduction” using two different selection markers to increase shRNA expression. Viral supernatants (3 ml) from plasmid-transfected Amphotrophic Phoenix φNX-A packaging cells (Kinsella, G. P. Nolan, Hum. Gene Ther. 7, 1405 (1996)) supplemented with polybrene were added to MEFs, seeded 1 day prior to infection at 100,000/well in 6-well culture plates, and infection was allowed to proceed for 24 hours. Cells were selected in medium containing puromycin, blasticidin, or both antibiotics, for 14 days.

Cell transfections. The open reading frame from Gentstorm plasmids (Invitrogen) containing human wt and A246E PS1 cDNAs was amplified using flanking PCR primers containing KpnI and XbaI sites at the 5′ and 3′ ends, respectively. The amplification products were inserted into the unique KpnI and XbaI sites of pcDNA3.1 (Invitrogen). Clones were confirmed by DNA sequencing and transfected into COS-7 and 3T3 cells using Lipofectamine 2000 (Invitrogen). After 24-36 h, transfected cells were treated with neomycin to select for stable transformants.

In vitro import assay. Human PS1 was transcribed using a reticulocyte lysate system and imported into isolated mitochondria as described previously (Leuenberger et al., EMBO J. 18, 4816 (1999)).

Crosslinking. In order to identify proteins that interact with presenilin, ER-MAM can be isolated and cross-linked with formaldehyde (or with a small panel of crosslinking agents), the cross-linked material can be solubilized with detergent, and then immunoprecipitated with antibodies to presenilin. A number of crosslinking compounds are commercially available, such as SFAD (Pierce, #27719), a bifunctional crosslinking agent that is photoinitiated and is reactive to amino groups and —CH bonds; other reagents contain groups that are reactive to carboxylates and sulfhydryl groups. Different contact times and concentrations of cross-linker can be used in order to reduce over-cross-linking. The immunoprecipitated proteins can then be subjected to tryptic digestion and mass spectrometry for identification. A small panel of these reagents can be used to cover different chemistries of potential targets (e.g., presenilin can react with the amino reactive end of a given cross linker, but the other protein may not present the proper functional group for the other reactive group on the linker).

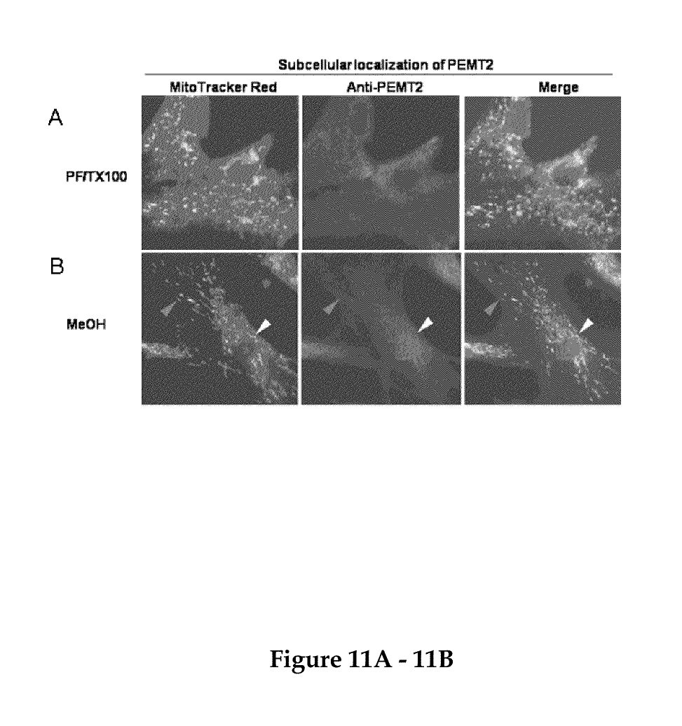

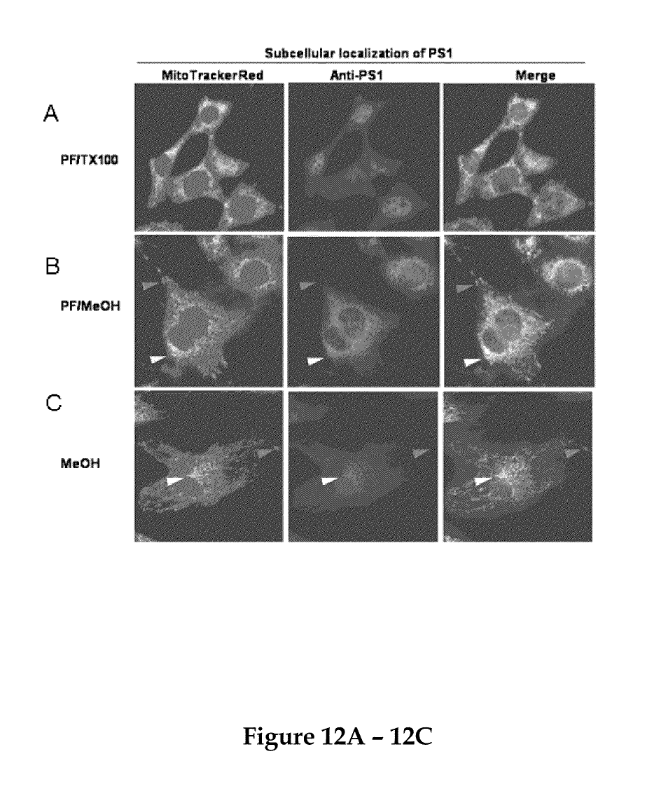

Immunocytochemistry to detect PEMT and Presenilin in cells. The subcellular localization of both PHMT2 and PS1 was sensitive to conditions used for fixation of samples in preparation for immunocytochemistry. Using paraformaldehyde (PF) fixation and permeabilization with Triton X-100 (TX100), PEMT was found to localize to diffuse or punctate structures that did not co-localize with any obvious subcellular compartment (FIG. 11A). However, when cells were treated with cold methanol (MeOH), PEMT co-localized with MTred-stained structures, especially in the perinuclear region (yellow arrowheads in FIG. 11B). Co-localization with MTred was less apparent in more distal regions of the cell (red arrowheads in FIG. 11B). The apparent localization of PEMT with perinuclear mitochondria may be due to the fact that upon dehydration by MeOH, this MAM-associated protein precipitated at or near adjacent mitochondria. An essentially identical result was obtained upon immunolocalization of PS1 in mouse 3T3 cell and human fibroblasts (FIG. 12). Finally, immunostaining of both PEMT and PS1 in the same cells showed a high degree of co-localization (using bath PF/TX100 [FIG. 12A] and MeOH [FIG. 12B] to permeabilize and fix the cells), indicating that PS1 is indeed enriched in the ER-MAM compartment (FIG. 12).

Detection of Mitochondria. Mitochondria were detected after loading the cells with 1 nM MitoTracker Red CMXRos (MTred; Invitrogen) in tissue culture medium (DMEM) for 20 min at 37° C. After washing the cells in medium twice for 10 min, immunolocalization was then performed, using three different methods to fix and permeabilize the cells: (1) fixation in 4% paraformaldehyde (PF) for 30 min at RT and permeabilization in either 0.1% or 0.4% Triton X-100 (TX100) for 15 min at RT; (2) fixation in 4% PF for 30 min at RT and permeabilization in chilled methanol for 20 min at −20° C.; and (3) fixation and permeabilization in chilled methanol for 20 min at −20° C. The fixed cells were then washed twice for 5 min in phosphate-buffered saline (PBS), and incubated in blocking solution (2.5% normal goat serum [NGS], 1% bovine serum albumin [BSA], and 0.1% TWEEN-20 in 1×PBS) in a humid chamber for 1 h at RT. Antibodies were used as recommended. Cells were imaged on an Olympus 1×70 inverted microscope. Red and green images were captured sequentially using a SPOT RT digital camera and merged using SPOT RT software (New York/New Jersey Scientific, Inc.).

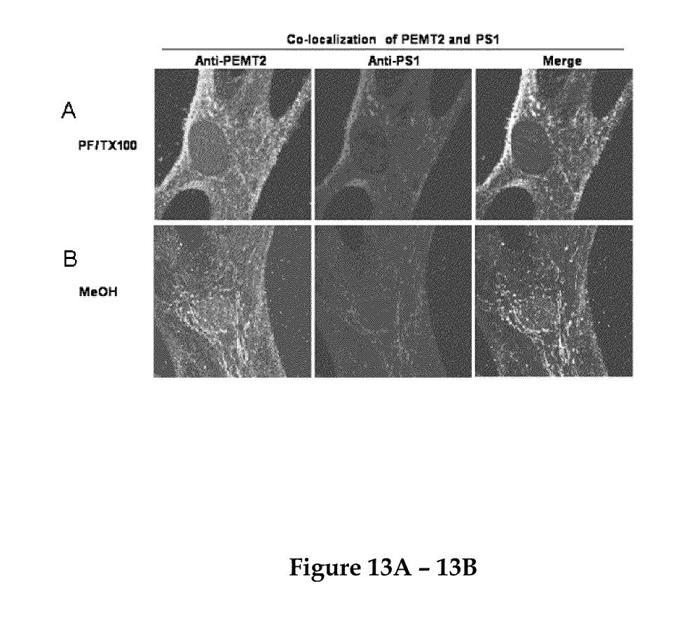

For simultaneous detection of PEMT and PS1 (FIG. 13), PEMT was detected by treating the cells first with rabbit anti-PEMT, then with mouse anti-rabbit IgG (“bridge” antibody), and finally with goat anti-mouse IgG conjugated to Alex Fluor 594 (red) (Invitrogen), while PS1 was detected by treating cells with rabbit anti-PS1 and then with goat anti-rabbit IgG conjugated to Alexa Fluor 488 (green). Detection using the reverse procedure (i.e. PEMT using goat anti-rabbit IgG and PS1 using mouse anti-rabbit followed by goat anti-mouse) yielded a similar result.

Immunohistochemistry to Detect Presenilin in Various Cells. The localization of PS1 and MitoTracker Red (MTred) was examined in other cells, using MTred to detect mitochondria and immunocytochemistry using antibodies directed against either the N- or C-terminus of PS1 in cells fixed and permeabilized with MeOH (FIG. 14). Co-localization of PS1 was detected with MTred in mouse 3T3 cells (FIG. 14A) and rat neurons (FIG. 14B): PS1 ca-localized with MTred in the perinuclear region and within the cell body (yellow arrowheads in FIGS. 14A and B), but not with mitochondria that were present in processes that extended from the cell body (red arrowheads in FIGS. 14A and 14B).

PS1 staining was observed at adherens junctions in the plasma membrane in confluent human 293T (FIG. 14C) and COS-7 cells, also as seen by others (Georgakopoulos et al., Mol. Cell. 4, 893 (1999; Marambaud et al., EMBO J. 21, 1948 (2002)), confirming a known location for PS1 when using MeOH for fixation and permeabilization.

Transfection of Presenilin in COS-7 Cells. Monkey COS-7 cells were transfected stably with a construct expressing either wild-type PS1 or the A246E mutation, and double-stained for MTred and tubulin (FIG. 7) to recapitulate the mitochondrial maldistribution phenotype often seen in FAD^{PS1} fibroblasts by expressing mutated PS1. Transfected cells were compared to untransfected cells or to controls expressing empty vector or wt-PS1.

The open reading frame from Genestorm plasmids (Invitrogen) containing human wt and A246E P51 cDNAs was amplified using flanking PCR primers containing KpnI and XbaI sites at the 5′ and 3′ ends, respectively. The amplification products were inserted into the unique KpnI and XbaI sites of pcDNA3.1 (Invitrogen). Clones were confirmed DNA sequencing and transfected into COS-7 and 3T3 cells using Lipofectamine 2000 (Invitrogen). After 24-36 h, transfected cells were treated with neomycin to select for stable transformants Cells were stained with MTred (red) and anti-tubulin (green).

γ-Secretase Activity Assays. Endogenous γ-secretase activity was determined by Western blotting to detect the amount of AICD derived from the cleavage of endogenous APP, as described. (Landman et al, Proc. Natl. Acad. Sci. USA 2006, 103:19524-19529). 50 μg of protein from each fraction was incubated in reaction buffer (10 mM Tris-HCl, 150 mM NaCl, 5 mM EDTA, pH 7.4) for 3 h at 37° C., followed by Western blotting with anti-APP. As a control, the same samples were assayed in the presence of 2 μM compound E ([(2S)-2-{[(3,5-difluorophenyl)acetyl]amino}-N-[(3S)-1-methyl-2-oxo-5-phenyl-2,3-dihydro-1H-1,4-benzodiazepin-3-yl]propanamide]; Alexis Biochemicals, ALX270-415-C250), a γ-secretase inhibitor (Hansson et al, J. Biol. Chem. 2004, 279:51654-51660). A FRET-based γ-secretase activity assay was used to detect cleavage of an exogenously-added secretase-specific peptide conjugated to two fluorescent reporter molecules (R&D Systems FP003) in serial dilutions of different subcellular fractions. As a control, the same samples were assayed in the presence of 2 μM compound E.

Visualization of PS1 using methanol fixation. Cells (80-90% confluent) are stained with MT Red, fixed and permeabilized by adding MeOH (previously frozen in dry ice) for 20 min at −20° C., and washed out with 1×PBS twice. Cells can also be washed, fixed, and permeabilized without staining with MT Red by adding frozen MeOH directly to the culture. Block cells and continue as with a standard immunofluorescence assay.

### Example 10

### Effects on APP

In order to test whether mutations in presenilin affect ER-to-PM trafficking of APP (Cai et al., (2003) Presenilin-1 regulates intracellular trafficking and cell surface delivery of β-amyloid precursor protein. J. Biol. Chem. 278:3446-3454), Western blots can be performed to detect both APP and Aβ in ER, ER-MAM, and mitochondria isolated from control and PS1-mutated cells.

### Example 11

### Presenilin Transgenic Mice

Transgenic mice that overexpress human PS1 with both the M146L and M146V mutations are available (Duff et al., (1996) Increased amyloid-β42(43) in brains of mice expressing mutant presenilin 1. Nature 383:710-713; Begley et al., (1999) Altered calcium homeostasis and mitochondrial dysfunction in cortical synaptic compartments of presenilin-1 mutant mice. J. Neurochem. 72:1030-1039). Mice in which PS1 has been knocked out are embryonic lethal (Handler et al., (2000) Presenilin-1 regulates neuronal differentiation during neurogenesis. Development 127:2593-2606), but PS2 KO mice are viable (Steiner et al., (1999) A loss of function mutation of presenilin-2 interferes with amyloid αβ-peptide production and notch signaling. J. Biol. Chem. 274:28669-28673). Also available are conditional PS1 knock out mice in which PS1 was eliminated selectively in excitatory neurons of the forebrain, beginning at postnatal day 18 (Yu et al., (2001) APP processing and synaptic plasticity in presenilin-1 conditional knockout mice. Neuron 31:713-726). A double-KO mouse in which the conditional loss of PS1 is on a PS2−/− background is also available for analysis (Sacra et al., (2004) Loss of presenilin function causes impairments of memory and synaptic plasticity followed by age dependent neurodegeneration. Neuron 42:23-36). With increasing age, the mutant mice develop striking neurodegeneration of the cerebral cortex and worsening impairments of memory similar to that seen in AD patients (Braak E, Braak H (1997) Alzheimer's disease: transiently developing dendritic changes in pyramidal cells of sector CA1 of the Ammon's horn. Acta Neuropathol. 93:323-325; Terry et al., (1991) Physical basis of cognitive alterations in Alzheimer's disease: synapse loss is the major correlate of cognitive impairment. Ann. Neurol. 30:572-580). Cortical neurons can be isolated from these mice and from appropriate controls of various ages and the distribution of mitochondria can be examined by staining with MitoTracker Red and anti-tubulin. ER, ER-MAM, and mitochondria can also be quantitated in these cells. COX and SDH histochemistry can be performed in freshly frozen brain tissue of the transgenic mice to see if there are alterations in respiratory chain function in neuronal cells. Immunohistochemistry to mitochondrial markers, such as TOM20 (a constitutively-expressed outer membrane marker), can indicate whether there is a change in the distribution and/or intensity of immunostain (indicative of altered organelle numbers) vs. controls.

### Example 12

### Studies of Brain Tissue

The analyses can be extended to a set of autoptic tissues from patients with FAD^{PS1}, SAD, and controls (Table 4). Initially, these morphological studies can be confined to the different fields of the hippocampal formation (HF), because this region of the paleocortex is invariably affected in both FAD and SAD. The distribution of mitochondria in the different neuronal compartments (perikaryon, dendrites, axons) can be examined to determine (1) whether there are the alterations in distribution of mitochondria observed in fibroblasts also present in neurons in FAD patients with documented mutations in PS1, and (2) whether there are there similar alterations in hippocampal neurons of patients with sporadic AD given the enrichment of ApoE in ER-MAM.

TABLE 4
BrainTissues Available for Studies

| | | | Time of | |
|---|---|---|---|---|
| | | Molecular | Brain | |
| Phenotype | Age | Defect | Removal | |
| FAD | 37 | PS1 Mutation | 6 | hours |
| FAD | 46 | PS1 Mutation | 10 | hours |
| FAD | 52 | PS1 Mutation | 12 | hours |
| FAD | 44 | PS1 Mutation | 18 | hours |
| SAD | 78 | Unknown | 14 | hours |
| SAD | 82 | Unknown | 6 | hours |
| Controls | 46-86 | None | 8-17 | hours |
| (4) | | | | |

Axonal defects, consisting of swellings that accumulated abnormal amounts of motor proteins, organelles, and vesicles, were found not only in transgenic mice bearing APP (K670N, M671L) and PS1 (A246E) mutations but also in the autoptic brains of patients with SAD (Stokin et al., (2005) Axonopathy and transport deficits early in the pathogenesis of Alzheimer's disease. Science 307:1282-1288). In the mice, these swellings, some of which were filled entirely by mitochondria, preceded amyloid deposition by more than a year (i.e. the swellings were not a response to amyloid) and appeared to be due to impaired kinesin mediated axonal transport (Stokin et al., (2005) Axonopathy and transport deficits early in the pathogenesis of Alzheimer's disease. Science 307:1282-1288). To answer these questions, mitochondria can be studied using specific immunological probes in neurons of the HF from AD patients and controls, similar to studies that previously performed (Bonilla et al., (1999) Mitochondrial involvement in Alzheimer's disease. Biochim. Biophys. Acta 1410:171-182). Clustering of mitochondria in the perinuclear region and aggregation of these organelles in the axons can be examined. The amount of ER, ER-MAM, PM, and mitochondria can be quantitated and the differential distribution of presenilin in these compartments can be determined

### Example 13

### Correlation with ApoE Status

Because ApoE is a component of ER-MAM, the ApoE allele status can be determined by PCR/RFLP analysis (Sorbi et al., (1994) ApoE allele frequencies in Italian sporadic and familial Alzheimer's disease. Neurosci. Lett. 177:100-102) and the genetics can be correlated with the quantitation of ER-MAM and of mitochondrial distribution to determine if the amount of communication between the ER and mitochondria and/or integrity of ER-MAM is different in patients and cells containing one or two ApoE4 alleles as compared to those containing ApoE2 or ApoE3 alleles. Plasmids over-expressing ApoE3 and ApoE4 can be transfected into human 293T cells to determine if there is a differential effect on ER-MAM and mitochondria.

Because human autoptic brain tissue can be used in the analysis, the time delay between death and autopsy can be examined to determine is there is an adverse affect on the ER-MAM localization of presenilin and on the mitochondrial mislocalization phenotype by sacrificing WT and PS1-mutant mice and harvesting brain and other somatic tissues after various time intervals at room temperature, ranging from 30 min to 18 hours (Table 4). For each sample, the amount of ER-MAM can be isolated, and the presence and total amount of presenilin in ER, ER-MAM, PM, and mitochondria can be quantitated by Western blotting. These analyses can indicate which autoptic samples are appropriate for use and whether they represent a good snapshot of what is actually occurring in the patients.

### Example 14

### Presenilin Complexes in ER-MAM

The role of in the ER-MAM subcompartment may be different than its role as a component of the γ-secretase complex located primarily in the plasma membrane. If so, ER-MAM localized presenilin functions as a solitary protein, or co-operates with partners other than those known to be part of the γ-secretase complex. A combination of blue-native gels, immunoprecipitation, and protein identification techniques can be used to determine whether presenilin interacts with other partners in the ER or the ER-MAM. If such partners are found, the effects of mutations in these presenilin binding partners on ER-MAM-localization can be determined. Given that presenilin in concentrated in the ER-MAM, Western blots of ER, ER-MAM, and mitochondrial fractions can be probed with antibodies to these proteins, as well as with anti-presenilin to determine if other components of the γ-secretase complex—APH1, nicastrin, and PEN2—are present in this compartment.

If one or more of these proteins are not present in the ER-MAM fraction, presenilin may have a function in ER-MAM different from that elsewhere in the cell. Alternatively, if all four proteins are in ER-MAM, it can mean that γ-secretase may be present in this compartment (Sato et al., (2007) Active γ-secretase complexes contain only one of each component. J. Biol. Chem. In press). Even if the components of the γ-secretase complex are in the ER-MAM, presenilin may still have another role in this compartment. To determine whether detected components of the γ-secretase complex in the ER-MAM are actually part a single complex, Westerns on blots of ER-MAM fractions separated on “blue-native PAGE” gels can be performed (Schagger H, von Jagow G (1991) Blue native electrophoresis for isolation of membrane protein complexes in enzymatically active form. Anal. Biochem. 199:223-231). In this system, large intact multi-subunit complexes can be separated by blue native polyacrylamide gel electrophoresis (BN PAGE) in the first dimension, and the constituents of the complexes can then be resolved by tricine-SDS-PAGE in the second dimension (Klement et al., (1995) Analysis of oxidative phosphorylation complexes in cultured human fibroblasts and amniocytes by blue-native electrophoresis using mitoplasts isolated with the help of digitonin. Anal. Biochem. 231:218-224). Both the first and second dimension gels can be analyzed by Western blot using anti-presenilin antibodies to see if presenilin is a constituent of a higher order complex, and by antibodies to the other components of the γ-secretase complex to see if they too are present. If the four components co-assemble, there can be co-migration of the Western bands for each component in the first dimension (i.e. BN-PAGE), and separation of the lane by SDS-PAGE in the second dimension can reveal the individual components with appropriate antibodies. Westerns of BN gels of the plasma membrane fraction can serve as a positive control for γ-secretase components (Manfredi et al., (2002) Rescue of a deficiency in ATP synthesis by transfer of MTATP6, a mitochondrial DNA-encoded gene, to the nucleus. Nat. Genet. 25:394-399; Ojaimi et al., (2002) An algal nucleus-encoded subunit of mitochondrial ATP synthase rescues a defect in the analogous human mitochondrial encoded subunit. Mol. Biol. Cell 13:3836-3844). Since PS1-mediated mitochondrial mislocalization was observed initially in primary human fibroblasts, these experiments can be performed initially on ER-MAM isolated from this tissue. However, it may be that the role of ER-MAM-localized presenilin differs in different tissues. For this reason, ER-MAM isolated from liver and brain (both from mouse and human, where available) can also be examined presenilin may associate with other as-yet-unidentified partners in ER-MAM; BN-PAGE can be used in this type of search as well. If there are “MAM-specific” presenilin partners on BN-PAGE, separation of a PS1-positive spot in the second dimension can reveal the constituent components of the complex as spots in the lane of unknown identity (seen by Coomassie or silver staining). Separation of a PS1-immunoprecipitated complex on one-dimensional SDS PAGE can achieve the same goal (a related approach can be to label presenilin with an affinity tag [H A, myc, FLAG, or His6] and immunoprecipitate a PS1-containing complex from isolated ER-MAM using an antibody to the affinity tag). The separated polypeptides can be excised from the gel and sequenced, by standard Edman degradation or by mass-spectrometry. Once PS1-associated candidates are identified, their biological relevance can be tested in a number of ways. Antibodies to a candidate can be used in SDS-PAGE, BN-PAGE, and in immunoprecipitation assays to see if the candidate is (1) concentrated in the ER-MAM and (2) associated with PS1. Knockdown of the candidate mRNA by RNAi can also knock down presenilin protein. A viable knockout mouse for the candidate gene may be available (Consortium TIMK (2007) A mouse for all reasons. Cell 128:9-13), which can used in further studies.

More than 30 proteins have been reported to associate with PS1 (Chen Q, Schubert D (2002) Presenilin-interacting proteins. Expert Rev. Mol. Med. 4:1-18), and a search for specific PS1-interacting partners in ER-MAM using immunoprecipitation may result in false positives. However, those searches were done on whole cell extracts. Presenilin binding partners can be identified in isolated ER-MAM, which can reduce the frequency of such false positives. Because the association of presenilin in a higher order complex may be weak (i.e. not observable on BNPAGE), isolated ER-MAM can be crosslinked (e.g. with formaldehyde) to bind presenilin to its partners, solubilize the fraction, and then co-immunoprecipitate with anti-presenilin antibodies. The crosslink can then be removed (e.g. by heating), run SDS-PAGE, and Westerns to detect presenilin can be performed, or bands from the gel can be isolated to identify them by mass spectrometry. The mass-spectrometry approach has the added advantage of allowing determination of the sequence and identity of the cross-linked proteins. This approach may be more useful than indirect methods, such as yeast 2-hybrid technology, because of the reduced rate of false positives. A related approach is to label presenilin with an affinity tag and immunoprecipitate a PS1-containing complex from isolated ER-MAM using an antibody to the tag.

The search for presenilin partners in ER-MAM is more difficult than merely confirming whether a protein is a constituent of a complex, because the unknown partners cannot be deduced by Western blot analysis in the 2nd dimension. BN PAGE using a higher amount of protein on the gels may have to be performed in order to see the partners by Coomassie or silver staining. Since only the ER-MAM fraction can be loaded on the BN-PAGE gels, rather than whole-cell extracts (as is normally done), the proteins of interest can be concentrated more than 20-fold, and specific bands may be visible in the second dimension. Thicker gels that can accommodate enough sample to identify the spots can be run. While Western blotting reveals only the polypeptide of interest, Coomassie/silver staining is nonspecific, and may reveal too many bands, even on an ER-MAM subfraction. Therefore, the analysis can also be performed on formaldehyde-crosslinked proteins, as described herein. As a complementary approach, ER-MAM can be isolated from the brains of PS1 knock-in mice or from PS1/PS2 double-knock-out mice vs. controls. BN-PAGE gels of KI or dKO vs. control ER-MAM run side-by-side can be performed to reveal those bands in the control that are missing in the mutated samples. Such missing bands can be authentic PS1 partners. Similar complementary analysis can be performed for PS2. Because degradation and/or loss of completely unrelated polypeptides in the absence of presenilin can result in false positives using this approach, BN-PAGE analyses of ER-MAM can be performed from cells in which either wild-type or mutant presenilin has been overexpressed and compared to untransfected control cells. If the partners are not rate limiting for assembly overexpressed presenilin can bring along higher levels of binding partners. These approaches or combinations thereof can be used to identify PS1-interacting proteins in the ER-MAM.

### Example 15

### Tracking Mitochondrial Mislocalization

The effect of presenilin mutations on anterograde and retrograde axonal transport of mitochondria, on retention and accumulation of mitochondria in nerve terminals, and on the dynamics of mitochondrial fusion and fission can be examined. In order to determine the relevance of these observations to AD, these studies can be conducted in primary neuronal cells derived from normal and FAD^{PS1} or FAD^{PS2} mice. The mitochondrial mislocalization phenotype can be due to (1) a reduced ability of mitochondria to move efficiently along microtubules, or (2) a reduced ability of mitochondria to attach to microtubules in the first place (or some combination of the two). To distinguish between these two possibilities, mitochondrial movement can be tracked in PS1-mutated cells, using a mitochondrially targeted photo-activatable GFP (“mitoDendra”) and live-cell imaging. Dendra is a monomeric variant of GFP (“dendGFP”) that changes from green to red fluorescent states when photoactivated by 488-nm light. Dendra is completely stable at 37° C., its photoconversion from green to red is both irreversible and high photostable, and it is not phototoxic (Gurskaya et al., (2006) Engineering of a monomeric green-to-red photo-activatable fluorescent protein induced by blue light. Nat. Biotechnol. 24:461-465). For some applications Dendra can be used instead of MitoTracker dyes, as these have several potential limitations due to their effects on mitochondrial membrane potential and oxidation (Buckman et al., (2001) MitoTracker labeling in primary neuronal and astrocytic cultures: influence of mitochondrial membrane potential and oxidants. J. Neurosci. Methods 104:165-176).

A mitochondrial-targeted Dendra construct in a pTurbo vector (mitoDendra) containing a cleavable N-terminal mitochondrial-targeting signal (MTS) derived from subunit VIII of cytochrome c oxidase (Rizzuto et al., (1989) A gene specifying subunit VIII of human cytochrome c oxidase is localized to chromosome 11 and is expressed in both muscle and non-muscle tissues. J. Biol. Chem. 264:10595-10600) can be used to target expressed Dendra into the mitochondrial matrix. When transfected into cells, mitoDendra normally fluoresces green. The optimal conditions to photoactivate mitoDendra at a defined region of interest (ROI) upon 488-nm laser excitation in confocal microscopy. A preliminary observation to detect the cells on the coverslip was performed at 3% mercury lamp intensity and scanning of the sample at 0.5% 488-nm excitation. Under these conditions, neither bleaching nor photoconversion to red was observed. Only 10 laser iterations were required to photoactivate mitoDendra to red and 20-30 to completely remove residual green fluorescence. To determine if the mitochondria are attached to microtubules, mitochondria can be visualized in living cells by colocalizing red mito-Dendra with TubulinTracker Green (a bi-acetylated version of Oregon Green 488 paclitaxel; Molecular Probes T34075). Multiple regions of interest can be defined in a single neuron, which can include one or several mitochondria at different cellular sites. Transport of multiple mitochondria in different neurons can be followed simultaneously and under the same experimental conditions by time-lapse photography, using confocal microphotography. Unique scan settings at each location (brightness, z-stack) can be defined independently. Several transport parameters can be studied, such as change in position, distance covered, and direction (i.e., distance of movement from an arbitrary origin point set at the cell nucleus). Only mitochondria that move unidirectionally for at least 3 consecutive frames are measured. Thus, transient transfection of cells (e.g. fibroblasts from patients; neurons from transgenic mice; cells and neurons stably-transfected with wt and mutated PS1 and with PS1 knockdown constructs) with mitoDendra can allow the movement of mitochondria containing the reporter (as a green signal) to be tracked.

Individual mitochondria can be converted to red fluorescence to track their movement in the cell body to determine whether they appear in a specified distance downstream in an axon, and how long it took to get there. Alternatively, mitochondria that are already in an axon can be photoconverted mitochondria to ask the same question and distinguish the dynamic behavior of initially perinuclear mitochondria that may not yet have attached to microtubules from that of mitochondria already attached and moving down axons. The mobilization and movement of mitochondria in the synapse/growth cone and the movement and distribution of tubular (i.e. fused) vs. punctate (fissioned) mitochondria can be examined ATP distribution and presenilin function in hippocampal neurons can also be examined in the context of loss of presenilin function.

Mitochondrial dynamics (and Ca2+ handling) in neurons under excitatory and non-excitatory conditions will also be examined. Treatment of neurons with glutamate alters mitochondrial shape (from elongated to punctate) and causes a rapid diminution in their movement (Rintoul et al. (2003) J. Neurosci 23:7881-7888). This effect is mediated by activation of the N-methyl D-aspartate (NMDA) subtype of glutamate receptors and requires the entry of calcium into the cytosol (Rintoul et al. (2003) J. Neurosci 23:7881-7888). Thus, it will be determined whether mitochondrial movement, distribution, and morphology are altered under excitatory and non-excitatory conditions in control vs. PS1-mutant neurons from transgenic mice, using both the mitoDendra constructs to visualize live cells and imaging of mitochondria in fixed cells.

### Example 16

### Effect of The Presenilin Mutation on The Interaction of Mitochondria with Microtubules In Vivo and In Vitro

Pathogenic mutations in PS1 that cause FAD are associated with phenotypes involving impaired mitochondrial movement. Indeed, tau, a substrate of GSK3β, is hyperphosphorylated in AD, and overexpressed tau causes mitochondrial clustering and a reduction in mitochondria in the neurites, due to an impairment in plus-end directed organellar transport (Ebneth et al., (1998) Overexpression of tau protein inhibits kinesin-dependent trafficking of vesicles, mitochondria, and endoplasmic reticulum: implications for Alzheimer's disease. J. Cell Biol., 143, 777-794). This possibility supported by (1) the observation that a PS1 mutation in a mouse PS1 knock-in model impairs GSK3β-mediated kinesin-based axonal transport and also increases tau phosphorylation (Pigino (2003) Alzheimer's presenilin 1 mutations impair kinesin-based axonal transport. J. Neurosci., 23, 4499-4508) and (2) the finding of axonal defects, consisting of swellings that accumulated abnormal amounts of microtubule-associated and molecular motor proteins, organelles, and vesicles, in transgenic mouse models of AD (Stokin et al., (2005) Axonopathy and transport deficits early in the pathogenesis of Alzheimer's disease. Science, 307, 1282-1288). More indirect support for the model derives from studies of APP and amyloid Aβ40-42. It has been found that upon overexpression, APP is targeted to mitochondria and impairs organellar function (Anandatheerthavarada et al., (2003) Mitochondrial targeting and a novel transmembrane arrest of Alzheimer's amyloid precursor protein impairs mitochondrial function in neuronal cells. J. Cell. Biol., 161, 41-54).

Mitochondrial movement can be examined along with interaction with microtubules and microtubule-based motors in PS1-ablated neurons focusing on the relationship between PS1, GSK3β, tau, and kinesins. Given confirmation that mitochondrial motility is defective, PS1-associated defects in mitochondrial distribution can be examined to determine if they affect energy mobilization, and the extent to which mitochondrial distribution defects contribute to neuronal dysfunction in PS1-ablated neurons.

The finding that presenilin is an ER-MAM-associated protein takes AD research in a new direction and provides new approaches to the treatment of familial and sporadic AD.

### Example 17

### Mitochondria Distributed in Neurons Bearing Normal and Mutated PS1

Observations in cultured fibroblasts and neurons will be investigated in the clinical situation. (a) Mitochondrial distribution and morphology will be examined in neuronal cells and tissues from normals and from patients and transgenic mice harboring mutations in PS1. (b) Using mitochondrially-targeted photoactivable fluorescent probes (“mitoDendra”) and live-cell imaging of neuronal cells, the effect of PS1 mutations on anterograde and retrograde axonal transport of mitochondria, on retention and accumulation of mitochondria in nerve terminals, and on the dynamics of mitochondrial fusion and fission will be examined. In order to determine the relevance of these observations to AD, these studies will be conducted mainly in neuronal cells derived from normal and FAD^{PS1} mice of different ages and under different excitatory states.

### Example 18

### Role of PS1 in ER-MAM

To address the mechanism of PS1's function in ER-MAM, (a) mitochondrial bioenergetics and redox signaling will be studied in PS1-mutant cells, (b) Ca2+ homeostasis in PS1— mutant cells will be analyzed using Ca2+ sensitive GFPs (“pericams”), (c) mitochondrial dynamics, neuronal transmission, and Ca2+ homeostasis will be examined after disrupting ER-mitochondrial interactions genetically in PACS2-KO mice, and (d) the role of PS1 in maintaining ER-MAM function will be assessed.

### Example 20

### PS1 and ER-MAM-Specific Protein Partners

To determine the mechanism by which PS1 is enriched in ER-MAM PS1 will be examined to determine if it interacts with other partners in the ER or ER-MAM subcompartments (using blue-native gels, immunoprecipitation, and protein identification techniques), the effects of mutations in PS1 binding partners on ER-MAM localization will be determined

### Example 21

### Analysis of PACS2-KO Mice

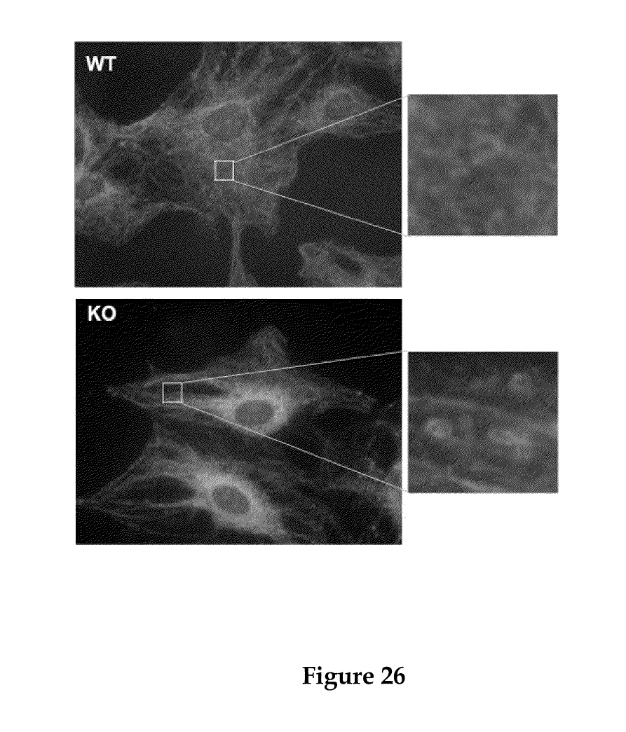

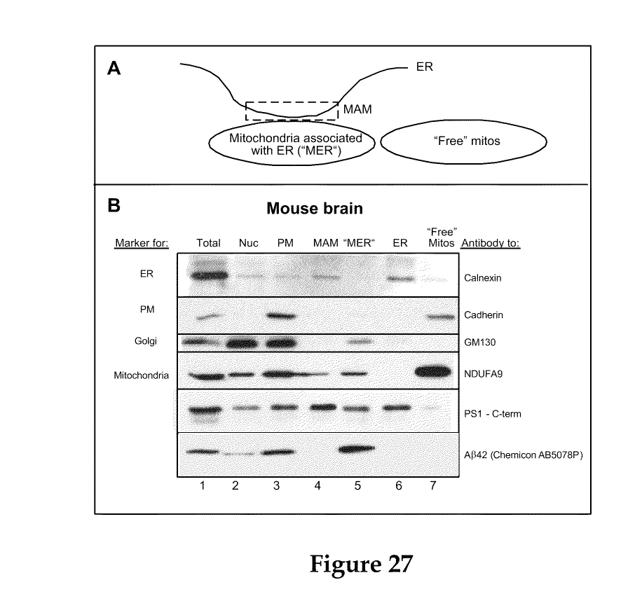

The only other protein previously known to play a role in ER-MAM integrity is phosphofurin acidic cluster sorting protein 2 (PACS2). PACS2 controls the apposition of mitochondria with the ER and appears to regulate of ER-mitochondrial communication via the ER-MAM. PACS2 is found predominantly in the perinuclear region of cells (Simmen et al. (2005) EMBO J. 24:717-729). To investigate if mutations in PACS2 can mimic the effects of mutated PS1 MEFs from PACS2-knockout mice were examined by double staining of MEFs with MT Red and anti-tubulin (Atkins et al. (2008) J. Biol. Chem. in press). Double staining showed a marked perinuclear localization of mitochondria in the PACS2-KO cells (FIG. 26). This result is similar to the results observed in FAD^{PS1} fibroblasts and in PS1-KD cells. They also showed an alteration in mitochondrial morphology wherein many mitochondria were “doughnut” shaped, possibly because they had detached from microtubules, allowing their tips to fuse. These results indicate that PS1 behaves like PACS2, and may function with PACS2 in the same pathway.

Mutations in APP and in the presenilin component of the γ-secretase, which processes APP to produce Aβ have been implicated in the etiology of FAD. Localization of PS1 to adherens junctions at the plasma membrane is consistent with its role in APP processing and cell signaling, since it places this component of γ-secretase complex in close proximity to PM-bound substrates, such as APP and Notch (Leissring et al. (2002) Proc. Natl. Acad. Sci. USA 99:4697-4702; Cupers et al. (2001) J. Neurochem. 78:1168-1178). Thus, the evidence in support of a role for PS1 in amyloid production in the pathogenesis of AD is strong. The finding that PS1 is also enriched in the ER-MAM, and affects the stability of this compartment and the distribution of mitochondria, point to an additional role for presenilins in the pathogenesis of the disease.

Without wishing to be bound by theory, there are several possible roles for ER-MAM-associated PS1. The possible roles described herein are examples and are not meant to be limiting. Other ER-MAM-associated PS1 function are also contemplated.

ER-MAM may be quantitatively the most important source of γ-secretase activity in the cell. Thus, one possibility is that ER-MAM-localized PS1 also functions as part of the γ-secretase complex, but is in a separate pool located in the ER-MAM (Ankarcrona M, Hultenby K (2002) Biochem. Biophys. Res. Commun 295:766-770; Hansson et al. (2005) J. Neurochem. 92:1010-1020). This possibility will be tested by determining whether all the components of the γ-secretase complex are present in ER-MAM.

A second possibility is that mutations in ER-MAM-localized PS1 affect the metabolism of APP by regulating APP trafficking within the secretory pathway (Naruse et al. (1998) Neuron 21:1213-1221; Kaether et al. (2002) J. Cell Biol. 158:551-561). This possibility will be tested by assaying for amyloid production in cells with compromised ER-mitochondrial trafficking.

A third possibility is that mutations in ER-MAM-localized PS1 affect localized [Ca2+] microdomains that ultimately affect neurotransmission (Rintoul et al. (2003) J. Neurosci 23:7881-7888). In this scenario, a localization of PS1 in ER-MAM can explain the various defects in Ca2+ homeostasis seen in cells from FAD patients (Ito et al. (1994) Proc. Natl. Scad. Sci. USA 91:534-538), in cell models (Leissring et al. (1999) J. Neurochem. 72:1061-1068; Leissring et al. (1999) J. Biol. Chem. 274:32535-32538), and in mouse models of FAD^{PS1} (Smith et al. (2005) Cell Calcium 38:427-437; Leissring M A, Akbari Y, Fanger C M, Cahalan M D, Mattson M P, LaFerla F M (2000) Capacitative calcium entry deficits and elevated luminal calcium content in mutant presenilin-1 knockin mice. J. Cell Biol. 149:793-798; Yoo et al. (2000) Neuron 27:561-572; Begley et al. (1999) J. Neurochem. 72:1030-1039; Barrow et al. (2000) Neurobiol. Dis. 7:119-126; Schneider et al. (2001) J. Biol. Chem. 276:11539-11544; Tu et al. (2006) Cell 126:981-993). This possibility will be tested by measuring [Ca2+] at or near ER, ER-MAM, and mitochondria. A fourth possibility is that mutations in ER-MAM-localized PS1 interfere with anchorage of mitochondria in the synapse or with the attachment of mitochondria to microtubules and/or their subsequent movement along microtubules (Chang D T, Reynolds I J (2006) Prog. Neurobiol. 80:241-268). These events are mediated by both ER and mitochondrial Ca2+, and mutated PS1 may prevent the delivery or retention mitochondria to appropriate sites within the cell (e.g. synapses). In one scenario, PS1 located in the ER-MAM regulates the machinery that is involved in mitochondrial movement, via a role in maintaining ER-mitochondrial bridges that allow for proper ER-mitochondrial communication, Ca2+ homeostasis, and binding of mitochondria to kinesin and hence to microtubules via, for example, the Ca2+-binding adapter Miro. Pathogenic mutations in PS1 would weaken or disrupt ER-mitochondrial communication, allowing for aberrant calcium spikes in the vicinity of mitochondria. A high local [Ca2+] can result in binding of Ca2+ to Miro, thereby preventing efficient attachment of mitochondria to microtubules. This can account for the perinuclear localization of mitochondria seen in PS1-mutant cells, the increase in the absolute amount of ER-MAM recovered from PS1-mutant cells, and the aberrant perinuclear accumulations of mitochondria in hippocampal regions of patients with FAD^{PS1}. This possibility will be tested by quantitating ER-MAM in normal vs. PS1-mutant cells, and by visualizing mitochondrial movement and distribution in normal and PS1 mutant cells.

### Example 22

### Mitochondrial Mislocalization in Disease

The mitochondrial mislocalization effect described herein takes AD research in a new direction, as it indicates a cause and effect relationship between altered mitochondrial dynamics and neurodegeneration. Mitochondrial mislocalization has now been found to play a role in the pathogenesis of other neurodegenerative diseases, including hereditary spastic paraplegia type 7 (Ferreirinha et al. (2004) J. Clin. Invest. 113:231-242), Charcot-Marie-Tooth disease types 2A (Zhao et al. (2001) Cell 105:587-597; Baloh et al. (2007) J. Neurosci. 27:422-430) and 4A (Niemann et al. (2005) J. Cell Biol 170:1067-1078), and autosomal dominant optic atrophy (Cipolat et al. (2004) Proc. Natl. Acad. Sci. USA 101:15927-15932). These results are supported by (1) the observation that a PS1 mutation (M146V) in a mouse PS1 knock-in model impairs axonal transport and also increases tau phosphorylation (Pigino et al. (2003) J. Neurosci. 23:4499-4508), (2) the finding of axonal defects, consisting of swellings that accumulate abnormal amounts of microtubule-associated and molecular motor proteins, organelles, and vesicles, in SAD patients and in transgenic mouse models of AD (Stokin et al. (2005) Science 307:1282-1288), and (3) the identification of a few rare patients with inherited frontotemporal dementia (Pick disease) (Dermaut et al. (2004) Ann. Neurol. 55:617-626; Halliday et al. (2005) Ann. Neurol. 57:139-143) and inherited dilated cardiomyopathy (Li et al. (2006) Am. J. Hum. Genet. 79:1030-1039) who had mutations in PS1 but who did not accumulate Aβ deposits in affected tissues; these “outlier” patients indicate that a clinical presentation due to mutations in PS1 can be “uncoupled” from the morphological hallmarks of AD. PS1 is physically and functionally associated with ER-MAM, and that mutations in PS1 which affect warrants further investigation.

In addition to showing how PS1 functions in ER-mitochondrial communication, the analysis of ER-MAM function can also be used to define a strategy for treating FAD^{PS1}. Because altered ER-MAM function is, in all or some aspects, the underlying pathogenetic cause of FAD, approaches to improve this function will be therapeutically valuable. Both the SCD1/DGAT2 FRET assay and the cinnamycin toxicity assay can be used in a large-scale chemical screen of PS1-mutant cells to identify compounds that rescue FRET and/or cinnamycin sensitivity in colorimetric assays.

### Example 23

### Cells and Tissue Analysis

Cells and/or tissues from one or more of the following sources will be used. The specific reagent(s) to be analyzed will depend on the analytical approach employed, based on the suitability of the model for analysis. All relevant control cells/tissues are also available including, but not limited to cells and tissues from human AD patients, skin fibroblasts from FAD^{PS1} and SAD patients, autoptic brain from FAD^{PS1} and SAD patients, cells and tissues from presenilin-mutant mice, transgenic mice expressing mutant human PS1 on a WT mouse background (PS1-Tg) (Duff et al. (1996) Nature 383:710-713), MEFs from knockout mice lacking PS1 (PS1-KO) (Donoviel et al. (1999) Genes Dev. 13:2801-2810), MEFs from knockout mice lacking both PS1 and PS2 (PS1/PS2-dKO) (Donoviel et al. (1999) Genes Dev. 13:2801-2810), MEFs from PS1/PS2-dKO mice expressing human WT PS1 (Donoviel et al. (1999) Genes Dev. 13:2801-2810), MEFs from PS1/PS2-dKO mice expressing human WT PS2 (Donoviel et al. (1999) Genes Dev. 13:2801-2810), MEFs from PS1/PS2-dKO mice expressing human D385A PS1 (“γ-secretase dead” mutant) (Donoviel et al. (1999) Genes Dev. 13:2801-2810), mice in which PS1 has been ablated conditionally in the forebrain of WT mice (PS1-cKO) (Yu et al. (2001) Neuron 31:713-726), mice in which PS1 has been ablated conditionally in the forebrain of PS2-KO mice (PS1/PS2-dKO) (Chen et al. (2008) J. Neurosci. Res. 86:1615-1625), frozen brain from PS1/PS2-dKO mice (Saura et al. (2004) Neuron 42:23-36; Chen et al. (2008) J. Neurosci. Res. 86:1615-1625), cells in which from PS1 expression has been knocked down by shRNA, PS1-KD 3T3 cells and CCL131 mouse neuroblastoma cells differentiated with retinoic acid, cells and tissues from PACS2-mutant mice, frozen brain, and MEFs from PACS2-KO mice (Myhill et al. (2008) Mol. Biol. Cell in press), and PACS2-knockdown cells by RNAi (Simmen et al. (2005) EMBO J. 24:717-729).

### Example 24

### Mitochondrial Distribution in Neurons Bearing Normal and Mutated PS1

Mitochondrial distribution and morphology in cells and tissues from normal and FAD^{PS1} patients and transgenic mice will be studied, and mitochondrial dynamics will be studied by live-cell imaging.

### Example 25

### Analysis of Mitochondrial Distribution and Morphology

The phenotype of mitochondrial mislocalization observed in FAD^{PS1} fibroblasts and in the hippocampus of an FAD^{PS1} patient indicate that PS1 plays a role in determining mitochondrial distribution, which may be relevant to the pathogenesis of FAD^{PS1}. PS1 is also present in ER-MAM in brain tissue, the effects observed in somatic cells (e.g. fibroblasts; PS1-knockdown cells) will be investigated in brain and in neuron. These tissues may be more clinically relevant in some aspects.

### Example 26

### Analysis of Other Mutations

Preliminary studies were performed in fibroblasts isolated from FAD^{PS1} patients with the A246E and M146L mutations. Fibroblasts from FAD patients with other PS1 mutations (lines EB [G209V], GF [I143T], WA [L418F]), and WL [H163R]), will be studied using methods including, but not limited to those methods described herein. A fibroblast line carrying a PS2 mutation (line DD [N141I]) and a line carrying a pathogenic mutation in APP will also be examined. In addition to examining ER-MAM in these cells, mutations in PS1 will be examined for their effect on ER-to-PM trafficking of APP (Cai et al. (2003) J. Biol. Chem. 278:3446-3454). Western blotting will be performed to detect both APP and Aβ (and the ratio of Aβ42:Aβ40) in the various subcellular fractions isolated from control and PS1-mutant cells.

### Example 27

### Transgenic Mice that Overexpress Human PS1 (M146L and M146V Mutations)

Mice in which PS1 has been knocked out are embryonic lethals (Handler et al. J (2000) Development 127:2593-2606), but PS2 KO mice are viable (Steiner et al. (1999) J. Biol. Chem. 274:28669-28673). Viable conditional PS1 knock-out mice in which PS1 was eliminated selectively in excitatory neurons of the forebrain, beginning at postnatal day 18 (Yu et al. (2001) Neuron 31:713-726) will be examined. A double-KO mouse in which the conditional loss of PS1 is on a PS2-null background (Yu et al. (2001) Neuron 31:713-726) as well as cells from a second, similar, dKO line (Saura et al. (2004) Neuron 42:23-36) will also be examined. PACS2-KO mice (Atkins et al. (2008) J. Biol. Chem. in press) from which neurons can also be obtained are also available.

Cortical neurons will be isolated from these mice and from appropriate controls and look at the distribution of mitochondria by staining with MT Red and anti-tubulin. ER, ER-MAM, and mitochondria in these cells will be quantitated. COX and SDH histochemistry will be performed in freshly-frozen brain tissue from the transgenic mice to determine if there are alterations in respiratory chain function in neuronal cells. Immunohistochemistry to mitochondrial markers, such as TOM20 (a constitutively expressed outer membrane marker), will indicate whether there is a change in the distribution and/or intensity of immunostain (indicative of altered organelle numbers) vs. controls.

### Example 28

### Studies of Brain Tissue

As described herein, alterations in mitochondrial morphology in the hippocampal formation of a single patient with FAD^{PS1} have been observed. These analyses will be extended to a larger set of autoptic tissues from patients with FAD^{PS1}, SAD, and controls. Initially, these morphological studies will be performed on the different fields of the hippocampal formation (HF), which is invariably affected in both FAD and SAD. The distribution of mitochondria in the different neuronal compartments (perikaryon, dendrites, axons) will be investigated to determine if: (1) the alterations in distribution of mitochondria observed in fibroblasts are also present in neurons of the HF in FAD patients with documented mutations in PS1 (2) there are similar alterations in hippocampal neurons of patients with sporadic AD. In this regard, axonal defects, consisting of swellings that accumulated abnormal amounts of motor proteins, organelles, and vesicles, were found not only in transgenic mice bearing APP (K670N, M671L) and PS1 (A246E) mutations but also in the autoptic brains of patients with SAD (Stokin et al. (2005) Science 307:1282-1288). In the mice, these swellings, some of which were filled entirely by mitochondria, preceded amyloid deposition by more than a year (i.e. the swellings were not a response to amyloid) and appeared to be due to impaired kinesin-mediated axonal transport (Stokin et al. (2005) Science 307:1282-1288). Mitochondria will be studied using specific immunological probes in neurons of the HF from AD patients and controls (Bonilla et al. (1999) Biochim. Biophys. Acta 1410:171-182) to look for clustering of mitochondria in the perinuclear region and for aggregation of these organelles in the axons. The amount of ER, ER-MAM, PM, and mitochondria will be quantitated and the differential distribution of PS1 in these compartments will be determined COX and SDH histochemistry will be performed on frozen tissue (as opposed to tissue fixed in formalin or paraffin), as described herein. Similar analyses on brain tissue from the M146L/V transgenic mice, the dKO mice, and appropriate controls will also be performed. Since mitochondrial morphology is altered, the expression of mitochondrial fission and fusion proteins (e.g. MFN1/2, FIS1, OPA1, DRP1) in PS1-mutant cells and tissues will be studied by Western blot analysis.

To determine whether the time delay between death and autopsy has an adverse affect on the ER-MAM localization of PS1 and on the mitochondrial mislocalization phenotype, WT and PS1-mutant mice will be sacrificed and brain and other somatic tissues will be harvested after various time intervals at room temperature, ranging from 30 min to 18 hours. For each sample, the amount of ER-MAM that can be isolated will be quantitated, and the presence and total amount of PS1 in ER, ER-MAM, PM, and mitochondria will be determined by Western blotting. These analyses will indicated which autoptic samples are appropriate for use and indicate which autoptic tissues represent a good snapshot of what is actually occurring in the patients.

### Example 29

Culturing of explanted primary mouse neurons. Culturing of explanted primary mouse neurons will be performed based on procedures already described (Friedman et al. (1993) Exp. Neurol. 119:72-78) that yield a relatively pure culture of neurons. To ensure that this is the case, immunostaining for α-internexin, an intermediate filament protein expressed by differentiated postmitotic neurons of the developing CNS, but not by neuroblasts or cells of glial lineage, will be performed (Fliegner et al. (1994) J. Comp. Neurol. 342:161-173).

### Example 30

Visualization of mitochondria, ER, and the cytoskeleton by confocal microscopy. Fibroblasts are first stained with MT Red and anti-tubulin antibody. A z-series (interval set to 1.4 μm to give non-overlapping sections) of images covering the total cell thickness is collected with a Zeiss LSM510 confocal microscope using a Plan-Neofluar, 0.9 NA objective lens. The pinhole is set to give an optical section of 1.4 μm. Excitation is at 488 nm (for green) and 543 nm (for red). This work will be done in the Imaging Core. Quantitation of mitochondrial distribution in cells. Confocal imaging z sections are projected into a single image. An area between the nucleus and the cell periphery, as determined by microtubule staining, is outlined, and the midpoint between the nucleus and the farthest point at the cell periphery is determined. Using the midpoint, the outlined area is then divided into two parts: regions proximal (A) and distal (B) to the nucleus. Mean grayness values of the MT Red stain are recorded for the proximal and distal parts. For quantification of mitochondria in the outer edges of a cell, the grayness value for the distal part is divided by the grayness value for the total area (proximal+distal). Calculation of grayness value for the total area=([GraynessA×AreaA]+[GraynessB×AreaB])/(AreaA+AreaB). This work will be done in the Imaging Core. Immunohistochemistry in brain. This will be performed on 10-μm-thick paraffin-embedded sections using the ABC method or by double-labeling methods with different fluorochromes (Tanji K, Bonilla E (2001) Methods Cell Biol. 65:311-332). Polyclonal antibodies against human COX II, ND1, ATPase 8, the ironsulfur (FeS) protein subunit of Complex III, and monoclonal (Molecular Probes) and polyclonal (Alpha Diagnostic) antibodies against TOM20 (Santa Cruz) will be used. For neuronal probes, commercially available (Sigma) monoclonal antibodies against MAP2, a perikaryon and dendritic marker, and monoclonal antibodies against MAPS, a marker for neuronal axons will be used. Additional sections will be stained with H-E for conventional microscopic study, with thioflavine S for localization of amyloid deposits, and with a modified Bielschowsky silver stain for evaluation of plaques and neurofibrillary tangles. The samples will be examined with an Olympus BX52 microscope equipped with deconvolution and 3-D reconstruction softwares. Other methods (e.g. COX and SDH histochemistry) may also be used.

### Example 31

### Mitochondrial Movement in Neurons by Live-Cell Imaging

Mutations in PS1 affect the movement and/or localization of mitochondria in fibroblasts from FAD^{PS1} patients, in COST cells transfected with mutated PS1, and in PS1-knockdown 3T3 and CCL131 neuroblastoma cells. Similar analysis will be performed in neurons, which are the clinically relevant tissue in FAD.

The effect of PS1 mutations on anterograde and retrograde axonal transport of mitochondria, on retention and accumulation of mitochondria in nerve terminals, and on the dynamics of mitochondrial fusion and fission will be analyzed. These studies will be conducted in primary neuronal cells derived from normal and FAD^{PS1} mice of different ages and under different excitatory states. The mitochondrial mislocalization phenotype can be due to either (1) a reduced ability of mitochondria to move efficiently along microtubules, or (2) a reduced ability of mitochondria to attach to microtubules in the first place (or some combination of the two). To distinguish between these two possibilities, mitochondrial movement in PS1-mutated cells will be tracked using mitochondrially-targeted photoactivatable GFP (“mitoDendra”) and live-cell imaging. Dendra is a monomeric variant of GFP (“dendGFP”) that changes from green to red fluorescent states when photoactivated by 488-nm light. Dendra is completely stable at 37° C., its photoconversion from green to red is both irreversible and high photostable, and it is not phototoxic (Gurskaya et al. (2006) Nat. Biotechnol. 24:461-465). For some applications Dendra can be used in place of MitoTracker dyes, as these have several potential limitations due to their effects on mitochondrial membrane potential and oxidation (Buckman et al. (2001) J. Neurosci. Methods 104:165-176).

To determine whether the mitochondria are attached to microtubules in living cells, colocalization of red mito-Dendra with TubulinTracker Green (a bi-acetylated version of Oregon Green 488 paclitaxel; Molecular Probes T34075) will be examined Multiple regions of interest can be defined in a single neuron, which can include one or several mitochondria at different cellular sites. Transport of multiple mitochondria in different neurons can be followed simultaneously and under the same experimental conditions by time-lapse confocal microphotography. Unique scan settings at each location (brightness, z-stack) can be defined independently. Several transport parameters can be studied, such as change in position, distance covered, and direction (i.e., distance of movement from an arbitrary origin point set at the cell nucleus). Only mitochondria that move unidirectionally for at least 3 consecutive frames are measured (see example in the Core 2 narrative). Thus, transient transfection of cells (e.g. fibroblasts from patients; neurons from transgenic mice; neuroblastoma cells and neurons stably-transfected with wt and mutated PS1 and with PS1-knockdown constructs) with mitoDendra will allow tracking of the movement of all mitochondria containing the reporter, as a green signal. In addition, photoconversion of individual mitochondria to red fluorescence will be used to track their movement. Individual mitochondria can be illuminated in the cell body to determine whether they appear in a specified distance downstream in an axon, and how long it took to get there.

Alternatively, mitochondria that are already in an axon can be photoconverted to ask the same question. In this way, the dynamic behavior of initially perinuclear mitochondria that may not yet have attached to microtubules can be compared to that of mitochondria already attached and moving down axons. The mobilization and movement of mitochondria in the synapse/growth cone will be examined

Mitochondrial dynamics (and Ca2+ handling) in neurons under excitatory and non-excitatory conditions will also be examined. Treatment of neurons with glutamate alters mitochondrial shape (from elongated to punctate) and causes a rapid diminution in their movement (Rintoul et al. (2003) J. Neurosci 23:7881-7888). This effect is mediated by activation of the N-methyl D-aspartate (NMDA) subtype of glutamate receptors and requires the entry of calcium into the cytosol (Rintoul et al. (2003) J. Neurosci 23:7881-7888). Thus, both the mitoDendra constructs (to visualize live cells) and imaging of mitochondria in fixed cells will be used to determine whether mitochondrial movement, distribution, and morphology are altered under excitatory and non-excitatory conditions in control vs. PS1-mutant neurons from transgenic mice. Glutamate induction of synaptic plasticity is age-dependent, that is, explanted rat neurons that are ˜18 days in vitro (DIV) behaved differently than did “younger ones (˜10 DIV) (Sapoznik et al. (2006) Learn. Mem. 13:719-727). Thus, the various assays will be performed on explanted mouse neurons (described herein) at different DIV.

### Example 32

### Transfection of MitoDendra

Transfection of mitoDendra into neurons will be performed as previously described (Nikolic et al. (1996) Genes Dev. 10:816-825; Ackerley et al. (2000) J. Cell Biol. 150:165-176) using Lipofectamine 2000 (Invitrogen) or the Promega profection mammalian transfection system. Typically, 10% of the cells are transfected, which provides a sufficient number of cells to allow for multiple measurements. However, to improve gene expression efficiency and to minimize non-specific toxicity derived from transfection approaches, an adenoviral vector mitoDendra construct will also be used into an adenoviral vector (Suhara et al. (2003) Neurobiol. Aging 24:437-451; Magrane et al. (2006) Exp. Cell Res. 312:996-1010; Magrane et al. (2005) J. Neurosci. 25:10960-10969). Neurons are imaged 36 hr after transfection. Visualization of mitochondrial movement in neurons.

### Example 33

### Neuronal Excitation

The protocol of Rintoul et al. will be used (Rintoul et al. (2003) J. Neurosci 23:7881-7888). Explanted neurons transfected with mitoDendra will be treated with 30 mM glutamate plus 1 mM glycine for 5 min as described (Rintoul et al. (2003) J. Neurosci 23:7881-7888), in the presence and absence of 5 mM MK801 (which blocks the effect of glutamate), and mitochondrial movement will be monitored. Similar experiments will be performed using 100 mM NMDA plus 1 mM glycine. Other controls will include monitoring movement in the presence of kainite (which depolarizes the plasma membrane) and the calcium ionophore 4-Br-A23187 (Sigma). Depolarization of mitochondrial with, for example, FCCP (carbonyl cyanide 4 [trifluoromethoxy]phenylhydrazone), will show the role of mitochondrial ATP synthesis on these processes.

### Example 33

### Role of PS1 in ER-MAM

The effect of PS1 mutations on mitochondrial bioenergetics will be assessed. How Ca2+ homeostasis is altered in PS1-mutated cells will be determined. The effect of disrupting ER-mitochondrial interactions on mitochondrial bioenergetics, on Ca2+ homeostasis, on the subcellular distribution of mitochondria, and on neuronal transmission will be examined. The effect of PS1 mutations on ER-MAM function will be examined

### Example 34

### Analysis of Calcium Homeostasis in Normal and PS1-Mutated Cells

The results described herein show a defect in ER-mitochondrial calcium trafficking in PS1-KD CCL131 neuroblastoma cells. Owing to its enrichment in ER-MAM, mutations in PS1 alter Ca2+ trafficking, not only between the ER and mitochondria, but also in other regions of the cell. It is known that organellar trafficking is known to take place through a low-affinity Ca2+ uniporter and through an electroneutral Ca2+/Na+-H+ antiporter (Pozzan et al. (1994) Physiol. Rev. 74:595-636), that mitochondrial Ca2+ uptake influences the kinetics and distribution of the cytoplasmic concentration of Ca2+ ([Ca2+]c) (Herrington et al. (1996) Neuron 16:219-228), and that PS1 plays a role in this trafficking (e.g. Tu et al. (2006) Cell 126:981-993)). This trafficking can now be monitored using fluorescent Ca2+ reporters targeted to appropriate locations in the cell.

Alterations in Ca2+ homeostasis in both cellular and animal models of FAD^{PS1} will be assessed using GFP-based calcium reporters (“pericams”) targeted to mitochondria and the cytosol. Pericams belong to a class of chimeric probes (Filippin et al. (2005) Cell Calcium 37:129-136) in which GFP derivatives (e.g. yellow YFP) are fused with a Ca2+ binding protein, such as calmodulin (CaM). In pericams, the linear sequence of YFP is cleaved, generating new N- and C-termini, while the original N- and C-termini are fused together (i.e. circular permutation). The linkage of CaM and the CaM-binding domain of myosin light chain kinase (M13) to the new N- and C-termini makes pericams sensitive to calcium (Pinton et al. (2007) Meth. Cell Biol. 80:297-325). They have a high affinity for Ca2+ (Kd ˜0.7 μM), which is favorable for sensing physiological Ca2+ changes. A “ratiometric pericam” has also been developed with an excitation wavelength that changes in a Ca2+-dependent manner (Nagai et al. (2001) Proc. Natl. Acad. Sci. USA 98:3197-3202).

By targeting a pericam to mitochondria while measuring cytosolic calcium with fura-2, ratiometric data that allows one to quantitate the amount of Ca2+ in both compartments can be obtained. The [Ca2+]c is quantitated spectrophotometrically but can also be visualized morphologically (see FIG. 25). As with the mitoDendra constructs, the pericam constructs will be inserted into adenoviral vectors to increase the efficiency of transfecting pericams into neurons. Initial transfections with pericams will be done in: (1) PS1/PS2-dKO MEFs and PS1-KD 3T3 cells; (2) PS1/PS2-dKO MEFs rescued with wild type or FAD^{PS1}-mutant P51; (3) neurons expressing WT or mutated PS1 maintained under excitatory vs. non-excitatory states; and (4) mitochondria isolated from WT and dKO MEFs and PS1-KD 3T3 cells. The following protocols will be employed:

(1) Simultaneous imaging of [Ca2+]c (with fura2 or with a nuclear-pericam) and [Ca2+]m (with a mito-pericam, inverse or ratiometric) in intact cells, followed by sequential treatment with IP3-linked agonists, Tg, and back addition of extracellular Ca2+. This protocol allows for quantitation of the [Ca2+]c and [Ca2+]m rise evoked by IP3-mediated and residual ER Ca2+ mobilization and by store-operated Ca2+ entry. Note: when using pericams to measure [Ca2+]c, it is targeted to the nucleus; [Ca2+]n is used as a surrogate of [Ca2+]c, because it is not feasible use pericams to monitor simultaneously [Ca2+]c with [Ca2+]m (Yi et al. (2004) J. Cell Biol. 167:661-672; Csordas G, Hajnoczky G (2001) Cell Calcium 29:249-262). Thus, by measuring separately the extranuclear and nuclear areas, [Ca2+]m can be determined simultaneously with [Ca2+]n.

(2) Repeat of (1) in cells injected with Ru360 or treated with FCCP to prevent mitochondrial Ca2+ uptake. This protocol tests the role of mitochondrial Ca2+ sequestration in the [Ca2+]c signal in both control and PS mutant cells and is useful to test the dependence of the mito-pericam signal on the ΔΨm and uniporter activity.

(3) Simultaneous imaging of [Ca2+]c (fura2 or rhod2) and [Ca2+]m in permeabilized cells and in isolated mitochondria treated with 1P3, and CaCl₂. In parallel measurements, ΔΨm will also be monitored. This protocol allows for direct stimulation of the IP3R and allows for comparison of the [Ca2+]m elevations evoked by IP3-induced Ca2+ release and by elevation of the bulk cytoplasmic Ca2+. The latter will clarify whether the IP3R mitochondrial Ca2+ transfer or the mitochondrial Ca2+ uptake mechanism was altered. (4) Repeat protocol (3) in cells incubated in the presence of an EGTA/Ca2+ buffer (200 M and 20 M, respectively), to prevent the IP3-induced [Ca2+]c rise while monitoring [Ca2+]m. This protocol specifically tests the local Ca2+ transfer between IP3R and mitochondria. (5) Measurement of the perimitochondrial [Ca2+] using a mitochondrial outer membrane targeted (TOM20) pericam construct. (6) Imaging of [Ca2+]m and perimitochondrial [Ca2+] at the level of single mitochondria in various subcellular regions (perinuclear and peripheral) corresponding to the altered mitochondrial distribution in PS1-mutant cells. (7) Imaging of Ca2+ under conditions of neuronal excitation. [Ca2+] can be monitored at different locations in normal neurons (e.g. cell body, axons at various distances from the cell body, synapses, dendrites), and the effect on the topographical distribution [Ca2+] of mutations in PS1 and/or the disruption of ER mitochondrial communication in these cells can be determined. By combining parallel measurements of Ca2+ (with pericams) with the assessment of mitochondrial movement and morphology (with mitoDendra), under both excitatory and nonexcitatory conditions, the “calcium hypothesis” for the pathogenesis of AD will be examined in a highly focused way.

Measuring [Ca2+]c in the “bulk” cytosol may underestimate the degree of alteration in Ca2+ homeostasis due to a change in [Ca2+] movement between the ER and mitochondria through the ER-MAM. There may be [Ca2+]c “microdomains” located at or near the ER-MAM that reflect changes in Ca2+ homeostasis in a biologically meaningful way but that cannot be detected in the “bulk” cytosol. Accordingly, new pericam constructs that are targeted to other compartments of the cell will be generated. A pericam targeted to the ER or to ER-MAM will permit measurement of alterations in [Ca2+] in these compartments. If PS1 affects the bridges, changes in [Ca2+] in the ER-MAM of PS1-mutated cells using a “MAM-pericam” will be observable (e.g. fusing the pericam to FACL4; “PS1-pericam” will not be used because PS1 is targeted to other compartments of the cell, such as the plasma membrane). One comparison in such an experiment will be to target a ER-MAM-pericam to PACS2-KD cells (Simmen et al. (2005) EMBO J. 24:717-729) and KO mice (Atkins et al. (2008) J. Biol. Chem. in press): an alteration in [Ca2+] in both PS1- and PACS2-mutant cells will indicate that both proteins are involved in building or maintaining ER-mitochondrial bridges, whereas different [Ca2+] values will indicate that both proteins are not involved in building or maintaining ER-mitochondrial bridges. Mitochondrial [Ca2+]m can be measured using a pericam targeted to the mitochondrial matrix. Given that local Ca2+ concentrations in the vicinity of Miro (which is anchored in the mitochondrial outer membrane [MOM]) affect the attachment of mitochondria to microtubules (via the kinesin adaptor Milton), [Ca2+]m will be measured not only in the mitochondrial matrix, but at the outer membrane as well. Accordingly, a MOM-targeted pericam will be generated by fusing the pericam to either TOM20, a MOM localized component of the mitochondrial importation machinery, or by fusing the pericam to Miro itself (if such a construct does not affect Miro's function). In this way, [Ca2+] can be measured in the actual vicinity of the MOM where the attachment of mitochondria to microtubules takes place. The various pericams will be transfected into control and PS1-mutant cells and the ratio of [Ca2+]c:[Ca2+]m(MAT), [Ca2+]c:[Ca2+]MAM, and [Ca2+]c:[Ca2+]m(MOM) will be determined. If mutated PS1 causes haploinsufficiency, the Ca2+ homeostasis defect will be rescued by overexpressing wt-PS1 into the cells. Similar experiments can be done in neurons and other cells from the mice.

### Example 35

### [Ca2+] Assays Under Neuronal Excitation

To define Ca2+ homeostasis in response to extracellular Ca2+ entry, 1 mM glutamate (Eggett et al. (2000) J. Neurochem. 74:1895-1902) will be administered. As a control glutamate with the addition of kynurenic acid, a non-specific glutamate receptor antagonist will be used. Glutamate stimulation with and without the addition of specific blockers (RU360; EMD Biosciences) or release (CGP37157; Tocris Cookson) (Brini et al. (1999) Nature Med. 5:951-954) of mitochondrial Ca2+ uptake will also be performed. In undifferentiated cells that do not express glutamate receptors, a short-term cytosolic Ca2+ peak can be attained with thapsigargin (1 μM; Sigma). Because of the variability in the number of cells that respond to glutamate stimulation and because amplitude and delay of the Ca2+ response may vary from cell to cell, more than one cell line may have to be analyzed in order to obtain statistically significant measurements (Eggett et al. (2000) J. Neurochem. 74:1895-1902). Alternatively, intracellular Ca2+ spikes can be generated by stimulation of P2X ion channels, which respond to micromolar concentrations of extracellular ATP (North R A (2002) Physiol. Rev. 82:1013-1067). This approach allows for the depolarization of a large number of cells irrespective of their state of differentiation. Analysis of variance will also be performed to compare the various cell lines. If the data are not normally distributed, either the Kruskal-Wallis or Mann-Whitney U tests will be utilized.

### Example 36

### Analyses of PACS2-KO cells in which ER-MAM Communication is Disrupted

PACS2 is a protein adaptor that controls ER-mitochondria contacts (Simmen et al. (2005) EMBO J. 24:717-729). Experimental disruption of the physical communication between the ER and mitochondria in PACS2-KO mice may mimic the many of the various phenotype seen in PS1-mutated cells, thereby indicating the role of PS1 (and PACS2) in ER mitochondrial communication and the pathogenesis of FAD^{PS1}. Loss of ER-MAM function—whether via mutated PS1 or mutated PACS2— may indeed be relevant to the pathogenesis of FAD^{PS1}.

The subcellular distribution of ER-MAM and of PS1, and the effects of altering ER-mitochondrial communication on neuronal transmission and on calcium homeostasis will be examined in normal and PS1-mutated mouse neurons using PACS2-KO mice.

Two types of experiments with PACS2-KO mice and MEFs will be performed using procedures described herein. (1) “Static” experiments in isolated cells and tissues. This will include (a) examination of mitochondrial morphology in fixed cells (using MT Red and antibodies to tubulin, PS1, and other relevant markers); (b) quantitation of the amount of ER, ER-MAM, PM, and mitochondria; (c) analysis of ER-MAM function; (d) analysis of mitochondrial bioenergetics; (e) determination of the distribution of PS1 (and other relevant markers, including APP and Aβ) in these compartments; (f) analysis of mitochondrial distribution in PACS2-KO brains; and (g) analysis of the expression of fusion/fission proteins. (2) “Dynamic” experiments in living cells. This will include (a) monitoring mitochondrial distribution and movement using MT Red and adenoviral-transfected mitoDendra; and (b) monitoring Ca2+ levels in various subcellular compartments using fluorescent Ca2+ reporters. All relevant methods are described herein.

### Example 37

### Analysis of the Role of PS1 in ER-MAM Function

Because the mitochondrial maldistribution observed in PS1-mutant cells was also observed in PACS2-KO cells, PS1 may play a role in maintaining ER-MAM integrity and effective ER-mitochondrial communication, and defects in ER-MAM function may play a role in the pathogenesis of the disease.

Antibodies to known ER-MAM components will be used to characterize further the association of PS1 with ER-MAM and the disposition of this compartment in neurons, an unexplored area. ER-MAM will be isolated from WT, PS1-KO, PS1/PS2-dKO, and PACS2-KO brain and the amount of ER-MAM obtained will be quantitated and compare to those obtained in other tissues (e.g. liver, muscle). Measuring the amount of ER-MAM indicates the qualitative nature of the ER-MAM compartment and provides little mechanistic insight into whether PS1 is required for ER-MAM function. Accordingly, such function will be assayed using three different approaches: phosphatidylethanolamine (PE) formation, sensitivity to cinnamycin, and fluorescence resonance energy transfer (FRET) in the ER-MAM. PE formation. The ER-MAM is a locus of phospholipid synthesis. Notably, phosphatidylserine (PS) is transported from the ER-MAM to mitochondria, where it is decarboxylated to form PE; the PE is then retransported back to the ER-MAM, where it is methylated to form phosphatidylcholine (PC) (Achleitner et al. (1999) Eur. J. Biochem. 264:545-553). If ER-MAM function is compromised, the rate of transport of PS from the ER-MAM to the mitochondria is reduced, and hence the production of PE inside of mitochondria is also reduced (Achleitner et al. (1999) Eur. J. Biochem. 264:545-553; Achleitner et al. (1995) J. Biol. Chem. 270:29836-29842; Wu W I, Voelker D R (2001) J. Biol. Chem. 276:7114-7121; Schumacher et al. (2002) J. Biol. Chem. 277:51033-51042). Consistent with this idea, cholesterol and phospholipids (for example, PE, PS, and PC) were selectively reduced an AD “double-transgenic” mouse model (i.e. mutations in both APP and PS1) (Yao et al. (2008) Nerochem. Res. in press). The conversion of PS to PE will be examined by adding 3H-Ser to WT and mutant cells and measuring the amount of 3H-PE (and 3H-PS) produced as a function of time (Achleitner et al. (1995) J. Biol. Chem. 270:29836-29842). Sensitivity to cinnamycin. Cinnamycin, also called Ro 09-0198, is a tetracyclic peptide antibiotic that is used to monitor the transbilayer movement of PE in biological membranes (Choung et al. (1988) Biochim. Biophys. Acta 940:171-179; Choung et al. (1988) Biochim. Biophys. Acta 940:180-187). Cinnamycin binds specifically to PE (Choung et al. (1988) Biochim. Biophys. Acta 940:171-179; Choung et al. (1988) Biochim. Biophys. Acta 940:180-187), and was used in a screen to identify mutants defective in PS transport through the ER-MAM (Emoto et al. (1999) Proc. Natl. Acad. Sci. USA 96:12400-12405). There is be a minimum inhibitory concentration (MIC) at which cinnamycin binds to PE in normal cells and kills them via cytolysis, whereas cinnamycin at the same concentration will have reduced binding to PE in AD cells, and not kill them. An easy way to distinguish between the two is by a “live-dead” assay (e.g. living cells are green whereas dead cells are red). Thus, the MIC in WT and PS1-mutant cells will be measured.

### Example 38

### FRET

Modified from Man et al. (2006) J. Lipid Res. 47:1928. Diglycerol acyltransferase 2 (DGAT2) and stearoyl-CoA desaturase 1 (SCD1, also called SCD) are both localized in the MAM and interact with each other in that compartment. Both DGAT2 fused to yellow fluorescent protein (DGAT2-YFP) and SCD1 fused to cyan fluorescent protein (SCD1-CFP) are expressed in cells. In one embodiment, both fusion proteins can be expressed from a bicistronic plasmid. YFP is detected by illuminating the cells at 488 nm and detecting at 560 nm. CFP is detected by illuminating the cells at 403 nm and detecting fluorescence at 470 nm. In cells expressing both YFP and CFP, a FRET will be observed by detecting yellow fluorescence at 560 nm upon illumination in the blue at 403 nm. In control cells co-expressing DGAT2-YFP and SCD1-CFP, this FRET will be observed and the degree of FRET (intensity; number of FRET-positive cells compared to all transfected cells) will serve as a baseline value. The same construct(s) can be transfected in PS1-mutant cells and the degree of FRET measured and compared to FRET values observed in control cells. If MAM integrity is altered, the kinetics of the apposition of DGAT2-YFP to SCD1-CFP will be increased, thereby causing a increase in FRET intensity and/or in the number of FRET-positive cells. Without being bound by theory, this increase can occur because FRET signal increases with the 6th power of the distance between the YFP and CFP moieties.

Diacylglycerol-O-acyltransferase 2 (DGAT2) and stearoyl-CoA desaturase 1 (SCD1)-form a dimeric complex in the ER-MAM (Man et al. (2006) J. Lipid Res. 47:1928-1939). By appending YFP to DGAT2 and CFP to SCD1, Man et al. (Man et al. (2006) J. Lipid Res. 47:1928-1939) demonstrated FRET between SCD1-CFP and DGAT2-YFP, indicating that the two proteins are adjacent to each other (within a few nm) in the ER-MAM. Normal cells will have a strong FRET signal, because in “thick” ER-MAM membranes DGAT2 and SCD1 can move laterally through the ER-MAM lipid and “find” each other easily. However, in “thick” ER-MAM from FAD^{PS1} patients, the FRET signal will be altered due to differences in traversing the membrane, and the FRET signal will be increased significantly (the signal falls off with the 6th power of the distance between the two interacting moieties). This increase in FRET can be exploited in a chemical screen to search for compounds that improve the FRET signal (indicative of improved ER-MAM integrity), as a treatment strategy in FAD^{PS1}.

Plasmids encoding SCD1-CFP and DGAT2-YFP (Man et al. (2006) J. Lipid Res. 47:1928-1939) have been verified to be functional (i.e. the YFP 488-nm fluorescence at 560 nm, and CFP 403-nm fluorescence at 470 nm have been detected). FRET will be examined in cells expressing both YFP and CFP by detecting fluorescence at 560 nm upon illumination in the blue at 403 nm. A construct in which both genes are on a bicistronic vector and are expressed stably will also be generated.

### Example 39

### Identification of PS1Partners in the ER-MAM

ER-MAM-localized PS1 may function either as a solitary protein, or co-operate with partners other than (or in addition to) those known to be part of the γ-secretase complex. The pleiotropic effects of mutations in PS1 in FAD^{PS1} patients (e.g. altered lipid, glucose, cholesterol, and Ca2+ metabolism) may indicate that PS1 functions with one or more partners.

PS1 will be investigated to determine if it interacts with other partners in the ER-MAM. If such partners are found, the effects of mutations in these PS1 binding partners on ER-MAM localization will be determined. Given that PS1 in concentrated in the ER-MAM, and that there is strong γ-secretase enzymatic activity in ER-MAM (FIG. 4), analysis will be performed to determine if the other components of the γ-secretase complex—APH1, nicastrin, and PEN2), as well as the regulatory molecules CD147 and TMP21—are present in this compartment as well.

Western blots of subcellular fractions (e.g. ER, ER-MAM, plasma membrane [PM], and mitochondrial fractions) from WT mouse tissues and cells will be probed with antibodies to these proteins, as well as with anti-PS1. As negative controls, Westerns on PS1/PS2 dKO mouse brains and/or PS1-KO MEFs will be performed. If differences are observe in the Western pattern in the ER-MAM fraction compared to other compartments, it can indicate that PS1 may have a function in ER-MAM different from that elsewhere in the cell. Alternatively, finding all the components of the γ-secretase complex in the ER-MAM can still not eliminate the possibility of another role for PS1 in this compartment. To determine whether all detected components of the γ-secretase complex in the ER-MAM are actually part a single complex, Westerns on blots of ER-MAM fractions from test and control (PS1-null) mouse brain separated on “blue-native PAGE” gels (Schagger H, von Jagow G (1991) Anal. Biochem. 199:223-231) will be performed. In this system, large intact multi-subunit complexes can be separated by blue native-polyacrylamide gel electrophoresis (BN PAGE) in the first dimension, and the constituents of the complexes can then be resolved by tricine-SDS-PAGE in the second dimension (Klement et al. (1995) Anal. Biochem. 231:218-224). Both the first and second dimension gels can be analyzed by Western blot using anti-PS1 antibodies to see if PS1 is a constituent of a higher order complex, and by antibodies to the other components of the γ-secretase complex to see if they too are present. If all the subunits co-assemble, there will be co-migration of the Western bands for each component in the first dimension (i.e. BN-PAGE), and separation of the lane by SDS-PAGE in the second dimension will reveal the individual components with appropriate antibodies. Westerns of BNPAGE gels of the plasma membrane fraction will serve as a positive control for authentic γ-secretase components. Since the role of ER-MAM-localized PS1 may differ in different tissues, search will be extended to ER-MAM isolated from liver and brain. BN-PAGE system has been previously characterized (Manfredi et al. (2002) Nat. Genet. 25:394-399; Ojaimi et al. (2002) Mol. Biol. Cell 13:3836-3844). PS1 may associate with other as-yet-unidentified partners in ER-MAM, and BN-PAGE can be used in this type of search as well. However, to isolate PS1-interacting proteins in ER-MAM, a more direct, two-tiered approach can be performed using: (1) tandem affinity-purification (TAP Tag) in cell culture, and (2) direct IP in lysates from WT and PS1/2-dKO mouse brains and PS1-KO cells.

### Example 40

### TAP Tag

TAP-tagging is a highly-selective tandem affinity purification method (Rigaut et al. (1999) Nature Biotechnol. 17:1030-1032). In brief, the “bait” gene of interest (i.e. PS1) is fused with two “tandem tags”—a calmodulin binding site followed by an IgG binding domain—with a tobacco etch virus (TEV) cleavage site located between the two tags. The construct is expressed in cells and PS1-associated complexes in the purified ER-MAM fraction are first isolated by its strong binding to IgG resin via the IgG-binding domain of the PS1 fusion protein. After washing, TEV protease is added to release the bound material (i.e. the tagged PS1 complexes). The eluate is then incubated with calmodulin coated beads in the presence of calcium. This second affinity step is required to remove the TEV protease as well as traces of contaminants remaining after the first affinity selection. After washing, the bound material is released with EGTA. The purification is monitored at each step by Western blot analysis. Finally, the candidate proteins are resolved on silver-stained SDS gels and identified by mass spectrometry. The procedure will also be performed using empty vector (negative control) and on plasma membrane fractions (positive control). This method (Rigaut et al. (1999) Nature Biotechnol. 17:1030-1032) has two advantages. First, by purification with two consecutive antibodies, false positives can be minimized, which is a problem in any co-immunoprecipitation-based method for isolating interacting proteins. Second, the method allows for the purification of protein complexes under mild conditions, preserving the interactions among the proteins that form part of the complex to be purified. As a backup approach, the method of Tsai and Carstens (Tsai A, Carstens R P (2006) Nature Protocols 1:2820-2827) in which a 2× Flag tag replaces the calmodulin tag will be used. In this case the Flag tagged PS1 complexes are purified further by binding to beads containing anti-Flag antibodies, which are then released from the beads with Flag peptides.

As described (Tsai A, Carstens R P (2006) Nature Protocols 1:2820-2827), cells will be transfected stably with a bicistronic vector plasmid containing the CMV-derived eukaryotic promoter upstream of PS1 with a downstream IRES sequence followed by an antibiotic selection marker (e.g. puroR or neoR). Isolated ER-MAM (up to 40 mg) will be mixed with IgG beads with gentle rotation for 4-16 h at 4° C. After washing, the bound IgG resin will then be treated with 100 U of TEV protease for 16 h at 4° C. to release Flag tagged PS1 complexes. The complexes containing solution will be separated from the IgG resin with a 1-ml Micro Bio-Spin column. Eluates will be pooled and mixed with anti-Flag resin (Sigma) with gentle rotation at 4° C. for 4 h, followed by washing the Flag-PS1 complex-bound beads with 1 ml of TBS wash buffer, 3× at 4° C. Finally, Flag-tagged PS1-associated complexes will be eluted from the resin with 3× Flag peptide in TBS buffer. The calmodulin method (Rigaut et al. (1999) Nature Biotechnol. 17:1030-1032) is fundamentally similar (Jorba et al. (2008) J. Gen. Virol. 89:520-524).

### Example 41

### Immunoprecipitation (IP)

In parallel to the TAP method, PS1 antibodies that have been proven effective in IP, and the PS1 knockout mice and cells will be used. The specific antibodies will be efficient to pull down PS1 and its interacting proteins. The ER-MAM from the forebrains of PS1/2-dKO mice, or from cultured blastocysts from PS1-KO mice will be used as negative controls. In this approach, ER-MAM from wild-type and dKO mouse brains (or WT and PS1-KO cells) will be purified as described herein, and anti PS1 antibody will be used to pull down PS1 and its interacting proteins. Two antibodies that have been tested: PS1-CTF (Sigma) (Serban et al. (2005) J. Biol. Chem. 280:36007-36012) and monoclonal antibody MAB5232 (Chemicon) (Laudon et al. (2005) J. Biol. Chem. in press; Nakaya et al. (2005) J. Biol. Chem. 280:19070-19077) will be used. The co-immunoprecipitated proteins will be separated by SDS-PAGE, and the bands that are unique to the WT lane will be excised and sent for mass spectrometry analysis. With both techniques, once PS1-associated candidates are identified, their biological relevance will be tested in a number of ways. Antibodies to a candidate can be used in SDS-PAGE, BN-PAGE, and in immunoprecipitation assays to see if the candidate is (1) concentrated in the ER-MAM and (2) associated with PS1. Knockdown of the candidate mRNA by RNAi will also knock down PS1 protein. A viable knock-out mouse for the candidate gene may be available (Consortium TIMK (2007) Cell 128:9-13), which can be used for further studies. Moreover, if antibodies against the candidate proteins are available, they will be used to reverse-IP PS1 from the ER-MAM preparation from WT and PS1-mutant mice/cells. If the antibodies against the candidate proteins are not available, myc-tagged candidates will be generated and a two-directional IP will be performed with the TAP construct of PS1 in transfected cell cultures. The same two approaches will be used on other subcellular fractions (e.g. bulk ER; PM) to look for differences in binding partners in different fractions. If differences are found among fractions, this comparison will prove informative regarding the function(s) that PS1 may play in different parts of the cell (e.g. cleavage of Notch in the PM vs. a role in mitochondrial distribution and lipid metabolism in the ER-MAM).

Any approach to find protein partners may produce false positives, however the TAP Tag method, which uses two affinity purification steps, minimizes this problem. Moreover, the use of two different but complementary TAP Tag methods—one involving a calmodulin tag, and the other a Flag tag—will yield the same partners with both procedures, and these partners are more likely to be authentic, with the “non overlapping” set of partners more likely to be spurious.

Most searches for protein-protein interactions are conducted on whole cell extracts. PS1 binding partners will be investigated specifically in isolated ER-MAM, which will reduce the frequency of such false positives. Moreover, by enriching for the correct fraction, the chance of finding the true positives is increased simply because there is more protein to start with, and thus less protein is likely to be lost in the washing process.

The issue of false negatives (i.e. failure to identify a partner due to weak interacting proteins) is more difficult to address, as it boils down to a tradeoff between the strength of the washing conditions and the number of proteins recovered. From a practical standpoint, the “strong” positives will be examined first to ensure that they are identified and verified. These new partners will then be used as “bait” in further rounds of TAP Tag to look for new partners that may have been missed the first time around, as proteins that interacted weakly with PS1 may interact strongly with others in the complex.

### Example 42

### PS Radiolabel Assay

Uniformly-labeled 3H-Ser is added to cells for various time intervals (e.g. 0, 1, 2, 4, 6 hours). The cells are killed and the lipids are concentrated by chloroform extraction. The extract is analyzed by thin layer chromatography to identify various lipids (e.g. phosphatidylserine, phosphatidylethanolamine, phosphatidylinositol, phosphatidylcholine, total triglycerides, sphingomyelin) using purified standards (identified by spraying the plate with iodine to reveal the bands/spots) and the ³H label is counted. The ³H data is plotted vs. time and normalized against any variation of protein content among samples. A reduction in slope for ³H-PE vs. time in test vs. control will be indicative of an ER-MAM transport defect.

### Example 43

### Mitochondrial Dynamics in Neurodegenerative Disease

Mutations in presenilin-1 (PS1) cause familial Alzheimer disease (FAD). The results described herein show that PS1 is highly concentrated in “bridges” connecting the endoplasmic reticulum and mitochondria. Mutated PS1 increases this communication, resulting in many of the biochemical features that are hallmarks of FAD. Studying this relationship will indicate pathogenesis and therapeutic approaches for this devastating disease.

Presenilin 1 (PS1) localizes to the plasma membrane (PM), where it contributes to processing and accumulation of extracellular β-amyloid as part of the γ-secretase complex. In addition to this well established function, the results described herein show that PS1 plays another role in the pathogenesis of AD. Previous studies have revealed that PS1 is targeted not only to the PM, but also to the endoplasmic reticulum (ER). The results described herein show that PS1 is enriched in a specific subcompartment of the ER that is associated intimately with mitochondria and that forms a physical bridge between the two organelles, called ER mitochondria-associated membranes (MAM). As described herein, defects in mitochondrial distribution and morphology, as well as alterations in bioenergetics, redox levels, and Ca2+ homeostasis have also exist in various PS1 mutant cells.

ER-MAM has known functions in calcium homeostasis and mitochondrial distribution, two processes that affect synaptic transmission, which is known to be compromised in AD patients. Since defects in the accumulation of mitochondria at the synapse and defects in mitochondrial fusion and fission impair synaptic transmission, mitochondrial distribution and morphology defects can contribute significantly to the pathogenesis of FAD^{PS1}. As described herein mutations in PS1 inhibit mitochondrial distribution and hence neuronal transmission through effects on mitochondrial-ER interactions. Since Ca2+ regulates the attachment of mitochondria to microtubules, the defects in mitochondrial distribution observed FAD^{PS1} cells can be due to defects in ER-MAM-mediated calcium homeostasis that alter axonal mitochondrial transport. Alternatively, since ER-MAM has been shown to contribute to the anchorage of mitochondria at sites of polarized cell surface growth, the accumulation of mitochondria in the nerve terminal can be compromised in PS1 mutants. These two models are not mutually exclusive. The specific aims described herein are designed to determine the mechanism(s) underlying defects in mitochondrial distribution in PS1 mutants, and to address the role of ER-MAM-targeted PS1 in those processes.

Without wishing to be bound by theory, mutations in PS1 may inhibit mitochondrial distribution and hence neuronal transmission through effects on mitochondrial-ER interactions, via potential alterations in Ca2+ homeostasis, axonal mitochondrial transport, and/or anchorage of the organelle in the synapse. The maldistribution of mitochondria would be deleterious in elongated neurons, where mitochondria travel vast distances on microtubules to provide ATP for energy-intensive processes at distal sites, including synapses. Mitochondrial distribution and morphology will be studied in neurons from normal and FAD^{PS1} patients and PS1-mutant transgenic mice and (b) the effect of PS1 mutations on mitochondrial dynamics will be analyzed (i.e. transport, retention, fusion, and fission) in these neurons under different excitation states, using mitochondrially-targeted photoactivable fluorescent probes (“mitoDendra”) and live-cell imaging. The role of PS1 in ER-MAM will be investigated by (a) studying mitochondrial bioenergetics and redox signaling, using well-established methodologies, (b) analyzing Ca2+ homeostasis in PS1-mutated cells, using Ca2+-sensitive GFPs (“pericams”), (c) examining mitochondrial dynamics, neuronal transmission, and Ca2+ homeostasis after disrupting ER-mitochondrial interactions genetically in PACS2-KO mice, and (d) assessing the role of PS1 in maintaining ER-MAM function. It will be investigated whether PS1 has ER-MAM specific protein partners, using a combination of blue native gels, immunoprecipitation, and protein identification methods.

### REFERENCES

Achleitner G, Gaigg B, Krasser A, Kainersdorfer E, Kohlwein S D, Perktold A, Zellnig G, Daum G (1999) Association between the endoplasmic reticulum and mitochondria of yeast facilitates interorganelle transport of phospholipids through membrane contact. Eur. J. Biochem. 264:545-553.
Achleitner G, Zweytick D, Trotter P J, Voelker D R, Daum G (1995) Synthesis and intracellular transport of aminoglycerophospholipids in permeabilized cells of the yeast, Saccharomyces cerevisiae. J. Biol. Chem. 270:29836-29842.
Ackerley S, Grierson A J, Brownlees J, Thornhill P, Anderton B H, Leigh P N, Shaw C E, Miller C C (2000) Glutamate slows axonal transport of neurofilaments in transfected neurons. J. Cell Biol. 150:165-176.
Alonso M T, Villalobos C, Chamero P, Alvarez J, Garcia-Sancho J (2006) Calcium microdomains in mitochondria and nucleus. Cell Calcium 40:513-525.
Anandatheerthavarada H K, Biswas G, Robin M A, Avadhani N G. (2003) Mitochondrial targeting and a novel transmembrane arrest of Alzheimer's amyloid precursor protein impairs mitochondrial function in neuronal cells. J. Cell. Biol., 161, 41-54.
Ancolio, K. et al., Proc. Natl. Acad. Sci. USA 96:4119-4124 (1999)
Andreyev A Y, Kushnareva Y E, Starkov A A (2005) Mitochondrial metabolism of reactive oxygen species. Biochemistry (Moscow) 70:200-214.
Ankarcrona M, Hultenby K (2002) Presenilin-1 is located in rat mitochondria. Biochem. Biophys. Res. Commun 295:766-770.
Annaert et al., J. Cell Biol. 147, 277 (1999).
Annaert W G, Levesque L, Craessaerts K, Dierinck I, Snellings G, Westaway D, George-Hyslop P S, Cordell B, Fraser P, De Strooper B (1999) Presenilin 1 controls secretase processing of amyloid precursor protein in pre-Golgi compartments of hippocampal neurons. J. Cell Biol. 147:277-294.
Ardail D, Popa I, Bodennec J, Louisot P, Schmitt D, Portoukalian J (2003) The mitochondria-associated endoplasmic-reticulum subcompartment (MAM fraction) of rat liver contains highly active sphingolipid specific glycosyltransferases. Biochem. J. 371:1013-1019.
Ardailefal., Biochem. J. 371, 1013 (2003).
Atkins K M, Thomas L, Youker R T, Harriff M J, Pissani F, You H, Thomas G (2008) HIV-1 Nef binds PACS-2 to assemble a multikinase cascade that triggers major histocompatibility complex class I (MHC-I) down-regulation: analysis using short interfering RNA and knock-out mice. J. Biol. Chem. in press:
Baki L, Marambaud P, Efthimiopoulos S, Georgakopoulos A, Wen P, Cui W, Shioi J, Koo E, Ozawa M, Friedrich V L, Jr., Robakis N K (2001) Presenilin-1 binds cytoplasmic epithelial cadherin, inhibits cadherin/p120 association, and regulates stability and function of the cadherin/catenin adhesion complex. Proc. Natl. Acad. Sci. USA 98:2381-6.
Baloh R H, Schmidt R E, Pestronk A, Milbrandt J (2007) Altered axonal mitochondrial transport in the pathogenesis of Charcot-Marie-Tooth disease from mitofusin 2 mutations. J. Neurosci. 27:422-430.
Barrow P A, Empson R M, Gladwell S J, Anderson C M, Killick R, Yu X, Jefferys J G, Duff K (2000) Functional phenotype in transgenic mice expressing mutant human presenilin-1. Neurobiol. Dis. 7:119-126.
Barry F A, Graham G J, Fry M J, Gibbins J M. (2003) Regulation of glycogen synthase kinase 3 in human platelets: a possible role in platelet function? FEBS Lett., 553, 173-178.
Begley J G, Duan W, Chan S, Duff K, Mattson M P (1999) Altered calcium homeostasis and mitochondrial dysfunction in cortical synaptic compartments of presenilin-1 mutant mice. J. Neurochem. 72:1030-1039.
Berridge M J (2002) The endoplasmic reticulum: a multifunctional signaling organelle. Cell Calcium 32:235-249.
Bezprozvanny, I. & Mattson, M. P. (2008) Trends Neurosci. 31, 454-463.
Bijur G N, Jope R S. (2003) Glycogen synthase kinase-3β is highly activated in nuclei and mitochondria. Neuroreport., 14, 2415-2419.
Bionda C, Portoukalian J, Schmitt D, Rodriguez-Lafrasse C, Ardail D (2004) Subcellular compartmentalization of ceramide metabolism: ER-MAM (mitochondria-associated membrane) and/or mitochondria? Biochem. J. 382:527-533.
Bionda, J. Portoukalian, D. Schmitt, C. Rodriguez-Lafriasse, D. Ardail, Biochem. J. 382, 527 (2004)
Bonilla E, Tanji K, DiMauro S, Schon E A. (1997) Neurologic and neuropathologic features of mitochondrial encephalomyopathies. In: Mitochondria and Free Radicals in Neurodegenerative Diseases (eds. M F Beal, N Howell, I Bodis-Wollner), pp 271-279. Wiley-Liss, Inc., New York.
Borchelt et al., Neuron, 1996, 17, 1005-1013
Braak E, Braak H (1997) Alzheimer's disease: transiently developing dendritic changes in pyramidal cells of sector CA1 of the Ammon's horn. Acta Neuropathol. 93:323-325.
Breckenridge D G, Stojanovic M, Marcellus R C, Shore G C (2003) Caspase cleavage product of BAP31 induces mitochondrial fission through endoplasmic reticulum calcium signals, enhancing cytochrome c release to the cytosol. J. Cell Biol. 160:1115-11127.
Brini M, Pinton P, King M P, Davidson M, Schon E A, Rizzuto R (1999) A calcium signaling defect in the pathogenesis of a mitochondrial DNA inherited oxidative phosphorylation deficiency. Nature Med. 5:951-954.
Brock R, Hamelers I H, Jovin T M (1999) Comparison of fixation protocols for adherent cultured cells applied to a GFP fusion protein of the epidermal growth factor receptor. Cytometry 35:353-362.
Broeckhoven C, De Deyn P P (2004) A novel presenilin 1 mutation associated with Pick's disease but not b-amyloid plaques. Ann. Neurol. 55:617-626.
Browman, D. T., Resek, M. E., Zajchowski, L. D., & Robbins, S. M. (2006) J. Cell Sci. 119, 3149-3160.
Brunkan A L, Goate A M (2005) Presenilin function and -secretase activity. J. Neurochem. 93:769-792.
Buckman J F, Hernandez H, Kress G J, Votyakova T V, Pal S, Reynolds I J (2001) MitoTracker labeling in primary neuronal and astrocytic cultures: influence of mitochondrial membrane potential and oxidants. J. Neurosci. Methods 104:165-176.
Busciglio J, Hartmann H, Lorenzo A, Wong C, Baumann K, Sommer B, Staufenbiel M, Yankner B A (1997) Neuronal localization of presenilin-1 and association with amyloid plaques and neurofibrillary tangles in Alzheimer's disease. J. Neurosci. 17:5101-5107.
Cai C, Lin P, Cheung K H, Li N, Levchook C, Pan Z, Ferrante C, Boulianne G L, Foskett J K, Danielpour D, Ma J (2006) The presenilin-2 loop peptide perturbs intracellular Ca2+ homeostasis and accelerates apoptosis. J. Biol. Chem. 281:16649-16655.
Cai D, Leem J Y, Greenfield J P, Wang P, Kim B S, Wang R, Lopes K O, Kim S H, Zheng H, Greengard P, Sisodia S S, Thinakaran G, Xu H (2003) Presenilin-1 regulates intracellular trafficking and cell surface delivery of b-amyloid precursor protein. J. Biol. Chem. 278:3446-3454.
Cai Q, Gerwin C, Sheng Z H (2005) Syntabulin-mediated anterograde transport of mitochondria along neuronal processes. J. Cell Biol. 170:959-969.
Capell A, Saffrich R, Olivo J C, Meyn L, Walter J, Grunberg J, Mathews P, Nixon R, Dotti C, Haass C (1997) Cellular expression and proteolytic processing of presenilin proteins is developmentally regulated during neuronal differentiation. J. Neurochem. 69:2432-2440.
Carter, D. A. et al., Nat. Genet. 2:255-256 (1992)
Chada S R, Hollenbeck P J (2003) Mitochondrial movement and positioning in axons: the role of growth factor signaling. J. Exp. Biol. 206:1985-1992.
Chang D T, Reynolds I J (2006) Mitochondrial trafficking and morphology in healthy and injured neurons. Prog. Neurobiol. 80:241-268.
Chang D T, Rintoul G L, Pandipati S, Reynolds I J (2006) Mutant huntingtin aggregates impair mitochondrial movement and trafficking in cortical neurons. Neurobiol. Dis. 22:388-400.
Chartier-Harlin, M. C. et al., Nature 353:844-846 (1991)
Chen F, Hasegawa H, Schmitt-Ulms G, Kawarai T, Bohm C, Katayama T, Gu Y, Sanjo N, Glista M, Rogaeva E, Wakutani Y, Pardossi-Piquard R, Ruan X, Tandon A, Checler F, Marambaud P, Hansen K, Westaway D, St George-Hyslop P, Fraser P (2006) TMP21 is a presenilin complex component that modulates g-secretase but not g-secretase activity. Nature 440:1208-1212.
Chen Q, Nakajima A, Choi S H, Xiong X, Tang Y P (2008) Loss of presenilin function causes Alzheimer's disease-like neurodegeneration in the mouse. J. Neurosci. Res. 86:1615-1625.
Chen Q, Schubert D (2002) Presenilin-interacting proteins. Expert Rev. Mol. Med. 4:1-18.
Choung S Y, Kobayashi T, Inoue J, Takemoto K, Ishitsuka H, Inoue K (1988) Hemolytic activity of a cyclic peptide Ro09-0198 isolated from Streptoverticillium. Biochim. Biophys. Acta 940:171-179.
Chui et al., Nature Med., 1999, 5, 560-564
Cipolat S, Martins de Brito O, Dal Zilio B, Scorrano L (2004) OPA1 requires mitofusin 1 to promote mitochondrial fusion. Proc. Natl. Acad. Sci. USA 101:15927-15932.
Citron et al., Nature Med., 1997, 3, 67-72; Chui et al., Nature Med., 1999, 5, 560-564 Consortium TIMK (2007) A mouse for all reasons. Cell 128:9-13.
Coppola M, Pizzigoni A, Banfi S, Bassi M T, Casari G, Incerti B. (2000) Identification and characterization of YME1L1, a novel paraplegin-related gene. Genomics, 66, 48-54.
Corder E H, Saunders A M, Strittmatter W J, Schmechel D E, Gaskell P C, Small G W, Roses A D, Haines J L, Pericak-Vance M A (1993) Gene dose of apolipoprotein E type 4 allele and the risk of Alzheimer's disease in late onset families. Science 261:921-923.
Csordas G, Hajnoczky G (2001) Sorting of calcium signals at the junctions of endoplasmic reticulum and mitochondria. Cell Calcium 29:249-262.
Csordas G, Renken C, Varnai P, Walter L, Weaver D, Buttle K F, Balla T, Mannella C A, Hajnoczky G (2006) Structural and functional features and significance of the physical linkage between ER and mitochondria. J. Cell Biol. 174:915-921.
Cui Z, Vance J E, Chen M H, Voelker D R, Vance D E (1993) Cloning and expression of a novel phosphatidylethanolamine N-methyltransferase. A specific biochemical and cytological marker for a unique membrane fraction in rat liver. J. Biol. Chem. 268:16655-16663.
Cupers P, Bentahir M, Craessaerts K, Orlans I, Vanderstichele H, Saftig P, De Strooper B, Annaert W (2001) The discrepancy between presenilin subcellular localization and g secretase processing of amyloid precursor protein. J. Cell Biol. 154:731-740.
Cupers P, Orlans I, Craessaerts K, Annaert W, De Strooper B (2001) The amyloid precursor protein (APP)-cytoplasmic fragment generated by g-secretase is rapidly degraded but distributes partially in a nuclear fraction of neurones in culture. J. Neurochem. 78:1168-1178.
De Strooper B (2003) Aph-1, Pen-2, and nicastrin with presenilin generate an active g-secretase complex. Neuron 38:9-12.
De Strooper B, Beullens M, Contreras B, Levesque L, Craessaerts K, Cordell B, Moechars D, Bollen M, Fraser P, George-Hyslop P S, Van Leuven F (1997) Phosphorylation, subcellular localization, and membrane orientation of the Alzheimer's disease-associated presenilins. J. Biol. Chem. 272:3590-3598.
De Strooper et al., Nature, 1998, 391, 387-390
De Vos K, Severin F, Van Herreweghe F, Vancompernolle K, Goossens V, Hyman A, Grooten J (2000) Tumor necrosis factor induces hyperphosphorylation of kinesin light chain and inhibits kinesin-mediated transport of mitochondria. J. Cell Biol. 149:1207-1214.
Delettre C, Lenaers G, Griffoin J M, Gigarel N, Lorenzo C, Belenguer P, Pelloquin L, Grosgeorge J, Turc-Carel C, Perret E, Astarie-Dequeker C, Lasquellec L, Arnaud B, Dermaut B, Kumar-Singh S, Engelborghs S, Theuns J, Rademakers R, Saerens J, Pickut B A, Peeters K, Van Den Broeck M, Vennekens K, Claes S, Cruts M, Cras P, Martin J J, Van Devi G, Fotiou A, Jyrinji D, Tycko B, DeArmand S, Rogaeva E, Song Y Q, Medieros H, Liang Y, Orlacchio A, Williamson J, St George-Hyslop P, Mayeux R (2000) Novel presenilin 1 mutations associated with early onset of dementia in a family with both early-onset and late-onset Alzheimer disease. Arch. Neurol. 57:1454-1457.
Dillon C, Goda Y (2005) The actin cytoskeleton: integrating form and function at the synapse. Annu. Rev. Neurosci. 28:25-55.
DiMauro S, Schon E A. (2003) Mitochondrial respiratory-chain diseases. N. Engl. J. Med., 348, 2656-2668.
Doan A, Thinakaran G, Borchelt D R, Slunt H H, Ratovitsky T, Podlisny M, Selkoe D J, Seeger M, Gandy S E, Price D L, Sisodia S S. (1996) Protein topology of presenilin 1. Neuron, 17, 1023-1030.
Dong H, Martin M V, Chambers S, Csernansky J G (2007) Spatial relationship between synapse loss and b-amyloid deposition in Tg2576 mice. J. Comp. Neurol. 500:311-321.
Donoviel D B, Hadjantonakis A K, Ikeda M, Zheng H, Hyslop P S, Bernstein A (1999) Mice lacking both presenilin genes exhibit early embryonic patterning defects. Genes Dev. 13:2801-2810.
Ducommun B, Kaplan J, Hamel C P (2000) Nuclear gene OPA1, encoding a mitochondrial dynamin-related protein, is mutated in dominant optic atrophy. Nat. Genet. 26:207-210.
Duff K, Eckman C, Zehr C, Yu X, Prada C M, Perez-tur J, Hutton M, Buee L, Harigaya Y, Yager D, Morgan D, Gordon M N, Holcomb L, Refolo L, Zenk B, Hardy J, Younkin S (1996) Increased amyloid-b42(43) in brains of mice expressing mutant presenilin 1. Nature 383:710-713.
Duff K, Eckman C, Zehr C, Yu X, Prada C M, Perez-tur J, Hutton M, Buee L, Harigaya Y, Yager D, Morgan D, Gordon M N, Holcomb L, Refolo L, Zenk B, Hardy J, Younkin S (1996) Increased amyloid-b42(43) in brains of mice expressing mutant presenilin 1. Nature 383:710-713.
Eautier-Stein et al., Nucl. Acids Res. 31, 5238 (2003).
Ebneth A, Godemann R, Stamer K, Illenberger S, Trinczek B, Mandelkow E. (1998) Overexpression of tau protein inhibits kinesin-dependent trafficking of vesicles, mitochondria, and endoplasmic reticulum: implications for Alzheimer's disease. J. Cell Biol., 143, 777-794.
Eckman, C. B. et al., Hum. Mol. Genet. 6:2087-2089 (1997)
Efthimiopoulos S, Shao Z, Wisniewski T, Robakis N K (2002) A presenilin-1/g-secretase cleavage releases the E-cadherin intracellular domain and regulates disassembly of adherens junctions. EMBO J. 21:1948-1956.
Eggett C J, Crosier S, Manning P, Cookson M R, Menzies F M, McNeil C J, Shaw P J (2000) Development and characterisation of a glutamate-sensitive motor neurone cell line. J. Neurochem. 74:1895-1902.
Emoto K, Kuge O, Nishijima M, Umeda M (1999) Isolation of a Chinese hamster ovary cell mutant defective in intramitochondrial transport of phosphatidylserine. Proc. Natl. Acad. Sci. USA 96:12400-12405.
Endo S, Ishiguro S, Tamai M (1999) Possible mechanism for the decrease of mitochondrial aspartate aminotransferase activity in ischemic and hypoxic rat retinas. Biochim. Biophys. Acta 1450:385-396.
Feldkamp T, Kribben A, Weinberg J M (2005) Assessment of mitochondrial membrane potential in proximal tubules after hypoxia-reoxygenation. Am. J. Physiol. Renal Physiol. 288:F1092-F1102.
Ferreirinha F, Quattrini A, Pirozzi M, Valsecchi V, Dina G, Broccoli V, Auricchio A, Piemonte F, Tozzi G, Gaeta L, Casari G, Ballabio A, Rugarli E I (2004) Axonal degeneration in paraplegin deficient mice is associated with abnormal mitochondria and impairment of axonal transport. J. Clin. Invest. 113:231-242.
Filippin L, Abad M C, Gastaldello S, Magalhaes P J, Sandona D, Pozzan T (2005) Improved strategies for the delivery of GFP-based Ca2+ sensors into the mitochondrial matrix. Cell Calcium 37:129-136.
Fliegner K H, Kaplan M P, Wood T L, Pintar J E, Liem R K (1994) Expression of the gene for the neuronal intermediate filament protein alpha-internexin coincides with the onset of neuronal differentiation in the developing rat nervous system. J. Comp. Neurol. 342:161-173.
Foster, L. J. & Chan, Q. W. (2007) Subcell. Biochem. 43, 35-47.
Fransson A, Ruusala A, Aspenstrom P (2003) Atypical Rho GTPases have roles in mitochondrial homeostasis and apoptosis. J. Biol. Chem. 278:6495-6502.
Fransson S, Ruusala A, Aspenstrom P (2006) The atypical Rho GTPases Miro-1 and Miro-2 have essential roles in mitochondrial trafficking. Biochem. Biophys. Res. Commun 344:500-510.
Friede R L, Ho K C (1977) The relation of axonal transport of mitochondria with microtubules and other axoplasmic organelles. J. Physiol. 265:507-519.
Friedman W J, Ibanez C F, Hallbook F, Persson H, Cain L D, Dreyfus C F, Black I B (1993) Differential actions of neurotrophins in the locus coeruleus and basal forebrain. Exp. Neurol. 119:72-78.
Gaigg B, Simbeni R, Hrastnik C, Paltauf F, Daum G (1995) Characterization of a microsomal subfraction associated with mitochondria of the yeast, Saccharomyces cerevisiae. Involvement in synthesis and import of phospholipids into mitochondria. Biochim. Biophys. Acta 1234:214-220.
Gandy S (2005) The role of cerebral amyloid b accumulation in common forms of Alzheimer disease. J. Clin. Invest. 115:1121-1129.
Ganes et. al., 1995, Nature 373:523
Georgakopoulos A, Marambaud P, Efthimiopoulos S, Shioi J, Cui W, Li H C, Schutte M, Gordon R, Holstein G R, Martinelli G, Mehta P, Friedrich V L, Jr., Robakis N K (1999) Presenilin-1 forms complexes with the cadherin/catenin cell-cell adhesion system and is recruited to intercellular and synaptic contacts. Mol. Cell. 4:893-902.
Giannakopoulos P, Kovari E, Buee L, Shioi J, H of P R, Robakis N K, Bouras C (1999) Presenilin-1 expression in Pick's disease. Acta Neuropathol (Ber1) 98:488-492.
Glater E E, Megeath L J, Stowers R S, Schwarz T L (2006) Axonal transport of mitochondria requires Milton to recruit kinesin heavy chain and is light chain independent. J. Cell Biol. 173:545-557.
Goate A. M. et al., Nature 349:704-706 (1991)
Goedert M, Spillantini M G (2006) A century of Alzheimer's disease. Science 314:777-781.
Goedert, M. & Spillantini, M. G. (2006) Science 314, 777-781.
Goetz J G, Nabi I R (2006) Interaction of the smooth endoplasmic reticulum and mitochondria. Biochem. Soc. Trans. 34:370-373.
Goetz, I. R Nabi, Biochem. Soc. Trans. 34, 370 (2006).
Gómez-Ramos, P. & Asunción Morán, M. (2007) J. Alzheimers Dis. 11, 53-59.
Grimm, M. O., Grimm, H. S., Pätzold, A. J., Zinser, E. G., Halonen, R., Duering, M., Tschäpe, J. A., De Strooper, B., Müller, U., Shen, J., et al. (2005) Nat. Cell Biol. 7, 1118-1123.
Grziwa, B., Grimm, M. O., Masters, C. L., Beyreuther, K., Hartmann, T., & Lichtenthaler, S. F. (2003) J. Biol. Chem. 278, 6803-6808.
Gu Y, Sanjo N, Chen F, Hasegawa H, Petit A, Ruan X, Li W, Shier C, Kawarai T, Schmitt-Ulms G, Westaway D, St George-Hyslop P, Fraser PE (2004) The presenilin proteins are components of multiple membrane-bound complexes that have different biological activities. J. Biol. Chem. 279:31329-31336.
Guo Q, Fu W, Sopher B L, Miller M W, Ware C B, Martin G M, Mattson M P. (1999) Increased vulnerability of hippocampal neurons to excitotoxic necrosis in presenilin-1 mutant knock-in mice. Nature Med., 5, 101-106.
Guo Q, Sebastian L, Sopher B L, Miller M W, Ware C B, Martin G M, Mattson M P. (1999) Increased vulnerability of hippocampal neurons from presenilin-1 mutant knock-in mice to amyloid β-peptide toxicity: central roles of superoxide production and caspase activation. J. Neurochem., 72, 1019-1029.
Guo X, Macleod G T, Wellington A, Hu F, Panchumarthi S, Schoenfield M, Marin L, Charlton M P, Atwood H L, Zinsmaier K E (2005) The GTPase dMiro is required for axonal transport of mitochondria to Drosophila synapses. Neuron 47:379-393.
Gurskaya N G, Verkhusha V V, Shcheglov A S, Staroverov D B, Chepurnykh T V, Fradkov A F, Lukyanov S, Lukyanov K A (2006) Engineering of a monomeric green-to-red photoactivatable fluorescent protein induced by blue light. Nat. Biotechnol. 24:461-465.
Hajnoczky G, Csordas G, Yi M (2002) Old players in a new role: mitochondria-associated membranes, VDAC, and ryanodine receptors as contributors to calcium signal propagation from endoplasmic reticulum to the mitochondria. Cell Calcium 32:363-377.
Halliday G M, Song Y J, Lepar G, Brooks W S, Kwok J B, Kersaitis C, Gregory G, Shepherd C E, Rahimi F, Schofield P R, Kril J J (2005) Pick bodies in a family with presenilin-1 Alzheimer's disease. Ann. Neurol. 57:139-143.
Hancock J F (2006) Lipid rafts: contentious only from simplistic standpoints. Nature Rev. Mol. Cell. Biol. 7:456-462.
Handler M, Yang X, Shen J (2000) Presenilin-1 regulates neuronal differentiation during neurogenesis. Development 127:2593-2606.
Hansson C A, Frykman S, Farmery M R, Tjernberg L O, Nilsberth C, Pursglove S E, Ito A, Winblad B, Cowburn R F, Thyberg J, Ankarcrona M (2004) Nicastrin, presenilin, APH 1, and PEN-2 form active -secretase complexes in mitochondria. J. Biol. Chem. 279:51654-51660.
Hansson E M, Stromberg K, Bergstedt S, Yu G, Naslund J, Lundkvist J, Lendahl U (2005) Aph-1 interacts at the cell surface with proteins in the active -secretase complex and membrane-tethered Notch. J. Neurochem. 92:1010-1020.
Hardy J, Selkoe D J (2002) The amyloid hypothesis of Alzheimer's disease: progress and problems on the road to therapeutics. Science 297:353-356.
Hayashi T, Su T P (2007) Sigma-1 receptor chaperones at the ER-mitochondrion interface regulate Ca(2+) signaling and cell survival. Cell 131:596-610.
Hayashi, T. & Fujimoto, M. (2010) Mol. Pharmacol. 77, 517-528.
Hayashi, T. P. Su, Cell 131, 596 (2007).
Hayashi, T., Rizzuto, R., Hajnoczky, G., & Su, T. P. (2009) Trends Cell Biol. 19, 81-88.
Hayrapetyan V, Rybalchenko V, Rybalchenko N, Koulen P (2008) The N-terminus of presenilin-2 increases single channel activity of brain ryanodine receptors through direct protein-protein interaction. Cell Calcium in press:
Hendriks, L. et al., Nat. Genet. 1:218-221 (1992)
Herreman, A., Serneels, L., Annaert, W., Collen, D., Schoonjans, L., & De Strooper, B. (2000) Nat. Cell Biol. 2, 461-462.
Herrington J, Park Y B, Babcock D F, Hille B (1996) Dominant role of mitochondria in clearance of large Ca2+ loads from rat adrenal chromaffin cells. Neuron 16:219-228.
Hirai K, Aliev G, Nunomura A, Fujioka H, Russell R L, Atwood C S, Johnson A B, Kress Y, Vinters H V, Tabaton M, Shimohama S, Cash A D, Siedlak S L, Harris P L, Jones P K, Petersen R B, Perry G, Smith M A (2001) Mitochondrial abnormalities in Alzheimer's disease. J. Neurosci. 21:3017-3023.
Holcomb et al., Nature Med., 1998, 4, 97-100
Hollenbeck P J (1996) The pattern and mechanism of mitochondrial transport in axons. Front. Biosci. 1:d91-d102.
Hollenbeck P J (2005) Mitochondria and neurotransmission: evacuating the synapse. Neuron 47:331-333.
Hollenbeck P J, Saxton W M (2005) The axonal transport of mitochondria. J. Cell Sci. 118:5411-5419.
Ito E, Oka K, Etcheberrigaray R, Nelson T J, McPhie D L, Tofel-Grehl B, Gibson G E, Alkon D L (1994) Internal Ca2+ mobilization is altered in fibroblasts from patients with Alzheimer disease. Proc. Natl. Scad. Sci. USA 91:534-538.
Jia, C. L. Moulson, Z. Pei, J. H. Miner, P. A. Watkins, J. Bio Chem. 282, 20573 (2007).
Jones, C. T. et al., Nat. Genet. 1:306-309 (1992)
Jorba N, Area E, Ortin J (2008) Oligomerization of the influenza virus polymerase complex in vivo. J. Gen. Virol. 89:520-524.
Jousset H, Frieden M, Demaurex N (2007) STIM1 knockdown reveals that store-operated Ca2+ channels located close to sarco/endoplasmic Ca2+ ATPases (SERCA) pumps silently refill the endoplasmic reticulum. J. Biol. Chem. 282:11456-11464.
Kaasik A, Veksler V, Boehm E, Novotova M, Minajeva A, Ventura-Clapier R (2001) Energetic crosstalk between organelles: architectural integration of energy production and utilization. Circ. Res. 89:153-159.
Kaether C, Lammich S, Edbauer D, Ertl M, Rietdorf J, Capell A, Steiner H, Haass C (2002) Presenilin-1 affects trafficking and processing of APP and is targeted in a complex with nicastrin to the plasma membrane. J. Cell Biol. 158:551-561.
Kaether C, Schmitt S, Willem M, Haass C (2006) Amyloid precursor protein and Notch intracellular domains are generated after transport of their precursors to the cell surface. Traffic 7:408-415.
Kamal A, Almenar-Queralt A, LeBlanc J F, Roberts E A, Goldstein L S. (2001) Kinesin-mediated axonal transport of a membrane compartment containing β secretase and presenilin-1 requires APP. Nature, 414, 643-648.
Kamal A, Stokin G B, Yang Z, Xia C H, Goldstein L S. (2000) Axonal transport of amyloid precursor protein is mediated by direct binding to the kinesin light chain subunit of kinesin-I. Neuron, 28, 449-459.
Kamino, K. et al., Am. J. Hum. Genet. 51:998-1014 (1992)
Kim, S. H., Lah, J. J., Thinakaran, G., Levey, A., & Sisodia, S. S. (2000) Neurobiol. Dis. 7, 99-117.
Kimura N, Nakamura S I, Honda T, Takashima A, Nakayama H, Ono F, Sakakibara I, Doi K, Kawamura S, Yoshikawa Y (2001) Age-related changes in the localization of presenilin-1 in cynomolgus monkey brain. Brain Res. 922:30-41.
King T D, Bijur G N, Jope R S. (2001) Caspase-3 activation induced by inhibition of mitochondrial complex I is facilitated by glycogen synthase kinase-3β and attenuated by lithium. Brain Res., 919, 106-114.
Kinsella T M, Nolan G P (1996) Episomal vectors rapidly and stably produce high-titer recombinant retrovirus. Hum. Gene Ther. 7:1405-1413.
Kinsella, G. P. Nolan, Hum. Gene Ther. 7, 1405 (1996).
Klement P, Nijtmans L G, Van den Bogert C, Houstk J (1995) Analysis of oxidative phosphorylation complexes in cultured human fibroblasts and amniocytes by blue native-electrophoresis using mitoplasts isolated with the help of digitonin. Anal. Biochem. 231:218-224.
Klivenyi P, Starkov A A, Calingasan N Y, Gardian G, Browne S E, Yang L, Bubber P, Gibson G E, Patel M S, Beal M F (2004) Mice deficient in dihydrolipoamide dehydrogenase show increased vulnerability to MPTP, malonate and 3-nitropropionic acid neurotoxicity. J. Neurochem. 88:1352-1360.
Kopach O, Kruglikov I, Pivneva T, Voitenko N, Fedirko N (2008) Functional coupling between ryanodine receptors, mitochondria and Ca(2+) ATPases in rat submandibular acinar cells. Cell Calcium 43:469-481.
Kopach, I. Kruglikov, T. Pivneva, N. Voitenko, N. Fedirko, Cell Calcium in press (2007).
Kottgen et al., EMBO J. 24, 705 (2005).
Köttgen M, Benzing T, Simmen T, Tauber R, Buchholz B, Feliciangeli S, Huber T B, Schermer B, Kramer-Zucker A, Hopker K, Simmen K C, Tschucke C C, Sandford R, Kim E, Thomas G, Walz G (2005) Trafficking of TRPP2 by PACS proteins represents a novel mechanism of ion channel regulation. EMBO J. 24:705-716.
Kristian T, Hopkins I B, McKenna M C, Fiskum G (2006) Isolation of mitochondria with high respiratory control from primary cultures of neurons and astrocytes using nitrogen cavitation. J. Neurosci. Meth. 152:136-143.
Lai J C, Cooper A J (1986) Brain alpha-ketoglutarate dehydrogenase complex: kinetic properties, regional distribution, and effects of inhibitors. J. Neurochem. 47:1376-1386.
Lamb et al., Nature Neurosci., 1999, 2, 695-697
Laudon H, Hansson E M, Melen K, Bergman A, Farmery M R, Winblad B, Lendahl U, von Heijne G, Naslund J (2005) A nine transmembrane domain topology for presenilin 1. J. Biol. Chem. in press:
Lee, S. J., Liyanage, U., Bickel, P. E., Xia, W., Lansbury, P. T., Jr., & Kosik, K. S. (1998) Nat. Med. 4, 730-734.
Leissring M A, Akbari Y, Fanger C M, Cahalan M D, Mattson M P, LaFerla F M (2000)
Capacitative calcium entry deficits and elevated luminal calcium content in mutant presenilin-1 knockin mice. J. Cell Biol. 149:793-798.
Leissring M A, Murphy M P, Mead T R, Akbari Y, Sugarman M C, Jannatipour M, Anliker B, Muller U, Saftig P, De Strooper B, Wolfe M S, Golde T E, LaFerla F M (2002) A physiologic signaling role for the g-secretase-derived intracellular fragment of APP. Proc. Natl. Acad. Sci. USA 99:4697-4702.
Leissring M A, Parker I, LaFerla F M (1999) Presenilin-2 mutations modulate amplitude and kinetics of inositol 1, 4, 5-trisphosphate-mediated calcium signals. J. Biol. Chem. 274:32535-32538.
Leissring M A, Paul B A, Parker I, Cotman C W, LaFerla F M (1999) Alzheimer's presenilin-1 mutation potentiates inositol 1,4,5-trisphosphate-mediated calcium signaling in Xenopus oocytes. J. Neurochem. 72:1061-1068.
Leong S F, Lai J C, Lim L, Clark J B (1984) The activities of some energy-metabolising enzymes in nonsynaptic (free) and synaptic mitochondria derived from selected brain regions. J. Neurochem. 42:1306-1312.
Leuenberger, N. A. Bally, G. Schatz, C. M. Koehler, EM30 J. 18,48 16 (1999).
Levitan D, Greenwald I (1998) Effects of SEL-12 presenilin on LIN-12 localization and function in Caenorhabditis elegans. Development 125:3599-3606.
Levy E. et al., Science 248:1124-1126 (1990)
Lewin T M, Van Horn C G, Krisans S K, Coleman R A (2002) Rat liver acyl-CoA synthetase 4 is a peripheral-membrane protein located in two distinct subcellular organelles, peroxisomes, and mitochondrial-associated membrane. Arch. Biochem. Biophys. 404:263-270.
Lewin, C. G. Van Horn, S. K. Krisans, R. A, Coleman, Arch. Bioohem. Biophys. 404, 263 (2002)
Li D, Parks S B, Kushner J D, Nauman D, Burgess D, Ludwigsen S, Partain J, Nixon R R, Allen C N, Irwin R P, Jakobs P M, Litt M, Hershberger R E (2006) Mutations of Presenilin genes in dilated cardiomyopathy and heart failure. Am. J. Hum. Genet. 79:1030-1039.
Li X, Greenwald I. (1998) Additional evidence for an eight-transmembrane-domain topology for Caenorhabditis elegans and human presenilins. Proc. Natl. Acad. Sci. USA, 95, 7109-7114.
Li Z, Okamoto K, Hayashi Y, Sheng M (2004) The importance of dendritic mitochondria in the morphogenesis and plasticity of spines and synapses. Cell 119:873-887.
Macdonald, J. L. & Pike, L. J. (2005) J. Lipid Res. 46, 1061-1067.
Magrane J, Christensen R A, Rosen K M, Veereshwarayya V, Querfurth H W (2006) Dissociation of ERK and Akt signaling in endothelial cell angiogenic responses to beta amyloid. Exp. Cell Res. 312:996-1010.
Magrane J, Rosen K M, Smith R C, Walsh K, Gouras G K, Querfurth H W (2005) Intraneuronal b-amyloid expression downregulates the Akt survival pathway and blunts the stress response. J. Neurosci. 25:10960-10969.
Makino, A., Baba, T., Fujimoto, K., Iwamoto, K., Yano, Y., Terada, N., Ohno, S., Sato, S. B., Ohta, A., Umeda, M., et al. (2003) J. Biol. Chem. 278, 3204-3209.
Man W C, Miyazaki M, Chu K, Ntambi J (2006) Colocalization of SCD1 and DGAT2: implying preference for endogenous monounsaturated fatty acids in triglyceride synthesis. J. Lipid Res. 47:1928-1939.
Man, M. Miyazaki, K. Chu, J. Ntambi, J. Lipid Res 47, 1928 (2006).
Manfredi G, Fu J, Ojaimi J, Sadlock J E, Kwong J Q, Guy J, Schon E A (2002) Rescue of a deficiency in ATP synthesis by transfer of MTATP6, a mitochondrial DNA encoded gene, to the nucleus. Nat. Genet. 25:394-399.
Manfredi G, Spinazzola A, Checcarelli N, Naini A (2001) Assay of mitochondrial ATP synthesis in animal cells. Methods Cell Biol. 65:133-145.
Marambaud P, Shioi J, Serban G, Georgakopoulos A, Sarner S, Nagy V, Baki L, Wen P, Efthimiopoulos S, Shao Z, Wisniewski T, Robakis N K (2002) A presenilin-1/secretase cleavage releases the Ecadherin intracellular domain and regulates disassembly of adherens junctions. EMBO J. 21:1948-1956.
Marambaud P, Wen P H, Dutt A, Shioi J, Takashima A, Siman R, Robakis N K (2003) A CBP binding transcriptional repressor produced by the PS1/b-cleavage of N-cadherin is inhibited by PS1 FAD mutations. Cell 114:635-645.
Martínez-Abundis, E., Correa, F., Pavan, N., & Zazueta, C. (2009) FEBS J. 276, 5579-5588. Mattson M P (2004) Pathways towards and away from Alzheimer's disease. Nature 430:631-639.
Mattson M P, LaFerla F M, Chan S L, Leissring M A, Shepel P N, Geiger J D (2000) Calcium signaling in the ER: its role in neuronal plasticity and neurodegenerative disorders. Trends Neurosci. 23:222-229.
Medema R H (2004) Optimizing RNA interference for application in mammalian cells. Biochem. J. 380:593-603.
Melan M A, Sluder G (1992) Redistribution and differential extraction of soluble proteins in permeabilized cultured cells. Implications for immunofluorescence microscopy. J. Cell Sci. 101:731-743.
Mellgren, R. L. (2008) J. Biochem. Biophys. Methods 70, 1029-1036.
Mendes et al, J. Biol. Chem. 2 80, 40892 (2005).
Migliaccio, C. V. Nicchitta, G. Blohel, J. Cell Biol. 117, 15 (1992).
Miller K E, Sheetz M P (2004) Axonal mitochondrial transport and potential are correlated. J. Cell Sci. 117:2791-2804.
Morfini G, Pigino G, Beffert U, Busciglio J, Brady S T. (2002) Fast axonal transport misregulation and Alzheimer's disease. Neuromolecular Med., 2, 89-99.
Morfini G, Szebenyi G, Elluru R, Ratner N, Brady S T (2002) Glycogen synthase kinase 3 phosphorylates kinesin light chains and negatively regulates kinesin-based motility. EMBO J. 21:281-293.
Morrow, I. C. & Parton, R. G. (2005) Traffic 6, 725-740.
Mullan, M. et al Nat. Genet. 1:345-347 (1992)
Murphy, E. J., Huang, H. M., Cowburn, R. F., Lannfelt, L., & Gibson, G. E. (2006) Brain Res. Bull. 69, 79-85.
Murrell, J. et al., Science 254:97-99 (1991)
Myhill N, Lynes E M, Nanji J A, Blagoveshchenskaya A D, Fei H, Simmen K C, Cooper T J, Thomas G, Simmen T (2008) The subcellular distribution of calnexin is mediated by PACS-2. Mol. Biol. Cell in press:
Nagai T, Sawano A, Park E S, Miyawaki A (2001) Circularly permuted green fluorescent proteins engineered to sense Ca2+. Proc. Natl. Acad. Sci. USA 98:3197-3202.
Nakai T, Yamasaki A, Sakaguchi M, Kosaka K, Mihara K, Amaya Y, Miura S. (1999) Membrane topology of Alzheimer's disease-related presenilin 1. Evidence for the existence of a molecular species with a seven membrane-spanning and one membrane-embedded structure. J. Biol. Chem., 274, 23647-23658.
Nakano et al., Eur. J. Neurosci., 1999, 11, 2577-2581
Nakaya Y, Yamane T, Shiraishi H, Wang H Q, Matsubara E, Sato T, Dolios G, Wang R, De Strooper B, Shoji M, Komano H, Yanagisawa K, Ihara Y, Fraser P, St George-Hyslop P, Nishimura M (2005) Random mutagenesis of presenilin-1 identifies novel mutants exclusively generating long amyloid b-peptides. J. Biol. Chem. 280:19070-19077.
Nangaku M, Sato-Yoshitake R, Okada Y, Noda Y, Takemura R, Yamazaki H, Hirokawa N (1994) KIF1B, a novel microtubule plus end-directed monomeric motor protein for transport of mitochondria. Cell 79:1209-1220.
Naruse S, Thinakaran G, Luo J J, Kusiak J W, Tomita T, Iwatsubo T, Qian X, Ginty D D, Price D L, Borchelt D R, Wong P C, Sisodia S S (1998) Effects of PS1 deficiency on membrane protein trafficking in neurons. Neuron 21:1213-1221.
Nelson O, Tu H, Lei T, Bentahir M, de Strooper B, Bezprozvanny I (2007) Familial Alzheimer diseaselinked mutations specifically disrupt Ca2+ leak function of presenilin 1. J. Clin. Invest. 117:1230-1239.
Niemann A, Ruegg M, La Padula V, Schenone A, Suter U (2005) Ganglioside-induced differentiation associated protein 1 is a regulator of the mitochondrial network: new implications for Charcot-Marie-Tooth disease. J. Cell Biol 170:1067-1078.
Nikolic M, Dudek H, Kwon Y T, Ramos Y F, Tsai L H (1996) The cdk5/p35 kinase is essential for neurite outgrowth during neuronal differentiation. Genes Dev. 10:816-825.
North R A (2002) Molecular physiology of P2X receptors. Physiol. Rev. 82:1013-1067.
Ojaimi J, Pan J, Santra S, Snell W J, Schon E A (2002) An algal nucleus-encoded subunit of mitochondrial ATP synthase rescues a defect in the analogous human mitochondrial encoded subunit. Mol. Biol. Cell 13:3836-3844.
Orme M H, Giannini A L, Vivanco M D, Kypta R M. (2003) Glycogen synthase kinase-3 and Axin function in a β-catenin-independent pathway that regulates neurite outgrowth in neuroblastoma cells. Mol. Cell. Neurosci., 24, 673-86.
Ostrom, R. S. & Liu, X. (2007) Meth. Mol. Biol. 400, 459-468.
Pack-Chung E, Meyers M B, Pettingell W P, Moir R D, Brownawell A M, Cheng I, Tanzi R E, Kim T W (2000) Presenilin 2 interacts with sorcin, a modulator of the ryanodine receptor. J. Biol. Chem. 275:14440-14445.
Pani, A., Dessi, S., Diaz, G., La Colla, P., Abete, C., Mulas, C., Angius, F., Cannas, M. D., Orru, C. D., Cocco, P. L., et al. (2009) J. Alzheimers Dis. 18, 829-841.
Pasternak S H, Bagshaw R D, Guiral M, Zhang S, Ackerley C A, Pak B J, Callahan J W, Mahuran D J (2003) Presenilin-1, nicastrin, amyloid precursor protein, and g-secretase activity are co-localized in the lysosomal membrane. J. Biol. Chem. 278:26687-26694.
Peacock, M. L. et al., Ann. Neurol. 35:432-438 (1994)
Peacock, M. L. et al., Neurology 43:1254-1256 (1993)
Pellegrini L, Passer B J, Canelles M, Lefterov I, Ganjei J K, Fowlkes B J, Koonin E V, D'Adamio L. (2001) PAMP and PARL, two novel putative metalloproteases interacting with the COOH-terminus of Presenilin-1 and -2. J. Alzheimers Dis., 3, 181-190.
Pereira A J, Dalby B, Stewart R J, Doxsey S J, Goldstein L S (1997) Mitochondrial association of a plus end-directed microtubule motor expressed during mitosis in Drosophila. J. Cell Biol. 136:1081-1090.
Pettegrew, J. W., Panchalingam, K., Hamilton, R. L., & McClure, R. J. (2001) Neurochem. Res. 26, 771-782.
Pickel V M, Clarke C L, Meyers M B (1997) Ultrastructural localization of sorcin, a 22 kDa calcium binding protein, in the rat caudate-putamen nucleus: association with ryanodine receptors and intracellular calcium release. J. Comp. Neurol. 386:625-634.
Pigino G, Morfini G, Pelsman A, Mattson M P, Brady S T, Busciglio J (2003) Alzheimer's presenilin 1 mutations impair kinesin-based axonal transport. J. Neurosci. 23:4499-4508.
Pilling A D, Horiuchi D, Lively C M, Saxton W M (2006) Kinesin-1 and dynein are the primary motors for fast transport of mitochondria in Drosophila motor axons. Mol. Biol. Cell 17:2057-2068.
Pinton P, Remessi A, Romagnoli A, Prandini A, Rizzuto R (2007) Biosensors for the detection of calcium and pH. Meth. Cell Biol. 80:297-325.
Ponting C P, Hutton M, Nyborg A, Baker M, Jansen K, Golde T E (2002) Identification of a novel family of presenilin homologues. Hum. Mol. Genet. 11:1037-1044.
Pottekat, A. K. Menon, J. Biol. Chem. 279,15743 (2004).
Pozzan T, Rizzuto R, Volpe P, Meldolesi J (1994) Molecular and cellular physiology of intracellular calcium stores. Physiol. Rev. 74:595-636.
Pratico, D. & Delanty, N. (2000) Am. J. Med. 109, 577-585.
Priller C, Dewachter I, Vassallo N, Paluch S, Pace C, Kretzschmar H A, Van Leuven F, Herms J (2007) Mutant presenilin 1 alters synaptic transmission in cultured hippocampal neurons. J. Biol. Chem. 282:1119-1127.
Prinz W A, Grzyb L, Veenhuis M, Kahana J A, Silver P A, Rapoport T A (2000) Mutants affecting the structure of the cortical endoplasmic reticulum in Saccharomyces cerevisiae. J. Cell Biol. 150:461-474.
Puglielli, L., Konopka, G., Pack-Chung, E., Ingano, L. A., Berezovska, O., Hyman, B. T., Chang, T. Y., Tanzi, R. E., & Kovacs, D. M. (2001) Nat. Cell Biol. 3, 905-912.
Registre M, Goetz J G, St Pierre P, Pang H, Lagace M, Bouvier M, Le P U, Nabi I R (2004) The gene product of the gp78/AMFR ubiquitin E3 ligase cDNA is selectively recognized by the 3F3A antibody within a subdomain of the endoplasmic reticulum. Biochem. Biophys. Res. Commun 320:1316-1322.
Rideout H J, Stefanis L (2002) Proteasomal inhibition-induced inclusion formation and death in cortical neurons require transcription and ubiquitination. Mol. Cell. Neurosci. 21:223-238.
Rigaut G, Shevchenko A, Rutz B, Wilm M, Mann M, Seraphin B (1999) A generic protein purification method for protein complex characterization and proteome exploration. Nature Biotechnol. 17:1030-1032.
Rintoul G L, Filiano A J, Brocard J B, Kress G J, Reynolds I J (2003) Glutamate decreases mitochondrial size and movement in primary forebrain neurons. J. Neurosci 23:7881-7888.
Ris L, Dewachter I, Reverse D, Godaux E, Van Leuven F (2003) Capacitative calcium entry induces hippocampal long term potentiation in the absence of presenilin-1. J. Biol. Chem. 278:44393-44399.
Rizzuto R, Carrington W, Tuft R A (1998) Digital imaging microscopy of living cells. Trends Cell Biol. 8:288-292.
Rizzuto R, Nakase H, Darras B, Francke U, Fabrizi G M, Mengel T, Walsh F, Kadenbach B, DiMauro S, Schon E A (1989) A gene specifying subunit VIII of human cytochrome c oxidase is localized to chromosome 11 and is expressed in both muscle and non-muscle tissues. J. Biol. Chem. 264:10595-10600.
Rizzuto R, Pinton P, Carrington W, Fay F S, Fogarty K E, Lifshitz L M, Tuft R A, Pozzan T (1998) Close contacts with the endoplasmic reticulum as determinants of mitochondrial Ca2+ responses. Science 280:1763-1766.
Robb-Gaspers L D, Burnett P, Rutter G A, Denton R M, Rizzuto R, Thomas A P (1998) Integrating cytosolic calcium signals into mitochondrial metabolic responses. EMBO J. 17:4987-5000.
Roberson E D, Scearce-Levie K, Palop J J, Yan F, Cheng I H, Wu T, Gerstein H, Yu G Q, Mucke L (2007) Reducing endogenous tau ameliorates amyloid beta-induced deficits in an Alzheimer's disease mouse model. Science 316:750-754.
Rogaeva E, Meng Y, Lee J H, Gu Y, Kawarai T, Zou F, Katayama T, Baldwin C T, Cheng R, Hasegawa H, Chen F, Shibata N, Lunetta K L, Pardossi-Piquard R, Bohm C, Wakutani Y, Cupples L A, Cuenco K T, Green R C, Pinessi L, Rainero I, Sorbi S, Bruni A, Duara R, Friedland R P, Inzelberg R, Hampe W, Bujo H, Song Y Q, Andersen O M, Willnow T E, Graff-Radford N, Petersen R C, Dickson D, Der S D, Fraser P E, Schmitt Ulms G, Younkin S, Mayeux R, Farrer L A, P. S G-H (2007) The neuronal sortilin-related receptor SORL1 is genetically associated with Alzheimer disease. Nature Genet. in press:
Rose I A, O'Connell E L (1967) Mechanism of aconitase action. I. The hydrogen transfer reaction. J. Biol. Chem. 242:1870-1879.
Rosenthal R E, Hamud F, Fiskum G, Varghese P J, Sharpe S (1987) Cerebral ischemia and reperfusion: prevention of brain mitochondrial injury by lidoflazine. J. Cereb. Blood Flow Metab. 7:752-758.
Rube D A, van der Bliek A M (2004) Mitochondrial morphology is dynamic and varied. Mol. Cell. Biochem. 256:331-339.
Runz H, Rietdorf J, Tomic I, de Bernard M, Beyreuther K, Pepperkok R, Hartmann T (2002) Inhibition of intracellular cholesterol transport alters presenilin localization and amyloid precursor protein processing in neuronal cells. J. Neurosci. 22:1679-1689.
Rusinol A E, Cui Z, Chen M H, Vance J E (1994) A unique mitochondria-associated membrane fraction from rat liver has a high capacity for lipid synthesis and contains pre Golgi secretory proteins including nascent lipoproteins. J. Biol. Chem. 269:27494-27502.
Rusinol, A. E., Cui, Z., Chen, M. H., & Vance, J. E. (1994) J. Biol. Chem. 269, 27494-27502.
Rusinol, Z. Cui, M. H. Chen, J. E. Vance, J. Bid. Chem. 269, 27494 (1994). Rybalchenko V, Hwang S Y, Rybalchenko N, Koulen P (2008) The cytosolic N-terminus of presenilin-1 potentiates mouse ryanodine receptor single channel activity. Int. J. Biochem. Cell Biol. 40:84-97.
Santama N, Er C P, Ong L L, Yu H (2004) Distribution and functions of kinectin isoforms. J. Cell Sci. 117:4537-4549.
Sapoznik S, Ivenshitz M, Segal M (2006) Age-dependent glutamate induction of synaptic plasticity in cultured hippocampal neurons. Learn. Mem. 13:719-727.
Sato T, Diehl T S, Narayanan S, Funamoto S, Ihara Y, De Strooper B, Steiner H, Haass C, Wolfe M S (2007) Active -secretase complexes contain only one of each component. J. Biol. Chem. In press:
Saura C A, Choi S Y, Beglopoulos V, Malkani S, Zhang D, Shankaranarayana Rao B S, Chattarji S, Kelleher R J, 3rd, Kandel E R, Duff K, Kirkwood A, Shen J (2004) Loss of presenilin function causes impairments of memory and synaptic plasticity followed by age dependent neurodegeneration. Neuron 42:23-36.
Schagger H, von Jagow G (1991) Blue native electrophoresis for isolation of membrane protein complexes in enzymatically active form. Anal. Biochem. 199:223-231.
Scheff S W, Price D A, Schmitt F A, Mufson E J (2006) Hippocampal synaptic loss in early Alzheimer's disease and mild cognitive impairment. Neurobiol. Aging 27:1372-1384.
Schneider I, Reverse D, Dewachter I, Ris L, Caluwaerts N, Kuiperi C, Gilis M, Geerts H, Kretzschmar H, Godaux E, Moechars D, Van Leuven F, Herms J (2001) Mutant presenilins disturb neuronal calcium homeostasis in the brain of transgenic mice, decreasing the threshold for excitotoxicity and facilitating long-term potentiation. J. Biol. Chem. 276:11539-11544.
Schon, E. A. & Area-Gomez, E. (2010) J. Alzheimers Dis., in press.
Schumacher M M, Choi J Y, Voelker D R (2002) Phosphatidylserine transport to the mitochondria is regulated by ubiquitination. J. Biol. Chem. 277:51033-51042.
Schwarzman A L, Singh N, Tsiper M, Gregori L, Dranovsky A, Vitek M P, Glabe C G, St George-Hyslop P H, Goldgaber D (1999) Endogenous presenilin 1 redistributes to the surface of lamellipodia upon adhesion of Jurkat cells to a collagen matrix. Proc. Natl. Acad. Sci. USA 96:7932-7937.
Selkoe D J (2002) Alzheimer's disease is a synaptic failure. Science 298:789-791.
Serban G, Kouchi Z, Baki L, Georgakopoulos A, Litterst C M, Shioi J, Robakis N K (2005) Cadherins mediate both the association between PS1 and beta-catenin and the effects of PS1 on beta-catenin stability. J. Biol. Chem. 280:36007-36012.
Shah Z H, Hakkaart G A, Arku B, de Jong L, van der Spek H, Grivell L A, Jacobs H T. (2000) The human homologue of the yeast mitochondrial AAA metalloprotease Yme1p complements a yeast yme1 disruptant. FEBS Lett., 478, 267-70.
Shen et al., Cell, 1997, 89, 629-639
Shen J, Kelleher R J, III (2007) The presenilin hypothesis of Alzheimer's disease: Evidence for a loss-of function pathogenic mechanism. Proc. Natl. Acad. Sci. USA 104:403-409.
Shepherd D, Garland P B (1969) The kinetic properties of citrate synthase from rat liver mitochondria. Biochemical J. 114:597-610.
Sherrington et al., Nature 375, 754 (1995).
Siman R, Reaume A G, Savage M J, Trusko S, Lin Y G, Scott R W, Flood D G. (2000) Presenilin-1 P264L knock-in mutation: differential effects on 43 production, amyloid deposition, and neuronal vulnerability. J. Neurosci., 20, 8717-8726.
Siman R, Velji J (2003) Localization of presenilin-nicastrin complexes and gamma-secretase activity to the trans-Golgi network. J. Neurochem. 84:1143-1153.
Simmen T, Aslan J E, Blagoveshchenskaya A D, Thomas L, Wan L, Xiang Y, Feliciangeli S F, Hung C H, Crump C M, Thomas G (2005) PACS-2 controls endoplasmic reticulum mitochondria communication and Bid-mediated apoptosis. EMBO J. 24:717-729.
Simmen, T., Lynes, E. M., Gesson, K., & Thomas, G. (2010) Biochim. Biophys. Acta, in press.
Simons, K. & Vaz, W. L. (2004) Annu. Rev. Biophys. Biomol. Struct. 33, 269-295.
Simrnenetal., EMBO J. 24, 717 (2005).
Sims N R (1990) Rapid isolation of metabolically active mitochondria from rat brain and subregions using Percoll density gradient centrifugation. J. Neurochem. 55:698-707.
Singh N, Talalayeva Y, Tsiper M, Romanov V, Dranovsky A, Colflesh D, Rudamen G, Vitek M P, Shen J, Yang X, Goldgaber D, Schwarzman A L (2001) The role of Alzheimer's disease-related presenilin 1 in intercellular adhesion. Exp. Cell Res. 263:1-13.
Smaili S S, Hsu Y T, Carvalho A C, Rosenstock T R, Sharpe J C, Youle R J (2003) Mitochondria, calcium and pro-apoptotic proteins as mediators in cell death signaling. Brazil. J. Med. Biol. Res. 36:183-190.
Smith I F, Green K N, LaFerla F M (2005) Calcium dysregulation in Alzheimer's disease: recent advances gained from genetically modified animals. Cell Calcium 38:427-437.
Sorbi S, Nacmias B, Forleo P, Latorraca S, Gobbini I, Bracco L, Piacentini S, Amaducci L (1994) ApoE allele frequencies in Italian sporadic and familial Alzheimer's disease. Neurosci. Lett. 177:100-102.
Starkov A A, Fiskum G, Chinopoulos C, Lorenzo B J, Browne S E, Patel M S, Beal M F (2004) Mitochondrial alpha-ketoglutarate dehydrogenase complex generates reactive oxygen species. J. Neurosci. 24:7779-7788.
Stefani, M. & Liguri, G. (2009) Curr. Alz. Res. 6, 15-29.
Steiner H, Duff K, Capell A, Romig H, Grim M G, Lincoln S, Hardy J, Yu X, Picciano M, Fechteler K, Citron M, Kopan R, Pesold B, Keck S, Baader M, Tomita T, Iwatsubo T, Baumeister R, Haass C (1999) A loss of function mutation of presenilin-2 interferes with amyloid b-peptide production and notch signaling. J. Biol. Chem. 274:28669-28673.
Stokin G B, Lillo C, Falzone T L, Brusch R G, Rockenstein E, Mount S L, Raman R, Davies P, Masliah E, Williams D S, Goldstein L S (2005) Axonopathy and transport deficits early in the pathogenesis of Alzheimer's disease. Science 307:1282-1288.
Stone S J, Vance J E (2000) Phosphatidylserine synthase-1 and -2 are localized to mitochondria associated membranes. J. Biol. Chem. 275:34534-34540.
Stone, J. E. Vance, J. Bid. Chem. 275, 34534 (2000).
Stowers R S, Megeath U, Gorska-Andrzejak J, Meinertzhagen I A, Schwarz T L (2002) Axonal transport of mitochondria to synapses depends on milton, a novel Drosophila protein. Neuron 36:1063-1077.
Su, B., Wang, X., Bonda, D., Perry, G., Smith, M., & Zhu, X. (2010) Mol. Neurobiol. 41, 87-96.
Suhara T, Magrane J, Rosen K, Christensen R, Kim H S, Zheng B, McPhie D L, Walsh K, Querfurth H (2003) Ab42 generation is toxic to endothelial cells and inhibits eNOS function through an Akt/GSK-3b signaling-dependent mechanism. Neurobiol. Aging 24:437-451.
Sun F C, Wei S, Li C W, Chang Y S, Chao C C, Lai Y K (2006) Localization of GRP78 to mitochondria under the unfolded protein response. Biochem. J. 396:31-39.
Takashima A, Murayama M, Murayama O, Kohno T, Honda T, Yasutake K, Nihonmatsu N, Mercken M, Yamaguchi H, Sugihara S, Wolozin B. (1998) Presenilin 1 associates with glycogen synthase kinase-3β and its substrate tau. Proc. Natl. Acad. Sci. USA, 95, 9637-9641.
Takashima A, Sato M, Mercken M, Tanaka S, Kondo S, Honda T, Sato K, Murayama M, Noguchi K, Nakazato Y, Takahashi H (1996) Localization of Alzheimer-associated presenilin 1 in transfected COS-7 cells. Biochem. Biophys. Res. Commun. 227:423-426.
Tanaka Y, Kanai Y, Okada Y, Nonaka S, Takeda S, Harada A, Hirokawa N (1998) Targeted disruption of mouse conventional kinesin heavy chain, kif5B, results in abnormal perinuclear clustering of mitochondria. Cell 93:1147-1158.
Tanji K, Bonilla E (2001) Optical imaging techniques (histochemical, immunohistochemical, and in situ hybridization staining methods) to visualize mitochondria. Methods Cell Biol. 65:311-332.
Tanji K, Vu T H, Schon E A, DiMauro S, Bonilla E. (1999) Kearns-Sayre syndrome: unusual pattern of expression of subunits of the respiratory chain in the cerebellar system. Ann. Neurol., 45, 377-383.
Tarassishin L, Yin Y I, Bassit B, Li Y M (2004) Processing of Notch and amyloid precursor protein by g-secretase is spatially distinct. Proc. Natl. Acad. Sci. USA 101:17050-17055.
Tatebayashi Y, Hague N, Tung Y C, Iqbal K, Grundke-Iqbal I. (2004) Role of tau phosphorylation by glycogen synthase kinase-3{β} in the regulation of organelle transport. J. Cell Sci., 117, 1653-1663.
Terry R D, Masliah E, Salmon D P, Butters N, DeTeresa R, Hill R, Hansen L A, Katzman R (1991) Physical basis of cognitive alterations in Alzheimer's disease: synapse loss is the major correlate of cognitive impairment. Ann. Neurol. 30:572-580.
Tesco G, Tanzi R E. (2000) GSK3 β forms a tetrameric complex with endogenous PS1-CTF/NTF and βcatenin. Effects of the D257/D385A and FAD-linked mutations. Ann. NY Acad. Sci., 920, 227-232.
Trinczek B, Ebneth A, Mandelkow EM, Mandelkow E. (1999) Tau regulates the attachment/detachment but not the speed of motors in microtubule-dependent transport of single vesicles and organelles. J. Cell Sci., 112, 2355-2367.
Tsai A, Carstens R P (2006) An optimized protocol for protein purification in cultured mammalian cells using a tandem affinity purification approach. Nature Protocols 1:2820-2827.
Tu H, Nelson O, Bezprozvanny A, Wang Z, Lee S F, Hao Y H, Serneels L, De Strooper B, Yu G, Bezprozvanny I (2006) Presenilins form ER Ca2+ leak channels, a function disrupted by familial Alzheimer's disease-linked mutations. Cell 126:981-993.
Urano, Y., Hayashi, I., Isoo, N., Reid, P. C., Shibasaki, Y., Noguchi, N., Tomita, T., Iwatsubo, T., Hamakubo, T., & Kodama, T. (2005) J. Lipid. Res 46, 904-912.
Vance D E, Walkey C J, Cui Z (1997) Phosphatidylethanolamine N-methyltransferase from liver. Biochim. Biophys. Acta 1348:142-150.
Vance J E (1990) Phospholipid synthesis in a membrane fraction associated with mitochondria. J. Biol. Chem. 265:7248-7256.
Vance, C. J. Walkey, Z. Cui, Biochim. Biophys Acta 1348, 142 (1997).
Vance, J Biol. Chem. 265, 7248 (1990).
Vance, J. E. (1990) J. Biol. Chem. 265, 7248-7256.
Vance, J. E. (2003) Prog. Nucl. Acid Res. Mol. Biol. 75, 69-111.
Vance, J. E. (2008) J. Lipid Res. 49, 1377-1387.
Vetrivel K S, Cheng H, Kim S H, Chen Y, Barnes N Y, Parent A T, Sisodia S S, Thinakaran G (2005) Spatial segregation of g-secretase and substrates in distinct membrane domains. J. Biol. Chem. 280:25892-25900.
Vetrivel K S, Cheng H, Lin W, Sakurai T, Li T, Nukina N, Wong P C, Xu H, Thinakaran G (2004) Association of -secretase with lipid rafts in post-Golgi and endosome membranes. J. Biol. Chem. 279:44945-44954.
Villani G, Attardi G (2007) Polarographic assays of respiratory chain complex activity. Methods Cell Biol. 80:121-133.
Voelker, D. R. (2000) Biochim. Biophys. Acta 1486, 97-107.
Walsh D M, Selkoe D J (2004) Deciphering the molecular basis of memory failure in Alzheimer's disease. Neuron 44:181-193.
Walter J, Capell A, Grunberg J, Pesold B, Schindzielorz A, Prior R, Podlisny M B, Fraser P, Hyslop P S, Selkoe D J, Haass C (1996) The Alzheimer's disease-associated presenilins are differentially phosphorylated proteins located predominantly within the endoplasmic reticulum. Mol. Med. 2:673-691.
Walther, T. C. & Farese, R. V., Jr. (2009) Biochim. Biophys. Acta 1791, 459-466.
Wang, W., Yang, L., & Huang, H. W. (2007) Biophys. J. 92, 2819-2830.
Weihofen A, Binns K, Lemberg M K, Ashman K, Martoglio B (2002) Identification of signal peptide peptidase, a presenilin-type aspartic protease. Science 296:2215-2218.
Wolfe M S, Kopan R (2004) Intramembrane proteolysis: theme and variations. Science 305:1119-1123.
Wong et al., Nature, 1997, 387, 288-292
Wu W I, Voelker D R (2001) Characterization of phosphatidylserine transport to the locus of phosphatidylserine decarboxylase 2 in permeabilized yeast. J. Biol. Chem. 276:7114-7121.
Xu X, Shi Y, Wu X, Gambetti P, Sui D, Cui M Z. (1999) Identification of a novel PSD 95/Dlg/ZO-1 (PDZ)-like protein interacting with the C terminus of presenilin-1. J. Biol. Chem., 274, 32543-32546.
Xu X, Shi Y C, Gao W, Mao G, Zhao G, Agrawal S, Chisolm G M, Sui D, Cui M Z. (2002) The novel presenilin-1-associated protein is a proapoptotic mitochondrial protein. J. Biol. Chem., 277, 48913-48922.
Yao J K, Wengenack T M, Curran G L, Poduslo J F (2008) Reduced Membrane Lipids in the Cortex of Alzheimer's Disease Transgenic Mice. Nerochem. Res. in press:
Yi M, Weaver D, Hajnoczky G (2004) Control of mitochondrial motility and distribution by the calcium signal: a homeostatic circuit. J. Cell Biol. 167:661-672.
Yoo A S, Cheng I, Chung S, Grenfell T Z, Lee H, Pack-Chung E, Handler M, Shen J, Xia W, Tesco G, Saunders A J, Ding K, Frosch M P, Tanzi R E, Kim T W (2000) Presenilin mediated modulation of capacitative calcium entry. Neuron 27:561-572.
Yu H, Saura C A, Choi S Y, Sun L D, Yang X, Handler M, Kawarabayashi T, Younkin L, Fedeles B, Wilson M A, Younkin S, Kandel E R, Kirkwood A, Shen J (2001) APP processing and synaptic plasticity in presenilin-1 conditional knockout mice. Neuron 31:713-726.
Zenisek D, Matthews G (2000) The role of mitochondria in presynaptic calcium handling at a ribbon synapse. Neuron 25:229-237.
Zhang H, Das S, Li Q Z, Dragatsis I, Repa J, Zeitlin S, Hajnoczky G, Bezprozvanny I (2008) Elucidating a normal function of huntingtin by functional and microarray analysis of huntingtin-null mouse embryonic fibroblasts. BMC Neurosci. in press:
Zhang Y, Oko R, van der Hoorn F A (2004) Rat kinesin light chain 3 associates with spermatid mitochondria. Dev. Biol. 275:23-33.
Zhao C, Takita J, Tanaka Y, Setou M, Nakagawa T, Takeda S, Yang H W, Terada S, Nakata T, Takei Y, Saito M, Tsuji S, Hayashi Y, Hirokawa N (2001) Charcot-Marie Tooth disease type 2A caused by mutation in a microtubule motor KIF1Bb. Cell 105:587-597.
Zheng, Y. Z., Berg, K. B., & Foster, L. J. (2009) J. Lipid Res. 50, 988-998.
Zhou S, Zhou H, Walian P J, Jap B K (2005) CD147 is a regulatory subunit of the -secretase complex in Alzheimer's disease amyloid -peptide production. Proc. Natl. Acad. Sci. USA 102:7499-7504.

What is claimed is:

1. A method for determining whether a subject is predisposed to having a neurodegenerative disease or disorder, the method comprising:
(a) obtaining a biological sample from a subject, and
(b) testing the biological sample to determine whether it exhibits increased endoplasmic reticulum-mitochondrial-associated membrane integrity as compared to endoplasmic reticulum-mitochondrial-associated membrane integrity exhibited in a biological sample obtained from a non-affected individual.

2. The method of claim 1, wherein the biological sample comprises one or more cells.

3. The method of claim 1, wherein the neurodegenerative disease or disorder is Alzheimer's disease.

4. The method of claim 1, wherein the biological sample comprises neuronal cells, fibroblasts, blood cells, or epithelial cells.

5. The method of claim 1, wherein the biological sample comprises a blood sample, a biopsy sample, an autopsy sample, a tissue sample or a urine sample.

6. The method of claim 1, wherein the testing comprises:
(a) determining the ratio of perinuclear mitochondria to non-perinuclear mitochondria in cells of the biological sample (sample ratio), and
(b) comparing the ratio to a reference ratio determined for a normal subject, wherein if the sample ratio is greater than the reference ratio, then the subject is predisposed to having a neurodegenerative disease or disorder.

7. The method of claim 1, wherein the testing comprises:
(a) determining the ratio of punctate mitochondria to non-punctate mitochondria in cells of the biological sample (sample ratio), and
(b) comparing the ratio to a reference ratio determined for a normal subject, wherein if the sample ratio is greater than the reference ratio, then the subject is predisposed to having a neurodegenerative disease or disorder.

8. The method of claim 1, wherein the testing comprises:
(a) determining the amount of communication between the ER and mitochondria in the biological sample (sample amount), and
(b) comparing the amount to a reference amount determined for a normal subject, wherein if the sample amount is more than the reference amount, then the subject is predisposed to having a neurodegenerative disease or disorder.

9. The method of claim 1, wherein the testing comprises:
(a) determining the activity of an ER-MAM-associated protein in the biological sample (sample activity), and
(b) comparing the activity to a reference activity determined for a normal subject, wherein if the sample activity is more than the reference activity, then the subject is predisposed to having a neurodegenerative disease or disorder.

10. The method of claim 1, wherein the testing comprises:
(a) determining the activity of an ER-MAM-associated protein in the biological sample (sample activity), and
(b) comparing the activity to a reference activity determined for a normal subject, wherein if the sample activity is less than the reference activity, then the subject is predisposed to having a neurodegenerative disease or disorder.

11. The method of claim 1, wherein the testing comprises:
(a) determining the rate of conversion of phosphatidylserine to phosphatidylethanolamine in cells of the biological sample (sample rate), and
(b) comparing the rate to a reference rate determined for a normal subject, wherein if the sample rate is more than the reference rate or altered relative to the reference rate, then the subject is predisposed to having a neurodegenerative disease or disorder.

12. The method of claim 1, wherein the testing comprises:
(a) determining the amount of presenilin in endoplasmic reticulum-mitochondrial-associated membrane in the biological sample (sample amount), and
(b) comparing the amount to a reference amount determined for a normal subject, wherein if the sample amount is more than the reference amount, then the subject is predisposed to having a neurodegenerative disease or disorder.

13. The method of claim 1, wherein the testing comprises:
(a) determining the amount mitochondrial movement in the cells of biological sample (sample amount), and
(b) comparing the amount to a reference amount determined for a normal subject, wherein if the sample amount is less than the reference amount, then the subject is predisposed to having a neurodegenerative disease or disorder.

14. The method of claim 1, wherein the testing comprises:
(a) determining the amount of cinnamycin required to kill a fixed percentage of cells in a reference biological sample from a normal subject, and
(b) comparing the amount of cinnamycin required to kill the fixed percentage of cells in the biological sample, wherein if the amount of cinnamycin required to kill the fixed percentage of cells in the biological sample is less or different than the amount of cinnamycin required to kill the fixed percentage of cells in the reference sample, then the subject is predisposed to having a neurodegenerative disease or disorder.

15. The method of claim 1, wherein the testing comprises:
(a) determining the amount reactive oxygen species in the cells of biological sample (sample amount), and
(b) comparing the amount to a reference amount determined for a normal subject, wherein if the sample amount is greater than the reference amount, then the subject is predisposed to having a neurodegenerative disease or disorder.

16. The method of claim 1, wherein the testing comprises:
(a) determining the amount of association of a first ER-MAM-associated protein and a second ER-MAM-associated protein in cells of the biological sample (sample amount), and
(b) comparing the sample amount to a reference amount determined from a biological sample taken from a normal subject, wherein if the sample amount is more than the reference amount, then the subject is predisposed to having a neurodegenerative disease or disorder.

17. The method of claim 1, wherein the testing comprises:
(a) expressing a first ER-MAM-associated protein fused to a fluorophore and a second ER-MAM-associated protein fused to a fluorophore in cells of the biological sample,
(b) illuminating the transfected cells with an appropriate wavelength to excite the first fluorophore, and
(c) measuring the amount of fluorescence resonance energy transfer to the second fluorophore, and
(d) comparing the amount to a reference amount determined for a normal subject, wherein if the sample amount is more than the reference amount, then the subject is predisposed to having a neurodegenerative disease or disorder.

18. The method of claim 1, wherein the testing comprises:
(a) expressing a first ER-MAM-associated protein fused to a fluorophore and a second ER-MAM-associated protein fused to a fluorophore in cells of the biological sample,
(b) illuminating the transfected cells with an appropriate wavelength to excite the first fluorophore, and
(c) measuring the amount of fluorescence resonance energy transfer to the second fluorophore, and
(d) comparing the amount to a reference amount determined for a normal subject, wherein if the sample amount is less than the reference amount, then the subject is predisposed to having a neurodegenerative disease or disorder.

19. The method of claim 17, wherein the first ER-MAM-associated protein fused to a fluorophore is DGAT2-YFP and the second ER-MAM-associated protein fused to a fluorophore is SCD1-CFP.

20. The method of any of claim 9, 10, 17 or 18, wherein the ER-MAM-associated protein is any of Acyl-CoA:cholesterol acyltransferase (ACAT1); Acyl-CoA desaturase (stearoyl-CoA desaturase 1); Apolipoprotein E; Autocrine motility factor receptor 2 (GP78); β-galactoside α(2-3) sialyltransferase (SIAT4); β-galactoside α(2-6) sialyltransferase (SIAT1); β-1,4 N-acetylgalactosaminyltransferase 1 (SIAT2); β-1,4-galactosyltransferase 6 (lactosyl-ceramide synthase); Ceramide glucosyltransferase; Diacylglycerol O-acyltransferase; Fatty acid-CoA ligase, long-chain 1 (FACL1) (acyl-CoA synthetase 1); Fatty acid-CoA ligase, long-chain 4 (FACL4) (acyl-CoA synthetase 4); Fatty acid transport protein 4 (FATP4); Glucose-6-phosphatase; Glucose-regulated protein 78-kDa (BiP); Inositol 1,4,5-triphosphate receptor, type 3 (IP3R3); Microsomal triglyceride transfer protein large subunit; N-acetylglucosaminyl-phosphatidylinositol de-N-acetylase; Opioid receptor, sigma1; Phosphatidylethanolamine N-methyltransferase 2 (PEMT); Phosphatidylserine synthase 1 (PSS1); Phosphatidylserine synthase 2 (PSS2); Phosphofurin acidic cluster sorting protein 2; Presenilin 1; Presenilin 2; Ryanodine Receptor type 1; Ryanodine Receptor type 2; Ryanodine Receptor type 3; Amyloid beta precursor protein; Basigin/CD147/EMMPRIN; Cytomegalus virus-encoded vMIA protein fr unspliced exon I UL37 mRNA, N-term frag; Glucose-regulated protein 75-kDa (GRP75; Mortalin-2); Membrane bound O-acyltransferase domain containing 2.

21. The method of claim 1, wherein the method further comprises:
(a) determining the amount cholesterol in the biological sample (sample amount), and
(b) comparing the amount to a reference amount determined for a normal subject, wherein if the sample amount is more than the reference amount, then the subject is predisposed to having a neurodegenerative disease or disorder.

22. The method of claim 1, wherein the method further comprises:
(a) determining the amount phosphatidylethanolamine in the biological sample (sample amount), and
(b) comparing the amount to a reference amount determined for a normal subject, wherein if the sample amount is more than the reference amount, then the subject is predisposed to having a neurodegenerative disease or disorder.

23. The method of claim 1, wherein the method further comprises:
(a) determining the amount cytosolic free calcium in cells of the biological sample (sample amount), and
(b) comparing the amount to a reference amount determined for a normal subject, wherein if the sample amount is greater than the reference amount, then the subject is predisposed to having a neurodegenerative disease or disorder.

24. The method of claim 1, wherein the method further comprises:
(a) determining the amount mitochondrial calcium in cells of the biological sample (sample amount), and
(b) comparing the amount to a reference amount determined for a normal subject, wherein if the sample amount is greater than the reference amount, then the subject is predisposed to having a neurodegenerative disease or disorder.

25. The method of claim 1, wherein the method further comprises:
(a) determining the amount of glucose metabolism in cells of the biological sample (sample amount), and
(b) comparing the amount to a reference amount determined for a normal subject, wherein if the sample amount is less than the reference amount, then the subject is predisposed to having a neurodegenerative disease or disorder.

26. The method of claim 1, wherein the method further comprises:
(a) determining the rate of ATP biosynthesis in cells of the biological sample (sample rate), and
(b) comparing the rate to a reference rate determined for a normal subject, wherein if the sample rate is less than the reference amount, then the subject is predisposed to having a neurodegenerative disease or disorder.

27. A method for determining whether a compound ameliorates a neurodegenerative disease or disorder in a subject, the method comprising testing a test biological sample to determine the compound is capable of increasing endoplasmic reticulum-mitochondrial-associated membrane integrity.

28. The method of claim 27, wherein the testing comprises:
(a) contacting the test biological sample with a compound,
(b) measuring a rate of conversion of phosphatidylserine to phosphatidylethanolamine in the test biological sample of step (a),
(c) comparing the rate of conversion of phosphatidylserine to phosphatidylethanolamine measured in step (b) to a rate of conversion of phosphatidylserine to phosphatidylethanolamine measured in a reference biological sample that has not been contracted with the compound, wherein a decrease or alteration in rate of conversion of phosphatidylserine to phosphatidylethanolamine measured in the test biological sample of step (b) relative to rate of conversion of phosphatidylserine to phosphatidylethanolamine measured in the reference biological sample indicates that the compound ameliorates a neurodegenerative disease or disorder in a subject.

29. The method of claim 27, wherein the testing comprises:
(a) contacting the test biological sample with a compound and a fixed amount of cinnamycin,
(b) determining the percentage of cell survival in the test biological sample, and
(c) comparing the percentage to the percentage of cell survival to the percentage of cell survival in a reference biological sample contacted with the fixed amount of cinnamycin that has not been contacted with the compound, wherein if the percentage of cell survival in the test biological sample is more than or different than the reference biological sample, then the compound ameliorates a neurodegenerative disease or disorder in a subject.

30. The method of claim 27, wherein the testing comprises:
(a) contacting the test biological sample with a compound,
(b) measuring the amount of association between a first ER-MAM-associated protein and a second ER-MAM-associated protein in the test biological sample of step (a), and
(c) comparing the amount of association between the first ER-MAM-associated protein and the second ER-MAM-associated protein measured in step (b) to the amount of association between the first ER-MAM-associated protein and the second ER-MAM-associated protein measured in a reference biological sample that has not been contracted with a compound, wherein an decrease in the amount of association between the first ER-MAM-associated protein and the second ER-MAM-associated protein measured in the test biological sample of step (b) relative to the amount of association between the first ER-MAM-associated protein and the second ER-MAM-associated protein measured in the reference biological sample indicates that the compound ameliorates a neurodegenerative disease or disorder in a subject.

31. The method of claim 27, wherein the testing comprises:
(a) contacting the test biological sample with a compound,
(b) measuring the amount of association between Diacylglycerol-O-acyltransferase 2 (DGAT2) and stearoyl-CoA desaturase 1 (SCD1) in the test biological sample of step (a), and
(c) comparing the amount of association between Diacylglycerol-O-acyltransferase 2 (DGAT2) and stearoyl-CoA desaturase 1 (SCD1) measured in step (b) to amount of association between Diacylglycerol-O-acyltransferase 2 (DGAT2) and stearoyl-CoA desaturase 1 (SCD1) measured in a reference biological sample that has not been contracted with a compound, wherein an decrease in the amount of association between Diacylglycerol-O-acyltransferase 2 (DGAT2) and stearoyl-CoA desaturase 1 (SCD1) measured in the test biological sample of step (b) relative to the amount of association between Diacylglycerol-O-acyltransferase 2 (DGAT2) and stearoyl-CoA desaturase 1 (SCD1) measured in the reference biological sample indicates that the compound ameliorates a neurodegenerative disease or disorder in a subject.

32. The method of claim 27, wherein the testing comprises:
(a) contacting the test biological sample with a compound,
(b) measuring the amount reactive oxygen species in the biological sample of step (a), and
(c) comparing the amount reactive oxygen species measured in step (b) to the amount reactive oxygen species measured in a reference biological sample that has not been contracted with a compound, wherein a decrease in the amount of amount reactive oxygen species measured in the test biological sample of step (b) relative to the amount of amount reactive oxygen species measured in the reference biological sample indicates that the compound ameliorates a neurodegenerative disease or disorder in a subject.

33. The method of claim 27, wherein the testing comprises:
(a) contacting the test biological sample with a compound,
(b) measuring the ratio of perinuclear mitochondria to non-perinuclear mitochondria in the test biological sample of step (a), and
(c) comparing the ratio of perinuclear mitochondria to non-perinuclear mitochondria measured in step (b) to a ratio of perinuclear mitochondria to non-perinuclear mitochondria measured in a reference biological sample that has not been contracted with a compound, wherein an reduction in the ratio of perinuclear mitochondria to non-perinuclear mitochondria measured in the biological sample of step (b) relative to the ratio of perinuclear mitochondria to non-perinuclear mitochondria measured in the reference biological sample indicates that the compound ameliorates a neurodegenerative disease or disorder in a subject.

34. The method of claim 27, wherein the testing comprises:
(a) contacting the test biological sample with a compound,
(b) measuring the amount of movement of mitochondria in the biological sample of step (a), and
(c) comparing the amount of movement of mitochondria measured in step (b) to a amount of movement of mitochondria in a reference biological sample that has not been contracted with a compound, wherein an increase in the amount of movement of mitochondria measured in the test biological sample of step (b) relative to the amount of movement of mitochondria measured in the reference biological sample indicates that the compound ameliorates a neurodegenerative disease or disorder in a subject.

35. The method of claim 27, wherein the testing comprises:
(a) contacting the test biological sample with a compound,
(b) measuring the amount of communication between the ER and mitochondria in the biological sample of step (a), and
(c) comparing the amount of communication between the ER and mitochondria measured in step (b) an amount of communication between the ER and mitochondria measured in a reference biological sample that has not been contracted with a compound, wherein a increase in the amount of communication between the ER and mitochondria measured in the test biological sample of step (b) relative to the amount of communication between the ER and mitochondria measured in the reference biological sample indicates that the compound ameliorates a neurodegenerative disease or disorder in a subject.

36. The method of claim 27, wherein the testing comprises:
(a) contacting the test biological sample with a compound,
(b) measuring the ratio of perinuclear mitochondria to non-perinuclear mitochondria in the biological sample of step (a), and
(c) comparing the ratio of perinuclear mitochondria to non-perinuclear mitochondria measured in step (b) an ratio of perinuclear mitochondria to non-perinuclear mitochondria measured in a reference biological sample comprising normal human cells that has not been contracted with a compound, wherein a reduction in the ratio of perinuclear mitochondria to non-perinuclear mitochondria measured in the test biological sample of step (b) relative to the amount ratio of perinuclear mitochondria to non-perinuclear mitochondria measured in the reference biological sample indicates that the compound ameliorates a neurodegenerative disease or disorder in a subject.

37. The method of claim 27, wherein the testing comprises:
(a) contacting the test biological sample with a compound,
(b) measuring the amount of localization of an ER-MAM-associated protein to ER-MAM in the biological sample of step (a), and
(c) comparing the amount of localization of an ER-MAM-associated protein to ER-MAM measured in step (b) with an amount of localization of an ER-MAM-associated protein to ER-MAM measured in a reference biological sample that has not been contacted with a compound, wherein an increase in the amount of localization of an ER-MAM-associated protein to ER-MAM measured in the test biological sample of step (b) relative to amount of localization of an ER-MAM-associated protein to ER-MAM measured in the reference biological sample indicates that the compound ameliorates a neurodegenerative disease or disorder in a subject.

38. The method of claim 27, wherein the testing comprises:
(a) contacting the test biological sample with a compound,
(b) measuring the amount of localization of an ER-MAM-associated protein to ER-MAM in the biological sample of step (a), and
(c) comparing the amount of localization of an ER-MAM-associated protein to ER-MAM measured in step (b) with an amount of localization of an ER-MAM-associated protein to ER-MAM measured in a reference biological sample that has not been contacted with a compound, wherein an decrease in the amount of localization of an ER-MAM-associated protein to ER-MAM measured in the test biological sample of step (b) relative to amount of localization of an ER-MAM-associated protein to ER-MAM measured in the reference biological sample indicates that the compound ameliorates a neurodegenerative disease or disorder in a subject.

39. The method of claim 27, wherein the testing comprises:
(a) contacting the test biological sample with a compound,
(b) measuring the activity of an ER-MAM-associated protein in the biological sample of step (a), and
(c) comparing the activity of an ER-MAM-associated protein measured in step (b) with the activity of an ER-MAM-associated protein measured in a reference biological sample that has not been contacted with a compound, wherein an decrease in the activity of an ER-MAM-associated protein measured in the test biological sample of step (b) relative to the activity of an ER-MAM-associated protein measured in the reference biological sample indicates that the compound ameliorates a neurodegenerative disease or disorder in a subject.

40. The method of claim 27, wherein the testing comprises:
(a) contacting the test biological sample with a compound,
(b) measuring the activity of an ER-MAM-associated protein in the biological sample of step (a), and
(c) comparing the activity of an ER-MAM-associated protein measured in step (b) with the activity of an ER-MAM-associated protein measured in a reference biological sample that has not been contacted with a compound, wherein an increase in the activity of an ER-MAM-associated protein measured in the test biological sample of step (b) relative to the activity of an ER-MAM-associated protein measured in the reference biological sample indicates that the compound ameliorates a neurodegenerative disease or disorder in a subject.

41. The method of any of claim 28-40, wherein the biological sample comprises a cell.

42. The method of claim 41, wherein the cell is a normal cell.

43. The method of claim 41, wherein the cell has an Alzheimer's disease mutation.

44. The method of claim 43, wherein the Alzheimer's disease mutation is mutation is APP V717 I APP V717F, APP V717G, APP A682G, APP K/M670/671N/L, APP A713V, APP A713T, APP E693G, APP T673A, APP N665D, APP 1716V, APP V715M, PS1 113Δ4, PS1 A79V, PS1 V82L, PS1 V96F, PS1 113Δ4, PS1 Y115C, PS1 Y115H, PS1 T116N, PS1 P117L, PS1 E120D, PS1 E120K, PS1 E123K, PS1 N135D, PS1 M139, PS1 I M139T, PS1 M139V, I 143F, PS1 1143T, PS1 M461, PS1 I M146L, PS1 M146V, PS1 H163R, PS1 H163Y, PS1 S169P, PS1 S169L, PS1 L171P, PS1 E184D, PS1 G209V, PS1 I 213T, PS1 L219P, PS1 A231T, PS1 A231V, PS1 M233T, PS1 L235P, PS1 A246E, PS1 L250S, PS1 A260V, PS1 L262F, PS1 C263R, PS1 P264L, PS1 P267S, PS1 R269G, PS1 R269H, PS1 E273A, PS1 R278T, PS1 E280A, PS1 E280G, PS1 L282R, PS1 A285V, PS1 L286V, PS1 S290C (Δ9), PS1 E318G, PS1 G378E, PS1 G384A, PS1 L392V, PS1 C410Y, PS1 L424R, PS1 A426P, PS1 P436S, PS1 P436Q, PS2 R62H, PS2 N141I, PS2 V148I, PS2 M293V or any combination thereof.

45. The method of claim 41, wherein the cell expressed exogenous presenilin-1 or presenilin-2.

46. The method of claim 41, wherein the cell does not express presenilin-1 or presenilin-2

47. The method of claim 41, wherein the cell expresses reduced levels of presenilin-1 or presenilin-2

48. The method of claim 41, wherein the cell is from a subject having Alzheimer's disease.

49. The method of any of claim 30, 37, 38 or 39, wherein the ER-MAM-associated protein is any of Acyl-CoA:cholesterol acyltransferase (ACAT1); Acyl-CoA desaturase (stearoyl-CoA desaturase 1); Apolipoprotein E; Autocrine motility factor receptor 2 (GP78); β-galactoside α(2-3) sialyltransferase (SIAT4); β-galactoside α(2-6) sialyltransferase (SIAT1); β-1,4 N-acetylgalactosaminyltransferase 1(SIAT2); (3-1,4-galactosyltransferase 6 (lactosyl-ceramide synthase); Ceramide glucosyltransferase; Diacylglycerol O-acyltransferase; Fatty acid-CoA ligase, long-chain 1 (FACL1) (acyl-CoA synthetase 1); Fatty acid-CoA ligase, long-chain 4 (FACL4) (acyl-CoA synthetase 4); Fatty acid transport protein 4 (FATP4); Glucose-6-phosphatase; Glucose-regulated protein 78-kDa (BiP); Inositol 1,4,5-triphosphate receptor, type 3 (IP3R3); Microsomal triglyceride transfer protein large subunit; N-acetylglucosaminyl-phosphatidylinositol de-N-acetylase; Opioid receptor, sigma1; Phosphatidylethanolamine N-methyltransferase 2 (PEMT); Phosphatidylserine synthase 1 (PSS1); Phosphatidylserine synthase 2 (PSS2); Phosphofurin acidic cluster sorting protein 2; Presenilin 1; Presenilin 2; Ryanodine Receptor type 1; Ryanodine Receptor type 2; Ryanodine Receptor type 3; Amyloid beta precursor protein; Basigin/CD147/EMMPRIN; Cytomegalus virus-encoded vMIA protein fr unspliced exon I UL37 mRNA, N-term frag; Glucose-regulated protein 75-kDa (GRP75; Mortalin-2); Membrane bound O-acyltransferase domain containing 2.

50. The method of claim 27, wherein the neurodegenerative disease or disorder is Alzheimer's disease.
